SEQUENCE LISTING (4 sequences)

SEQ ID NO: 1 - protein, 222 aa, Persephonella marina
GWSYHGEHGPEHWGDLKDEYIMCKIGKNQSPVDINRIVDAKLKPIKIEYRAGATKVLNNGHTIKVSYEPGSYIVVDGIKFELKQFHFHAPSEHKLKGQHYPFEAHFVHADKHGNLAVIGVFFKEGRENPILEKIWKVMPENAGEEVKLAHKINAEDLLPKDRDYYRYSGSLTTPPCSEGVRWIVMEEEMEMSKEQIEKFRKIMGGDTNRPVQPLNARMIMEK

SEQ ID NO: 2 - protein, 226 aa, synthetic construct
GGVGHWSYHGETGPQHWGDLKNEYIMCKIGKNQSPVDISRIVEAELEKIKINYSSGGSSITNNGHTIKVSYEPGSYIIVDGIRFELKQFHFHAPSEHTIKGKSYPFEAHFVHADKDGNLAVIGVIFKEGKKNPIIEKIWENLPEAGKTIKLAHKINAYDLLPKKKKYYRYSGSLTTPPCSEGVRWIVMEEEMELSKEQIEKFRKLMGGDTNRPVQPLNARMIMEMD

SEQ ID NO: 3 - protein, 234 aa, Persephonella marina
HHHHHHPRHEWSYEGEKGPEHWAQLKPEFFWCKLKNQSPINIDKKYKVKANLPKLNLYYKTAKESEVVNNGHTIQINIKEDNTLNYLGEKYQLKQFHFHTPSEHTIEKKSYPFEAHFVHADKHGNLAVIGVFFKEGRENPILEKIWKVMPENAGEEVKLAHKINAEDLLPKDRDYYRYSGSLTTPPCTEGVRWIVLKKPISISKQQLEKLKSVMVNPNNRPVQEINSRWIIEGF

SEQ ID NO: 4 - protein, 225 aa, Persephonella hydrogeniphila
AGSWSYHGATGPEHWGDLKDEYIMCKIGKNQSPVDISRIAEADLEDIKFHYSPGATSLLNNGHTIKVSYEPGSYIVVDGIRFELKQFHFHAPSEHTVKGKSYPFEAHFVHADKDGNLAVIGVLFKEGKENPVIKKLWENLPSHTGEKVKLAHKINAYDLLPKRKKYYRYSGSLTTPPCSEGVRWLVMTTEMEMSKEQIEKFRKLMGGDTNRPVQPVNARMILIKD

(19) United States
(12) Patent Application Publication
(10) Pub. No.: US 2026/0185074 A1
(43) Pub. Date: Jul. 2, 2026

(54) CARBONIC ANHYDRASE VARIANTS AND POLYNUCLEOTIDES ENCODING SAME

(71) Applicant: Novozymes A/S, Bagsvaerd (DK)

(72) Inventors: Esben Peter FRIIS, Herlev (DK); Pengfei TIAN, Copenhagen Oe (DK); Trine Holst SOERENSEN, Copenhagen (DK); Weijian LAI, Beijing (CN); Lars ANDERSON, Maimoe (SE); Jesper FRICKMANN, Raleigh, NC (US); Mateusz Henryk DYLA, Kongens Lyngby (DK); Stefanie NEUN, Copenhagen (DK)

(73) Assignee: Novozymes A/S, Bagsvaerd (DK)

(21) Appl. No.: 19/130,040

(22) PCT Filed: Nov. 30, 2023
(86) PCT No.: PCT/US2023/081794

§ 371 Date: May 14, 2025

Related U.S. Application Data
(60) Provisional application No. 63/385,574, filed on Nov. 30, 2022.

(51) Int. Cl. C12N 9/88; C12M 1/40; C12P 7/40

(52) U.S. Cl. CPC C12N 9/88; C12M 21/18; C12P 7/40; C12Y 402/01001

(57) ABSTRACT

The present invention relates to carbonic anhydrase variants. The present invention also relates to polynucleotides encoding the variants; nucleic acid constructs, vectors, and host cells comprising the polynucleotides; and methods of using the variants.

### REFERENCE TO A SEQUENCE LISTING

This application contains a Sequence Listing in computer readable form, which is incorporated herein by reference.

### FIELD OF THE INVENTION

The present invention relates to carbonic anhydrase variants, polynucleotides encoding the variants, methods of producing the variants, and methods of using the variants. The invention also relates to bioreactors for extracting carbon dioxide and compositions useful for such extraction processes.

### BACKGROUND OF THE INVENTION

Carbon dioxide (CO₂) emissions are a major contributor to the phenomenon of global warming. CO₂ is a by-product of combustion and it creates operational, economic, and environmental problems. CO₂ emissions may be controlled by capturing CO₂ gas before emitted into the atmosphere. There are several chemical approaches to control the CO₂ emissions (A. Kohl and R. Nielsen, Gas Purification, 5^{th} ed., Gulf Professional Publishing, Houston, TX, 1997). However, many of these approaches have drawbacks such as high energy consumption, slow processes, and use of ecologically questionable or toxic compounds.

An enzyme-based approach using the capability of carbonic anhydrase to catalyse the conversion of CO₂ to bicarbonate at a very high rate (turnover is up to 10⁶ molecules of CO₂ per second), overcomes the reaction rates and environmental issues in relation to CO₂ capture. Technical solutions for extracting CO₂ from gases, such as combustion gases or respiration gases, using carbonic anhydrases have been described in WO 2006/089423, U.S. Pat. No. 6,524,842, WO 2004/007058, WO 2004/028667, US 2004/0029257, U.S. Pat. No. 7,132,090, WO 2005/114417, U.S. Pat. No. 6,143,556, WO 2004/104160, US 2005/0214936, WO 2008/095057. Generally, these techniques operate by bringing a soluble or immobilized carbonic anhydrase into contact with CO₂ which either may be in a gas phase or a liquid phase. In the presence of water, carbonic anhydrase catalyses the conversion of CO₂ into bicarbonate ions which may be further protonated or deprotonated to carbonic acid and/or carbonate ions depending on the pH of the medium. The ions may either be utilized to facilitate growth of algae or microorganisms that utilize bicarbonate/carbonate as a carbon source, to induce a pH change in a surrounding medium or supply buffering capacity, to provide bicarbonate/carbonate as an active agent for subsequent chemical processes, or precipitated as a carbonate salt, or converted back into pure CO₂, which can then be used (for example in enhanced oil recovery, for production of urea, for food and beverage processing, or to supply CO₂ to greenhouses or cultivation ponds), released (for example from a contained life support environment such as a submarine, spacecraft, or artificial lung), compressed (for example for transportation through pipelines), or stored (such as in geological or deep oceanic formations or saline aquifers).

Mammalian, plant and prokaryotic carbonic anhydrases (alpha- and beta-class CAs) generally function at physiological temperatures (37° C.) or lower temperatures. The temperature of combustion gasses or the liquids into which they are dissolved may, however, easily exceed the temperature optimum for the carbonic anhydrase used to capture the CO₂. One of the drawbacks of using enzyme-based solutions is that extensive cooling may be needed in CO₂ extraction processes prior to contacting the CO₂-containing gas/liquid with the carbonic anhydrase, and cooling is an energy consuming process. Consequently, there is a need for more heat-stable carbonic anhydrases when the enzyme is to be used under industrially relevant conditions.

A carbonic anhydrase obtainable from Persephonella marina is described in WO2012/025577. A metagenomic carbonic anhydrase isolated from the Logatchev hydrothermal is described in WO2018/017792. The present invention provides carbonic anhydrase variants with improved properties compared to its parent.

### SUMMARY OF THE INVENTION

The present invention relates to isolated carbonic anhydrase variants, comprising a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1, wherein the variant has at least 65%, e.g., at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, SEQ ID NO: 2, SEQ ID NO: 3, Or SEQ ID NO: 4, or the mature polypeptide sequence thereof, and wherein the variant has carbonic anhydrase activity.

The present invention also relates to isolated polynucleotides encoding the variants; nucleic acid constructs, vectors, and host cells comprising the polynucleotides; and methods of producing the variants.

The present invention also relates to methods of using the carbonic anhydrase variants for extraction of carbon dioxide from a carbon dioxide-containing medium.

### BRIEF DESCRIPTION OF THE FIGURES

FIG. 1 shows a schematic diagram of a counter-current gas-liquid contactor. In a typical counter-current gas-liquid contactor, the CO₂-rich inlet gas (e.g., a mixed gas comprising CO₂) enters the bottom of the contactor and travels upwards, while the CO₂-lean inlet liquid enters the top of the contactor and flows downwards. The liquid flow can be in the form of droplets, such as a spray, or as a continuous stream or sheet of liquid, such as flows along a surface, or a combination of these. Inside the gas-liquid contactor, CO₂ from the CO₂-rich inlet gas is absorbed by the liquid and carried out from the contactor as a CO₂-rich outlet liquid. CO₂-rich liquid means the liquid comprises an increased amount of CO₂ in its dissolved gaseous form or any ionic form of CO₂, such as bicarbonate, or a reaction product of CO₂ and a compound in the inlet absorption liquid, relative to the CO₂-lean liquid. CO₂-lean gas, from which CO₂ has been partially or completely removed, exits the contactor as the outlet gas. A counter-current gas-liquid contactor is the most common type of gas-liquid contactor used industrially. The internals of the contactor can be largely empty with liquid sprayed down from the top and gas flowing up from the bottom, or, more commonly, the contactor contains different types of packing materials to increase the residence time of gas and liquid inside the contactor and promote a large surface area of interaction between the gas and liquid. The liquid is typically delivered onto the packing materials from spray nozzles or other types of openings designed to deliver the liquid in a uniform fashion over the packing material. The liquid is comprised of absorption compounds, water, and other components that may be needed to optimize the process. For example, the liquid can comprise CA in soluble form or as small particles of suspended biocatalyst that flow through the contactor. The packing material may be coated with or have CA attached to it.

FIG. 2 shows a schematic diagram of a co-current gas-liquid contactor. In a typical co-current gas-liquid contactor, the CO₂-rich inlet gas and CO₂-lean inlet liquid enter the contactor at the same end (e.g., the top) and exit at the same end. The outlet gas preferably exits the contactor at a location above any sump present for collection of the outlet liquid. This type of contactor can provide a lower pressure drop compared to a counter-current contactor, because the gas and liquid flows are both moving in the same direction, however the efficiency of gas-liquid interaction may be lower compared to a counter-current design. The internals and function of the co-current contactor are as described for FIG. 1, with the exception that the gas and liquid are flowing in the same direction.

FIG. 3 shows a schematic diagram of a perpendicular-flow gas-liquid contactor. In a perpendicular gas-liquid contactor, the gas typically travels in an overall horizontal direction from the inlet gas to the outlet gas while the liquid overall travels vertically, from inlet liquid at the top to outlet liquid at the bottom to take advantage of gravity. This contactor design can take advantage of specialized liquid delivery systems, such as those generating flat sheets of liquid, to create high gas-liquid contact in a compact design that does not require tall vertical structures. The contactor can have internal baffles or packing materials to enhance gas-liquid contact and control gas and liquid flows. The internals and function of the co-current contactor are as described for FIG. 1, with the exception that the overall gas and liquid flows are perpendicular to each other.

FIG. 4 shows a schematic diagram of a membrane gas-liquid contactor. A membrane contactor utilizes a gas permeable membrane (dotted line) to separate the gas flow from the liquid flow. The diagram shows counter-current flow of gas and liquid, though co-current and perpendicular-type flows are also possible. The CO₂-rich inlet gas comes in contact with the membrane and CO₂, preferably selectively, passes across the membrane into the CO₂ absorption liquid. Membranes used in these contactors can be microporous, allowing the surface of the liquid to be exposed to the gas through pores in the membrane that are small enough to prevent the liquid from passing through due to physical phenomena, such as surface tension. Alternatively, the membranes can be non-porous, yet made from CO₂-gas permeable materials. Microporous membranes may provide faster CO₂ absorption rates, while non-porous membranes may minimize liquid losses to evaporation in the gas stream. While passing through the contactor, the inlet liquid becomes enriched in CO₂, such that the outlet liquid is CO₂-rich by comparison, and the inlet gas becomes depleted in CO₂, such that the outlet gas is CO₂-lean. The diagram only shows a representation of the basic functional unit of a membrane contactor, which in operational form contains many layers of stacked membranes or bundles of tubular, or hollow-fiber, membranes arranged in suitable housing with dividers and control mechanisms to optimally direct the gas and liquid flows. Membrane contactors are used for large industrial gas scrubbing applications as well as for small units, such as for CO₂ removal during dialysis in which the semi-permeable membrane may separate two liquids, such as CO₂-rich blood and a CO₂-lean buffer solution capable of absorbing excess CO₂ from the blood. CA can be present in soluble or suspended particulate form in the liquid and can be immobilized on or in the membrane.

FIG. 5 shows a schematic diagram of a bubble-tank gas-liquid contactor. The system shown is a batch-mode bubble-tank gas-liquid contactor, in which a stream of inlet gas comprising CO₂ is bubbled (or sparged) through a fixed amount of absorption liquid. CO₂ is absorbed into the liquid such that the outlet gas is depleted in CO₂ compared to the inlet gas. In batch mode, eventually the liquid will reach a maximum CO₂ absorption capacity, and the CO₂-rich inlet gas can be directed to another batch reactor containing CO₂-lean absorption liquid, or the batch reactor can be emptied and filled with fresh CO₂-lean absorption liquid, or the inlet gas flow can be stopped while the batch reactor is changed from absorption mode to CO₂ desorption mode, such as by applying heat, sweep gas or vacuum to the batch reactor, to release absorbed CO₂ from the CO₂-rich liquid. This type of contactor can, for example, be used for producing solid precipitated forms of CO₂, such as carbonates, like calcium, magnesium, and manganese carbonate. Gas delivery nozzles that produce very small gas bubbles can enhance the gas-liquid contact and improve CO₂ absorption efficiency. A bubble-tank contactor can be enclosed equipment or can operate in open environments, such as bubbling CO₂-rich gas streams into algae ponds. CA can be present in soluble or suspended particulate form in the liquid and can be immobilized on the surfaces of structures or packing immersed in or exposed to the liquid.

FIG. 6 shows a schematic diagram of a batch-mode stirred-tank gas-liquid contactor. In this type of contactor the CO₂-rich gas is exposed to the surface of a liquid leading to CO₂ gas absorption into the liquid. The liquid may be quiescent or may be mixed by some means to cause movement of the liquid and liquid components. The principle of a stirred-tank contactor can apply to controlled enclosed equipment or can apply to open environments, such the absorption of CO₂ from air into a body of water, like an ocean. CA can be present in soluble or suspended particulate form in the liquid and can be immobilized on the surfaces of structures, mixers or packing immersed in or exposed to the liquid.

FIG. 7 shows a schematic diagram of an integrated CO₂ scrubbing system. In the system shown, CO₂-rich feed gas (1) enters near the bottom of the absorber (2) and flows upwards where it comes in contact with CO₂-lean absorption liquid (3) that enters the absorber near the top. Scrubbed gas (4), from which CO₂ has been removed, exits the absorber at the top. CO₂-rich absorption liquid (5) exits at the bottom of the absorber and (optionally) passes through a biocatalyst recovery unit (6) which separates the catalyst for recycling (7) and redelivery into the absorber along with the CO₂-lean absorption liquid (3). The main amount of CO₂-rich absorption liquid (5) exits the (optional) biocatalyst recovery unit (6) and travels to an optional temperature regulator (e.g., heat exchanger) (8) where the CO₂-rich absorption liquid is pre-heated before traveling to the desorber (9). The CO₂-rich absorption liquid enters near the top of the desorber and flows downwards. Heat is supplied to the desorber by any suitable means, e.g., a re-boiler, and optionally another desorption driving force such as a sweep gas (10) or vacuum (13), or a combination of these applied to the desorber causing extracted CO₂ to be released from the absorption liquid and exit the desorber, (optionally) passing through an absorption liquid condenser (11) to remove absorption liquid vapor from the gas stream, and (optionally) passing through a sweep gas condenser (12) to remove sweep gas compounds from the CO₂ gas stream prior to release, compression and/or use of the purified CO₂ gas (14). Sweep gas compounds separated in the sweep gas condenser (12) are optionally recycled and fed back to the desorber along with the provision of fresh sweep gas (10). CO₂-lean absorption liquid (15) exits the desorber, and (optionally) passes through a second biocatalyst recovery unit (16), which separates the biocatalyst for recycling (17) and redelivery into the desorber along with the CO₂-rich absorption liquid (5), before (optionally) passing through a temperature regulator (8), and (optionally) passing through a secondary CO₂ desorber (18) before returning to the absorber (3). Although the secondary CO₂ desorber may function by any of the known means of desorption, a preferred mode of operation for the secondary CO₂ desorber is as a secondary air sweep desorber utilizing a membrane-based design, in which CO₂-lean sweep gas (22), such as air is contacted with the CO₂-lean liquid (15) to further remove residual CO₂ remaining in the CO₂-lean liquid (15) and provide a very CO₂-lean liquid (3) for re-entry to the absorber. The secondary sweep gas (23) exiting the secondary CO₂ desorber (18) can be released to the atmosphere or can be used for a purpose, such as to supply air for combustion, e.g., when the CO₂ scrubber is installed at a power plant. Depleted biocatalyst and/or other depleted components of the absorption liquid can be added at various points in the process, such as at the locations indicated (20 and 21). Removal of samples for process monitoring and control of liquid levels as well as removal of insoluble contaminants can be carried out at various points in the process, such as at the locations indicated (24 and 25). Although not depicted in the diagram, it is understood that pumps to provide and control liquid flow, blowers to provide and control gas flow, and all relevant valves, meters, instrumentation and equipment for process control and monitoring can be installed and utilized at the needed locations. CA can be present in soluble or suspended particulate form in the liquid as it flows through the system and can be immobilized on the surfaces of structures, mixers or packing material immersed in or exposed to the liquid.

FIG. 8 is a multiple sequence alignment (MSA) of the mature carbonic anhydarase parent enzymes of SEQ ID NOs: 1-4 described herein.

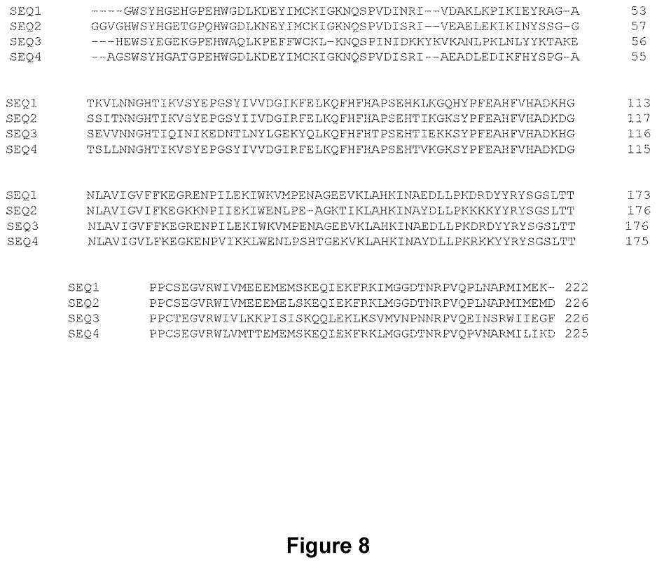

### DEFINITIONS

In accordance with this detailed description, the following definitions apply. Note that the singular forms “a,” “an,” and “the” include plural references unless the context clearly dictates otherwise. Unless defined otherwise or clearly indicated by context, all technical and scientific terms used herein have the same meaning as commonly understood by one of ordinary skill in the art to which this invention belongs.

Carbonic Anhydrase: The term “carbonic anhydrase” means an enzyme of EC 4.2.1.1 having carbonic anhydrase activity.

Carbonic Anhydrase Activity: The term “carbonic anhydrase activity” or “CA activity” is defined herein as an EC 4.2.1.1 activity which catalyzes the conversion between carbon dioxide and bicarbonate [CO₂+H₂O⇄HCO₃⁻+H⁺]. For purposes of the present invention, CA activity may be determined according to the procedure described in WO2018/017792 (the content of which is incorporated herein by reference). One unit of CA activity is defined after Wilbur [1 U=(1/t_{c})−(1/tᵤ)×1000] where U is units and t_{c} and tᵤ represent the time in seconds for the catalyzed and uncatalyzed reaction, respectively (Wilbur, 1948, J. Biol. Chem. 176:147-154).

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

CO₂-lean/CO₂-rich: The terms “CO₂-lean” and “CO₂-rich” carrier liquid are terms used in the present invention to describe the relative amount of carbon (e.g., in the form of dissolved CO₂, chemically reacted CO₂, bicarbonate, carbonic acid and/or carbonate salt) present in the carrier liquid as it circulates through the process. As used herein, the term “CO₂-lean carrier liquid” generally refers to carrier liquid entering an absorption module. The term “CO₂-rich carrier liquid” generally refers to a carrier liquid entering a desorption module. It is understood that the term “CO₂-lean carrier liquid” can also be applied to carrier liquid exiting a desorption module, and the term “CO₂-rich carrier liquid” can also be applied to carrier liquid exiting an absorption module. CO₂-rich carrier liquid contains more carbon compared to CO₂-lean carrier liquid within a system at a given point in time.

CO₂-containing medium: The term “CO₂-containing medium” is used to describe any material which contains at least 0.001% CO₂, preferably at least 0.01%, more preferably at least 0.1%, more preferably at least 1%, more preferably at least 5%, most preferably 10%, even more preferred at least 20%, and even most preferably at least 50% CO₂. Preferably the CO₂-containing medium has a temperature between 5° C. and 110° C., more preferably between 10° C. and 100° C., more preferably between 20° C. and 95° C., more preferably between 30° C. and 90° C., more preferably between 40° C. and 85° C., more preferably between 50° C. and 80° C., more preferably between 55° C. and 75° C. and most preferably between 60° C. and 70° C. at any pressure. CO₂-containing media are in particular gaseous phases (including gas mixtures), liquids or multiphase mixtures, but may also be solid. A CO₂-containing gaseous phase is for example raw natural gas obtainable from oil wells, gas wells, and condensate wells, syngas generated by the gasification of a carbon containing fuel (e.g., methane) to a gaseous product comprising CO and H₂, or emission streams from combustion processes, e.g., from carbon based electric generation power plants, or from flue gas stacks from such plants, industrial furnaces, stoves, ovens, or fireplaces or from airplane or car exhausts. A CO₂-containing gaseous phase may alternatively be ambient air (including hot (above 40° C.) air, e.g., desert air), or from respiratory processes in mammals (such as the CO₂-containing gas phase in an artificial lung), living plants and other CO₂ emitting species, in particular from green-houses. A CO₂-containing gas phase may also be off-gas, from aerobic or anaerobic fermentation, such as brewing, fermentation to produce useful products such as ethanol, or the production of biogas. Such fermentation processes can occur at elevated temperatures if they are facilitated by thermophilic microorganisms, which are for example encountered in the production of biogas. A CO₂-containing gaseous phase may alternatively be a gaseous phase enriched in CO₂ for the purpose of use or storage. The above-described gaseous phases may also occur as multiphase mixtures, where the gas co-exists with a certain degree of fluids (e.g., water or other solvents) and/or solid materials (e.g., ash or other particles). CO₂-containing liquids are any solution or fluid, in particular aqueous liquids, containing measurable amounts of CO₂, preferably at one of the levels mentioned above at any pressure. CO₂-containing liquids may be obtained by passing a CO₂-containing gas or solid (e.g., dry ice or soluble carbonate containing salt) into the liquid. CO₂-containing fluids may also be compressed CO₂ liquid (that contains contaminants, such as dry-cleaning fluid), supercritical CO₂, or CO₂ solvent liquids, like ionic liquids. A CO₂-containing liquid may also be referred to as a “carrier liquid”. A CO₂-containing liquid may also include compounds capable of improving the CO₂-containing capacity of the liquid, such as HCO₃⁻(KHCO₃ or NaHCO₃), CO₃²⁻(Na₂CO₃ or K₂CO₃), HPO₄²⁻(K₂HPO₄ or Na₂HPO₄) or MDEA or Tris.

CO₂ extraction: The term “CO₂ extraction” is to be understood as a reduction of carbon from a CO₂-containing medium. Such an extraction may be performed from one medium to another, e.g., gas to liquid, liquid to gas, gas to liquid to gas, liquid to liquid or liquid to solid, but the extraction may also be the conversion of CO₂ to bicarbonate, carbonate or carbonic acid within the same medium or the conversion of bicarbonate to CO₂ within the same medium. The term CO₂ capture is also used to indicate extraction of CO₂ from one medium to another or conversion of CO₂ to bicarbonate/carbonate or conversion of bicarbonate/carbonate to CO₂.

Coding sequence: The term “coding sequence” means a polynucleotide, which directly specifies the amino acid sequence of a variant. The boundaries of the coding sequence are generally determined by an open reading frame, which begins with a start codon such as ATG, GTG or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences involved in regulation of expression of a polynucleotide in a specific organism or in vitro. Each control sequence may be native (i.e., from the same gene) or heterologous (i.e., from a different gene) to the polynucleotide encoding the variant, and native or heterologous to each other. Such control sequences include, but are not limited to leader, polyadenylation, prepropeptide, propeptide, signal peptide, promoter, terminator, enhancer, and transcription or translation initiator and terminator sequences. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a variant.

Expression: The term “expression” includes any step involved in the production of a variant including, but not limited to, transcription, post-transcriptional modification, translation, post-translational modification, and secretion.

Expression vector: An “expression vector” refers to a linear or circular DNA construct comprising a DNA sequence encoding a variant, which coding sequence is operably linked to a suitable control sequence capable of effecting expression of the DNA in a suitable host. Such control sequences may include a promoter to effect transcription, an optional operator sequence to control transcription, a sequence encoding suitable ribosome binding sites on the mRNA, enhancers and sequences which control termination of transcription and translation.

Extension: The term “extension” means an addition of one or more amino acids to the amino and/or carboxyl terminus of a variant, wherein the “extended” variant has carbonic anhydrase activity.

Fragment: The term “fragment” means a variant having one or more amino acids absent from the amino and/or carboxyl terminus of the variant; wherein the fragment has carbonic anhydrase activity.

Fusion polypeptide: The term “fusion polypeptide” is a polypeptide in which one polypeptide is fused at the N-terminus and/or the C-terminus of a variant of the present invention. A fusion polypeptide is produced by fusing a polynucleotide encoding another polypeptide to a polynucleotide of the present invention, or by fusing two or more polynucleotides of the present invention together. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779). A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3:568-576; Svetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6:240-248; and Stevens, 2003, Drug Discovery World 4:35-48.

Heterologous: The term “heterologous” means, with respect to a host cell, that a polypeptide or nucleic acid does not naturally occur in the host cell. The term “heterologous” means, with respect to a polypeptide or nucleic acid, that a control sequence, e.g., promoter, of a polypeptide or nucleic acid is not naturally associated with the polypeptide or nucleic acid, i.e., the control sequence is from a gene other than the gene encoding the mature polypeptide.

Heat-stable: The term “heat-stable” or “thermostable” as used in reference to a carbonic anhydrase indicates that the enzyme is functional or active (i.e., can perform catalysis) at an elevated temperature, i.e., above 45° C., preferably above 50° C., more preferably above 55° C., more preferably above 60° C., even more preferably above 65° C., most preferably above 70° C., most preferably above 75° C., most preferably above 80° C., most preferably above 85° C. most preferably above 90° C., and even most preferably above 100° C. In a preferred embodiment the carbonic anhydrase displays optimum activity at one of the temperatures indicated above, i.e., the enzyme's temperature optimum is at one of the temperatures indicated above. The temperature stability of the carbonic anhydrase can be increased to some extent by way of formulation, e.g., by combination with stabilizing chemicals or by immobilization of the enzyme or by chemical modification, e.g., cross-linking, to preserve the enzyme in its active three-dimensional shape. In order for an enzyme to be considered as heat-stable it remains active after at least 15 minutes, preferably for at least 2 hours, more preferably for at least 24 hours, more preferably for at least 7 days, more preferably for at least 10 days, even more preferably for at least 14 days, most preferably for at least 30 days, even most preferably for at least 50 days at the elevated temperature. The level of activity may be measured using an assay described in WO2018/017792, e.g., after incubation for the given time in 1 M NaHCO₃ buffer at pH 8 at the given elevated temperature. The activity may be compared with the enzyme activity prior to the temperature elevation, thereby obtaining the residual activity of the enzyme after the heat treatment. Preferably, the residual activity is at least 30% after the given time at the elevated temperature, more preferably at least 40%, more preferably at least 50%, more at least 60%, even more preferably at least 70%, most preferably at least 80%, even most preferably the residual activity is at least 90%, and absolutely most preferred the level of residual activity is at least equal to or unchanged after the given time at the elevated temperature.

Host Strain or Host Cell: A “host strain” or “host cell” is an organism into which an expression vector, phage, virus, or other DNA construct, including a polynucleotide encoding a variant has been introduced. Exemplary host strains are microorganism cells (e.g., bacteria, filamentous fungi, and yeast) capable of expressing the polypeptide of interest and/or fermenting saccharides. The term “host cell” includes protoplasts created from cells.

Improved property: The term “improved property” means a characteristic associated with a variant that is improved compared to the parent. Such improved properties include, but are not limited to, catalytic efficiency, catalytic rate, chemical stability, oxidation stability, pH activity, pH stability, specific activity, stability under storage conditions, substrate binding, substrate cleavage, substrate specificity, substrate stability, surface properties, thermal activity, and thermostability.

Introduced: The term “introduced” in the context of inserting a nucleic acid sequence into a cell, means “transfection”, “transformation” or “transduction,” as known in the art.

Isolated: The term “isolated” means a variant, nucleic acid, cell, or other specified material or component that is separated from at least one other material or component, including but not limited to, other proteins, nucleic acids, cells, etc. An isolated polypeptide, nucleic acid, cell or other material is thus in a form that does not occur in nature. An isolated polypeptide includes, but is not limited to, a culture broth containing the secreted variant expressed in a host cell.

Mature polypeptide: The term “mature polypeptide” means a polypeptide in its mature form following N-terminal processing and/or C-terminal processing (e.g., removal of signal peptide). In one embodiment, the mature polypeptide is amino acids 1 to 222 of SEQ ID NO: 1. In another embodiment the mature polypeptide is amino acids 1 to 226 of SEQ ID NO: 2. In another embodiment the mature polypeptide is amino acids 11 to 235 of SEQ ID NO: 3. In another embodiment the mature polypeptide is amino acids 1 to 225 of SEQ ID NO: 4.

Mature polypeptide coding sequence: The term “mature polypeptide coding sequence” means a polynucleotide that encodes a mature polypeptide having carbonic anhydrase activity.

Mutant: The term “mutant” means a polynucleotide encoding a variant.

Native: The term “native” means a nucleic acid or polypeptide naturally occurring in a host cell.

Nucleic acid: The term “nucleic acid” encompasses DNA, RNA, heteroduplexes, and synthetic molecules capable of encoding a variant. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences that encode a particular amino acid sequence. Unless otherwise indicated, nucleic acid sequences are presented in 5′-to-3′ orientation.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, and which comprises one or more control sequences operably linked to the nucleic acid sequence.

Operably linked: The term “operably linked” means that specified components are in a relationship (including but not limited to juxtaposition) permitting them to function in an intended manner. For example, a regulatory sequence is operably linked to a coding sequence such that expression of the coding sequence is under control of the regulatory sequence.

Parent or parent carbonic anhydrase: The term “parent” or “parent carbonic anhydrase” means a carbonic anhydrase to which an alteration is made to produce the enzyme variants of the present invention.

Purified: The term “purified” means a nucleic acid, variant or cell that is substantially free from other components as determined by analytical techniques well known in the art (e.g., a purified variant or nucleic acid may form a discrete band in an electrophoretic gel, chromatographic eluate, and/or a media subjected to density gradient centrifugation). A purified nucleic acid or variant is at least about 50% pure, usually at least about 60%, about 65%, about 70%, about 75%, about 80%, about 85%, about 90%, about 91%, about 92%, about 93%, about 94%, about 95%, about 96%, about 97%, about 98%, about 99%, about 99.5%, about 99.6%, about 99.7%, about 99.8% or more pure (e.g., percent by weight or on a molar basis). In a related sense, a composition is enriched for a molecule when there is a substantial increase in the concentration of the molecule after application of a purification or enrichment technique. The term “enriched” refers to a compound, variant, cell, nucleic acid, amino acid, or other specified material or component that is present in a composition at a relative or absolute concentration that is higher than a starting composition.

In one aspect, the term “purified” as used herein refers to the variant or cell being essentially free from components (especially insoluble components) from the production organism. In other aspects, the term “purified” refers to the variant being essentially free of insoluble components (especially insoluble components) from the native organism from which it is obtained. In one aspect, the variant is separated from some of the soluble components of the organism and culture medium from which it is recovered. The variant may be purified (i.e., separated) by one or more of the unit operations filtration, precipitation, or chromatography.

Accordingly, the variant may be purified such that only minor amounts of other proteins, in particular, other polypeptides, are present. The term “purified” as used herein may refer to removal of other components, particularly other proteins and most particularly other enzymes present in the cell of origin of the polypeptide. The variant may be “substantially pure”, i.e., free from other components from the organism in which it is produced, e.g., a host organism for recombinantly produced variant. In one aspect, the polypeptide is at least 40% pure by weight of the total polypeptide material present in the preparation. In one aspect, the polypeptide is at least 50%, 60%, 70%, 80% or 90% pure by weight of the total polypeptide material present in the preparation. As used herein, a “substantially pure polypeptide” may denote a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which the polypeptide is natively or recombinantly associated.

It is, therefore, preferred that the substantially pure variant is at least 92% pure, preferably at least 94% pure, more preferably at least 95% pure, more preferably at least 96% pure, more preferably at least 97% pure, more preferably at least 98% pure, even more preferably at least 99% pure, most preferably at least 99.5% pure by weight of the total polypeptide material present in the preparation. The variant of the present invention is preferably in a substantially pure form (i.e., the preparation is essentially free of other polypeptide material with which it is natively or recombinantly associated). This can be accomplished, for example by preparing the variant by well-known recombinant methods or by classical purification methods.

Recombinant: The term “recombinant” is used in its conventional meaning to refer to the manipulation, e.g., cutting and rejoining, of nucleic acid sequences to form constellations different from those found in nature. The term recombinant refers to a cell, nucleic acid, variant or vector that has been modified from its native state. Thus, for example, recombinant cells express genes that are not found within the native (non-recombinant) form of the cell, or express native genes at different levels or under different conditions than found in nature. The term “recombinant” is synonymous with “genetically modified” and “transgenic”.

Recover: The terms “recover” or “recovery” means the removal of a polypeptide from at least one fermentation broth component selected from the list of a cell, a nucleic acid, or other specified material, e.g., recovery of the polypeptide from the whole fermentation broth, or from the cell-free fermentation broth, by polypeptide crystal harvest, by filtration, e.g., depth filtration (by use of filter aids or packed filter medias, cloth filtration in chamber filters, rotary-drum filtration, drum filtration, rotary vacuum-drum filters, candle filters, horizontal leaf filters or similar, using sheed or pad filtration in framed or modular setups) or membrane filtration (using sheet filtration, module filtration, candle filtration, microfiltration, ultrafiltration in either cross flow, dynamic cross flow or dead end operation), or by centrifugation (using decanter centrifuges, disc stack centrifuges, hydro cyclones or similar), or by precipitating the polypeptide and using relevant solid-liquid separation methods to harvest the polypeptide from the broth media by use of classification separation by particle sizes. Recovery encompasses isolation and/or purification of the polypeptide.

Sequence identity: The relatedness between two amino acid sequences or between two nucleotide sequences is described by the parameter “sequence identity”.

For purposes of the present invention, the sequence identity between two amino acid sequences is determined as the output of “longest identity” using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 6.6.0 or later. The parameters used are a gap open penalty of 10, a gap extension penalty of 0.5, and the EBLOSUM62 (EMBOSS version of BLOSUM62) substitution matrix. In order for the Needle program to report the longest identity, thenobrief option must be specified in the command line. The output of Needle labeled “longest identity” is calculated as follows:

$$( Identical Residues \times 100 ) / ( Length of Alignment - Total Number of Gaps in Alignment )$$

For purposes of the present invention, the sequence identity between two polynucleotide sequences is determined as the output of “longest identity” using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 6.6.0 or later. The parameters used are a gap open penalty of 10, a gap extension penalty of 0.5, and the EDNAFULL (EMBOSS version of NCBI NUC4.4) substitution matrix. In order for the Needle program to report the longest identity, the nobrief option must be specified in the command line. The output of Needle labeled “longest identity” is calculated as follows:

$$( Identical Deoxyribonucleotides \times 100 ) / ( Length of Alignment - Total Number of Gaps in Alignment )$$

Signal Peptide: A “signal peptide” is a sequence of amino acids attached to the N-terminal portion of a protein, which facilitates the secretion of the protein outside the cell. The mature form of an extracellular protein lacks the signal peptide, which is cleaved off during the secretion process.

Subsequence: The term “subsequence” means a polynucleotide having one or more nucleotides absent from the 5′ and/or 3′ end of a mature polypeptide coding sequence; wherein the subsequence encodes a fragment having carbonic anhydrase activity.

Syngas: The term “syngas” or “synthesis gas” is used to describe a gas mixture that contains varying amounts of carbon monoxide and hydrogen generated by the gasification of a carbon containing fuel (e.g., methane or natural gas) to a gaseous product with a heating value. CO₂ is produced in the syngas reaction and must be removed to increase the heating value.

Variant: The term “variant” means a polypeptide having carbonic anhydrase activity comprising a substitution, an insertion (including extension), and/or a deletion (e.g., truncation), at one or more positions. A substitution means replacement of the amino acid occupying a position with a different amino acid; a deletion means removal of the amino acid occupying a position; and an insertion means adding 1-5 amino acids (e.g., 1-3 amino acids, in particular, 1 amino acid) adjacent to and immediately following the amino acid occupying a position.

Wild-type: The term “wild-type” in reference to an amino acid sequence or nucleic acid sequence means that the amino acid sequence or nucleic acid sequence is a native or naturally-occurring sequence. As used herein, the term “naturally-occurring” refers to anything (e.g., proteins, amino acids, or nucleic acid sequences) that is found in nature. Conversely, the term “non-naturally occurring” refers to anything that is not found in nature (e.g., recombinant nucleic acids and protein sequences produced in the laboratory or modification of the wild-type sequence).

### Conventions for Designation of Variants

For purposes of the present invention, the polypeptide disclosed in SEQ ID NO: 1 is used to determine the corresponding amino acid positions in another carbonic anhydrase. The amino acid sequence of another carbonic anhydrase is aligned with the polypeptide disclosed in SEQ ID NO: 1, and based on the alignment, the amino acid position number corresponding to any amino acid residue in the polypeptide disclosed in SEQ ID NO: 1 is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are gap open penalty of 10, gap extension penalty of 0.5, and the EBLOSUM62 (EMBOSS version of BLOSUM62) substitution matrix.

In describing the variants of the present invention, the nomenclature described below is adapted for ease of reference. The accepted IUPAC single letter or three letter amino acid abbreviation is employed.

Substitutions. For an amino acid substitution, the following nomenclature is used: Original amino acid, position, substituted amino acid. Accordingly, the substitution of threonine at position 226 with alanine is designated as “Thr226Ala” or “T226A”. Multiple mutations are separated by addition marks (“+”), e.g., “Gly205Arg+Ser411Phe” or “G205R+S411F”, representing substitutions at positions 205 and 411 of glycine (G) with arginine (R) and serine(S) with phenylalanine (F), respectively. Deletions. For an amino acid deletion, the following nomenclature is used: Original amino acid, position, *. Accordingly, the deletion of glycine at position 195 is designated as “Gly195*” or “G195*”. Multiple deletions are separated by addition marks (“+”), e.g., “Gly195*+Ser411*” or “G195*+S411*”.

Insertions. For an amino acid insertion, the following nomenclature is used: Original amino acid, position, original amino acid, inserted amino acid. Accordingly, the insertion of lysine after glycine at position 195 is designated “Gly195GlyLys” or “G195GK”. An insertion of multiple amino acids is designated [Original amino acid, position, original amino acid, inserted amino acid #1, inserted amino acid #2; etc.]. For example, the insertion of lysine and alanine after glycine at position 195 is indicated as “Gly195GlyLysAla” or “G195GKA”.

In such cases the inserted amino acid residue(s) are numbered by the addition of lower case letters to the position number of the amino acid residue preceding the inserted amino acid residue(s). In the above example, the sequence would thus be:

| Parent: | Variant: |
|---|---|
| 195 | 195 195a 195b |
| G | G - K - A |

Multiple alterations. Variants comprising multiple alterations are separated by addition marks (“+”), e.g., “Arg170Tyr+Gly195Glu” or “R170Y+G195E” representing a substitution of arginine and glycine at positions 170 and 195 with tyrosine and glutamic acid, respectively.

Different alterations. Where different alterations can be introduced at a position, the different alterations are separated by a comma, e.g., “Arg170Tyr, Glu” represents a substitution of arginine at position 170 with tyrosine or glutamic acid. Thus, “Tyr167Gly, Ala+Arg170Gly, Ala” designates the following variants:

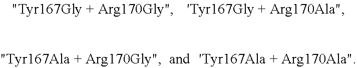

### DETAILED DESCRIPTION OF THE INVENTION

The present invention relates to carbonic anhydrase variants, comprising a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1, wherein the variant has at least 65%, e.g., at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, SEQ ID NO: 2, SEQ ID NO: 3, or SEQ ID NO: 4, or the mature polypeptide sequence thereof, and wherein the variant has carbonic anhydrase activity.

The variants may further comprise an extension of one or more amino acids at the N-terminal and/or C-terminal ends.

Alternatively, the variants may further comprise a truncation of one or more amino acids at the N-terminal and/or C-terminal ends.

### Variants

In an embodiment, the variant has a sequence identity of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%, but less than 100%, to the amino acid sequence of the parent carbonic anhydrase.

In another embodiment, the variant has at least 65%, e.g., at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the polypeptide of SEQ ID NO: 1, or the mature polypeptide thereof.

In another embodiment, the variant has at least 65%, e.g., at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the polypeptide of SEQ ID NO: 2, or the mature polypeptide thereof.

In another embodiment, the variant has at least 65%, e.g., at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the polypeptide of SEQ ID NO: 3, or the mature polypeptide thereof.

In another embodiment, the variant has at least 65%, e.g., at least 70%, at least 75%, at least 80%, at least 85%, at least 90%, at least 91%, at least 92%, at least 93%, at least 94%, at least 95%, such as at least 96%, at least 97%, at least 98%, or at least 99%, but less than 100%, sequence identity to the polypeptide of SEQ ID NO: 4, or the mature polypeptide thereof.

In one aspect, the number of alterations (e.g., substitutions) in the variants of the present invention is 1-20, e.g., 1-10 and 1-5, such as 1, 2, 3, 4, 5, 6, 7, 8, 9 or 10 alterations.

In another aspect, a variant comprises a substitution at one or more positions corresponding to positions a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, a variant comprises a substitution at two or more positions corresponding to positions a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, a variant comprises a substitution at three or more positions corresponding to positions a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, a variant comprises a substitution at four or more positions corresponding to positions a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, a variant comprises a substitution at five or more positions corresponding to positions 1 a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, a variant comprises a substitution at six or more positions (e.g., 5, 6, 7, 8, 9, 10, 11, 12, 13, 14, 15 or more positions) corresponding to positions a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 1 of the polypeptide of SEQ ID NO: 1. In another aspect, the amino acid at a position corresponding to position 1 is substituted with Ala, Arg, Asn, Asp, Cys, Gln, Glu, Gly, His, Ile, Leu, Lys, Met, Phe, Pro, Ser, Thr, Trp, Tyr, or Val, preferably with Met or Asp. In another aspect, the variant comprises or consists of the substitution G1M or G1D corresponding to the polypeptide of SEQ ID NO: 1. In another aspect, the variant comprises or consists of a substitution at a position corresponding to position 1 of the polypeptide of SEQ ID NO: 1, together with a substitution at one or more positions described herein, such as a substitution at one or more positions corresponding to positions 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 3 of the polypeptide of SEQ ID NO: 1. In another aspect, the amino acid at a position corresponding to position 3 is substituted with Ala, Arg, Asn, Asp, Cys, Gln, Glu, Gly, His, Ile, Leu, Lys, Met, Phe, Pro, Ser, Thr, Trp, Tyr, or Val, preferably with Gly or Cys. In another aspect, the variant comprises or consists of the substitution S3C or S3G corresponding to the polypeptide of SEQ ID NO: 1. In another aspect, the variant comprises or consists of a substitution at a position corresponding to position 3 of the polypeptide of SEQ ID NO: 1, together with a substitution at one or more positions described herein, such as a substitution at one or more positions corresponding to positions 1, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., S3C+H8C; S3G+D18P+D39L+E186K+E188P+M189L).

In another aspect, the variant comprises or consists of a substitution at a position corresponding to position 5 of the polypeptide of SEQ ID NO: 1. In another aspect, the amino acid at a position corresponding to position 1 is substituted with Ala, Arg, Asn, Asp, Cys, Gln, Glu, Gly, His, Ile, Leu, Lys, Met, Phe, Pro, Ser, Thr, Trp, Tyr, or Val, preferably with Arg or Ser. In another aspect, the variant comprises or consists of the substitution H5R or H5S corresponding to the polypeptide of SEQ ID NO: 1. In another aspect, the variant comprises or consists of a substitution at a position corresponding to position 5 of the polypeptide of SEQ ID NO: 1, together with a substitution at one or more positions described herein, such as a substitution at one or more positions corresponding to positions 1, 3, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., H5S+D18P; H5S+H112N; H5R+G6P+E19P; H5R+G6P+E19P+K151E).

In another aspect, the variant comprises or consists of a substitution at a position corresponding to position 6 of the polypeptide of SEQ ID NO: 1. In another aspect, the amino acid at a position corresponding to position 6 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro. In another aspect, the variant comprises or consists of the substitution G6P corresponding to the polypeptide of SEQ ID NO: 1. In another aspect, the variant comprises or consists of a substitution at a position corresponding to position 6 of the polypeptide of SEQ ID NO: 1, together with a substitution at one or more positions described herein, such as a substitution at one or more positions corresponding to positions 1, 3, 5, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., H5R+G6P+E19P; H5R+G6P+E19P+K151E).

In another aspect, the variant comprises or consists of a substitution at a position corresponding to position 7 of the polypeptide of SEQ ID NO: 1. In another aspect, the amino acid at a position corresponding to position 7 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys, Asp, His, Gln or Arg. In another aspect, the variant comprises or consists of the substitution E7K, E7D, E7H, E7Q or E7R corresponding to the polypeptide of SEQ ID NO: 1. In another aspect, the variant comprises or consists of a substitution at a position corresponding to position 7 of the polypeptide of SEQ ID NO: 1, together with a substitution at one or more positions described herein, such as a substitution at one or more positions corresponding to positions 1, 3, 5, 6, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., E7K+D18P+D39L+E186K+E188P+M189L; E7K+D18P, D39L+K55Y+N59V+K64L+Q84E+E186K+E188P+M189L+M220L; E7K+D18P+D39L+K64L+E186K+E188P+M189L; E7K+D18P+D39L+K64L+E186K+E188P+M189L+K198L; E7K+D18P+D39L+D156L+E186K+E188P+M189L+K198L; E7K+D18P+D39L+D156L+E186K+E188P+M189L; E7K+D18P+D39L+K55Y+E186K+E188P+M189L; E7K+D18P+D39L+E186K+E188P+M189L+K198L; E7K+D18P+D39L+K55Y+E186K+E188P+M189L+K198L; E7K+D18P+D39L+K55Y+K64L+D156L+E186K+E188P+M189L+K198L; E7K+D18P+D39L+K55Y+K64L+D156L+E186K+E188P+M189L; E7K+D18P+D39L+K55Y+K64L+D156L+E186K+E188P+M189L+K198L+M220L; E7K+D18P+D39L+K55Y+D156L+E186K+E188P+M189L; E7R+E186K+E221K; E7Q+E186K+E221K; E7K+E186K+E221K; E7D+E186K+E221K; E7H+E186K+E221K; E7K+D18P+D39L+K55Y+N59S+K64L+V75I+Q84E+R166Y+E186K+E188P+M189L+M218I+M220L; E7K+D18P+M22F+N28T+D39L+K55Y+N59S+K64L+V75I+Q84E+E178P+E186K+E188P+M189L+M220L; E7K+D18P+D39L+K55Y+N59S+K64L+V75I+Q84E+D156M+E186K+E188P+M189L+M220L; E7K+D18P+D39L+K55Y+N59S+K64L+V75I+Q84E+E145F+E186K+E188P+M189L+M220L; E7K+D18P+D39L+K55Y+N59S+K64L+V75I+Q84E+R166Y+E186K+E188P+M189L+M218L+M220L; E7K+D18P+N28F+D39L+K55Y+N59S+K64L+V75I+Q84E+S168A+E186K+E188P+M189L+M220L; E7K+D18P+K27T+D39L+K55Y+N59S+K64L+V75I+Q84E+N114R+E186K+E188P+M189L+M220L; E7K+D18P+R36N+D39L+K55Y+N59S+K64L+V75I+D76N+Q84E+R181T+E186K+E188P+M189L+M220L; E7K+D18P+D39L+K55Y+N59S+K64L+V75I+Q84I+E186K+E188P+M189L+M220L; E7K+D18P+D39L+K55Y+N59S+K64L+V75I+Q84M+E186K+E188P+M189L+M220L; E7K+D18P+D39L+K55Y+N59S+K64L+V75I+Q84F+E186K+E188P+M189L+M220L; E7K+D18P+D39L+K55Y+N59S+K64L+V75I+Q84E+E145L+E186K+E188P+M189L+M220L; E7K+D18P+D39L+K55Y+N59S+H61Y+K64L+V75I+Q84E+E186K+E188P+M189L+M220L; E7K+D18P+D39L+K55Y+N59S+K64L+V75I+Q84E+E186K+E188P+M189L+M220L+E221L; E7K+D18P+D39L+K55Y+N59S+K64L+S66F+V75I+Q84E+E186K+E188P+M189L+M220L; E7K+D18P+D39L+E48Y+K55Y+N59S+K64L+V75I+Q84E+E186K+E188P+M189L+M220L; E7K+D18P+D39L+K43P+K55Y+N59S+K64L+V75I+Q84E+E186K+E188P+M189L+M220L; E7K+D18P+M22Y+N28F+D39L+K55Y+N59S+K64L+V75I+Q84E+E186K+E188P+M189L+M220L; E7K+D18P+N28Y+D39L+K55Y+N59S+K64L+V75I+Q84E+S168A+E186K+E188P+M189L+M220L; E7K+D18P+D39L+K55Y+N59S+K64L+V75I+Q84E+R166Y+E186K+E188P+M189L+M218V+M220L; E7K+D18P+D39L+K55Y+N59S+K64L+V75I+Q84Y+E186K+E188P+M189L+M220L; E7K+D18P+D39L+K55Y+N59S+K64L+S66Y+V75I+Q84E+E186K+E188P+M189L+M220L; E7K+D18P+D39L+K55Y+N59S+K64L+S66Q+V75I+Q84E+E186K+E188P+M189L+M220L; E7K+D18P+D39L+K55Y+N59S+K64L+V75I+Q84W+E186K+E188P+M189L+M220L; E7K+D18P+D39L+K55Y+N59S+K64L+S66L+V75I+Q84E+E186K+E188P+M189L+M220L; E7K+D18P+D39L+K55Y+N59S+K64L+V75I+Q84E+G119V+E186K+E188P+M189L+M220L; E7K+D18P+D39L+K55Y+N59S+K64L+Y67W+V75I+Q84E+E186K+E188P+M189L+M220L; E7K+D18P+D39L+K55Y+N59S+K64L+V75I+Q84E+D156Q+E186K+E188P+M189L+M220L; E7K+D18P+D39L+K55Y+N59S+T62Y+K64L+V75I+Q84E+E186K+E188P+M189L+M220L; E7K+D18P+D39L+K55Y+N59S+K64L+S66I+V75I+Q84E+E186K+E188P+M189L+M220L; E7K+D18P+D39L+K55Y+N59S+K64L+V75I+Q84E+A104M+E186K+E188P+M189L+M220L; E7K+D18P+D39L+K55Y+N59S+K64L+E68T+V75I+Q84E+E186K+E188P+M189L+M220L; E7K+D18P+D39L+K55Y+N59S+K64L+V75I+L82F+Q84E+E186K+E188P+M189L+M220L; E7K+D18P+D39L+K55Y+N59S+K64L+E68H+V75I+Q84E+E186K+E188P+M189L+M220L; E7K+D18P+D39L+A51P+K55Y+N59S+K64L+V75I+Q84E+E186K+E188P+M189L+M220L; E7K+D18P+D39L+E48F+K55Y+N59S+K64L+V75I+Q84E+E186K+E188P+M189L+M220L; E7K+D18P+D39L+K55Y+N59S+K64L+V75I+Q84L+E186K+E188P+M189L+M220L; E7K+D18P+D39L+K55Y+N59S+K64L+S66W+V75I+Q84E+E186K+E188P+M189L+M220L; E7K+D18P+D39L+K55Y+N59S+H61W+K64L+V75I+Q84E+E186K+E188P+M189L+M220L; E7K+D18P+D39L+K55Y+N59S+K64L+V75I+Q84E+E186K+E188P+M189L+K198F+M220L; E7K+D18P+D39L+K55Y+N59S+K64L+E68D+V75I+Q84E+E186K+E188P+M189L+M220L; E7K+D18P+D39L+K55Y+N59S+K64L+V75I+Q84E+E155I+E186K+E188P+M189L+M220L; E7K+D18P+D39L+K55Y+N59S+K64L+V75I+Q84E+E186K+E188P+M189L+K198Y+M220L; E7K+D18P+D39L+K55Y+N59S+K64L+S66H+V75I+Q84E+E186K+E188P+M189L+M220L; E7K+D18P+D39L+K55Y+N59S+K64L+V75I+Q84E+D156N+E186K+E188P+M189L+M220L; E7K+D18P+D39L+K55Y+N59S+K64L+V75I+Q84E+L115Q+E186K+E188P+M189L+M220L; E7K+D18P+D39L+K55Y+N59S+K64L+V75I+Q84E+L115W+E186K+E188P+M189L+M220L; E7K+D18P+D39L+K43Y+K55Y+N59S+K64L+V75I+Q84E+E186K+E188P+M189L+M220L; E7K+D18P+D39L+K55Y+N59S+K64L+V75I+Q84E+L115Y+E186K+E188P+M189L+M220L; E7K+D18P+D39L+K55Y+N59S+K64L+S71A+V75I+Q84E+E186K+E188P+M189L+M220L; E7K+D18P+D39L+K55Y+N59S+K64L+V75I+Q84E+F87W+E186K+E188P+M189L+M220L; E7K+D18P+D39L+K55Y+N59S+K64L+Y67V+V75I+Q84E+E186K+E188P+M189L+M220L; E7K+D18P+D39L+K55Y+N59S+K64L+V75I+Q84E+L115I+E186K+E188P+M189L+M220L; E7K+D18P+D39L+K55Y+N59S+K64L+V75I+Q84E+H86W+E186K+E188P+M189L+M220L; E7K+D18P+D39L+K55Y+N59S+K64L+V75I+Q84E+H86I+E186K+E188P+M189L+M220L; E7K+D18P+D39L+K55Y+N59S+K64L+Y67F+V75I+Q84E+E186K+E188P+M189L+M220L; E7K+D18P+D39L+R50Y+K55Y+N59S+K64L+V75I+Q84E+E186K+E188P+M189L+M220L; E7K+D18P+D39L+K55Y+N59S+T62F+K64L+V75I+Q84E+E186K+E188P+M189L+M220L; E7K+D18P+D39L+K55Y+N59S+K64L+V75I+Q84E+A149H+E186K+E188P+M189L+M220L; E7K+D18P+D39L+K55Y+N59S+K64L+E68R+V75I+Q84E+E186K+E188P+M189L+M220L; E7K+D18P+D39L+K55Y+N59S+K64L+V75I+Q84E+E186K+E188P+M189L+N208L+M220L; E7K+D18P+D39L+K55Y+N59S+K64L+V75I+Q84E+M185F+E186K+E188P+M189L+M220L; E7K+D18P+D39L+K55Y+N59S+K64L+S66V+V75I+Q84E+E186K+E188P+M189L+M220L; E7K+D18P+D39L+K55Y+N59S+K64L+S66M+V75I+Q84E+E186K+E188P+M189L+M220L; E7K+D18P+D39L+K55Y+N59S+K64L+Y67I+V75I+Q84E+E186K+E188P+M189L+M220L; E7K+D18P+D39L+K55Y+N59S+K64L+V75I+Q84E+H86Y+E186K+E188P+M189L+M220L; E7K+D18P+D39L+K55Y+N59S+K64L+V75I+Q84E+E145Y+E186K+E188P+M189L+M220L; E7K+D18P+D39L+K43F+K55Y+N59S+K64L+V75I+Q84E+E186K+E188P+M189L+M220L; E7K+D18P+D39L+K55Y+N59S+K64L+V75I+Q84E+A104C+E186K+E188P+M189L+M220L; E7K+D18P+D39L+K55Y+N59S+K64L+V75I+Q84E+E178S+E186K+E188P+M189L+M220L; E7K+D18P+D39L+K55Y+N59S+K64L+V75I+Q84E+S168A+E178P+E186K+E188P+M189L+M220L; E7K+D18P+D39L+K55Y+N59S+K64L+V75I+Q84E+S168C+E186K+E188P+M189L+A216C+M220L; E7K+D18P+M22F+N28D+D39L+K55Y+N59S+K64L+V75I+Q84E+E178T+E186K+E188P+M189L+M220L; E7K+D18P+M22F+N28D+D39L+K55Y+N59S+K64L+V75I+Q84E+E178P+E186K+E188P+M189L+M220L; E7K+D18P+M22F+N28D+D39L+K55Y+N59S+K64L+V75I+Q84E+E178S+E186K+E188P+M189L+M220L; E7K+D18P+R36N+D39L+K55Y+N59S+K64L+V75I+D76K+Q84E+R181A+E186K+E188P+M189L+M220L; E7K+D18P+R36N+D39L+K55Y+N59S+K64L+V75I+D76N+Q84E+R181A+E186K+E188P+M189L+M220L; E7K+D18P+R36N+D39L+K55Y+N59S+K64L+V75I+Q84E+S168A+R181T+E186K+E188P+M189L+M218V+M220L).

In another aspect, the variant comprises or consists of a substitution at a position corresponding to position 8 of the polypeptide of SEQ ID NO: 1. In another aspect, the amino acid at a position corresponding to position 8 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys or Asn. In another aspect, the variant comprises or consists of the substitution H8C or H8N corresponding to the polypeptide of SEQ ID NO: 1. In another aspect, the variant comprises or consists of a substitution at a position corresponding to position 8 of the polypeptide of SEQ ID NO: 1, together with a substitution at one or more positions described herein, such as a substitution at one or more positions corresponding to positions 1, 3, 5, 6, 7, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., S3C+H8C; H8N+A154P+E186K+E221K).

In another aspect, the variant comprises or consists of a substitution at a position corresponding to position 12 of the polypeptide of SEQ ID NO: 1. In another aspect, the amino acid at a position corresponding to position 12 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises or consists of the substitution H12L corresponding to the polypeptide of SEQ ID NO: 1. In another aspect, the variant comprises or consists of a substitution at a position corresponding to position 12 of the polypeptide of SEQ ID NO: 1, together with a substitution at one or more positions described herein, such as a substitution at one or more positions corresponding to positions 1, 3, 5, 6, 7, 8, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., H12L+N35D+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 17 of the polypeptide of SEQ ID NO: 1. In another aspect, the amino acid at a position corresponding to position 1 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another aspect, the variant comprises or consists of the substitution K17S corresponding to the polypeptide of SEQ ID NO: 1. In another aspect, the variant comprises or consists of a substitution at a position corresponding to position 17 of the polypeptide of SEQ ID NO: 1, together with a substitution at one or more positions described herein, such as a substitution at one or more positions corresponding to positions 1, 3, 5, 6, 7, 8, 12, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 18 of the polypeptide of SEQ ID NO: 1. In another aspect, the amino acid at a position corresponding to position 18 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys, Glu, Arg, Gly, Ser or Pro. In another aspect, the variant comprises or consists of the substitution D18K, D18E, D18R, D18G, D18S or D18P corresponding to the polypeptide of SEQ ID NO: 1. In another aspect, the variant comprises or consists of a substitution at a position corresponding to position 18 of the polypeptide of SEQ ID NO: 1, together with a substitution at one or more positions described herein, such as a substitution at one or more positions corresponding to positions 1, 3, 5, 6, 7, 8, 12, 17, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., H5S+D18P; D18P+K41C; D18P+A53P; D18P+F102M; D18P+E188P; D18K+D39R+K64L; D18E+D39R+K147I; D18P+K43A+M218K; D18P+H112E+R126K; D18P+I130E+M189I; D18P+R36D+K43A+M218K; D18E+D39L+K64L+E186L; D18K+D39L+H99L+E186L; D18K+D39L+H99L+M218V; D18K+D39L+N141K+K147I; D18K+D39L+K147I+E186R; D18E+D39R+S177Y+E186R; D18K+D39L+E186K+M189L; D18K+D39R+E186R+E221R; D18E+D39L+E186L+M191V; D18E+D39L+M189L+M191V; D18K+K64L+E186R+M191V; D18K+H99L+E186R+M189L; D18E+N141K+E186R+M218V; D18E+K147I+E186R+M189I; D18E+D163K+E186R+E221R; D18K+D39L+K147I+E186R; D18E+S177Y+E186R+M189L; D18K+D39L+E186R+M189L; D18K+N141K+K147I+E186R; D18E+D39R+D163K+E186R; D18K+H99L+E186R+E221L; D18K+K64L+S177Y+E186R; D18K+D39L+E186K+E221R; D18E+D39L+N141K+E186K; D18K+D39L+N141K+E221L; D18P+K41C+K43A+M218K; D18K+D39L+E186R+M218V; D18K+D39L+S177Y+M189I; D18K+D39L+M189L+M191V; D18E+N141K+E186R+E221L; D18K+E186R+M191V+E221L; D18E+A53G+K64L+E186R; D18K+D39L+A53G+E186L; D18K+E186R+M189L+E221L; D18E+D39L+D163K+E221R; D18K+K64L+D163K+E186R; D18K+D39R+E186R+M218V; D18E+K147I+E186R+M191V; D18K+D163K+E186R+M189I; D18K+D39L+S177Y+E186R; D18E+K64L+E186R+M189I; D18E+D39L+S177Y+E186R; D18E+A53G+D163K+E186R; D18K+D39L+M189I+E221R; D18K+D39L+D163K+M218V; D18E+D39L+E186R+M189L; D18E+E186R+M189I+E221L; D18K+A53G+K147I+E186R; D18E+H99L+E186R+M218V; D18P+R36D+K41C+K43A+M218K; D18P+D39L+H99L+E186L+E188P; D18P+D39L+N141K+E186R+E188P; D18P+D39L+E186K+E188P+M189L; D18P+D39R+E186R+E188P+E221L; D18P+D39L+E188P+M189L+E221R; D18P+A53G+E186R+E188P+E221L; D18P+H99L+K147I+E186R+E188P; D18P+N141K+E186R+E188P+M218V; D18P+D39R+S177Y+E186R+E188P; D18P+E186R+E188P+M189I+E221L; D18P+D163K+E186R+E188P+M189I; D18P+N141K+E186R+E188P+E221R; D18P+D39L+K64L+E186R+E188P; D18P+E186R+E188P+M189L+M191V; D18P+D39L+A53G+E186R+E188P; D18P+D39L+H99L+E188P+M189I; D18P+D39L+E186L+E188P+M218V; D18P+K147I+E186R+E188P+M189L; D18P+H99L+S177Y+E186R+E188P; D18P+D39L+E186K+E188P+M189L; D18P+D39L+K147I+E188P+M191V; D18P+D39L+K64L+E186K+E188P; D18P+D39L+S177Y+E188P+M191V; D18P+D39L+E186K+E188P+M189L; D18P+K64L+E186R+E188P+E221R; D18P+D163K+E186R+E188P+M191V; D18P+D39K+D161N+E186K+E188P+E221K+K222*; D18P+D39K+K41C+D161N+E186K+E188P+E221K+K222*; D18P+D39K+K43A+D161N+E186K+E188P+M218K+E221K+K222*; D18P+D39K+K41C+K43A+D161N+E186K+E188P+M218K+E221K+K222*; D18P+R36D+D39K+K41C+K43A+D161N+E186K+E188P+M218K+E221K+K222*; D18P+D39L+N59L+V75I+E186K+E188P+M189L; D18P+D39L+K55Y+N59H+K64L+D156L+E186K+E188P+M189L+K198L+M220L; D18P+D39L+N59T+V75I+Q84E+E186K+E188P+M189L; D18P+D39L+N59T+V75I+E186K+E188P+M189L; D18P+D39L+K55Y+N59W+K64L+Q84G+D156L+E186K+E188P+M189L+K198L+M220L; D18P+D39L+N59S+K64L+V75I+E186K+E188P; D18P+D39L+N59I+K64L+V75I+E186K+E188P; D18P+D39L+N59S+V75I+E186K+E188P+M189L; D18P+D39L+N59T+K64L+V75I+E186K+E188P; D18P+D39L+K55Y+N59S+K64L+D156L+E186K+E188P+M189L+K198L+M220L; D18P+D39L+N59L+K64L+V75I+E186K+E188P; D18P+D39L+N59D+V75I+Q84E+E186K+E188P+M189L; D18P+D39L+N59H+K64L+V75I+E186K+E188P; D18P+D39L+N59H+K64L+E186K+E188P; D18P+D39L+E186K+E188P+M189L+K198L+M220L; D18P+D39L+N59R+V75I+E186K+E188P+M189L; D18P+D39L+N59I+K64L+E186K+E188P; D18P+D39L+N59L+K64L+E186K+E188P; D18P+D39L+K55Y+K64L+E186K+E188P+K198L; D18P+D39L+N59T+K64L+Q84E+E186K+E188P; D18P+D39L+N59S+K64L+E186K+E188P; D18P+D39L+K55Y+K64L+Q84G+D156L+E186K+E188P+M189L+K198L+M220L; D18P+D39L+N59T+K64L+V75I+Q84K+E186K+E188P; D18P+D39L+K55Y+K64L+V75I+D156L+E186K+E188P+M189L+K198L+M220L; D18P+D39L+N59T+K64L+E186K+E188P; D18P+D39L+K55Y+E186K+E188P+M189L+M220L; D18P+D39L+N59V+K64L+E186K+E188P; D18P+D39L+K55Y+K64L+E186K+E188P; D18P+D39L+K55Y+E186K+E188P+M189L+K198L; D18P+D39L+N59R+K64L+E186K+E188P; D18P+D39L+N59W+K64L+Q84G+E186K+E188P; D18P+D39L+N59V+V75I+E186K+E188P+M189L; D18P+D39L+N59H+V75I+E186K+E188P+M189L; D18P+D39L+K64L+E186K+E188P+M189L; D18P+D39L+K55Y+K64L+V75L+D156L+E186K+E188P+M189L+K198L+M220L; D18P+D39L+V75I+Q84A+E186K+E188P+M189L; D18P+D39L+N59D+V75I+E186K+E188P+M189L; D18P+D39L+N59W+V75I+Q84G+E186K+E188P+M189L; D18P+D39L+K55Y+K64L+D156L+E186K+E188P; D18P+D39L+N59W+K64L+V75I+Q84G+E186K+E188P; D18P+D39L+K64L+D156L+E186K+E188P+K198L; D18P+D39L+D156L+E186K+E188P+M189L+K198L; D18P+D39L+V75I+S168A+E186K+E188P+M189L; D18P+D39L+K55Y+K64L+D156L+S168A+E186K+E188P+M189L+K198L+M220L; D18P+D39L+K64L+S168A+E186K+E188P; D18P+D39L+N59T+K64L+Q84K+E186K+E188P; D18P+D39L+V75L+E186K+E188P+M189L; D18P+N28R+D39L+V75I+S168Q+E186K+E188P+M189L; D18P+N28H+D39L+K55Y+K64L+D156L+E186K+E188P+M189L+K198L+M220L; D18P+N28R+D39L+K64L+S168D+E186K+E188P; D18P+N28M+D39L+V75I+S168I+E186K+E188P+M189L; D18P+N28R+D39L+K64L+S168E+E186K+E188P; D18P+N28R+D39L+K64L+S168L+E186K+E188P; D18P+N28Q+D39L+K64L+S168K+E186K+E188P; D18P+N28L+D39L+V75I+S168I+E186K+E188P+M189L; D18P+N28Q+D39L+V75I+S168K+E186K+E188P+M189L; D18P+N28R+D39L+N59L+V75I+S168P+E186K+E188P+M189L; D18P+N28R+D39L+V75I+S168L+E186K+E188P+M189L; D18P+N28R+D39L+N59L+V75I+S168D+E186K+E188P+M189L; D18P+N28E+D39L+V75I+S168M+E186K+E188P+M189L; D18P+N28R+D39L+V75I+S168D+E186K+E188P+M189L; D18P+N28M+D39L+K64L+V75I+S168M+E186K+E188P; D18P+N28Q+D39L+N59L+V75I+S168Q+E186K+E188P+M189L; D18P+N28R+D39L+K64L+V75I+S168L+E186K+E188P; D18P+N28R+D39L+K64L+V75I+S168M+E186K+E188P; D18P+N28E+D39L+V75I+S168D+E186K+E188P+M189L; D18P+N28Q+D39L+K64L+S168T+E186K+E188P; D18P+N28Q+D39L+V75I+S168L+E186K+E188P+M189L; D18P+N28R+D39L+K64L+V75I+S168E+E186K+E188P; D18P+N28R+D39L+V75I+S168P+E186K+E188P+M189L; D18P+N28E+D39L+K64L+V75I+S168M+E186K+E188P; D18P+N28Q+D39L+K64L+S168L+E186K+E188P; D18P+N28R+D39L+K64L+V75I+S168P+E186K+E188P; D18P+N28R+D39L+K64L+V75I+S168Q+E186K+E188P; D18P+N28R+D39L+V75I+S168M+E186K+E188P+M189L; D18P+N28L+D39L+K64L+V75I+S168V+E186K+E188P; D18P+N28Q+D39L+K64L+V75I+S168L+E186K+E188P; D18P+N28Q+D39L+V75I+S168T+E186K+E188P+M189L; D18P+N28R+D39L+V75I+E186K+E188P+M189L; D18P+N28M+D39L+V75I+S168M+E186K+E188P+M189L; D18P+N28Q+D39L+V75I+S168Q+E186K+E188P+M189L; D18P+N28Q+D39L+V75I+S168P+E186K+E188P+M189L; D18P+N28H+D39L+K64L+E186K+E188P; D18P+N28H+D39L+N59R+V75I+E186K+E188P+M189L; D18P+N28H+D39L+N59L+V75I+E186K+E188P+M189L; D18P+D39L+K64L+E186K+E188P+M220L; D18P+D39L+K64L+E186K+E188P+K198L; D18P+D39L+K64L+D156L+E186K+E188P; D18P+D39L+K64L+E186K+E188P+K198L+M220L; D18P+D39L+K64L+D156L+E186K+E188P+M220L; D18P+D39L+K55Y+K64L+E186K+E188P+M220L; D18P+D39L+K64L+E186K+E188P+M189L+M220L; D18P+D39L+K55Y+K64L+E186K+E188P+M189L; D18P+D39L+K64L+D156L+E186K+E188P+K198L+M220L; D18P+D39L+K55Y+K64L+E186K+E188P+K198L+M220L; D18P+D39L+K64L+E186K+E188P+M189L+K198L+M220L; D18P+D39L+K55Y+K64L+D156L+E186K+E188P+M220L; D18P+D39L+K55Y+K64L+E186K+E188P+M189L+M220L; D18P+D39L+K55Y+K64L+D156L+E186K+E188P+K198L; D18P+D39L+K64L+D156L+E186K+E188P+M189L+K198L; D18P+D39L+K55Y+K64L+D156L+E186K+E188P+K198L+M220L; D18P+D39L+K64L+D156L+E186K+E188P+M189L+K198L+M220L; D18P+D39L+K55Y+K64L+D156L+E186K+E188P+M189L+M220L; D18P+D39L+K55Y+K64L+D156L+E186K+E188P+M189L+K198L; D18P+D39L+D156L+E186K+E188P+M189L+M220L; D18P+D39L+K64L+E186K+E188P+M189L+K198L; D18P+D39L+K55Y+D156L+E186K+E188P+M189L; D18P+D39L+K64L+D156L+E186K+E188P+M189L; D18P+D39L+D156L+E186K+E188P+M189L+K198L+M220L; D18P+D39L+K55Y+E186K+E188P+M189L+K198L+M220L; D18P+D39L+K55Y+D156L+E186K+E188P+M189L+M220L; D18P+D39L+K64L+D156L+E186K+E188P+M189L+M220L; D18P+D39L+K55Y+D156L+E186K+E188P+M189L+K198L; D18P+D39L+K55Y+K64L+E186K+E188P+M189L+K198L; D18P+D39L+K55Y+K64L+D156L+E186K+E188P+M189L; D18P+D39L+K55Y+D156L+E186K+E188P+M189L+K198L+M220L; D18P+D39L+K55Y+K64L+E186K+E188P+M189L+K198L+M220L; D18P+D39L+K55Y+K64L+D156L+E186K+E188P+M189L+K198L+M220L; D18P+N28R+D39L+K64L+S168P+E186K+E188P; D18P+N28E+D39L+K64L+S168D+E186K+E188P; D18P+N28R+D39L+K64L+E186K+E188P; D18P+N28Q+D39L+K64L+S168Q+E186K+E188P; D18P+N28R+D39L+K55Y+K64L+D156L+E186K+E188P+M189L+K198L+M220L; D18P+N28H+D39L+K64L+S168T+E186K+E188P; D18P+N28R+D39L+K64L+S168Q+E186K+E188P; D18P+N28M+D39L+K64L+S168M+E186K+E188P; D18P+N28S+D39L+K64L+E186K+E188P; D18P+N28R+D39L+K64L+S168M+E186K+E188P; D18P+N28Q+D39L+K64L+S168P+E186K+E188P; D18P+N28H+D39L+K64L+S168E+E186K+E188P; D18P+N28Q+D39L+K64L+S168E+E186K+E188P; D18P+N28L+D39L+K64L+S168V+E186K+E188P; D18P+N28Y+D39L+K64L+S168E+E186K+E188P; D18P+N28F+D39L+K64L+S168E+E178Q+E186K+E188P; D18P+N28M+D39L+K64L+S168I+E186K+E188P; D18P+N28Q+D39L+K64L+E186K+E188P; D18P+N28H+D39L+K64L+S168D+E186K+E188P; D18P+N28K+D39L+K64L+E186K+E188P; D18P+D39L+K64L+E178Q+E186K+E188P; D18P+N28L+D39L+K64L+S168I+E186K+E188P; D18P+N28E+D39L+K64L+S168M+E186K+E188P; D18P+D39L+N59D+K64L+E186K+E188P; D18P+D39L+N59D+K64L+Q84E+E186K+E188P; D18P+D39L+K64L+Q84G+E186K+E188P; D18P+D39L+K64L+Q84A+E186K+E188P; D18P+N28E+D39L+K64L+V75I+S168D+E186K+E188P; D18P+N28H+D39L+K64L+V75I+E186K+E188P; D18P+N28R+D39L+K64L+V75I+S168D+E186K+E188P; D18P+N28R+D39L+K64L+V75I+E186K+E188P; D18P+N28Q+D39L+K64L+V75I+S168Q+E186K+E188P; D18P+N28H+D39L+K64L+V75I+S168T+E186K+E188P; D18P+N28M+D39L+K64L+V75I+E186K+E188P; D18P+N28S+D39L+K64L+V75I+E186K+E188P; D18P+N28Q+D39L+K64L+V75I+S168P+E186K+E188P; D18P+N28H+D39L+K64L+V75I+S168E+E186K+E188P; D18P+N28Q+D39L+K64L+V75I+S168E+E186K+E188P; D18P+N28L+D39L+K64L+V75I+F87L+V146A+S168V+E186K+E188P; D18P+N28Y+D39L+K64L+V75I+S168E+E186K+E188P; D18P+N28F+D39L+K64L+V75I+S168E+E178Q+E186K+E188P; D18P+N28M+D39L+K64L+V75I+S168I+E186K+E188P; D18P+N28Q+D39L+K64L+V75I+E186K+E188P; D18P+N28H+D39L+K64L+V75I+S168D+E186K+E188P; D18P+N28K+D39L+K64L+V75I+E186K+E188P; D18P+D39L+K64L+V75I+E178Q+E186K+E188P; D18P+N28Q+D39L+K64L+V75I+S168K+E186K+E188P; D18P+N28L+D39L+K64L+V75I+S168I+E186K+E188P; D18P+D39L+K64L+V75I+S168A+E186K+E188P; D18P+N28Q+D39L+K64L+V75I+E186K+E188P+M189L+K198L+M220L; D18P+N28Q+D39L+K64L+V75I+S168T+E186K+E188P; D18P+D39L+N59R+K64L+V75I+E186K+E188P; D18P+D39L+N59T+K64L+V75I+Q84E+E186K+E188P; D18P+D39L+N59V+K64L+V75I+E186K+E188P; D18P+D39L+N59D+K64L+V75I+E186K+E188P; D18P+D39L+N59D+K64L+V75I+Q84E+E186K+E188P; D18P+D39L+K64L+V75I+Q84G+E186K+E188P; D18P+D39L+K64L+V75I+Q84A+E186K+E188P; D18P+D39L+K64L+V75I+E186K+E188P; D18P+D39L+K64L+V75L+E186K+E188P; D18P+N28E+D39L+D156L+S168D+E186K+E188P+M189L; D18P+N28H+D39L+V75I+E186K+E188P+M189L; D18P+N28H+D39L+V75I+S168T+E186K+E188P+M189L; D18P+N28M+D39L+V75I+S168M+E186K+E188P; D18P+N28S+D39L+V75I+E186K+E188P+M189L; D18P+N28Q+D39L+S168P+E186K+E188P+M189L; D18P+N28Q+D39L+K55Y+K64L+S168P+E186K+E188P+M189L; D18P+N28R+D39L+V75I+S168E+E186K+E188P+M189L; D18P+N28H+D39L+V75I+S168E+E186K+E188P+M189L; D18P+N28Q+D39L+V75I+S168E+E186K+E188P+M189L; D18P+N28L+D39L+V75I+S168V+E186K+E188P+M189L; D18P+N28Y+D39L+V75I+S168E+E186K+E188P+M189L; D18P+N28F+D39L+V75I+S168E+E178Q+E186K+E188P+M189L; D18P+N28Q+D39L+V75I+E186K+E188P+M189L; D18P+N28H+D39L+V75I+S168D+E186K+E188P+M189L; D18P+N28K+D39L+V75I+E186K+E188P+M189L; D18P+D39L+V75I+E178Q+E186K+E188P+M189L; D18P+N28Q+D39L+V75I+S168K+E186K+E188P+M189L+K198L+M220L; D18P+N28Q+D39L+V75I+S168K+E186K+E188P; D18P+N28Q+D39L+K55Y+K64L+N153D+D156L+S168T+E186K+E188P+M189L; D18P+D39L+N59T+V75I+Q84K+E186K+E188P+M189L; D18P+D39L+N59I+V75I+E186K+E188P+M189L; D18P+D39L+V75I+Q84G+E186K+E188P+M189L; D18P+D39L+V75I+Q84A+L171F+E186K+E188P+M189L; D18P+N28R+D39L+N59L+K64L+D156L+S168P+E186K+E188P+M189L; D18P+N28E+D39L+N59L+V75I+S168D+E186K+E188P+M189L; D18P+N28R+D39L+K64L+S168D+E186K+E188P+M189L; D18P+N28R+D39L+N59L+V75I+E186K+E188P+M189L; D18P+N28Q+D39L+N59L+K64L+S168Q+E186K+E188P+M189L; D18P+N28Q+D39L+N59L+D156L+S168Q+E186K+E188P+M189L; D18P+N28R+D39L+N59R+V75I+S168P+E186K+E188P+M189L; D18P+N28E+D39L+N59R+V75I+S168D+E186K+E188P+M189L; D18P+N28R+D39L+N59R+V75I+S168D+E186K+E188P+M189L; D18P+N28R+D39L+N59R+V75I+E186K+E188P+M189L; D18P+N28R+D39L+K55Y+K64L+D156L+S168P+E186K+E188P+M189L+K198L+M220L; D18P+D39L+K55Y+K64L+D156L+S168D+E186K+E188P+M189L+K198L+M220L; D18P+N28R+D39L+K55Y+K64L+S168D+E186K+E188P+M189L+K198L+M220L; D18P+N28R+D39L+K64L+D156L+S168D+E186K+E188P+M189L+K198L+M220L; D18P+N28Q+D39L+K55Y+K64L+D156L+S168Q+E186K+E188P+M189L+K198L+M220L; D18P+N28H+D39L+K55Y+K64L+D156L+S168T+E186K+E188P+M189L+K198L+M220L; D18P+D39L+K55Y+K64L+D156L+S168M+E186K+E188P+M189L+K198L+M220L; D18P+N28S+D39L+K55Y+K64L+D156L+E186K+E188P+M189L+K198L+M220L; D18P+N28R+D39L+K64L+D156L+S168M+E186K+E188P+M189L+K198L+M220L; D18P+N28Q+D39L+K55Y+K64L+D156L+S168P+E186K+E188P+M189L+K198L+M220L; D18P+N28R+D39L+S168E+E186K+E188P+M189L+K198L+M220L; D18P+N28H+D39L+K55Y+K64L+D156L+S168E+E186K+E188P+M189L+K198L+M220L; D18P+N28Q+D39L+K55Y+K64L+D156L+S168E+E186K+E188P+M189L+K198L+M220L; D18P+N28L+D39L+K55Y+K64L+D156L+S168V+E186K+E188P+M189L+K198L+M220L; D18P+N28Y+D39L+K55Y+K64L+D156L+S168E+E186K+E188P+M189L+K198L+M220L; D18P+N28F+D39L+K55Y+K64L+D156L+S168E+E178Q+E186K+E188P+M189L+K198L+M220L; D18P+N28Q+D39L+K55Y+K64L+D156L+E186K+E188P+M189L+K198L+M220L; D18P+N28H+D39L+K55Y+K64L+D156L+S168D+E186K+E188P+M189L+K198L+M220L; D18P+N28K+D39L+K55Y+K64L+D156L+E186K+E188P+M189L+K198L+M220L; D18P+D39L+K55Y+K64L+D156L+E178Q+E186K+E188P+M189L+K198L+M220L; D18P+N28Q+D39L+K55Y+K64L+D156L+S168K+E186K+E188P+M189L+K198L+M220L; D18P+N28L+D39L+K55Y+K64L+D156L+S168I+E186K+E188P+M189L+K198L+M220L; D18P+N28Q+D39L+K55Y+K64L+D156L+S168L+E186K+E188P+M189L+K198L+M220L; D18P+N28Q+D39L+K55Y+K64L+D156L+S168T+E186K+E188P+M189L+K198L+M220L; D18P+D39L+K55Y+N59L+K64L+D156L+E186K+E188P+M189L+K198L+M220L; D18P+D39L+K55Y+N59R+K64L+D156L+E186K+E188P+M189L+K198L+M220L; D18P+D39L+K55Y+N59T+K64L+Q84E+D156L+E186K+E188P+M189L+K198L+M220L; D18P+D39L+K55Y+N59T+K64L+D156L+E186K+E188P+M189L+K198L+M220L; D18P+D39L+K55Y+N59V+K64L+D156L+E186K+E188P+M189L+K198L+M220L; D18P+D39L+K55Y+N59T+K64L+Q84K+D156L+E186K+E188P+M189L+K198L+M220L; D18P+D39L+K55Y+N59D+K64L+D156L+E186K+E188P+M189L+K198L+M220L; D18P+D39L+K55Y+N59D+K64L+Q84E+D156L+E186K+E188P+M189L+K198L+M220L; D18P+D39L+K55Y+N59I+K64L+D156L+E186K+E188P+M189L+K198L+M220L; D18P+D39L+K55Y+K64L+Q84A+D156L+E186K+E188P+M189L+K198L+M220L; D18P+D39L+K55Y+K64L+V75L+G77D+D156L+E186K+E188P+M189L+K198L+M220L; D18P+N28E+D39L+K55Y+K64L+D156L+S168D+E186K+E188P+M189L+K198L+M220L; D18P+N28R+D39L+K55Y+K64L+D156L+S168D+E186K+E188P+M189L+K198L+M220L; D18P+N28R+D39L+K55Y+K64L+D156L+E186K+E188P+M189L+K198L+M220L; D18P+N28R+D39L+K55Y+K64L+D156L+S168L+E186K+E188P+M189L+K198L+M220L; D18P+N28R+D39L+K55Y+K64L+D156L+S168Q+E186K+E188P+M189L+K198L+M220L; D18P+N28M+D39L+K55Y+K64L+D156L+S168M+E186K+E188P+M189L+K198L+M220L; D18P+N28R+D39L+K55Y+K64L+D156L+S168M+E186K+E188P+M189L+K198L+M220L; D18P+N28R+D39L+K55Y+K64L+D156L+S168E+E186K+E188P+M189L+K198L+M220L; D18P+N28M+D39L+K55Y+K64L+D156L+S168I+E186K+E188P+M189L+K198L+M220L; D18P+N28E+D39L+K55Y+K64L+D156L+S168M+E186K+E188P+M189L+K198L+M220L; D18P+D39L+N59V+K64L+E186K+E188P+M189L+M220L; D18P+D39L+N59S+K64L+E186K+E188P+M189L+M220L; D18P+D39L+N59T+K64L+E186K+E188P+M189L+M220L; D18P+D39L+K64L+V75I+E186K+E188P+M189L+M220L; D18P+D39L+K64L+Q84E+E186K+E188P+M189L+M220L; D18P+D39L+K55Y+N59V+K64L+E186K+E188P+M189L+M220L; D18P+D39L+K55Y+N59S+K64L+E186K+E188P+M189L+M220L; D18P+D39L+K55Y+N59T+K64L+E186K+E188P+M189L+M220L; D18P+D39L+K55Y+K64L+V75I+E186K+E188P+M189L+M220L; D18P+D39L+K55Y+K64L+Q84E+E186K+E188P+M189L+M220L; D18P+D39L+N59V+K64L+V75I+E186K+E188P+M189L+M220L; D18P+D39L+N59V+K64L+Q84E+E186K+E188P+M189L+M220L; D18P+D39L+N59S+K64L+V75I+E186K+E188P+M189L+M220L; D18P+D39L+N59S+K64L+Q84E+E186K+E188P+M189L+M220L; D18P+D39L+N59T+K64L+V75I+E186K+E188P+M189L+M220L; D18P+D39L+N59T+K64L+Q84E+E186K+E188P+M189L+M220L; D18P+D39L+K64L+V75I+Q84E+E186K+E188P+M189L+M220L; D18P+D39L+K55Y+N59V+K64L+V75I+E186K+E188P+M189L+M220L; D18P+D39L+K55Y+N59V+K64L+Q84E+E186K+E188P+M189L+M220L; D18P+D39L+K55Y+N59S+K64L+V75I+E186K+E188P+M189L+M220L; D18P+D39L+K55Y+N59S+K64L+Q84E+E186K+E188P+M189L+M220L; D18P+D39L+K55Y+N59T+K64L+V75I+E186K+E188P+M189L+M220L; D18P+D39L+K55Y+N59T+K64L+Q84E+E186K+E188P+M189L+M220L; D18P+D39L+K55Y+K64L+V75I+Q84E+E186K+E188P+M189L+M220L; D18P+D39L+N59V+K64L+V75I+Q84E+E186K+E188P+M189L+M220L; D18P+D39L+N59S+K64L+V75I+Q84E+E186K+E188P+M189L+M220L; D18P+D39L+N59T+K64L+V75I+Q84E+E186K+E188P+M189L+M220L; D18P+D39L+K55Y+N59V+K64L+V75I+Q84E+E186K+E188P+M189L+M220L; D18P+D39L+K55Y+N59S+K64L+V75I+Q84E+E186K+E188P+M189L+M220L; D18P+D39L+K55Y+N59T+K64L+V75I+Q84E+E186K+E188P+M189L+M220L; D18P+D39L+K55Y+K64L+V75I+E186K+E188P; D18P+D39L+K64L+V75I+Q84E+E186K+E188P; D18P+D39L+K55Y+N59V+K64L+V75I+E186K+E188P; D18P+D39L+K55Y+N59S+K64L+V75I+E186K+E188P; D18P+D39L+K55Y+N59T+K64L+V75I+E186K+E188P; D18P+D39L+K55Y+K64L+V75I+Q84E+E186K+E188P; D18P+D39L+N59V+K64L+V75I+Q84E+E186K+E188P; D18P+D39L+N59S+K64L+V75I+Q84E+E186K+E188P; D18P+D39L+K55Y+N59V+K64L+V75I+Q84E+E186K+E188P; D18P+D39L+K55Y+N59S+K64L+V75I+Q84E+E186K+E188P; D18P+D39L+K55Y+N59T+K64L+V75I+Q84E+E186K+E188P; D18P+D39L+K55Y+V75I+E186K+E188P+M189L; E7K+D18P+D39L+E186K+E188P+M189L+M220L; E7K+D18P+D39L+E186K+E188P+M189L+K198L+M220L; E7K+D18P+D39L+D156L+E186K+E188P+M189L+M220L; E7K+D18P+D39L+K55Y+E186K+E188P+M189L+M220L; E7K+D18P+D39L+K64L+E186K+E188P+M189L+M220L; E7K+D18P+D39L+K64L+D156L+E186K+E188P+M189L; E7K+D18P+D39L+K55Y+K64L+E186K+E188P+M189L; E7K+D18P+D39L+D156L+E186K+E188P+M189L+K198L+M220L; E7K+D18P+D39L+K55Y+E186K+E188P+M189L+K198L+M220L; E7K+D18P+D39L+K64L+E186K+E188P+M189L+K198L+M220L; E7K+D18P+D39L+K55Y+D156L+E186K+E188P+M189L+M220L; E7K+D18P+D39L+K64L+D156L+E186K+E188P+M189L+M220L; E7K+D18P+D39L+K55Y+K64L+E186K+E188P+M189L+M220L; E7K+D18P+D39L+K55Y+H150R+D156L+E186K+E188P+M189L+K198L; E7K+D18P+D39L+K64L+D156L+E186K+E188P+M189L+K198L; E7K+D18P+D39L+K55Y+K64L+E186K+E188P+M189L+K198L; E7K+D18P+D39L+K55Y+D156L+E186K+E188P+M189L+K198L+M220L; E7K+D18P+D39L+K64L+D156L+E186K+E188P+M189L+K198L+M220L; E7K+D18P+D39L+K55Y+K64L+E186K+E188P+M189L+K198L+M220L; E7K+D18P+D39L+K55Y+K64L+D156L+E186K+E188P+M189L+M220L; E7K+D18P+D39L+K55Y+N59V+K64L+E186K+E188P+M189L+M220L; E7K+D18P+D39L+K55Y+N59S+K64L+E186K+E188P+M189L+M220L; E7K+D18P+D39L+K55Y+N59T+K64L+E186K+E188P+M189L+M220L; E7K+D18P+D39L+K55Y+K64L+V75I+E186K+E188P+M189L+M220L; E7K+D18P+D39L+K55Y+K64L+Q84E+E186K+E188P+M189L+M220L; E7K+D18P+D39L+K55Y+N59V+K64L+V75I+E186K+E188P+M189L+M220L; E7K+D18P+D39L+K55Y+N59S+K64L+V75I+E186K+E188P+M189L+M220L; E7K+D18P+D39L+K55Y+N59S+K64L+Q84E+E186K+E188P+M189L+M220L; E7K+D18P+D39L+K55Y+N59T+K64L+V75I+E186K+E188P+M189L+M220L; E7K+D18P+D39L+K55Y+N59T+K64L+Q84E+E186K+E188P+M189L+M220L; E7K+D18P+D39L+K55Y+K64L+V75I+Q84E+E186K+E188P+M189L+M220L; E7K+D18P+D39L+K55Y+N59V+K64L+V75I+Q84E+E186K+E188P+M189L+M220L; E7K+D18P+D39L+K55Y+N59S+K64L+V75I+Q84E+E186K+E188P+M189L+M220L; E7K+D18P+D39L+K55Y+N59T+K64L+V75I+Q84E+E186K+E188P+M189L+M220L).

In another aspect, the variant comprises or consists of a substitution at a position corresponding to position 19 of the polypeptide of SEQ ID NO: 1. In another aspect, the amino acid at a position corresponding to position 19 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro. In another aspect, the variant comprises or consists of the substitution E19P corresponding to the polypeptide of SEQ ID NO: 1. In another aspect, the variant comprises or consists of a substitution at a position corresponding to position 19 of the polypeptide of SEQ ID NO: 1, together with a substitution at one or more positions described herein, such as a substitution at one or more positions corresponding to positions 1, 3, 5, 6, 7, 8, 12, 17, 18, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., H5R+G6P+E19P; H5R+G6P+E19P+K151E).

In another aspect, the variant comprises or consists of a substitution at a position corresponding to position 20 of the polypeptide of SEQ ID NO: 1. In another aspect, the amino acid at a position corresponding to position 20 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe. In another aspect, the variant comprises or consists of the substitution Y20F corresponding to the polypeptide of SEQ ID NO: 1. In another aspect, the variant comprises or consists of a substitution at a position corresponding to position 20 of the polypeptide of SEQ ID NO: 1, together with a substitution at one or more positions described herein, such as a substitution at one or more positions corresponding to positions 1, 3, 5, 6, 7, 8, 12, 17, 18, 19, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+Y20F+D39L+E186K+E188P+M189L).

In another aspect, the variant comprises or consists of a substitution at a position corresponding to position 21 of the polypeptide of SEQ ID NO: 1. In another aspect, the amino acid at a position corresponding to position 21 is substituted with Ala, Arg, Asn, Asp, Cys, Gln, Glu, Gly, His, Ile, Leu, Lys, Met, Phe, Pro, Ser, Thr, Trp, Tyr, or Val I, preferably with Ala. In another aspect, the variant comprises or consists of the substitution 121A corresponding to the polypeptide of SEQ ID NO: 1. In another aspect, the variant comprises or consists of a substitution at a position corresponding to position 21 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 22 of the polypeptide of SEQ ID NO: 1. In another aspect, the amino acid at a position corresponding to position 22 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro, Phe, Trp, Tyr or Asn. In another aspect, the variant comprises or consists of the substitution M22P, M22F, M22W, M22Y or M22N corresponding to the polypeptide of SEQ ID NO: 1. In another aspect, the variant comprises or consists of a substitution at a position corresponding to position 22 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., M22N+A89E+K151P+E188P+D206P).

In another aspect, the variant comprises or consists of a substitution at a position corresponding to position 25 of the polypeptide of SEQ ID NO: 1. In another aspect, the amino acid at a position corresponding to position 25 is substituted with Ala, Arg, Asn, Asp, Cys, Gln, Glu, Gly, His, Ile, Leu, Lys, Met, Phe, Pro, Ser, Thr, Trp, Tyr, or Val, preferably with Thr. In another aspect, the variant comprises or consists of the substitution 125T corresponding to the polypeptide of SEQ ID NO: 1. In another aspect, the variant comprises or consists of a substitution at a position corresponding to position 25 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 27 of the polypeptide of SEQ ID NO: 1. In another aspect, the amino acid at a position corresponding to position 27 is substituted with Ala, Arg, Asn, Asp, Cys, Gln, Glu, Gly, His, Ile, Leu, Lys, Met, Phe, Pro, Ser, Thr, Trp, Tyr, or Val, preferably with Thr or Tyr. In another aspect, the variant comprises or consists of the substitution K27T or K27Y corresponding to the polypeptide of SEQ ID NO: 1. In another aspect, the variant comprises or consists of a substitution at a position corresponding to position 27 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 28 of the polypeptide of SEQ ID NO: 1. In another aspect, the amino acid at a position corresponding to position 28 is substituted with Ala, Arg, Asn, Asp, Cys, Gln, Glu, Gly, His, Ile, Leu, Lys, Met, Phe, Pro, Ser, Thr, Trp, Tyr, or Val, preferably with Arg, Glu, Leu, Gln, Met, Ser, Tyr, Phe, Lys, Thr, Asp, Phe or His. In another aspect, the variant comprises or consists of the substitution N28R, N28E, N28L, N28Q, N28M, N28S, N28Y, N28F, N28K, N28T, N28F, N28D or N28H corresponding to the polypeptide of SEQ ID NO: 1. In another aspect, the variant comprises or consists of a substitution at a position corresponding to position 28 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 35 of the polypeptide of SEQ ID NO: 1. In another aspect, the amino acid at a position corresponding to position 35 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises or consists of the substitution N35D corresponding to the polypeptide of SEQ ID NO: 1. In another aspect, the variant comprises or consists of a substitution at a position corresponding to position 35 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., N35D+Y72F+V74E+K83E+Y100F+K111E+K123E+E132D+E140D+K151E+D163E++Y165F+E190D+E194D+V211T+L214I+E221K; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; N35D+Y72F+V74E+K83E+Y100F+K111E+K123E+E132D+E140D+K151E+R162L+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; H12L+N35D+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 36 of the polypeptide of SEQ ID NO: 1. In another aspect, the amino acid at a position corresponding to position 36 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp, Leu, Ser, Asn or Ile. In another aspect, the variant comprises or consists of the substitution R36D, R36L, R36S, R36N or R36I corresponding to the polypeptide of SEQ ID NO: 1. In another aspect, the variant comprises or consists of a substitution at a position corresponding to position 36 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., R36D+K41C; R36D+K43A; R36D+M218K; R36D+K41C+K43A; R36D+K41C+M218K; R36D+K43A+M218K; D18P+R36D+K43A+M218K; R36D+K41C+K43A+M218K; D18P+R36D+K41C+K43A+M218K; R36D+D39K+K43A+D161N+E186K+E188P+M218K+E221K+K222*; R36D+D39K+K41C+K43A+D161N+E186K+E188P+M218K+E221K+K222*; D18P+R36D+D39K+K41C+K43A+D161N+E186K+E188P+M218K+E221K+K222*; R36L+Y72F+V74E+K83E+Y100F+K111E+K123E+E132D+E140D+K151E+D163E+Y165F+E190D+E194D+V211T+L214I+M218I+E221R; R36L+Y72F+V74E+K83E+Y100F+K111E+K123E+E132D+E140D+K151E+D163E+Y165F+E190D+E194D+V211T+L214I+M218L+E221R; R36I+Y72F+V74E+K83E+Y100F+K111E+K123E+E132D+E140D+K151E+D163E+Y165F+E190D+E194D+V211T+L214I+M218L+E221R; R36I+Y72F+V74E+K83E+Y100F+K111E+K123E+G125A+E132D+E140D+A149P+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of an insertion or one or more amino acids at a position corresponding to position 36 of the polypeptide of SEQ ID NO: 1. In another aspect, the inserted amino acid is selected from Ala, Arg, Asn, Asp, Cys, Gln, Glu, Gly, His, Ile, Leu, Lys, Met, Phe, Pro, Ser, Thr, Trp, Tyr, and Val, preferably Glu, Asn, Ala, Val, Trp, Phe or Asp. In another aspect, the variant comprises or consists of the insertion R36RE, R36RN, R36RA, R36RV, R36RW, R36RF, or R36RD corresponding to the polypeptide of SEQ ID NO: 1. In another aspect, the variant comprises or consists of the insertion of two amino acids at a position corresponding to position 36 of the polypeptide of SEQ ID NO: 1 (e.g., selected from Ala, Arg, Asn, Asp, Cys, Gln, Glu, Gly, His, Ile, Leu, Lys, Met, Phe, Pro, Ser, Thr, Trp, Tyr, and Val, preferably Glu, Asn, Ala, Val, Trp, Phe, Lys or Asp). In another aspect, the variant comprises or consists of the insertion R36REN, R36RAY, R36RAV, R36RAW, R36RAF, R36REK, R36RKN or R36RAD corresponding to the polypeptide of SEQ ID NO: 1. In another aspect, the variant comprises or consists of an insertion or one or more amino acids at a position corresponding to position 36 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; and/or a deletion at a position corresponding to position 222 of the polypeptide of SEQ ID NO: 1 (e.g., E7K+D18P+R36REN+D39L+K55Y+N59S+K64L+V75I+Q84E+E186K+E188P+M189L+A216G+M218Y+M220L+E221K; E7K+D18P+R36REN+D39L+K55Y+N59S+K64L+V75I+Q84E+E186K+E188P+M189L+A216G+R217N+M218Y+M220L+E221V; E7K+D18P+R36REN+D39L+K55Y+N59S+K64L+V75I+Q84E+E186K+E188P+M189L+A216G+R217N+M218Y+M220L+E221T; E7K+D18P+R36REN+D39L+K55Y+N59S+K64L+V75I+Q84E+E186K+E188P+M189L+A216G+R217N+M218Y+M220L; E7K+D18P+R36REN+D39L+K55Y+N59S+K64L+V75I+Q84E+E186K+E188P+M189L+A216G+R217N+M218A+M220L+E221V; E7K+D18P+R36REN+D39L+K55Y+N59S+K64L+V75I+Q84E+E186K+E188P+M189L+A216G+R217N+M218T+M220L+E221K; E7K+D18P+R36RAY+D39L+K55Y+N59S+K64L+V75I+Q84E+E186K+E188P+M189L+M220L; E7K+D18P+R36RAV+D39L+K55Y+N59S+K64L+V75I+Q84E+E186K+E188P+M189L+M220L; E7K+D18P+R36REN+D39L+K41C+K55Y+N59S+K64L+V75I+Q84E+E186K+E188P+M189L+M220L; E7K+D18P+R36REN+D39L+K55Y+N59S+K64L+V75I+Q84E+E186K+E188P+M189L+A216G+M218A+M220L+E221V; E7K+D18P+R36REN+D39L+K55Y+N59S+K64L+V75I+Q84E+E186K+E188P+M189L+A216G+R217N+M218Y+M220L+E221K; E7K+D18P+R36REN+D39L+K55Y+N59S+K64L+V75I+Q84E+E186K+E188P+M189L+A216G+R217N+M218T+M220L+E221T; E7K+D18P+R36REN+D39L+K55Y+N59S+K64L+V75I+Q84E+R181I+E186K+E188P+M189L+M220L; E7K+D18P+R36RAW+D39L+K55Y+N59S+K64L+V75I+Q84E+E186K+E188P+M189L+M220L; E7K+D18P+R36REN+D39L+K55Y+N59S+K64L+V75I+D76N+Q84E+L95F+E186K+E188P+M189L+M220L; E7K+D18P+R36REN+D39L+K55Y+N59S+K64L+V75I+Q84E+R166A+R181L+E186K+E188P+M189L+M220L; D18P+R36REN+D39L+K64L+V75I+D76N+L95F+E186K+E188P; D18P+R36RAW+D39L+K64L+V75I+E186K+E188P; E7K+D18P+R36REN+D39L+K55Y+N59S+K64L+V75I+D76N+I78V+Q84E+L95V+E186K+E188P+M189L+M220L; E7K+D18P+R36REN+D39L+K55Y+N59S+K64L+V75I+I78V+Q84E+L95F+E186K+E188P+M189L+M220L; D18P+R36RAF+D39L+K64L+V75I+E186K+E188P; E7K+D18P+R36REN+D39L+K55Y+N59S+K64L+V75I+D76N+I78V+Q84E+L95I+E186K+E188P+M189L+M220L; E7K+D18P+R36REN+D39L+K55Y+N59S+K64L+V75I+Q84E+E186K+E188P+M189L+A216G+M218I+M220L+E221T; E7K+D18P+R36REN+D39L+K55Y+N59S+K64L+V75I+I78V+Q84E+L95V+E186K+E188P+M189L+M220L; D18P+R36REN+D39L+K64L+V75I+R181I+E186K+E188P; E7K+D18P+R36REN+D39L+K55Y+N59S+K64L+V75I+I78V+Q84E+L95I+E186K+E188P+M189L+M220L; D18P+R36RAY+D39L+K64L+V75I+E186K+E188P; D18P+R36REN+D39L+K64L+V75I+D76N+I78V+L95V+E186K+E188P; E7K+D18P+R36REN+D39L+K55Y+N59S+K64L+V75I+Q84E+R181L+E186K+E188P+M189L+M220L; D18P+R36REN+D39L+K64L+V75I+I78V+L95I+E186K+E188P; D18P+R36REN+D39L+K64L+V75I+E186K+E188P+A216G+M218I+E221T; E7K+D18P+R36REN+D39L+K55Y+N59S+K64L+V75I+Q84E+R166M+R181L+E186K+E188P+M189L+M220L; D18P+R36REN+D39L+K64L+V75I+D76N+I78V+L95I+E186K+E188P; D18P+R36REN+D39L+K64L+V75I+I78V+L95V+E186K+E188P; D18P+R36REN+D39L+K64L+V75I+I78V+L95F+E186K+E188P; D18P+R36REN+D39L+K64L+V75I+R181L+E186K+E188P; E7K+D18P+R36REN+D39L+K55Y+N59S+K64L+V75I+Q84E+E186K+E188P+M189L+A216G+M218Y+M220L+E221T; D18P+R36REN+D39L+K64L+V75I+R181T+E186K+E188P; E7K+D18P+R36REN+D39L+K55Y+N59S+K64L+V75I+Q84E+E186K+E188P+M189L+A216G+M218S+M220L+E221T; D18P+R36REN+D39L+K64L+V75I+E186K+E188P+A216G+M218Y+E221T; D18P+R36REN+D39L+K64L+V75I+R166Y+R181T+E186K+E188P; D18P+R36REN+D39L+K64L+V75I+E186K+E188P+A216G+M218A; D18P+R36REN+D39L+K64L+V75I+E186K+E188P+A216G+M218Y+E221K; D18P+R36REN+D39L+K64L+V75I+E186K+E188P+A216G+M218A+E221T; E7K+D18P+R36REN+D39L+K55Y+N59S+K64L+V75I+Q84E+E186K+E188P+M189L+A216G+M218A+M220L+E221T; D18P+R36REN+D39L+K64L+V75I+E186K+E188P+A216G+M218A+E221V; D18P+R36REN+D39L+K64L+V75I+E186K+E188P+A216G+M218A+E221K; E7K+D18P+R36REN+D39L+K55Y+N59S+K64L+V75I+Q84E+E186K+E188P+M189L+A216G+M218N+M220L+E221T; E7K+D18P+R36REN+D39L+K55Y+N59S+K64L+V75I+Q84E+R166M+R181T+E186K+E188P+M189L+M220L; E7K+D18P+R36REN+D39L+K55Y+N59S+K64L+V75I+Q84E+R166E+E186K+E188P+M189L+M220L; E7K+D18P+R36REN+D39L+K55Y+N59S+K64L+V75I+Q84E+E186K+E188P+M189L+A216G+M218A+M220L; E7K+D18P+R36REN+D39L+K55Y+N59S+K64L+V75I+Q84E+E186K+E188P+M189L+A216G+M218N+M220L; E7K+D18P+R36REN+D39L+K55Y+N59S+K64L+V75I+Q84E+R166K+R181T+E186K+E188P+M189L+M220L; D18P+R36REN+D39L+K64L+V75I+E186K+E188P+A216G+M218S; D18P+R36REN+D39L+K64L+V75I+E186K+E188P+A216G+M218N; E7K+D18P+R36REN+D39L+K55Y+N59S+K64L+V75I+Q84E+E186K+E188P+M189L+A216G+M218S+M220L; E7K+D18P+R36REN+D39L+K55Y+N59S+K64L+V75I+Q84E+R166Y+R181T+E186K+E188P+M189L+M220L; E7K+D18P+R36REN+D39L+K55Y+N59S+K64L+V75I+Q84E+E186K+E188P+M189L+A216G+M218A+M220L+E221K; D18P+R36REN+D39L+K64L+V75I+E186K+E188P+A216G+R217N+M218Y; E7K+D18P+R36REN+D39L+K55Y+N59S+K64L+V75I+Q84E+R181T+E186K+E188P+M189L+M220L; D18P+R36REN+D39L+K64L+V75I+E186K+E188P+A216N+E221T; D18P+R36REN+D39L+K64L+V75I+E186K+E188P+A216N+M218A+E221T; D18P+R36S+R36RAD+I37A+D39K+K41C+K43A+K64L+V75I+E186K+E188P; D18P+R36REN+D39L+K64L+V75I+E186K+E188P+A216N+M218Y+E221K; D18P+R36REN+D39L+K64L+V75I+E186K+E188P+A216N+M218A; E7K+D18P+R36REN+D39L+K55Y+N59S+K64L+V75I+Q84E+E186K+E188P+M189L+A216N+M218S+M220L+E221T; E7K+D18P+R36REN+D39L+K55Y+N59S+K64L+V75I+Q84E+E186K+E188P+M189L+A216G+R217N+M218V+M220L+E221K; E7K+D18P+R36REN+D39L+K55Y+N59S+K64L+V75I+Q84E+E186K+E188P+M189L+A216G+R217N+M218L+M220L; E7K+D18P+R36REN+D39L+K55Y+N59S+K64L+V75I+Q84E+E186K+E188P+M189L+A216G+M218N+M220L+E221K; E7K+D18P+R36REK+D39L+K55Y+N59S+K64L+V75I+Q84E+R181T+E186K+E188P+M189L+M220L; D18P+R36REN+D39L+K41C+K64L+V75I+E186K+E188P; D18P+R36REN+D39L+K64L+V75I+E186K+E188P+A216G+M218N+E221K; D18P+R36REN+D39L+K64L+V75I+E186K+E188P+A216N+M218G+E221V; D18P+R36REN+D39L+K64L+V75I+E186K+E188P+A216G+M218G; E7K+D18P+R36RKN+D39L+K55Y+N59S+K64L+V75I+Q84E+R181T+E186K+E188P+M189L+M220L; E7K+D18P+R36REN+D39L+K55Y+N59S+K64L+V75I+Q84E+E186K+E188P+M189L+A216N+M220L+E221T; D18P+R36REN+D39L+K64L+V75I+E186K+E188P+A216G+R217N+M218L; E7K+D18P+R36REN+D39L+K55Y+N59S+K64L+V75I+Q84E+E186K+E188P+M189L+A216G+M218G+M220L+E221R; E7K+D18P+R36REN+D39L+K55Y+N59S+K64L+V75I+Q84E+E186K+E188P+M189L+A216N+M218A+M220L+E221T; E7K+D18P+R36REN+D39L+K55Y+N59S+K64L+V75I+Q84E+E186K+E188P+M189L+A216G+M218G+M220L+E221T; E7K+D18P+R36REN+D39L+K55Y+N59S+K64L+V75I+Q84E+E186K+E188P+M189L+A216N+M218Y+M220L+E221K; E7K+D18P+R36REN+D39L+K55Y+N59S+K64L+V75I+Q84E+E186K+E188P+M189L+A216N+R217N+M218Y+M220L; E7K+D18P+R36REN+D39L+K55Y+N59S+K64L+V75I+Q84E+E186K+E188P+M189L+A216G+M218G+M220L+E221V; E7K+D18P+R36S+R36RAD+I37A+D39K+K41C+K43A+K55Y+N59S+K64L+V75I+Q84E+E186K+E188P+M189L+M220L; E7K+D18P+R36REN+D39L+K55Y+N59S+K64L+V75I+Q84E+E186K+E188P+M189L+A216N+M218G+M220L+E221V; E7K+D18P+R36REN+D39L+K55Y+N59S+K64L+V75I+Q84E+E186K+E188P+M189L+A216N+R217N+M218Y+M220L+E221K; E7K+D18P+R36REN+D39L+K55Y+N59S+K64L+V75I+Q84E+E186K+E188P+M189L+A216N+M218A+M220L; E7K+D18P+R36REN+D39L+K55Y+N59S+K64L+V75I+Q84E+E186K+E188P+M189L+A216G+M218G+M220L+E221K; E7K+D18P+R36REN+D39L+K55Y+N59S+K64L+V75I+Q84E+E186K+E188P+M189L+A216G+R217N+M218F+M220L+E221V).

In another aspect, the variant comprises or consists of a substitution at a position corresponding to position 37 of the polypeptide of SEQ ID NO: 1. In another aspect, the amino acid at a position corresponding to position 37 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala or Val. In another aspect, the variant comprises or consists of the substitution I37A or I37V corresponding to the polypeptide of SEQ ID NO: 1. In another aspect, the variant comprises or consists of a substitution at a position corresponding to position 37 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+I37V+D39L+E186K+E188P+M189L).

In another aspect, the variant comprises or consists of a substitution at a position corresponding to position 38 of the polypeptide of SEQ ID NO: 1. In another aspect, the amino acid at a position corresponding to position 38 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys. In another aspect, the variant comprises or consists of the substitution V38C corresponding to the polypeptide of SEQ ID NO: 1. In another aspect, the variant comprises or consists of a substitution at a position corresponding to position 38 of the polypeptide of SEQ ID NO: 1, together with a substitution at one or more positions described herein, such as a substitution at one or more positions corresponding to positions 11, 3, 5, 6, 7, 8, 12, 17, 18, 19, 20, 21, 22, 25, 27, 28, 35, 36, 37, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., V38C+A40C).

In another aspect, the variant comprises or consists of a substitution at a position corresponding to position 39 of the polypeptide of SEQ ID NO: 1. In another aspect, the amino acid at a position corresponding to position 39 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala, Cys, Glu, Phe, Gly, Lys, Leu, Met, Asn, Pro, Arg, Ser, Trp or Tyr. In another aspect, the variant comprises or consists of the substitution D39A, D39C, D39E, D39F, D39G, D39K, D39L, D39M, D39N, D39P, D39R, D39S, D39W or D39Y corresponding to the polypeptide of SEQ ID NO: 1. In another aspect, the variant comprises or consists of a deletion at a position corresponding to position 39 of the polypeptide of SEQ ID NO: 1 (e.g., D39*). In another aspect, the variant comprises or consists of a substitution or deletion at a position corresponding to position 39 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., 39L+E48R; D39L+R50P; D39L+S66G; D39L+D76L; D39L+S91H; D39L+E132I; D39L+E140A; D39L+W182K; D39L+E190G; D39L+E221I; D39L+E197V; D39K+K222*; D18K+D39R+K64L; D18E+D39R+K147I; D39K+K41C+K222*; D39R+K64L+E221L; D39R+K147I+M191V; D39R+E186R+M191V; D39L+E186K+M191V; D18E+D39L+K64L+E186L; D18K+D39L+H99L+E186L; D18K+D39L+H99L+M218V; D18K+D39L+N141K+K147I; D18K+D39L+K147I+E186R; D18E+D39R+S177Y+E186R; D18K+D39L+E186K+M189L; D18E+D39L+E186L+M191V; D18K+D39R+E186R+E221R; D18E+D39L+M189L+M191V; D39R+A53G+K64L+E186R; D39L+A53G+N141K+S177Y; D39L+A53G+E186R+E221R; D39L+A53G+M189L+E221L; D39L+K64L+N141K+D163K; D39R+K64L+S177Y+M218V; D39L+K64L+M189I+M191V; D39R+H99L+D163K+E186R; D39L+H99L+E186K+M218V; D39R+N141K+E186R+M189I; D39L+N141K+E186R+M189I; D39L+N141K+E186L+M218V; D39K+D161N+E188P+K222*; D39L+S177Y+E186R+E221R; D18K+D39R+E186R+M218V; D39R+K64L+K147I+E186R; D39L+D163K+S177Y+E186R; D39R+K147I+E186R+E221R; D18K+D39L+E186R+M189L; D18E+D39R+D163K+E186R; D39L+H99L+E186R+M189I; D39L+A53G+E186R+M189L; D39L+K147I+E186L+E221R; D18K+D39L+E186K+E221R; D39L+K147I+D163K+E186K; D18K+D39L+N141K+E221L; D18E+D39L+N141K+E186K; D39L+A53G+E186R+M189I; D39L+H99L+D163K+E221L; D39L+D163K+E186K+E221L; D39L+D163K+E186R+M191V; D39L+D163K+E186R+E221L; D39K+E186K+E221K+K222*; D18K+D39L+E186R+M218V; D18K+D39L+S177Y+M189I; D39L+K64L+E186L+M189L; D39L+S177Y+E186K+M191V; D18K+D39L+M189L+M191V; D39L+H99L+K147I+M189L; D39L+K147I+E186R+M218V; D39R+E186R+M189I+M191V; D39L+K64L+N141K+E221R; D39L+H99L+E186R+E221R; D39L+E186R+M191V+E221L; D39L+S177Y+E186K+E221R; D39L+D163K+E186R+M218V; D39L+K64L+S177Y+E186R; D39R+K64L+S177Y+E186R; D39R+K64L+S177Y+E221R; D39R+K64L+S177Y+E186K; D39R+E186R+M189L+E221R; D39L+D163K+E186L+M189I; D18K+D39L+A53G+E186L; D39L+H99L+N141K+E221R; D39L+K147I+E186L+E221L; D39L+H99L+E186K+E221R; D39L+N141K+E186R+M191V; D39R+E186R+M191V+M218V; D18K+D39L+K147I+E186R; D18E+D39L+D163K+E221R; D39L+E186R+M191V+E221R; D39L+K64L+H99L+E186R; D39L+K64L+K147I+M189L; D39L+K64L+D163K+M191V; D18K+D39L+S177Y+E186R; D39R+A53G+H99L+E186R; D18E+D39L+S177Y+E186R; D18K+D39L+M189I+E221R; D39L+K64L+M218V+E221L; D39L+K147I+S177Y+E221L; D39L+A53G+K64L+H99L; D18K+D39L+D163K+M218V; D39L+K147I+E186R+M189I; D18E+D39L+E186R+M189L; D39R+H99L+E186R+M191V; D18P+D39L+H99L+E186L+E188P; D18P+D39L+N141K+E186R+E188P; D18P+D39R+E186R+E188P+E221L; D18P+D39L+E186K+E188P+M189L; D18P+D39L+E188P+M189L+E221R; D18P+D39R+S177Y+E186R+E188P; D18P+D39L+K147I+E188P+M191V; D18P+D39L+K64L+E186R+E188P; D18P+D39L+A53G+E186R+E188P; D18P+D39L+H99L+E188P+M189I; D18P+D39L+E186K+E188P+M189L; D39K+K41C+E186K+E221K+K222*; D18P+D39L+E186L+E188P+M218V; D39K+K41C+D161N+E188P+K222*; D18P+D39L+K64L+E186K+E188P; D18P+D39L+S177Y+E188P+M191V; D18P+D39L+E186K+E188P+M189L; D39L+D163K+E186R+M191V+M218V+E221R; D39K+D161N+E186K+E188P+E221K+K222*; D39L+D163K+E186K+M191V+M218V+E221R; D39K+K41C+D161N+E186K+E188P+E221K+K222*; D18P+D39K+D161N+E186K+E188P+E221K+K222*; D18P+D39K+K41C+D161N+E186K+E188P+E221K+K222*; D39K+K43A+D161N+E186K+E188P+M218K+E221K+K222*; D18P+D39K+K43A+D161N+E186K+E188P+M218K+E221K+K222*; R36D+D39K+K43A+D161N+E186K+E188P+M218K+E221K+K222*; D39K+K41C+K43A+D161N+E186K+E188P+M218K+E221K+K222*; R36D+D39K+K41C+K43A+D161N+E186K+E188P+M218K+E221K+K222*; D18P+D39K+K41C+K43A+D161N+E186K+E188P+M218K+E221K+K222*; D18P+R36D+D39K+K41C+K43A+D161N+E186K+E188P+M218K+E221K+K222*). In another aspect, the variant comprises or consists of a deletion or substitution at a position corresponding to position 39 of the polypeptide of SEQ ID NO: 1 (e.g., D39*, D39A, D39C, D39E, D39F, D39G, D39K, D39L, D39M, D39N, D39P, D39R, D39S, D39W or D39Y), together with a substitution at position 186 of the polypeptide of SEQ ID NO: 1 (e.g., E186K, E186L or E186R). In another aspect, the variant comprises or consists of D39*+E186K, D39*+E186L, or D39*+E186R. In another aspect, the variant comprises or consists of D39A+E186K, D39A+E186L, or D39A+E186R. In another aspect, the variant comprises or consists of D39C+E186K, D39C+E186L, or D39C+E186R. In another aspect, the variant comprises or consists of D39E+E186K, D39E+E186L, or D39E+E186R. In another aspect, the variant comprises or consists of D39F+E186K, D39F+E186L or D39F+E186R. In another aspect, the variant comprises or consists of D39G+E186K, D39G+E186L, or D39G+E186R. In another aspect, the variant comprises or consists of D39K+E186K, D39K+E186L, or D39K+E186R. In another aspect, the variant comprises or consists of D39L+E186K, D39L+E186L, or D39L+E186R. In another aspect, the variant comprises or consists of D39M+186K, D39M+E186L, or D39M+E186R. In another aspect, the variant comprises or consists of D39N+E186K, D39N+E186L, or D39N+E186R. In another aspect, the variant comprises or consists of D39P+E186K, D39P+E186L, or D39P+E186R. In another aspect, the variant comprises or consists of D39R+E186K, D39R+E186L, or D39R+E186R. In another aspect, the variant comprises or consists of D39S+E186K, D39S+E186L, or D39S+E186R. In another aspect, the variant comprises or consists of D39W+E186K, D39W+E186L, or D39W+E186R. In another aspect, the variant comprises or consists of D39Y+E186K, D39Y+E186L, or D39Y+E186R.

In another aspect, the variant comprises or consists of a substitution at a position corresponding to position 40 of the polypeptide of SEQ ID NO: 1. In another aspect, the amino acid at a position corresponding to position 40 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys. In another aspect, the variant comprises or consists of the substitution A40C corresponding to the polypeptide of SEQ ID NO: 1. In another aspect, the variant comprises or consists of a substitution at a position corresponding to position 40 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., V38C+A40C).

In another aspect, the variant comprises or consists of a substitution at a position corresponding to position 41 of the polypeptide of SEQ ID NO: 1. In another aspect, the amino acid at a position corresponding to position 41 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys, Ala, Met, Asn, Ser, Asp, Leu or Gly. In another aspect, the variant comprises or consists of the substitution K41C, K41A, K41M, K41N, K41S, K41D, K41L or K41G corresponding to the polypeptide of SEQ ID NO: 1. In another aspect, the variant comprises or consists of a substitution at a position corresponding to position 41 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+K41C; R36D+K41C; K41C+K43A; K41C+M218K; R36D+K41C+K43A; R36D+K41C+M218K; D39K+K41C+K222*; K41C+K43A+M218K; K41C+D161N+E188P; K41C+E186K+E221K; D18P+K41C+K43A+M218K; R36D+K41C+K43A+M218K; D18P+R36D+K41C+K43A M218K; K41C+D161N+E186K+E188P E221K; D39K+K41C+E186K+E221K K222*; D39K+K41C+D161N+E188P K222*; D39K+K41C+D161N+E186K+E188P+E221K+K222*; D18P+D39K+K41C+D161N+E186K+E188P+E221K+K222*; D39K+K41C+K43A+D161N+E186K+E188P+M218K+E221K+K222*; D18P+D39K+K41C+K43A+D161N+E186K+E188P+M218K+E221K+K222*; R36D+D39K+K41C+K43A+D161N+E186K+E188P+M218K+E221K+K222*; D18P+R36D+D39K+K41C+K43A+D161N+E186K+E188P+M218K+E221K+K222*).

In another aspect, the variant comprises or consists of a substitution at a position corresponding to position 43 of the polypeptide of SEQ ID NO: 1. In another aspect, the amino acid at a position corresponding to position 43 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala, His, Phe, Pro or Tyr. In another aspect, the variant comprises or consists of the substitution K43A, K43H, K43F, K43P or K43Y corresponding to the polypeptide of SEQ ID NO: 1. In another aspect, the variant comprises or consists of a substitution at a position corresponding to position 43 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., R36D+K43A; K41C+K43A; K43A+M218K; D18P+K43A+M218K; R36D+K41C+K43A; R36D+K43A+M218K; K41C+K43A+M218K; D18P+R36D+K43A+M218K; R36D+K41C+K43A+M218K; D18P+K41C+K43A+M218K; D18P+R36D+K41C+K43A+M218K; D39K+K43A+D161N+E186K+E188P+M218K+E221K+K222*; D18P+D39K+K43A+D161N+E186K+E188P+M218K+E221K+K222*; R36D+D39K+K43A+D161N+E186K+E188P+M218K+E221K+K222*; D39K+K41C+K43A+D161N+E186K+E188P+M218K+E221K+K222*; R36D+D39K+K41C+K43A+D161N+E186K+E188P+M218K+E221K+K222*; D18P+D39K+K41C+K43A+D161N+E186K+E188P+M218K+E221K+K222*).

In another aspect, the variant comprises or consists of a substitution at a position corresponding to position 46 of the polypeptide of SEQ ID NO: 1. In another aspect, the amino acid at a position corresponding to position 46 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another aspect, the variant comprises or consists of the substitution K46Y corresponding to the polypeptide of SEQ ID NO: 1. In another aspect, the variant comprises or consists of a substitution at a position corresponding to position 46 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 48 of the polypeptide of SEQ ID NO: 1. In another aspect, the amino acid at a position corresponding to position 48 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp, Ser, Asn, Tyr, Val, Phe or Arg. In another aspect, the variant comprises or consists of the substitution E48D, E48S, E48N, E48Y, E48V, E48F or E48R corresponding to the polypeptide of SEQ ID NO: 1. In another aspect, the variant comprises or consists of a substitution at a position corresponding to position 48 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+E48R).

In another aspect, the variant comprises or consists of a substitution at a position corresponding to position 49 of the polypeptide of SEQ ID NO: 1. In another aspect, the amino acid at a position corresponding to position 49 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys. In another aspect, the variant comprises or consists of the substitution Y49C corresponding to the polypeptide of SEQ ID NO: 1. In another aspect, the variant comprises or consists of a substitution at a position corresponding to position 49 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y49C+S71C).

In another aspect, the variant comprises or consists of a substitution at a position corresponding to position 50 of the polypeptide of SEQ ID NO: 1. In another aspect, the amino acid at a position corresponding to position 50 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys, Tyr or Pro. In another aspect, the variant comprises or consists of the substitution R50K, R50Y or R50P corresponding to the polypeptide of SEQ ID NO: 1. In another aspect, the variant comprises or consists of a substitution at a position corresponding to position 50 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+R50P).

In another aspect, the variant comprises or consists of a substitution at a position corresponding to position 51 of the polypeptide of SEQ ID NO: 1. In another aspect, the amino acid at a position corresponding to position 51 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro, Ser, Thr or Cys. In another aspect, the variant comprises or consists of the substitution A51P, A51S, A51T or A51C corresponding to the polypeptide of SEQ ID NO: 1. In another aspect, the variant comprises or consists of a substitution at a position corresponding to position 51 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+A51P+E186K+E188P+M189L; D18P+D39L+A51S+E186K+E188P+M189L; A51T+Y72F+V74E+K83E+Y100F+K111E+K123E+E132D+E140D+K151E+D163E+Y165F+E190D+E194D+V211T+L214I+E221R; A51C+K151C; A51C+E68C; A51C+Y72C+K151C).

In another aspect, the variant comprises or consists of a substitution at a position corresponding to position 52 of the polypeptide of SEQ ID NO: 1. In another aspect, the amino acid at a position corresponding to position 52 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val or Cys. In another aspect, the variant comprises or consists of the substitution G52V or G52C corresponding to the polypeptide of SEQ ID NO: 1. In another aspect, the variant comprises or consists of a substitution at a position corresponding to position 52 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+G52V+E186K+E188P+M189L; G52C+I152C).

In another aspect, the variant comprises or consists of a substitution at a position corresponding to position 53 of the polypeptide of SEQ ID NO: 1. In another aspect, the amino acid at a position corresponding to position 53 is substituted with Ala, Arg, Asn, Asp, Cys, Gln, Glu, Gly, His, Ile, Leu, Lys, Met, Phe, Pro, Ser, Thr, Trp, Tyr, or Val, preferably with Gly or Pro. In another aspect, the variant comprises or consists of the substitution A53G or A53P corresponding to the polypeptide of SEQ ID NO: 1. In another aspect, the variant comprises or consists of a substitution at a position corresponding to position 53 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+A53P; A53G+K147I+E186K; D39R+A53G+K64L+E186R; D39L+A53G+N141K+S177Y; D39L+A53G+E186R+E221R; D39L+A53G+M189L+E221L; A53G+K147I+E186R+M218V; A53G+E186R+M189L+E221R; D39L+A53G+E186R+M189I; D39L+A53G+E186R+M189L; A53G+D163K+E186R+E221R; A53G+N141K+E186R+M191V; D18E+A53G+K64L+E186R; D18K+D39L+A53G+E186L; A53G+N141K+E186R+E221L; D39R+A53G+H99L+E186R; D18E+A53G+D163K+E186R; D39L+A53G+K64L+H99L; A53G+H99L+S177Y+E186R; A53G+E186R+M189I+M218V; D18K+A53G+K147I+E186R; D18P+D39L+A53G+E186R+E188P; D18P+A53G+E186R+E188P+E221L).

In another aspect, the variant comprises or consists of a substitution at a position corresponding to position 54 of the polypeptide of SEQ ID NO: 1. In another aspect, the amino acid at a position corresponding to position 54 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys or Ala. In another aspect, the variant comprises or consists of the substitution T54K or T54A corresponding to the polypeptide of SEQ ID NO: 1. In another aspect, the variant comprises or consists of a substitution at a position corresponding to position 54 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+T54K+E186K+E188P+M189L; D18P+D39L+T54A+E186K+E188P+M189L).

In another aspect, the variant comprises or consists of a substitution at a position corresponding to position 55 of the polypeptide of SEQ ID NO: 1. In another aspect, the amino acid at a position corresponding to position 55 is substituted with Ala, Arg, Asn, Asp, Cys, Gln, Glu, Gly, His, Ile, Leu, Lys, Met, Phe, Pro, Ser, Thr, Trp, Tyr, or Val, preferably with His, Tyr or Phe. In another aspect, the variant comprises or consists of the substitution K55H, K55Y or K55F corresponding to the polypeptide of SEQ ID NO: 1. In another aspect, the variant comprises or consists of a substitution at a position corresponding to position 55 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 57 of the polypeptide of SEQ ID NO: 1. In another aspect, the amino acid at a position corresponding to position 57 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val. In another aspect, the variant comprises or consists of the substitution L57V corresponding to the polypeptide of SEQ ID NO: 1. In another aspect, the variant comprises or consists of a substitution at a position corresponding to position 57 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+L57V+E186K+E188P+M189L).

In another aspect, the variant comprises or consists of a substitution at a position corresponding to position 58 of the polypeptide of SEQ ID NO: 1. In another aspect, the amino acid at a position corresponding to position 58 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises or consists of the substitution N58D corresponding to the polypeptide of SEQ ID NO: 1. In another aspect, the variant comprises or consists of a substitution at a position corresponding to position 58 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., N58D+S177M).

In another aspect, the variant comprises or consists of a substitution at a position corresponding to position 59 of the polypeptide of SEQ ID NO: 1. In another aspect, the amino acid at a position corresponding to position 59 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp, Trp, Ile, Arg, His, Ser, Thr, Val or Leu. In another aspect, the variant comprises or consists of the substitution N59D, N59S, N59R, N59H, N59I, N59T, N59V, N59W or N59L corresponding to the polypeptide of SEQ ID NO: 1. In another aspect, the variant comprises or consists of a substitution at a position corresponding to position 59 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 60 of the polypeptide of SEQ ID NO: 1. In another aspect, the amino acid at a position corresponding to position 60 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises or consists of the substitution G60D corresponding to the polypeptide of SEQ ID NO: 1. In another aspect, the variant comprises or consists of a substitution at a position corresponding to position 60 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 61 of the polypeptide of SEQ ID NO: 1. In another aspect, the amino acid at a position corresponding to position 61 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr, Trp or Phe. In another aspect, the variant comprises or consists of the substitution H61Y, H61W or H61F corresponding to the polypeptide of SEQ ID NO: 1. In another aspect, the variant comprises or consists of a substitution at a position corresponding to position 61 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 62 of the polypeptide of SEQ ID NO: 1. In another aspect, the amino acid at a position corresponding to position 62 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asn, Tyr, Trp, Phe or Met. In another aspect, the variant comprises or consists of the substitution T62N, T62Y, T62W, T62F or T62M corresponding to the polypeptide of SEQ ID NO: 1. In another aspect, the variant comprises or consists of a substitution at a position corresponding to position 62 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., T62M+V117F).

In another aspect, the variant comprises or consists of a substitution at a position corresponding to position 64 of the polypeptide of SEQ ID NO: 1. In another aspect, the amino acid at a position corresponding to position 64 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile, Gln, or Leu. In another aspect, the variant comprises or consists of the substitution K64I, K64Q or K64L corresponding to the polypeptide of SEQ ID NO: 1. In another aspect, the variant comprises or consists of a substitution at a position corresponding to position 64 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., K64L+E186R; D18K+D39R K64L; D39R+K64L E221L; D18K+K64L+E186R+M191V; D39R+A53G+K64L+E186R; D39L+K64L+N141K+D163K; D39R+K64L+S177Y+M218V; K64L+H99L+N141K+E186R; K64L+N141K+E186R+M189L; K64L+D163K+E186R+M189L; K64L+S177Y+E186R+E221L; K64L+S177Y+M218V+E221L; D39L+K64L+H99L+E186R; D18K+K64L+S177Y+E186R; D39R+K64L+K147I+E186R; D39L+K64L+E186L+M189L; K64L+H99L+E186R+M218V; D39L+K64L+N141K+E221R; D18E+A53G+K64L+E186R; D39L+K64L+S177Y+E186R; D39R+K64L+S177Y+E186R; K64L+S177Y+E186R+M218V; K64L+S177Y+E186K+M189L; D39R+K64L+S177Y+E221R; K64L+S177Y+E186K+E221R; D39R+K64L+S177Y+E186K; K64L+S177Y+E186R+E221R; D39L+K64L+K147I+M189L; D39L+K64L+D163K+M191V; D18K+K64L+D163K+E186R; D18E+K64L+E186R+M189I; K64L+K147I+E186R+M218V; D39L+K64L+M218V+E221L; D39L+A53G+K64L+H99L; D18P+D39L+K64L+E186K+E188P; D18P+D39L+K64L+E186R+E188P; D18P+K64L+E186R+E188P+E221R; D18P+D39L+K64L+V75I+E186K+E188P+M218V+M220L; D18P+D39L+K64L+V75I+Q84Y+E186K+E188P; D18P+D39L+K64L+V75I+Q84F+E186K+E188P; D18P+D39L+K64L+S66W+V75I+E186K+E188P; D18P+D39L+H61Y+K64L+V75I+E186K+E188P; D18P+D39L+K64L+S66F+V75I+E186K+E188P; D18P+D39L+K64L+V75I+E186K+E188P+E221V; D18P+D39L+K64L+S66V+V75I+E186K+E188P; D18P+D39L+K64L+V75I+S177W+E186K+E188P; D18P+D39L+K64L+V75I+T173W+E186K+E188P; D18P+D39L+H61F+K64L+V75I+E186K+E188P; D18P+M22F+N28D+D39L+K64L+V75I+E178T+E186K+E188P; D18P+M22F+N28S+D39L+K64L+V75I+E178P+E186K+E188P; D18P+D39L+T62W+K64L+V75I+E186K+E188P; D18P+D39L+K64L+V75I+G119S+E186K+E188P; D18P+D39L+K64L+V75I+G119A+E186K+E188P; D18P+R36N+D39L+K64L+V75I+S168A+R181T+E186K+E188P+M218I+M220L; D18P+D39L+K64L+V75I+R181I+E186K+E188P; D18P+D39L+E48V+K64L+V75I+E186K+E188P; D18P+D39L+K64L+V75I+G119I+E186K+E188P; D18P+D39L+E48Y+K64L+V75I+E186K+E188P; D18P+D39L+E48F+K64L+V75I+E186K+E188P; D18P+D39L+K64L+V75I+A104L+E186K+E188P; D18P+D39L+K64L+V75I+A149H+E186K+E188P; D18P+D39L+K43H+K64L+V75I+E186K+E188P; D18P+D39L+K64L+V75I+G119C+E186K+E188P; D18P+D39L+K64L+V75I+Q84W+E186K+E188P; D18P+D39L+K64L+V75I+A104V+E186K+E188P; D18P+D39L+K43Y+K64L+V75I+E186K+E188P; D18P+M22Y+N28F+D39L+K64L+V75I+E186K+E188P; D18P+N28Y+D39L+K64L+V75I+S168A+E186K+E188P; D18P+M22F+D39L+K64L+V75I+E178Q+E186K+E188P; D18P+D39L+K64L+V75I+H88Y+E186K+E188P; D18P+N28S+D39L+K64L+V75I+S168C+E186K+E188P+A216C; D18P+D39L+K43F+K64L+V75I+E186K+E188P; D18P+D39L+K64L+V75I+E178S+E186K+E188P; D18P+R36N+D39L+K64L+V75I+D76N+R181A+E186K+E188P; D18P+R36N+D39L+K64L+V75I+S168A+R181T+E186K+E188P+M218V+M220L).

In another aspect, the variant comprises or consists of a substitution at a position corresponding to position 66 of the polypeptide of SEQ ID NO: 1. In another aspect, the amino acid at a position corresponding to position 66 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu, Ile, Phe, His, Met, Val, Trp, Tyr, Gln or Gly. In another aspect, the variant comprises or consists of the substitution S66L, S66I, S66V, S66Q, S66Y, S66F, S66H, S66M, S66W or S66G corresponding to the polypeptide of SEQ ID NO: 1. In another aspect, the variant comprises or consists of a substitution at a position corresponding to position 66 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+S66G).

In another aspect, the variant comprises or consists of a substitution at a position corresponding to position 67 of the polypeptide of SEQ ID NO: 1. In another aspect, the amino acid at a position corresponding to position 67 is substituted with Ala, Arg, Asn, Asp, Cys, Gln, Glu, Gly, His, Ile, Leu, Lys, Met, Phe, Pro, Ser, Thr, Trp, Tyr, or Val, preferably with Met, Ile, Phe, Trp or Val. In another aspect, the variant comprises or consists of the substitution Y67M, Y67I, Y67F, Y67W or Y67V corresponding to the polypeptide of SEQ ID NO: 1. In another aspect, the variant comprises or consists of a substitution at a position corresponding to position 67 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 68 of the polypeptide of SEQ ID NO: 1. In another aspect, the amino acid at a position corresponding to position 68 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys, His, Asp, Arg or Thr. In another aspect, the variant comprises or consists of the substitution E68C, E68H, E68D, E68R or E68T corresponding to the polypeptide of SEQ ID NO: 1. In another aspect, the variant comprises or consists of a substitution at a position corresponding to position 68 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., A51C+E68C).

In another aspect, the variant comprises or consists of a substitution at a position corresponding to position 71 of the polypeptide of SEQ ID NO: 1. In another aspect, the amino acid at a position corresponding to position 71 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala or Cys. In another aspect, the variant comprises or consists of the substitution S71C or S71A corresponding to the polypeptide of SEQ ID NO: 1. In another aspect, the variant comprises or consists of a substitution at a position corresponding to position 71 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y49C+S71C).

In another aspect, the variant comprises or consists of a substitution at a position corresponding to position 72 of the polypeptide of SEQ ID NO: 1. In another aspect, the amino acid at a position corresponding to position 72 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe, Cys, Ser, Thr or Glu. In another aspect, the variant comprises or consists of the substitution Y72F, Y72C, Y72S, Y72T or Y72E corresponding to the polypeptide of SEQ ID NO: 1. In another aspect, the variant comprises or consists of a substitution at a position corresponding to position 72 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 74 of the polypeptide of SEQ ID NO: 1. In another aspect, the amino acid at a position corresponding to position 74 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu. In another aspect, the variant comprises or consists of the substitution V74E corresponding to the polypeptide of SEQ ID NO: 1. In another aspect, the variant comprises or consists of a substitution at a position corresponding to position 74 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 75 of the polypeptide of SEQ ID NO: 1. In another aspect, the amino acid at a position corresponding to position 75 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile or Leu. In another aspect, the variant comprises or consists of the substitution V75I or V75L corresponding to the polypeptide of SEQ ID NO: 1. In another aspect, the variant comprises or consists of a substitution at a position corresponding to position 75 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+V75I+E186K+E188P+M189L; V75I+E186K+E221K; V75L+E186K+E221K).

In another aspect, the variant comprises or consists of a substitution at a position corresponding to position 76 of the polypeptide of SEQ ID NO: 1. In another aspect, the amino acid at a position corresponding to position 76 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu, Asn or Lys. In another aspect, the variant comprises or consists of the substitution D76L, D76K or D76N corresponding to the polypeptide of SEQ ID NO: 1. In another aspect, the variant comprises or consists of a substitution at a position corresponding to position 76 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+D76L).

In another aspect, the variant comprises or consists of a substitution at a position corresponding to position 77 of the polypeptide of SEQ ID NO: 1. In another aspect, the amino acid at a position corresponding to position 77 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises or consists of the substitution G77D corresponding to the polypeptide of SEQ ID NO: 1. In another aspect, the variant comprises or consists of a substitution at a position corresponding to position 77 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 78 of the polypeptide of SEQ ID NO: 1. In another aspect, the amino acid at a position corresponding to position 78 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val, Glu or Lys. In another aspect, the variant comprises or consists of the substitution 178V, 178E or 178K corresponding to the polypeptide of SEQ ID NO: 1. In another aspect, the variant comprises or consists of a substitution at a position corresponding to position 78 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 79 of the polypeptide of SEQ ID NO: 1. In another aspect, the amino acid at a position corresponding to position 79 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile or Val. In another aspect, the variant comprises or consists of the substitution K791 or K79V corresponding to the polypeptide of SEQ ID NO: 1. In another aspect, the variant comprises or consists of a substitution at a position corresponding to position 79 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 80 of the polypeptide of SEQ ID NO: 1. In another aspect, the amino acid at a position corresponding to position 80 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another aspect, the variant comprises or consists of the substitution F80Y corresponding to the polypeptide of SEQ ID NO: 1. In another aspect, the variant comprises or consists of a substitution at a position corresponding to position 80 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+F80Y+E186K+E188P+M189L).

In another aspect, the variant comprises or consists of a substitution at a position corresponding to position 81 of the polypeptide of SEQ ID NO: 1. In another aspect, the amino acid at a position corresponding to position 81 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another aspect, the variant comprises or consists of the substitution E81Y corresponding to the polypeptide of SEQ ID NO: 1. In another aspect, the variant comprises or consists of a substitution at a position corresponding to position 81 of the polypeptide of SEQ ID NO: 1, together with a substitution at one or more positions described herein, such as a substitution at one or more positions corresponding to positions 11, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 82 of the polypeptide of SEQ ID NO: 1. In another aspect, the amino acid at a position corresponding to position 82 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe or Pro. In another aspect, the variant comprises or consists of the substitution L82P or L82F corresponding to the polypeptide of SEQ ID NO: 1. In another aspect, the variant comprises or consists of a substitution at a position corresponding to position 82 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 83 of the polypeptide of SEQ ID NO: 1. In another aspect, the amino acid at a position corresponding to position 83 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu or Ile. In another aspect, the variant comprises or consists of the substitution K83E or K83I corresponding to the polypeptide of SEQ ID NO: 1. In another aspect, the variant comprises or consists of a substitution at a position corresponding to position 83 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 84 of the polypeptide of SEQ ID NO: 1. In another aspect, the amino acid at a position corresponding to position 84 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe, Ala, Lys, Gly, Glu, Ile, Met, Leu, Trp or Tyr. In another aspect, the variant comprises or consists of the substitution Q84F, Q84A, Q84K, Q84G, Q84E, Q84M, Q84I, Q84L, Q84W or Q84Y corresponding to the polypeptide of SEQ ID NO: 1. In another aspect, the variant comprises or consists of a substitution at a position corresponding to position 84 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 85 of the polypeptide of SEQ ID NO: 1. In another aspect, the amino acid at a position corresponding to position 85 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys. In another aspect, the variant comprises or consists of the substitution F85C corresponding to the polypeptide of SEQ ID NO: 1. In another aspect, the variant comprises or consists of a substitution at a position corresponding to position 85 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., F85C+F106C).

In another aspect, the variant comprises or consists of a substitution at a position corresponding to position 86 of the polypeptide of SEQ ID NO: 1. In another aspect, the amino acid at a position corresponding to position 86 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile, Tyr or Trp. In another aspect, the variant comprises or consists of the substitution H86I, H86Y or H86W corresponding to the polypeptide of SEQ ID NO: 1. In another aspect, the variant comprises or consists of a substitution at a position corresponding to position 86 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 87 of the polypeptide of SEQ ID NO: 1. In another aspect, the amino acid at a position corresponding to position 87 is substituted with Ala, Arg, Asn, Asp, Cys, Gln, Glu, Gly, His, Ile, Leu, Lys, Met, Phe, Pro, Ser, Thr, Trp, Tyr, or Val, preferably with Trp or Leu. In another aspect, the variant comprises or consists of the substitution F87W or F87L corresponding to the polypeptide of SEQ ID NO: 1. In another aspect, the variant comprises or consists of a substitution at a position corresponding to position 87 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 88 of the polypeptide of SEQ ID NO: 1. In another aspect, the amino acid at a position corresponding to position 87 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another aspect, the variant comprises or consists of the substitution H88Y corresponding to the polypeptide of SEQ ID NO: 1. In another aspect, the variant comprises or consists of a substitution at a position corresponding to position 88 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 89 of the polypeptide of SEQ ID NO: 1. In another aspect, the amino acid at a position corresponding to position 89 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu, Tyr, Ser, Thr, Cys, Val or Phe. In another aspect, the variant comprises or consists of the substitution A89E, A89Y, A89S, A89T, A89C, A89V or A89F corresponding to the polypeptide of SEQ ID NO: 1. In another aspect, the variant comprises or consists of a substitution at a position corresponding to position 89 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., M22N+A89E+K151P+E188P+D206P; A89V+E186K+E221K; A89T+E186K+E221K; A89S+E186K+E221K).

In another aspect, the variant comprises or consists of a substitution at a position corresponding to position 91 of the polypeptide of SEQ ID NO: 1. In another aspect, the amino acid at a position corresponding to position 91 is substituted with Ala, Arg, Asn, Asp, Cys, Gln, Glu, Gly, His, Ile, Leu, Lys, Met, Phe, Pro, Ser, Thr, Trp, Tyr, or Val, preferably with His. In another aspect, the variant comprises or consists of the substitution S91H corresponding to the polypeptide of SEQ ID NO: 1. In another aspect, the variant comprises or consists of a substitution at a position corresponding to position 91 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+S91H).

In another aspect, the variant comprises or consists of a substitution at a position corresponding to position 94 of the polypeptide of SEQ ID NO: 1. In another aspect, the amino acid at a position corresponding to position 94 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu, Met, or Thr. In another aspect, the variant comprises or consists of the substitution K94L, K94M, or K94T corresponding to the polypeptide of SEQ ID NO: 1. In another aspect, the variant comprises or consists of a substitution at a position corresponding to position 94 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., K94T+H112N+M189V; K94T+H112N+M218K; K94T+M189V+M218K).

In another aspect, the variant comprises or consists of a substitution at a position corresponding to position 95 of the polypeptide of SEQ ID NO: 1. In another aspect, the amino acid at a position corresponding to position 95 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile, Val, Phe, or Tyr. In another aspect, the variant comprises or consists of the substitution L95I, L95V, L95F, or L95Y corresponding to the polypeptide of SEQ ID NO: 1. In another aspect, the variant comprises or consists of a substitution at a position corresponding to position 95 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., L95I+F102L+H112D; L95I+F102L+H112K; L95I+H112K+R126K; L95I+H112K+E188P; L95I+H112D+E188P).

In another aspect, the variant comprises or consists of a substitution at a position corresponding to position 96 of the polypeptide of SEQ ID NO: 1. In another aspect, the amino acid at a position corresponding to position 96 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asn or Asp. In another aspect, the variant comprises or consists of the substitution K96N or K96D corresponding to the polypeptide of SEQ ID NO: 1. In another aspect, the variant comprises or consists of a substitution at a position corresponding to position 96 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 98 of the polypeptide of SEQ ID NO: 1. In another aspect, the amino acid at a position corresponding to position 98 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys. In another aspect, the variant comprises or consists of the substitution Q98K corresponding to the polypeptide of SEQ ID NO: 1. In another aspect, the variant comprises or consists of a substitution at a position corresponding to position 98 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Q98K+F102L+H112K; Q98K+H112K+R126K; Q98K+H112D+E188P; Q98K+A142K+E186K; Q98K+E186K+M191V).

In another aspect, the variant comprises or consists of a substitution at a position corresponding to position 99 of the polypeptide of SEQ ID NO: 1. In another aspect, the amino acid at a position corresponding to position 99 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr, Pro or Leu. In another aspect, the variant comprises or consists of the substitution H99Y, H99P or H99L corresponding to the polypeptide of SEQ ID NO: 1. In another aspect, the variant comprises or consists of a substitution at a position corresponding to position 99 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18K+D39L+H99L+E186L; D18K+D39L+H99L+M218V; D18K+H99L+E186R+M189L; D39R+H99L+D163K+E186R; D39L+H99L+E186K+M218V; K64L+H99L+N141K+E186R; D18K+H99L+E186R+E221L; D39L+H99L+E186R+M189I; D39L+H99L+D163K+E221L; H99L+K147I+E186R+E221R; D39L+H99L+K147I+M189L; K64L+H99L+E186R+M218V; D39L+H99L+E186R+E221R; H99L+N141K+E186R+E221L; D39L+H99L+N141K+E221R; D39L+H99L+E186K+E221R; H99L+D163K+E186R+M189L; D39L+K64L+H99L+E186R; D39R+A53G+H99L+E186R; D39L+A53G+K64L+H99L; D39R+H99L+E186R+M191V; A53G+H99L+S177Y+E186R; D18E+H99L+E186R+M218V; D18P+D39L+H99L+E186L+E188P; D18P+H99L+K147I+E186R+E188P; D18P+D39L+H99L+E188P+M189I; D18P+H99L+S177Y+E186R+E188P).

In another aspect, the variant comprises or consists of a substitution at a position corresponding to position 100 of the polypeptide of SEQ ID NO: 1. In another aspect, the amino acid at a position corresponding to position 100 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe. In another aspect, the variant comprises or consists of the substitution Y100F corresponding to the polypeptide of SEQ ID NO: 1. In another aspect, the variant comprises or consists of a substitution at a position corresponding to position 100 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+E132D+E140D+K151E+D163E+Y165F E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221RX).

In another aspect, the variant comprises or consists of a substitution at a position corresponding to position 102 of the polypeptide of SEQ ID NO: 1. In another aspect, the amino acid at a position corresponding to position 102 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu or Met. In another aspect, the variant comprises or consists of the substitution F102L or F102M corresponding to the polypeptide of SEQ ID NO: 1. In another aspect, the variant comprises or consists of a substitution at a position corresponding to position 102 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+F102M; F102L+P129L; F102L+M220L; L95I+F102L+H112D; L95I+F102L+H112K; Q98K+F102L+H112K; F102L+H112K+E188P; F102M+R126K+M191V; F102L+E186K+M191V; F102M+M189I+M191V).

In another aspect, the variant comprises or consists of a substitution at a position corresponding to position 104 of the polypeptide of SEQ ID NO: 1. In another aspect, the amino acid at a position corresponding to position 104 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu, Cys, Met or Val. In another aspect, the variant comprises or consists of the substitution A104L, A104M, A104C or A104V corresponding to the polypeptide of SEQ ID NO: 1. In another aspect, the variant comprises or consists of a substitution at a position corresponding to position 104 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 105 of the polypeptide of SEQ ID NO: 1. In another aspect, the amino acid at a position corresponding to position 105 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe. In another aspect, the variant comprises or consists of the substitution H105F corresponding to the polypeptide of SEQ ID NO: 1. In another aspect, the variant comprises or consists of a substitution at a position corresponding to position 105 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 106 of the polypeptide of SEQ ID NO: 1. In another aspect, the amino acid at a position corresponding to position 106 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys. In another aspect, the variant comprises or consists of the substitution F106C corresponding to the polypeptide of SEQ ID NO: 1. In another aspect, the variant comprises or consists of a substitution at a position corresponding to position 106 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., F85C+F106C).

In another aspect, the variant comprises or consists of a substitution at a position corresponding to position 109 of the polypeptide of SEQ ID NO: 1. In another aspect, the amino acid at a position corresponding to position 109 is substituted with Ala, Arg, Asn, Asp, Cys, Gln, Glu, Gly, His, Ile, Leu, Lys, Met, Phe, Pro, Ser, Thr, Trp, Tyr, or Val, preferably with Gln. In another aspect, the variant comprises or consists of the substitution A109Q corresponding to the polypeptide of SEQ ID NO: 1. In another aspect, the variant comprises or consists of a substitution at a position corresponding to position 109 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+A109Q+E186K+E188P+M189L).

In another aspect, the variant comprises or consists of a substitution at a position corresponding to position 110 of the polypeptide of SEQ ID NO: 1. In another aspect, the amino acid at a position corresponding to position 110 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser. In another aspect, the variant comprises or consists of the substitution D110S corresponding to the polypeptide of SEQ ID NO: 1. In another aspect, the variant comprises or consists of a substitution at a position corresponding to position 110 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D110S+N114P+R166Y).

In another aspect, the variant comprises or consists of a substitution at a position corresponding to position 111 of the polypeptide of SEQ ID NO: 1. In another aspect, the amino acid at a position corresponding to position 111 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu. In another aspect, the variant comprises or consists of the substitution K111E corresponding to the polypeptide of SEQ ID NO: 1. In another aspect, the variant comprises or consists of a substitution at a position corresponding to position 111 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; H12L+N35D+Y72F+V74E+K83E+Y100F+K111E+K123E+E132D+E140D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+G125D+E132D+E140D+K151E+D163E+Y165F+E190D+E194D+V211T+L214I+E221R).

In another aspect, the variant comprises or consists of a substitution at a position corresponding to position 112 of the polypeptide of SEQ ID NO: 1. In another aspect, the amino acid at a position corresponding to position 112 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu, Asp, Lys, or Asn. In another aspect, the variant comprises or consists of the substitution H112E, H112D, H112K, or H112N corresponding to the polypeptide of SEQ ID NO: 1. In another aspect, the variant comprises or consists of a substitution at a position corresponding to position 112 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., H5S+H112N; H112E+M218K; D18P+H112E+R126K; K94T+H112N+M189V; K94T+H112N+M218K; L95I+F102L+H112D; L95I+F102L+H112K; L95I+H112K+R126K; L95I+H112D+E188P; L95I+H112K+E188P; Q98K+F102L+H112K; Q98K+H112K+R126K; Q98K+H112D+E188P; F102L+H112K+E188P; H112E+R126K+M191V; H112D+I130E+E188P; H112D+E188P+M189I).

In another aspect, the variant comprises or consists of a substitution at a position corresponding to position 114 of the polypeptide of SEQ ID NO: 1. In another aspect, the amino acid at a position corresponding to position 114 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu, Arg, Gly or Pro. In another aspect, the variant comprises or consists of the substitution N114L, N114R, N114G or N114P corresponding to the polypeptide of SEQ ID NO: 1. In another aspect, the variant comprises or consists of a substitution at a position corresponding to position 114 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D110S+N114P+R166Y).

In another aspect, the variant comprises or consists of a substitution at a position corresponding to position 115 of the polypeptide of SEQ ID NO: 1. In another aspect, the amino acid at a position corresponding to position 115 is substituted with Ala, Arg, Asn, Asp, Cys, Gln, Glu, Gly, His, Ile, Leu, Lys, Met, Phe, Pro, Ser, Thr, Trp, Tyr, or Val, preferably with Gln, Trp, Tyr or Ile. In another aspect, the variant comprises or consists of the substitution L115W, L115Y, L115I or L115Q corresponding to the polypeptide of SEQ ID NO: 1. In another aspect, the variant comprises or consists of a substitution at a position corresponding to position 115 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 117 of the polypeptide of SEQ ID NO: 1. In another aspect, the amino acid at a position corresponding to position 117 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe. In another aspect, the variant comprises or consists of the substitution V117F corresponding to the polypeptide of SEQ ID NO: 1. In another aspect, the variant comprises or consists of a substitution at a position corresponding to position 117 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., T62M+V117F).

In another aspect, the variant comprises or consists of a substitution at a position corresponding to position 118 of the polypeptide of SEQ ID NO: 1. In another aspect, the amino acid at a position corresponding to position 118 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val or Leu. In another aspect, the variant comprises or consists of the substitution I118V or I118L corresponding to the polypeptide of SEQ ID NO: 1. In another aspect, the variant comprises or consists of a substitution at a position corresponding to position 118 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 88, 89, 91, 94, 95, 96, 98, 99, 100, 102, 104, 105, 106, 109, 110, 111, 112, 114, 115, 117, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+I118V+E186K+E188P+M189L; I118L+E186K+E221K; G60D+I118L+E186K+E221K; I118V+E186K+E221K).

In another aspect, the variant comprises or consists of a substitution at a position corresponding to position 119 of the polypeptide of SEQ ID NO: 1. In another aspect, the amino acid at a position corresponding to position 119 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala, Ile, Cys, Thr, Ser or Val. In another aspect, the variant comprises or consists of the substitution G119A, G119S, G119T, G119C, G119I or G119V corresponding to the polypeptide of SEQ ID NO: 1. In another aspect, the variant comprises or consists of a substitution at a position corresponding to position 119 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 121 of the polypeptide of SEQ ID NO: 1. In another aspect, the amino acid at a position corresponding to position 121 is substituted with Ala, Arg, Asn, Asp, Cys, Gln, Glu, Gly, His, Ile, Leu, Lys, Met, Phe, Pro, Ser, Thr, Trp, Tyr, or Val, preferably with Met or Leu. In another aspect, the variant comprises or consists of the substitution F121M or F121L corresponding to the polypeptide of SEQ ID NO: 1. In another aspect, the variant comprises or consists of a substitution at a position corresponding to position 121 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 88, 89, 91, 94, 95, 96, 98, 99, 100, 102, 104, 105, 106, 109, 110, 111, 112, 114, 115, 117, 118, 119, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+F121M+E186K+E188P+M189L; D18P+D39L+F121L+E186K+E188P+M189L).

In another aspect, the variant comprises or consists of a substitution at a position corresponding to position 122 of the polypeptide of SEQ ID NO: 1. In another aspect, the amino acid at a position corresponding to position 122 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr. In another aspect, the variant comprises or consists of the substitution F122Y corresponding to the polypeptide of SEQ ID NO: 1. In another aspect, the variant comprises or consists of a substitution at a position corresponding to position 122 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 123 of the polypeptide of SEQ ID NO: 1. In another aspect, the amino acid at a position corresponding to position 123 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu. In another aspect, the variant comprises or consists of the substitution K123E corresponding to the polypeptide of SEQ ID NO: 1. In another aspect, the variant comprises or consists of a substitution at a position corresponding to position 123 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 88, 89, 91, 94, 95, 96, 98, 99, 100, 102, 104, 105, 106, 109, 110, 111, 112, 114, 115, 117, 118, 119, 121, 122, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 124 of the polypeptide of SEQ ID NO: 1. In another aspect, the amino acid at a position corresponding to position 124 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys. In another aspect, the variant comprises or consists of the substitution E124C corresponding to the polypeptide of SEQ ID NO: 1. In another aspect, the variant comprises or consists of a substitution at a position corresponding to position 124 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., E124C+K193C).

In another aspect, the variant comprises or consists of a substitution at a position corresponding to position 125 of the polypeptide of SEQ ID NO: 1. In another aspect, the amino acid at a position corresponding to position 125 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser, Ala, or Asp. In another aspect, the variant comprises or consists of the substitution G125S, G125A, or G125D corresponding to the polypeptide of SEQ ID NO: 1. In another aspect, the variant comprises or consists of a substitution at a position corresponding to position 125 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+G125S+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; Y72F+V74E+K83E+Y100F+K111E+K123E+G125D+E132D+E140D+K151E+D163E+Y165F+E190D+E194D+V211T+L214I+E221R).

In another aspect, the variant comprises or consists of a substitution at a position corresponding to position 126 of the polypeptide of SEQ ID NO: 1. In another aspect, the amino acid at a position corresponding to position 126 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys. In another aspect, the variant comprises or consists of the substitution R126K corresponding to the polypeptide of SEQ ID NO: 1. In another aspect, the variant comprises or consists of a substitution at a position corresponding to position 126 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+H112E+R126K; L95I+H112K+R126K; Q98K+H112K+R126K; F102M+R126K+M191V; H112E+R126K+M191V).

In another aspect, the variant comprises or consists of a substitution at a position corresponding to position 127 of the polypeptide of SEQ ID NO: 1. In another aspect, the amino acid at a position corresponding to position 127 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro, Leu, Cys, Met, Lys, Gln, Thr, Ala, Arg or Val. In another aspect, the variant comprises or consists of the substitution E127P, E127L, E127C, E127M, E127K, E127Q, E127T, E127A, E127R or E127V corresponding to the polypeptide of SEQ ID NO: 1. In another aspect, the variant comprises or consists of a substitution at a position corresponding to position 127 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 129 of the polypeptide of SEQ ID NO: 1. In another aspect, the amino acid at a position corresponding to position 129 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises or consists of the substitution P129L corresponding to the polypeptide of SEQ ID NO: 1. In another aspect, the variant comprises or consists of a substitution at a position corresponding to position 129 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., F102L+P129L).

In another aspect, the variant comprises or consists of a substitution at a position corresponding to position 130 of the polypeptide of SEQ ID NO: 1. In another aspect, the amino acid at a position corresponding to position 130 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu. In another aspect, the variant comprises or consists of the substitution I130E corresponding to the polypeptide of SEQ ID NO: 1. In another aspect, the variant comprises or consists of a substitution at a position corresponding to position 130 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+I130E+M189I; H112D+I130E+E188P; I130E+A142K+E186K).

In another aspect, the variant comprises or consists of a substitution at a position corresponding to position 132 of the polypeptide of SEQ ID NO: 1. In another aspect, the amino acid at a position corresponding to position 132 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile or Asp. In another aspect, the variant comprises or consists of the substitution E132I or E132D corresponding to the polypeptide of SEQ ID NO: 1. In another aspect, the variant comprises or consists of a substitution at a position corresponding to position 132 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+E132I; Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 133 of the polypeptide of SEQ ID NO: 1. In another aspect, the amino acid at a position corresponding to position 133 is substituted with Ala, Arg, Asn, Asp, Cys, Gln, Glu, Gly, His, Ile, Leu, Lys, Met, Phe, Pro, Ser, Thr, Trp, Tyr, or Val, preferably with Met, Ile, or Val. In another aspect, the variant comprises or consists of the substitution K133M, K133I, or K133V corresponding to the polypeptide of SEQ ID NO: 1. In another aspect, the variant comprises or consists of a substitution at a position corresponding to position 133 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 135 of the polypeptide of SEQ ID NO: 1. In another aspect, the amino acid at a position corresponding to position 135 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises or consists of the substitution W135L corresponding to the polypeptide of SEQ ID NO: 1. In another aspect, the variant comprises or consists of a substitution at a position corresponding to position 135 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 137 of the polypeptide of SEQ ID NO: 1. In another aspect, the amino acid at a position corresponding to position 137 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys. In another aspect, the variant comprises or consists of the substitution V137K corresponding to the polypeptide of SEQ ID NO: 1. In another aspect, the variant comprises or consists of a substitution at a position corresponding to position 137 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., V137K+H150V+M189V).

In another aspect, the variant comprises or consists of a substitution at a position corresponding to position 139 of the polypeptide of SEQ ID NO: 1. In another aspect, the amino acid at a position corresponding to position 139 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys. In another aspect, the variant comprises or consists of the substitution P139C corresponding to the polypeptide of SEQ ID NO: 1. In another aspect, the variant comprises or consists of a substitution at a position corresponding to position 139 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., P139C+E144C).

In another aspect, the variant comprises or consists of a substitution at a position corresponding to position 140 of the polypeptide of SEQ ID NO: 1. In another aspect, the amino acid at a position corresponding to position 140 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala or Asp. In another aspect, the variant comprises or consists of the substitution E140A or E140D corresponding to the polypeptide of SEQ ID NO: 1. In another aspect, the variant comprises or consists of a substitution at a position corresponding to position 140 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+E140A; Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 141 of the polypeptide of SEQ ID NO: 1. In another aspect, the amino acid at a position corresponding to position 141 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys, Thr, Glu or Gln. In another aspect, the variant comprises or consists of the substitution N141K, N141E, N141T or N141Q corresponding to the polypeptide of SEQ ID NO: 1. In another aspect, the variant comprises or consists of a substitution at a position corresponding to position 141 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18K+D39L+N141K+K147I; D18E+N141K+E186R+M218V; D39L+A53G+N141K+S177Y; D39L+K64L+N141K+D163K; D39R+N141K+E186R+M189I; D39L+N141K+E186R+M189I; D39L+N141K+E186L+M218V; K64L+H99L+N141K+E186R; K64L+N141K+E186R+M189L; D18K+N141K+K147I+E186R; D18E+D39L+N141K+E186K; D18K+D39L+N141K+E221L; A53G+N141K+E186R+M191V; D39L+K64L+N141K+E221R; D18E+N141K+E186R+E221L; H99L+N141K+E186R+E221L; N141K+D163K+S177Y+E186R; D39L+H99L+N141K+E221R; A53G+N141K+E186R+E221L; D39L+N141K+E186R+M191V; N141K+S177Y+E186R+M189L; D18P+D39L+N141K+E186R+E188P; D18P+N141K+E186R+E188P+M218V; D18P+N141K+E186R+E188P+E221R).

In another aspect, the variant comprises or consists of a substitution at a position corresponding to position 142 of the polypeptide of SEQ ID NO: 1. In another aspect, the amino acid at a position corresponding to position 142 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys. In another aspect, the variant comprises or consists of the substitution A142K corresponding to the polypeptide of SEQ ID NO: 1. In another aspect, the variant comprises or consists of a substitution at a position corresponding to position 142 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Q98K+A142K+E186K; I130E+A142K+E186K; A142K+E186K+M191V).

In another aspect, the variant comprises or consists of a substitution at a position corresponding to position 144 of the polypeptide of SEQ ID NO: 1. In another aspect, the amino acid at a position corresponding to position 144 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys. In another aspect, the variant comprises or consists of the substitution E144C corresponding to the polypeptide of SEQ ID NO: 1. In another aspect, the variant comprises or consists of a substitution at a position corresponding to position 144 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., P139C+E144C).

In another aspect, the variant comprises or consists of a substitution at a position corresponding to position 145 of the polypeptide of SEQ ID NO: 1. In another aspect, the amino acid at a position corresponding to position 145 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu, Phe or Tyr. In another aspect, the variant comprises or consists of the substitution E145L, E145F or E145Y corresponding to the polypeptide of SEQ ID NO: 1. In another aspect, the variant comprises or consists of a substitution at a position corresponding to position 145 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 146 of the polypeptide of SEQ ID NO: 1. In another aspect, the amino acid at a position corresponding to position 146 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala. In another aspect, the variant comprises or consists of the substitution V146A corresponding to the polypeptide of SEQ ID NO: 1. In another aspect, the variant comprises or consists of a substitution at a position corresponding to position 146 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 147 of the polypeptide of SEQ ID NO: 1. In another aspect, the amino acid at a position corresponding to position 147 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile, Glu, or Met. In another aspect, the variant comprises or consists of the substitution K147I, K147E, or K147M corresponding to the polypeptide of SEQ ID NO: 1. In another aspect, the variant comprises or consists of a substitution at a position corresponding to position 147 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., K147E+K151C; D18E+D39R+K147I; D39R+K147I+M191V; A53G+K147I+E186K; D18K+D39L+N141K+K147I; D18K+D39L+K147I+E186R; D18E+K147I+E186R+M189I; A53G+K147I+E186R+M218V; D39R+K147I+E186R+E221R; D18K+N141K+K147I+E186R; D39L+K147I+D163K+E186K; D39L+K147I+E186L+E221R; H99L+K147I+E186R+E221R; D39R+K64L+K147I+E186R; D39L+H99L+K147I+M189L; D39L+K147I+E186R+M218V; K147I+S177Y+E186R+E221L; D39L+K147I+E186L+E221L; K147I+D163K+E186R+E221L; D18K+D39L+K147I+E186R; D39L+K64L+K147I+M189L; D18E+K147I+E186R+M191V; K147I+E186R+M189L+M191V; K64L+K147I+E186R+M218V; D39L+K147I+S177Y+E221L; D39L+K147I+E186R+M189I; D18K+A53G+K147I+E186R; D18P+H99L+K147I+E186R+E188P; D18P+K147I+E186R+E188P+M189L; D18P+D39L+K147I+E188P+M191V).

In another aspect, the variant comprises or consists of a substitution at a position corresponding to position 148 of the polypeptide of SEQ ID NO: 1. In another aspect, the amino acid at a position corresponding to position 148 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro. In another aspect, the variant comprises or consists of the substitution L148P corresponding to the polypeptide of SEQ ID NO: 1. In another aspect, the variant comprises or consists of a substitution at a position corresponding to position 148 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 149 of the polypeptide of SEQ ID NO: 1. In another aspect, the amino acid at a position corresponding to position 149 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu, Pro, His, or Asp. In another aspect, the variant comprises or consists of the substitution A149L, A149P, A149H, or A149D corresponding to the polypeptide of SEQ ID NO: 1. In another aspect, the variant comprises or consists of a substitution at a position corresponding to position 149 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R).

In another aspect, the variant comprises or consists of a substitution at a position corresponding to position 150 of the polypeptide of SEQ ID NO: 1. In another aspect, the amino acid at a position corresponding to position 150 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val, Arg or Ser. In another aspect, the variant comprises or consists of the substitution H150V, H150R or H150S corresponding to the polypeptide of SEQ ID NO: 1. In another aspect, the variant comprises or consists of a substitution at a position corresponding to position 150 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., V137K+H150V+M189V).

In another aspect, the variant comprises or consists of a substitution at a position corresponding to position 151 of the polypeptide of SEQ ID NO: 1. In another aspect, the amino acid at a position corresponding to position 151 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys, Glu, or Pro. In another aspect, the variant comprises or consists of the substitution K151C, K151E, or K151P corresponding to the polypeptide of SEQ ID NO: 1. In another aspect, the variant comprises or consists of a substitution at a position corresponding to position 151 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., K147E+K151C; H5R+G6P+E19P+K151E; M22N+A89E+K151P+E188P+D206P; Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 152 of the polypeptide of SEQ ID NO: 1. In another aspect, the amino acid at a position corresponding to position 152 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys or Leu. In another aspect, the variant comprises or consists of the substitution I152C or I152L corresponding to the polypeptide of SEQ ID NO: 1. In another aspect, the variant comprises or consists of a substitution at a position corresponding to position 152 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., G52C+I152C; D18P+D39L+I152L+E186K+E188P+M189L).

In another aspect, the variant comprises or consists of a substitution at a position corresponding to position 153 of the polypeptide of SEQ ID NO: 1. In another aspect, the amino acid at a position corresponding to position 153 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises or consists of the substitution N153D corresponding to the polypeptide of SEQ ID NO: 1. In another aspect, the variant comprises or consists of a substitution at a position corresponding to position 153 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ

In another aspect, the variant comprises or consists of a substitution at a position corresponding to position 154 of the polypeptide of SEQ ID NO: 1. In another aspect, the amino acid at a position corresponding to position 154 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro, Val, Leu or Ile. In another aspect, the variant comprises or consists of the substitution A154P, A154V, A154L or A154I corresponding to the polypeptide of SEQ ID NO: 1. In another aspect, the variant comprises or consists of a substitution at a position corresponding to position 154 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+A154P+E186K+E188P+M189L).

In another aspect, the variant comprises or consists of a substitution at a position corresponding to position 155 of the polypeptide of SEQ ID NO: 1. In another aspect, the amino acid at a position corresponding to position 155 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala, Ile or Arg. In another aspect, the variant comprises or consists of the substitution E155A, E155I or E155R corresponding to the polypeptide of SEQ ID NO: 1. In another aspect, the variant comprises or consists of a substitution at a position corresponding to position 155 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+E155A+E186K+E188P+M189L; D18P+D39L+E155R+E186K+E188P+M189L).

In another aspect, the variant comprises or consists of a substitution at a position corresponding to position 156 of the polypeptide of SEQ ID NO: 1. In another aspect, the amino acid at a position corresponding to position 156 is substituted with Ala, Arg, Asn, Asp, Cys, Gln, Glu, Gly, His, Ile, Leu, Lys, Met, Phe, Pro, Ser, Thr, Trp, Tyr, or Val, preferably with Met, Asn, Gln or Leu. In another aspect, the variant comprises or consists of the substitution D156M, D156N, D156Q or D156L corresponding to the polypeptide of SEQ ID NO: 1. In another aspect, the variant comprises or consists of a substitution at a position corresponding to position 156 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 161 of the polypeptide of SEQ ID NO: 1. In another aspect, the amino acid at a position corresponding to position 161 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asn. In another aspect, the variant comprises or consists of the substitution D161N corresponding to the polypeptide of SEQ ID NO: 1. In another aspect, the variant comprises or consists of a substitution at a position corresponding to position 161 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D161N+E188P; K41C+D161N+E188P; D39K+D161N+E188P+K222*; D161N+E186K+E188P+E221K; K41C+D161N+E186K+E188P+E221K; D39K+K41C+D161N+E188P+K222*; D39K+D161N+E186K+E188P+E221K+K222*; D39K+K41C+D161N+E186K+E188P+E221K+K222*; D18P+D39K+D161N+E186K+E188P+E221K+K222*; D18P+D39K+K41C+D161N+E186K+E188P+E221K+K222*; D39K+K43A+D161N+E186K+E188P+M218K+E221K+K222*; D18P+D39K+K43A+D161N+E186K+E188P+M218K+E221K+K222*; R36D+D39K+K43A+D161N+E186K+E188P+M218K+E221K+K222*; D39K+K41C+K43A+D161N+E186K+E188P+M218K+E221K+K222*; R36D+D39K+K41C+K43A+D161N+E186K+E188P+M218K+E221K+K222*; D18P+D39K+K41C+K43A+D161N+E186K+E188P+M218K+E221K+K222*; D18P+R36D+D39K+K41C+K43A+D161N+E186K+E188P+M218K+E221K+K222*).

In another aspect, the variant comprises or consists of a substitution at a position corresponding to position 162 of the polypeptide of SEQ ID NO: 1. In another aspect, the amino acid at a position corresponding to position 162 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe, Met, or Leu. In another aspect, the variant comprises or consists of the substitution R162F, R162M, or R162L corresponding to the polypeptide of SEQ ID NO: 1. In another aspect, the variant comprises or consists of a substitution at a position corresponding to position 162 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., N35D+Y72F+V74E+K83E+Y100F+K111E+K123E+E132D+E140D+K151E+R162L+D163E+Y165F+E190D+E194D+V211T+L214I+E221R).

In another aspect, the variant comprises or consists of a substitution at a position corresponding to position 163 of the polypeptide of SEQ ID NO: 1. In another aspect, the amino acid at a position corresponding to position 163 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys, Thr, Leu, Arg, or Glu. In another aspect, the variant comprises or consists of the substitution D163K, D163T, D163L, D163R, or D163E corresponding to the polypeptide of SEQ ID NO: 1. In another aspect, the variant comprises or consists of a substitution at a position corresponding to position 163 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+K64L+N141K+D163K; D39R+H99L+D163K+E186R; K64L+D163K+E186R+M189L; D163K+S177Y+E186R+M218V; D18K+K64L+D163K+E186R; D39L+D163K+S177Y+E186R; D18E+D39R+D163K+E186R; D39L+H99L+D163K+E221L; D39L+K147I+D163K+E186K; D39L+D163K+E186K+E221L; D39L+D163K+E186R+M191V; D39L+D163K+E186R+E221L; A53G+D163K+E186R+E221R; D39L+D163K+E186R+M218V; D39L+D163K+E186L+M189I; N141K+D163K+S177Y+E186R; H99L+D163K+E186R+M189L; K147I+D163K+E186R+E221L; D18E+D39L+D163K+E221R; D39L+K64L+D163K+M191V; D18E+D163K+E186R+E221R; D18K+D163K+E186R+M189I; D18E+A53G+D163K+E186R; D18K+D39L+D163K+M218V; D18P+D163K+E186R+E188P+M191V; D18P+D163K+E186R+E188P+M189I; D39L+D163K+E186R+M191V+M218V+E221R; D39L+D163K+E186K+M191V+M218V+E221R; Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 165 of the polypeptide of SEQ ID NO: 1. In another aspect, the amino acid at a position corresponding to position 165 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe. In another aspect, the variant comprises or consists of the substitution Y165F corresponding to the polypeptide of SEQ ID NO: 1. In another aspect, the variant comprises or consists of a substitution at a position corresponding to position 165 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 166 of the polypeptide of SEQ ID NO: 1. In another aspect, the amino acid at a position corresponding to position 166 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala, Met, Lys, Glu, Phe, Val, Cys or Tyr. In another aspect, the variant comprises or consists of the substitution R166A, R166M, R166K, R166E, R166F, R166V, R166C or R166Y corresponding to the polypeptide of SEQ ID NO: 1. In another aspect, the variant comprises or consists of a substitution at a position corresponding to position 166 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D110S+N114P+R166Y).

In another aspect, the variant comprises or consists of a substitution at a position corresponding to position 168 of the polypeptide of SEQ ID NO: 1. In another aspect, the amino acid at a position corresponding to position 168 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asn, Cys, Ala, Asp, Glu, Leu, Pro, Gln, Thr, Val, Met, or Ile. In another aspect, the variant comprises or consists of the substitution S168N, S168C, S168A, S168D, S168E, S168L, S168K, S168P, S168Q, S168T, S168V, S168M, or S168I corresponding to the polypeptide of SEQ ID NO: 1. In another aspect, the variant comprises or consists of a substitution at a position corresponding to position 168 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 169 of the polypeptide of SEQ ID NO: 1. In another aspect, the amino acid at a position corresponding to position 169 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp. In another aspect, the variant comprises or consists of the substitution G169D corresponding to the polypeptide of SEQ ID NO: 1. In another aspect, the variant comprises or consists of a substitution at a position corresponding to position 169 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., G169D+E186R).

In another aspect, the variant comprises or consists of a substitution at a position corresponding to position 171 of the polypeptide of SEQ ID NO: 1. In another aspect, the amino acid at a position corresponding to position 171 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe. In another aspect, the variant comprises or consists of the substitution L171F corresponding to the polypeptide of SEQ ID NO: 1. In another aspect, the variant comprises or consists of a substitution at a position corresponding to position 171 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 173 of the polypeptide of SEQ ID NO: 1. In another aspect, the amino acid at a position corresponding to position 173 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val, Trp or Ile. In another aspect, the variant comprises or consists of the substitution T173V, T173W or T1731 corresponding to the polypeptide of SEQ ID NO: 1. In another aspect, the variant comprises or consists of a substitution at a position corresponding to position 173 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 177 of the polypeptide of SEQ ID NO: 1. In another aspect, the amino acid at a position corresponding to position 177 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr, Met, Trp or Phe. In another aspect, the variant comprises or consists of the substitution S177Y, S177M, S177W or S177F corresponding to the polypeptide of SEQ ID NO: 1. In another aspect, the variant comprises or consists of a substitution at a position corresponding to position 177 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., N58D+S177M; S177Y+E186R; S177Y+E221L; D18E+D39R+S177Y+E186R; D39L+A53G+N141K+S177Y; D39R+K64L+S177Y+M218V; D39L+S177Y+E186R+E221R; K64L+S177Y+E186R+E221L; K64L+S177Y+M218V+E221L; D163K+S177Y+E186R+M218V; S177Y+E186R+M191V+E221R; D39L+D163K+S177Y+E186R; D18K+K64L+S177Y+E186R; D18K+D39L+S177Y+M189I; D39L+S177Y+E186K+M191V; S177Y+E186R+M191V+M218V; D39L+S177Y+E186K+E221R; D39L+K64L+S177Y+E186R; D39R+K64L+S177Y+E186R; K64L+S177Y+E186R+M218V; K147I+S177Y+E186R+E221L; K64L+S177Y+E186K+M189L; D39R+K64L+S177Y+E221R; K64L+S177Y+E186K+E221R; D39R+K64L+S177Y+E186K; K64L+S177Y+E186R+E221R; N141K+D163K+S177Y+E186R; D18E+S177Y+E186R+M189L; N141K+S177Y+E186R+M189L; D18K+D39L+S177Y+E186R; D18E+D39L+S177Y+E186R; D39L+K147I+S177Y+E221L; A53G+H99L+S177Y+E186R; D18P+D39R+S177Y+E186R+E188P; D18P+D39L+S177Y+E188P+M191V; D18P+H99L+S177Y+E186R+E188P).

In another aspect, the variant comprises or consists of a substitution at a position corresponding to position 178 of the polypeptide of SEQ ID NO: 1. In another aspect, the amino acid at a position corresponding to position 178 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ser, Pro, Thr or Gln. In another aspect, the variant comprises or consists of the substitution E178S, E178P, E178T or E178Q corresponding to the polypeptide of SEQ ID NO: 1. In another aspect, the variant comprises or consists of a substitution at a position corresponding to position 178 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 181 of the polypeptide of SEQ ID NO: 1. In another aspect, the amino acid at a position corresponding to position 181 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ala, Thr, Ile, or Leu. In another aspect, the variant comprises or consists of the substitution R181A, R181T, R181I, or R181L corresponding to the polypeptide of SEQ ID NO: 1. In another aspect, the variant comprises or consists of a substitution at a position corresponding to position 181 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 182 of the polypeptide of SEQ ID NO: 1. In another aspect, the amino acid at a position corresponding to position 182 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys. In another aspect, the variant comprises or consists of the substitution W182K corresponding to the polypeptide of SEQ ID NO: 1. In another aspect, the variant comprises or consists of a substitution at a position corresponding to position 182 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+W182K).

In another aspect, the variant comprises or consists of a substitution at a position corresponding to position 185 of the polypeptide of SEQ ID NO: 1. In another aspect, the amino acid at a position corresponding to position 185 is substituted with Ala, Arg, Asn, Asp, Cys, Gln, Glu, Gly, His, Ile, Leu, Lys, Met, Phe, Pro, Ser, Thr, Trp, Tyr, or Val, preferably with Phe or Leu. In another aspect, the variant comprises or consists of the substitution M185F or M185L corresponding to the polypeptide of SEQ ID NO: 1. In another aspect, the variant comprises or consists of a substitution at a position corresponding to position 185 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 186 of the polypeptide of SEQ ID NO: 1. In another aspect, the amino acid at a position corresponding to position 186 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys, Leu, Arg. In another aspect, the variant comprises or consists of the substitution E186K, E186L, or E186R corresponding to the polypeptide of SEQ ID NO: 1. In another aspect, the variant comprises or consists of a substitution at a position corresponding to position 186 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., K64L+E186R; G169D+E186R; S177Y+E186R; E186R+M191V; E186K+M191V; E186R+M191V; E186K+E221K; K41C+E186K+E221K; D39R+E186R+M191V; D39L+E186K+M191V; A53G+K147I+E186K; Q98K+A142K+E186K; Q98K+E186K+M191V; F102L+E186K+M191V; I130E+A142K+E186K; A142K+E186K+M191V; E186K+M191V+M218V; E186R+M191V+E221L; E186K+M191V+E221L; D18E+D39L+K64L+E186L; D18K+D39L+H99L+E186L; D18K+D39L+K147I+E186R; D18E+D39R+S177Y+E186R; D18K+D39L+E186K+M189L; D18K+D39R+E186R+E221R; D18E+D39L+E186L+M191V; D18K+K64L+E186R+M191V; D18K+H99L+E186R+M189L; D18E+N141K+E186R+M218V; D18E+K147I+E186R+M189I; D39R+A53G+K64L+E186R; D39L+A53G+E186R+E221R; D39R+H99L+D163K+E186R; D39L+H99L+E186K+M218V; D39R+N141K+E186R+M189I; D39L+N141K+E186R+M189I; D39L+N141K+E186L+M218V; D39L+S177Y+E186R+E221R; A53G+K147I+E186R+M218V; K64L+H99L+N141K+E186R; K64L+N141K+E186R+M189L; K64L+D163K+E186R+M189L; K64L+S177Y+E186R+E221L; D161N+E186K+E188P+E221K; D163K+S177Y+E186R+M218V; E186R+M189L+M218V+E221L; D18K+D39R+E186R+M218V; S177Y+E186R+M191V+E221R; D39L+E186R+M191V+E221R; A53G+E186R+M189L+E221R; D39R+E186R+M191V+M218V; D39R+K64L+K147I+E186R; D39L+H99L+E186K+E221R; H99L+K147I+E186R+E221R; D39L+D163K+E186K+E221L; D18K+D39L+E186R+M189L; D18K+N141K+K147I+E186R; D18E+D39R+D163K+E186R; D18K+H99L+E186R+E221L; D39L+K64L+H99L+E186R; D39L+A53G+E186R+M189L; D18K+K64L+S177Y+E186R; D39L+K147I+E186L+E221R; D18K+D39L+E186K+E221R; D39L+A53G+E186R+M189I; D39L+H99L+E186R+M189I; D18E+D39L+N141K+E186K; D39L+K147I+D163K+E186K; D39L+D163K+E186R+M191V; D39L+D163K+E186R+E221L; A53G+D163K+E186R+E221R; D39K+E186K+E221K+K222*; D18K+D39L+E186R+M218V; D39L+K64L+E186L+M189L; D39L+S177Y+E186K+M191V; D39L+K147I+E186R+M218V; K64L+H99L+E186R+M218V; A53G+N141K+E186R+M191V; D39R+E186R+M189I+M191V; D39L+H99L+E186R+E221R; D39L+E186R+M191V+E221L; D39L+N141K+E186R+M191V; D18E+N141K+E186R+E221L; S177Y+E186R+M191V+M218V; D39L+S177Y+E186K+E221R; D18K+E186R+M191V+E221L; D18E+A53G+K64L+E186R; A53G+N141K+E186R+E221L; D39L+D163K+E186R+M218V; D39L+K64L+S177Y+E186R; D39R+K64L+S177Y+E186R;

K64L+S177Y+E186R+M218V; K147I+S177Y+E186R+E221L; K64L+S177Y+E186K+M189L; K64L+S177Y+E186K+E221R; D39R+K64L+S177Y+E186K; K64L+S177Y+E186R+E221R; H99L+N141K+E186R+E221L; D39R+E186R+M189L+E221R; D39L+D163K+E186L+M189I; D18K+D39L+A53G+E186L; N141K+D163K+S177Y+E186R; D39L+K147I+E186L+E221L; E186R+M189I+M218V+E221R; H99L+D163K+E186R+M189L; K147I+D163K+E186R+E221L; D18K+E186R+M189L+E221L; D39L+D163K+S177Y+E186R; D18E+S177Y+E186R+M189L; D39R+K147I+E186R+E221R; D18K+D39L+K147I+E186R; N141K+S177Y+E186R+M189L; D18K+K64L+D163K+E186R; D18E+K147I+E186R+M191V; K147I+E186R+M189L+M191V; D18K+D163K+E186R+M189I; D18K+D39L+S177Y+E186R; D18E+K64L+E186R+M189I; D39R+A53G+H99L+E186R; K64L+K147I+E186R+M218V; D18E+D39L+S177Y+E186R; D18E+A53G+D163K+E186R; A53G+E186R+M189I+M218V; D39L+K147I+E186R+M189I; D18E+D39L+E186R+M189L; D18E+E186R+M189I+E221L; D39R+H99L+E186R+M191V; A53G+H99L+S177Y+E186R; D18E+H99L+E186R+M218V; D18K+A53G+K147I+E186R; D18P+D39L+H99L+E186L+E188P; D18P+D39L+N141K+E186R+E188P; D18P+D39L+E186K+E188P+M189L; D18P+D39R+E186R+E188P+E221L; D18P+H99L+K147I+E186R+E188P; D18P+A53G+E186R+E188P+E221L; D18P+D39R+S177Y+E186R+E188P; D18P+N141K+E186R+E188P+M218V; D18P+E186R+E188P+M189I+E221L; D18P+D163K+E186R+E188P+M189I; D18P+N141K+E186R+E188P+E221R; D18P+D39L+K64L+E186R+E188P; D18P+E186R+E188P+M189L+M191V; D18P+D39L+A53G+E186R+E188P; D18P+D39L+E186L+E188P+M218V; D18P+K147I+E186R+E188P+M189L; D18P+H99L+S177Y+E186R+E188P; K41C+D161N+E186K+E188P+E221K; D18P+D39L+E186K+E188P+M189L; D18P+D39L+K64L+E186K+E188P; D39K+K41C+E186K+E221K+K222*; D18P+K64L+E186R+E188P+E221R; D18P+D39L+E186K+E188P+M189L; D18P+D163K+E186R+E188P+M191V; D39K+D161N+E186K+E188P+E221K+K222*; D39L+D163K+E186K+M191V+M218V+E221R; D39L+D163K+E186R+M191V+M218V+E221R; D39K+K41C+D161N+E186K+E188P+E221K+K222*; D18P+D39K+D161N+E186K+E188P+E221K+K222*; D39K+K43A+D161N+E186K+E188P+M218K+E221K+K222*; D18P+D39K+K41C+D161N+E186K+E188P+E221K+K222*; D18P+D39K+K43A+D161N+E186K+E188P+M218K+E221K+K222*; R36D+D39K+K43A+D161N+E186K+E188P+M218K+E221K+K222*; D39K+K41C+K43A+D161N+E186K+E188P+M218K+E221K+K222*; R36D+D39K+K41C+K43A+D161N+E186K+E188P+M218K+E221K+K222*; D18P+D39K+K41C+K43A+D161N+E186K+E188P+M218K+E221K+K222*; D18P+R36D+D39K+K41C+K43A+D161N+E186K+E188P+M218K+E221K+K222*; E186K+1202T+E221K; E127K+E186K+E221K; E127Q+E186K+E221K; E127T+E186K+E221K; N141Q+E186K+E221K; E127A+E186K+E221K; E186K+1202L+E221K; E186K+1202V+E221K; A154I+E186K+E221K; N141T+E186K+E221K; N141E+E186K+E221K; E127R+E186K+E221K; A154V+E186K+E221K; E186K+1202A+E221K; A154P+E186K+E221K; A154L+E186K+E221K; N141T+R166C+E186K+E221K). In another aspect, the variant comprises or consists of a substitution at position 186 of the polypeptide of SEQ ID NO: 1 (e.g., E186K, E186L or E186R) together with a deletion or substitution at a position corresponding to position 39 of the polypeptide of SEQ ID NO: 1 (e.g., D39*, D39A, D39C, D39E, D39F, D39G, D39K, D39L, D39M, D39N, D39P, D39R, D39S, D39W or D39Y). In another aspect, the variant comprises or consists of D39*+E186K, D39*+E186L, or D39*+E186R. In another aspect, the variant comprises or consists of D39A+E186K, D39A+E186L, or D39A+E186R. In another aspect, the variant comprises or consists of D39C+E186K, D39C+E186L, or D39C+E186R. In another aspect, the variant comprises or consists of D39E+E186K, D39E+E186L, or D39E+E186R. In another aspect, the variant comprises or consists of D39F+E186K, D39F+E186L or D39F+E186R. In another aspect, the variant comprises or consists of D39G+E186K, D39G+E186L, or D39G+E186R. In another aspect, the variant comprises or consists of D39K+E186K, D39K+E186L, or D39K+E186R. In another aspect, the variant comprises or consists of D39L+E186K, D39L+E186L, or D39L+E186R. In another aspect, the variant comprises or consists of D39M+E186K, D39M+E186L, or D39M+E186R. In another aspect, the variant comprises or consists of D39N+E186K, D39N+E186L, or D39N+E186R. In another aspect, the variant comprises or consists of D39P+E186K, D39P+E186L, or D39P+E186R. In another aspect, the variant comprises or consists of D39R+E186K, D39R+E186L, or D39R+E186R. In another aspect, the variant comprises or consists of D39S+E186K, D39S+E186L, or D39S+E186R. In another aspect, the variant comprises or consists of D39W+E186K, D39W+E186L, or D39W+E186R. In another aspect, the variant comprises or consists of D39Y+E186K, D39Y+E186L, or D39Y+E186R.

In another aspect, the variant comprises or consists of a substitution at a position corresponding to position 187 of the polypeptide of SEQ ID NO: 1. In another aspect, the amino acid at a position corresponding to position 187 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile or Val. In another aspect, the variant comprises or consists of the substitution E187I or E187V corresponding to the polypeptide of SEQ ID NO: 1. In another aspect, the variant comprises or consists of a substitution at a position corresponding to position 187 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 188 of the polypeptide of SEQ ID NO: 1. In another aspect, the amino acid at a position corresponding to position 188 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val or Pro. In another aspect, the variant comprises or consists of the substitution E188V or E188P corresponding to the polypeptide of SEQ ID NO: 1. In another aspect, the variant comprises or consists of a substitution at a position corresponding to position 188 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+E188P; D161N+E188P; E188P+M218I; K41C+D161N+E188P; L95I+H112K+E188P; L95I+H112D+E188P; Q98K+H112D+E188P; F102L+H112K+E188P; H112D+I130E+E188P; H112D+E188P+M189I; D39K+D161N+E188P+K222*; D161N+E186K+E188P+E221K; D18P+D39L+H99L+E186L+E188P; D18P+D39L+N141K+E186R+E188P; D18P+D39L+E186K+E188P+M189L; D18P+D39R+E186R+E188P+E221L; D18P+D39L+E188P+M189L+E221R; D18P+D39L+K64L+E186K+E188P; D18P+H99L+K147I+E186R+E188P; D18P+A53G+E186R+E188P+E221L; D18P+N141K+E186R+E188P+M218V; D18P+D39R+S177Y+E186R+E188P; D18P+E186R+E188P+M189I+E221L; D18P+D163K+E186R+E188P+M189I; D18P+N141K+E186R+E188P+E221R; D18P+D39L+K64L+E186R+E188P; D18P+E186R+E188P+M189L+M191V; D18P+D39L+A53G+E186R+E188P; D18P+D39L+H99L+E188P+M189I; D18P+D39L+E186L+E188P+M218V; D18P+K147I+E186R+E188P+M189L; D18P+H99L+S177Y+E186R+E188P; K41C+D161N+E186K+E188P+E221K; M22N+A89E+K151P+E188P+D206P; D18P+D39L+E186K+E188P+M189L; D18P+D39L+K147I+E188P+M191V; D39K+K41C+D161N+E188P+K222*; D18P+D39L+S177Y+E188P+M191V; D18P+K64L+E186R+E188P+E221R; D18P+D163K+E186R+E188P+M191V; D18P+D39L+E186K+E188P+M189L; D39K+D161N+E186K+E188P+E221K+K222*; D39K+K41C+D161N+E186K+E188P+E221K+K222*; D18P+D39K+D161N+E186K+E188P+E221K+K222*; D39K+K43A+D161N+E186K+E188P+M218K+E221K+K222*; D18P+D39K+K41C+D161N+E186K+E188P+E221K+K222*; D18P+D39K+K43A+D161N+E186K+E188P+M218K+E221K+K222*; R36D+D39K+K43A+D161N+E186K+E188P+M218K+E221K+K222*; D39K+K41C+K43A+D161N+E186K+E188P+M218K+E221K+K222*; R36D+D39K+K41C+K43A+D161N+E186K+E188P+M218K+E221K+K222*; D18P+D39K+K41C+K43A+D161N+E186K+E188P+M218K+E221K+K222*; D18P+R36D+D39K+K41C+K43A+D161N+E186K+E188P+M218K+E221K+K222*; D18P+D39L+K64L+V75I+E186K+E188P+K198Y; D18P+D39L+T62Y+K64L+V75I+E186K+E188P; D18P+D39L+K46Y+K64L+V75I+E186K+E188P; D18P+D39L+K64L+V75I+G119T+E186K+E188P; D18P+D39L+K64L+V75I+E186K+E187I+E188P; D18P+M22F+N28D+D39L+K64L+V75I+E178P+E186K+E188P; D18P+D39L+K64L+S66I+V75I+E186K+E188P; D18P+D39L+K64L+V75I+E186K+E188P+E221L; D18P+D39L+K64L+V75I+E186I+E188P; D18P+M22F+N28D+D39L+K64L+V75I+E178S+E186K+E188P; D18P+D39L+K64L+V75I+E186K+E188P+K198F; D18P+D39L+K64L+V75I+E186K+E187V+E188P; D18P+D39L+K64L+V75I+H105F+E186K+E188P; D18P+M22F+N28T+D39L+K64L+V75I+E178P+E186K+E188P; D18P+D39L+K64L+V75I+E127P+E186K+E188P; D18P+D39L+K64L+V75I+E186K+E188P+K222L; D18P+K27Y+D39L+K64L+V75I+N114L+E186K+E188P+N215L; D18P+D39L+K64L+V75I+E186K+E188P+N208L; D18P+D39L+K64L+S66Y+V75I+E186K+E188P; D18P+D39L+N59S+K64L+V75I+E186K+E188P; D18P+D39L+K64L+V75I+R166Y+E186K+E188P+M218V+M220L; D18P+N28F+D39L+K64L+V75I+S168A+E186K+E188P; D18P+D39L+K64L+V75I+R166Y+E186K+E188P+M218I+M220L; D18P+D39L+K64L+V75I+E186K+E188P+E221Y; D18P+D39L+K64L+V75I+R166Y+E186K+E188P+M218I; D18P+D39L+K64L+V75I+R166Y+E186K+E188P+M218L; D18P+D39L+K64L+V75I+D163T+E186K+E188P; D18P+D39L+K64L+V75I+G119V+E186K+E188P; D18P+D39L+K64L+V75I+E186K+E188P+E221F; D18P+D39L+K43P+K64L+V75I+E186K+E188P; D18P+D39Y+K64L+V75I+E186K+E188P; D18P+K27T+D39L+K64L+V75I+N114R+E186K+E188P; D18P+R36N+D39L+K64L+V75I+D76K+R181A+E186K+E188P; D18P+M22F+N28F+D39L+K64L+V75I+E186K+E188P; D18P+R36N+D39L+K64L+V75I+D76K+R181T+E186K+E188P; D18P+R36N+D39L+K64L+V75I+D76N+R181T+E186K+E188P; D18P+M22Y+D39L+K64L+V75I+E178Q+E186K+E188P; D18P+M22W+D39L+K64L+V75I+E178Q+E186K+E188P; D18P+D39L+N59V+K64L+V75I+E186K+E188P).

In another aspect, the variant comprises or consists of a substitution at a position corresponding to position 189 of the polypeptide of SEQ ID NO: 1. In another aspect, the amino acid at a position corresponding to position 189 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile, Val, Leu. In another aspect, the variant comprises or consists of the substitution M189I, M189V, or M189L corresponding to the polypeptide of SEQ ID NO: 1. In another aspect, the variant comprises or consists of a substitution at a position corresponding to position 189 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+I130E+M189I; K94T+H112N+M189V; K94T+M189V+M218K; F102M+M189I+M191V; H112D+E188P+M189I; V137K+H150V+M189V; D18K+D39L+E186K+M189L; D18E+D39L+M189L+M191V; D18K+H99L+E186R+M189L; D18E+K147I+E186R+M189I; D39L+A53G+M189L+E221L; D39L+K64L+M189I+M191V; D39R+N141K+E186R+M189I; D39L+N141K+E186R+M189I; K64L+N141K+E186R+M189L; K64L+D163K+E186R+M189L; E186R+M189L+M218V+E221L; N141K+S177Y+E186R+M189L; D39L+K64L+K147I+M189L; A53G+E186R+M189L+E221R; D18K+D39L+E186R+M189L; D39L+A53G+E186R+M189L; D39L+H99L+E186R+M189I; D18K+D39L+M189L+M191V; D39L+H99L+K147I+M189L; D39R+E186R+M189I+M191V; K64L+S177Y+E186K+M189L; D39R+E186R+M189L+E221R; D39L+D163K+E186L+M189I; E186R+M189I+M218V+E221R; H99L+D163K+E186R+M189L; D18E+S177Y+E186R+M189L; K147I+E186R+M189L+M191V; D18K+D163K+E186R+M189I; D18E+K64L+E186R+M189I; D18K+D39L+M189I+E221R; A53G+E186R+M189I+M218V; D39L+K147I+E186R+M189I; D18E+D39L+E186R+M189L; D18E+E186R+M189I+E221L; D39L+K64L+E186L+M189L; D18K+D39L+S177Y+M189I; D39L+A53G+E186R+M189I; D18K+E186R+M189L+E221L; D18P+D39L+E186K+E188P+M189L; D18P+D39L+E188P+M189L+E221R; D18P+E186R+E188P+M189I+E221L; D18P+D163K+E186R+E188P+M189I; D18P+E186R+E188P+M189L+M191V; D18P+D39L+H99L+E188P+M189I; D18P+K147I+E186R+E188P+M189L; D18P+D39L+E186K+E188P+M189L; D18P+D39L+E186K+E188P+M189L; D18P+D39L+E186K+E188P+M189L).

In another aspect, the variant comprises or consists of a substitution at a position corresponding to position 190 of the polypeptide of SEQ ID NO: 1. In another aspect, the amino acid at a position corresponding to position 190 is substituted with Ala, Arg, Asn, Asp, Cys, Gln, Glu, Gly, His, Ile, Leu, Lys, Met, Phe, Pro, Ser, Thr, Trp, Tyr, or Val, preferably with Gly or Asp. In another aspect, the variant comprises or consists of the substitution E190G or E190D corresponding to the polypeptide of SEQ ID NO: 1. In another aspect, the variant comprises or consists of a substitution at a position corresponding to position 190 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+E190G; Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 191 of the polypeptide of SEQ ID NO: 1. In another aspect, the amino acid at a position corresponding to position 191 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val, Thr, Trp, His, Leu, Ala, Gly, Arg, Lys, Pro, Phe, Gln, Asn or Ile. In another aspect, the variant comprises or consists of the substitution M191V, M191A, M191H, M191G, M191L, M191K, M191P, M191N, M191R, M191T, M191F, M191Q, M191W or M1911 corresponding to the polypeptide of SEQ ID NO: 1. In another aspect, the variant comprises or consists of a substitution at a position corresponding to position 191 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., E186K+M191V; E186R+M191V; E186R+M191V; M191V+E221L; D39R+K147I+M191V; D39R+E186R+M191V; D39L+E186K+M191V; Q98K+E186K+M191V; F102M+R126K+M191V; F102L+E186K+M191V; F102M+M189I+M191V; H112E+R126K+M191V; A142K+E186K+M191V; E186K+M191V+M218V; E186R+M191V+E221L; E186K+M191V+E221L; D18E+D39L+E186L+M191V; D18E+D39L+M189L+M191V; D18K+K64L+E186R+M191V; D39L+K64L+M189I+M191V; S177Y+E186R+M191V+E221R; D39L+K64L+D163K+M191V; D39L+E186R+M191V+E221R; D39L+D163K+E186R+M191V; D39L+S177Y+E186K+M191V; D18K+D39L+M189L+M191V; A53G+N141K+E186R+M191V; D39R+E186R+M189I+M191V; D39L+E186R+M191V+E221L; S177Y+E186R+M191V+M218V; D18K+E186R+M191V+E221L; D39L+N141K+E186R+M191V; D39R+E186R+M191V+M218V; D18E+K147I+E186R+M191V; K147I+E186R+M189L+M191V; D39R+H99L+E186R+M191V; D18P+E186R+E188P+M189L+M191V; D18P+D39L+K147I+E188P+M191V; D18P+D39L+S177Y+E188P+M191V; D18P+D163K+E186R+E188P+M191V; D39L+D163K+E186K+M191V+M218V+E221R; D39L+D163K+E186R+M191V+M218V+E221R).

In another aspect, the variant comprises or consists of a substitution at a position corresponding to position 192 of the polypeptide of SEQ ID NO: 1. In another aspect, the amino acid at a position corresponding to position 192 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys. In another aspect, the variant comprises or consists of the substitution S192C corresponding to the polypeptide of SEQ ID NO: 1. In another aspect, the variant comprises or consists of a substitution at a position corresponding to position 192 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., E127C+S192C).

In another aspect, the variant comprises or consists of a substitution at a position corresponding to position 193 of the polypeptide of SEQ ID NO: 1. In another aspect, the amino acid at a position corresponding to position 193 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys. In another aspect, the variant comprises or consists of the substitution K193C corresponding to the polypeptide of SEQ ID NO: 1. In another aspect, the variant comprises or consists of a substitution at a position corresponding to position 193 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., E124C+K193C).

In another aspect, the variant comprises or consists of a substitution at a position corresponding to position 194 of the polypeptide of SEQ ID NO: 1. In another aspect, the amino acid at a position corresponding to position 194 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asp or Ala. In another aspect, the variant comprises or consists of the substitution E194D or E194A corresponding to the polypeptide of SEQ ID NO: 1. In another aspect, the variant comprises or consists of a substitution at a position corresponding to position 194 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 197 of the polypeptide of SEQ ID NO: 1. In another aspect, the amino acid at a position corresponding to position 197 is substituted with Ala, Arg, Asn, Asp, Cys, Gln, Glu, Gly, His, Ile, Leu, Lys, Met, Phe, Pro, Ser, Thr, Trp, Tyr, or Val, preferably with Val. In another aspect, the variant comprises or consists of the substitution E197V corresponding to the polypeptide of SEQ ID NO: 1. In another aspect, the variant comprises or consists of a substitution at a position corresponding to position 197 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+E197V).

In another aspect, the variant comprises or consists of a substitution at a position corresponding to position 198 of the polypeptide of SEQ ID NO: 1. In another aspect, the amino acid at a position corresponding to position 198 is substituted with Ala, Arg, Asn, Asp, Cys, Gln, Glu, Gly, His, Ile, Leu, Lys, Met, Phe, Pro, Ser, Thr, Trp, Tyr, or Val, preferably with Tyr, Phe or Leu. In another aspect, the variant comprises or consists of the substitution K198Y, K198F or K198L corresponding to the polypeptide of SEQ ID NO: 1. In another aspect, the variant comprises or consists of a substitution at a position corresponding to position 198 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 200 of the polypeptide of SEQ ID NO: 1. In another aspect, the amino acid at a position corresponding to position 200 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys. In another aspect, the variant comprises or consists of the substitution R200C corresponding to the polypeptide of SEQ ID NO: 1. In another aspect, the variant comprises or consists of a substitution at a position corresponding to position 200 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., A89C+R200C).

In another aspect, the variant comprises or consists of a substitution at a position corresponding to position 201 of the polypeptide of SEQ ID NO: 1. In another aspect, the amino acid at a position corresponding to position 201 is substituted with Ala, Arg, Asn, Asp, Cys, Gln, Glu, Gly, His, Ile, Leu, Lys, Met, Phe, Pro, Ser, Thr, Trp, Tyr, or Val, preferably with Met or Ile. In another aspect, the variant comprises or consists of the substitution K201M or K201I corresponding to the polypeptide of SEQ ID NO: 1. In another aspect, the variant comprises or consists of a substitution at a position corresponding to position 201 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 202 of the polypeptide of SEQ ID NO: 1. In another aspect, the amino acid at a position corresponding to position 202 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu, Val, Ala or Thr. In another aspect, the variant comprises or consists of the substitution I202L, I202V, I202A or I202T corresponding to the polypeptide of SEQ ID NO: 1. In another aspect, the variant comprises or consists of a substitution at a position corresponding to position 202 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+E186K+E188P+M189L+I202L).

In another aspect, the variant comprises or consists of a substitution at a position corresponding to position 203 of the polypeptide of SEQ ID NO: 1. In another aspect, the amino acid at a position corresponding to position 203 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu or Ile. In another aspect, the variant comprises or consists of the substitution M203L or M203I corresponding to the polypeptide of SEQ ID NO: 1. In another aspect, the variant comprises or consists of a substitution at a position corresponding to position 203 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 205 of the polypeptide of SEQ ID NO: 1. In another aspect, the amino acid at a position corresponding to position 205 is substituted with Ala, Arg, Asn, Asp, Cys, Gln, Glu, Gly, His, Ile, Leu, Lys, Met, Phe, Pro, Ser, Thr, Trp, Tyr, or Val, preferably with Glu. In another aspect, the variant comprises or consists of the substitution G205E corresponding to the polypeptide of SEQ ID NO: 1. In another aspect, the variant comprises or consists of a substitution at a position corresponding to position 205 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D18P+D39L+E186K+E188P+M189L+G205E).

In another aspect, the variant comprises or consists of a substitution at a position corresponding to position 206 of the polypeptide of SEQ ID NO: 1. In another aspect, the amino acid at a position corresponding to position 206 is substituted with Ala, Arg, Asn, Asp, Cys, Gln, Glu, Gly, His, Ile, Leu, Lys, Met, Phe, Pro, Ser, Thr, Trp, Tyr, or Val, preferably with Pro. In another aspect, the variant comprises or consists of the substitution D206P corresponding to the polypeptide of SEQ ID NO: 1. In another aspect, the variant comprises or consists of a substitution at a position corresponding to position 206 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., M22N+A89E+K151P+E188P+D206P).

In another aspect, the variant comprises or consists of a substitution at a position corresponding to position 207 of the polypeptide of SEQ ID NO: 1. In another aspect, the amino acid at a position corresponding to position 207 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asn. In another aspect, the variant comprises or consists of the substitution T207N corresponding to the polypeptide of SEQ ID NO: 1. In another aspect, the variant comprises or consists of a substitution at a position corresponding to position 207 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 208 of the polypeptide of SEQ ID NO: 1. In another aspect, the amino acid at a position corresponding to position 208 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises or consists of the substitution N208L corresponding to the polypeptide of SEQ ID NO: 1. In another aspect, the variant comprises or consists of a substitution at a position corresponding to position 208 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 211 of the polypeptide of SEQ ID NO: 1. In another aspect, the amino acid at a position corresponding to position 211 is substituted with Ala, Arg, Asn, Asp, Cys, Gln, Glu, Gly, His, Ile, Leu, Lys, Met, Phe, Pro, Ser, Thr, Trp, Tyr, or Val, preferably with Thr. In another aspect, the variant comprises or consists of the substitution V211T corresponding to the polypeptide of SEQ ID NO: 1. In another aspect, the variant comprises or consists of a substitution at a position corresponding to position 211 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+E132D+E140D+K151E+D163E+Y165F+E190D+E194D+V211T+L214I+M218L+E221R; N35D+Y72F+V74E+K83E+Y100F+K111E+K123E+E132D+E140D+K151E+D163E+Y165F+E190D+E194D+V211T+L214I+E221K; R36L+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 214 of the polypeptide of SEQ ID NO: 1. In another aspect, the amino acid at a position corresponding to position 214 is substituted with Ala, Arg, Asn, Asp, Cys, Gln, Glu, Gly, His, Ile, Leu, Lys, Met, Phe, Pro, Ser, Thr, Trp, Tyr, or Val, preferably with Ile. In another aspect, the variant comprises or consists of the substitution L214I corresponding to the polypeptide of SEQ ID NO: 1. In another aspect, the variant comprises or consists of a substitution at a position corresponding to position 214 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 215 of the polypeptide of SEQ ID NO: 1. In another aspect, the amino acid at a position corresponding to position 215 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises or consists of the substitution N215L corresponding to the polypeptide of SEQ ID NO: 1. In another aspect, the variant comprises or consists of a substitution at a position corresponding to position 215 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 216 of the polypeptide of SEQ ID NO: 1. In another aspect, the amino acid at a position corresponding to position 216 is substituted with Ala, Arg, Asn, Asp, Cys, Gln, Glu, Gly, His, Ile, Leu, Lys, Met, Phe, Pro, Ser, Thr, Trp, Tyr, or Val, preferably with Cys, Asn, Gly, His, Phe, or Pro. In another aspect, the variant comprises or consists of the substitution A216C, A216N, A216G, A216H, A216F, or A216P corresponding to the polypeptide of SEQ ID NO: 1. In another aspect, the variant comprises or consists of a substitution at a position corresponding to position 216 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 217 of the polypeptide of SEQ ID NO: 1. In another aspect, the amino acid at a position corresponding to position 217 is substituted with Ala, Arg, Asn, Asp, Cys, Gln, Glu, Gly, His, Ile, Leu, Lys, Met, Phe, Pro, Ser, Thr, Trp, Tyr, or Val, preferably with Asn. In another aspect, the variant comprises or consists of the substitution R217N corresponding to the polypeptide of SEQ ID NO: 1. In another aspect, the variant comprises or consists of a substitution at a position corresponding to position 217 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1.

In another aspect, the variant comprises or consists of a substitution at a position corresponding to position 218 of the polypeptide of SEQ ID NO: 1. In another aspect, the amino acid at a position corresponding to position 218 is substituted with Ala, Arg, Asn, Asp, Cys, Gln, Glu, Gly, His, Ile, Leu, Lys, Met, Phe, Pro, Ser, Thr, Trp, Tyr, or Val, preferably with Lys, Val, Ile, Phe, Thr, Ser, Asn, Ala, Tyr, Gly or Leu. In another aspect, the variant comprises or consists of the substitution M218K, M218V, M218I, M218N, M218S, M218F, M218A, M218Y, M218T, M218G or M218L corresponding to the polypeptide of SEQ ID NO: 1. In another aspect, the variant comprises or consists of a substitution at a position corresponding to position 218 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., R36D+M218K; K41C+M218K; K43A+M218K; H112E+M218K; E188P+M218I; D18P+K43A+M218K; R36D+K41C+M218K; R36D+K43A+M218K; K41C+K43A+M218K; K94T+H112N+M218K; K94T+M189V+M218K; E186K+M191V+M218V; D18P+R36D+K43A+M218K; D18K+D39L+H99L+M218V; D18E+N141K+E186R+M218V; R36D+K41C+K43A+M218K; D39R+K64L+S177Y+M218V; D39L+H99L+E186K+M218V; D39L+N141K+E186L+M218V; A53G+K147I+E186R+M218V; K64L+S177Y+M218V+E221L; D163K+S177Y+E186R+M218V; E186R+M189L+M218V+E221L; D18K+D39R+E186R+M218V; D39R+E186R+M191V+M218V; D18K+D39L+E186R+M218V; E186R+M189I+M218V+E221R; K64L+S177Y+E186R+M218V; D39L+D163K+E186R+M218V; K64L+H99L+E186R+M218V; D39L+K147I+E186R+M218V; D18P+K41C+K43A+M218K; S177Y+E186R+M191V+M218V; K64L+K147I+E186R+M218V; D39L+K64L+M218V+E221L; D18K+D39L+D163K+M218V; A53G+E186R+M189I+M218V; D18E+H99L+E186R+M218V; D18P+R36D+K41C+K43A+M218K; D18P+N141K+E186R+E188P+M218V; D18P+D39L+E186L+E188P+M218V; D39L+D163K+E186K+M191V+M218V+E221R; D39L+D163K+E186R+M191V+M218V+E221R; D39K+K43A+D161N+E186K+E188P+M218K+E221K+K222*; D18P+D39K+K43A+D161N+E186K+E188P+M218K+E221K+K222*; R36D+D39K+K43A+D161N+E186K+E188P+M218K+E221K+K222*; D39K+K41C+K43A+D161N+E186K+E188P+M218K+E221K+K222*; R36D+D39K+K41C+K43A+D161N+E186K+E188P+M218K+E221K+K222*; D18P+D39K+K41C+K43A+D161N+E186K+E188P+M218K+E221K+K222*; D18P+R36D+D39K+K41C+K43A+D161N+E186K+E188P+M218K+E221K+K222*; Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; R36L+Y72F+V74E+K83E+Y100F+K111E+K123E+E132D+E140D+K151E+D163E+Y165F+E190D+E194D+V211T+L214I+M218L+E221R; R36I+Y72F+V74E+K83E+Y100F+K111E+K123E+G125A+E132D+E140D+A149P+K151E+D163E+Y165F+E190D+E194D+V211T+L214I+M218I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R).

In another aspect, the variant comprises or consists of a substitution at a position corresponding to position 220 of the polypeptide of SEQ ID NO: 1. In another aspect, the amino acid at a position corresponding to position 220 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises or consists of the substitution M220L corresponding to the polypeptide of SEQ ID NO: 1. In another aspect, the variant comprises or consists of a substitution at a position corresponding to position 220 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., F102L+M220L).

In another aspect, the variant comprises or consists of a substitution at a position corresponding to position 221 of the polypeptide of SEQ ID NO: 1. In another aspect, the amino acid at a position corresponding to position 221 is substituted with Ala, Arg, Asn, Asp, Cys, Gln, Glu, Gly, His, Ile, Leu, Lys, Met, Phe, Pro, Ser, Thr, Trp, Tyr, or Val, preferably with Arg, Leu, Ile, Phe, Tyr, Thr, Val or Lys. In another aspect, the variant comprises or consists of the substitution E221R, E221L, E221I, E221F, E221Y, E221T, E221V or E221K corresponding to the polypeptide of SEQ ID NO: 1. In another aspect, the variant comprises or consists of a substitution at a position corresponding to position 221 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1 (e.g., D39L+E221I; S177Y+E221L; E186K+E221K; M191V+E221L; D39R+K64L+E221L; K41C+E186K+E221K; E186K+M191V+E221L; D18K+D39R+E186R+E221R; D39L+A53G+E186R+E221R; D39L+A53G+M189L+E221L; D39L+S177Y+E186R+E221R; K64L+S177Y+E186R+E221L; K64L+S177Y+M218V+E221L; D161N+E186K+E188P+E221K; E186R+M189L+M218V+E221L; D18E+D163K+E186R+E221R; S177Y+E186R+M191V+E221R; D18E+D39L+D163K+E221R; D39L+E186R+M191V+E221R; H99L+K147I+E186R+E221R; D39L+D163K+E186K+E221L; D18K+H99L+E186R+E221L; D39L+K147I+E186L+E221R; D18K+D39L+E186K+E221R; D18K+D39L+N141K+E221L; D39L+H99L+D163K+E221L; D39L+D163K+E186R+E221L; A53G+D163K+E186R+E221R; D39K+E186K+E221K+K222*; D39L+K64L+N141K+E221R; D39L+H99L+E186R+E221R; D39L+E186R+M191V+E221L; D18E+N141K+E186R+E221L; D39L+S177Y+E186K+E221R; D18K+E186R+M191V+E221L; K147I+S177Y+E186R+E221L; D39R+K64L+S177Y+E221R; K64L+S177Y+E186K+E221R; K64L+S177Y+E186R+E221R; H99L+N141K+E186R+E221L; D39R+E186R+M189L+E221R; D39L+H99L+N141K+E221R; D39L+K147I+E186L+E221L; D39L+H99L+E186K+E221R; E186R+M189I+M218V+E221R; K147I+D163K+E186R+E221L; A53G+N141K+E186R+E221L; D18K+E186R+M189L+E221L; D39R+K147I+E186R+E221R; A53G+E186R+M189L+E221R; D18K+D39L+M189I+E221R; D39L+K64L+M218V+E221L; D39L+K147I+S177Y+E221L; D18E+E186R+M189I+E221L; D18P+D39R+E186R+E188P+E221L; D18P+D39L+E188P+M189L+E221R; D18P+A53G+E186R+E188P+E221L; D18P+E186R+E188P+M189I+E221L; D18P+N141K+E186R+E188P+E221R; K41C+D161N+E186K+E188P+E221K; D39K+K41C+E186K+E221K+K222*; D18P+K64L+E186R+E188P+E221R; D39K+D161N+E186K+E188P+E221K+K222*; D39L+D163K+E186K+M191V+M218V+E221R; D39L+D163K+E186R+M191V+M218V+E221R; D39K+K41C+D161N+E186K+E188P+E221K+K222*; D18P+D39K+D161N+E186K+E188P+E221K+K222*; D39K+K43A+D161N+E186K+E188P+M218K+E221K+K222*; D18P+D39K+K41C+D161N+E186K+E188P+E221K+K222*; D18P+D39K+K43A+D161N+E186K+E188P+M218K+E221K+K222*; R36D+D39K+K43A+D161N+E186K+E188P+M218K+E221K+K222*; D39K+K41C+K43A+D161N+E186K+E188P+M218K+E221K+K222*; R36D+D39K+K41C+K43A+D161N+E186K+E188P+M218K+E221K+K222*; D18P+D39K+K41C+K43A+D161N+E186K+E188P+M218K+E221K+K222*; D18P+R36D+D39K+K41C+K43A+D161N+E186K+E188P+M218K+E221K+K222*; Y72F+V74E+K83E+Y100F+K111E+K123E+E132D+E140D+K151E+D163E+Y165F+E190D+E194D+V211T+L214I+M218L+E221R; R36L+Y72F+V74E+K83E+Y100F+K111E+K123E+E132D+E140D+K151E+D163E+Y165F+E190D+E194D+V211T+L214I+M218I+E221R; N35D+Y72F+V74E+K83E+Y100F+K111E+K123E+E132D+E140D+K151E+D163E+Y165F+E190D+E194D+V211T+L214I+E221K; Y72F+V74E+K83E+Y100F+K111E+K123E+G125S+E132D+E140D+A149D+K151E+D163E+Y165F+E190D+E194D+V211T+L214I+E221R; N35D+Y72F+V74E+K83E+Y100F+K111E+K123E+E132D+E140D+A149D+K151E+D163E+Y165F+E190D+E194D+V211T+L214I+E221R; N35D+Y72F+V74E+K83E+Y100F+K111E+K123E+E132D+E140D+K151E+D163E+Y165F+E190D+E194A+V211T+L214I+E221R; N35D+Y72F+V74E+K83E+Y100F+K111E+K123E+E132D+E140D+K151E+D163E+Y165F+E190D+E194D+V211T+L214I+E221R; R36L+Y72F+V74E+K83E+Y100F+K111E+K123E+E132D+E140D+K151E+D163E+Y165F+E190D+E194D+V211T+L214I+M218L+E221R; Y72F+V74E+K83E+Y100F+K111E+K123E+E132D+E140D+A149D+K151E+D163E+Y165F+E190D+E194D+V211T+L214I+E221R; Y72F+V74E+K83E+Y100F+K111E+K123E+G125A+E132D+E140D+A149P+K151E+D163E+Y165F+E190D+E194D+V211T+L214I+E221R; R36I+Y72F+V74E+K83E+Y100F+K111E+K123E+G125A+E132D+E140D+A149P+K151E+D163E+Y165F+E190D+E194D+V211T+L214I+M218I+E221R; N35D+Y72F+V74E+K83E+Y100F+K111E+K123E+E132D+E140D+K151E+R162L+D163E+Y165F+E190D+E194D+V211T+L214I+E221R; Y72F+V74E+K83E+Y100F+K111E+K123E+E132D+E140D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218I+E221R; Y72F+V74E+K83E+Y100F+K111E+K123E+E132D+E140D+A149P+K151E+D163E+Y165F+E190D+E194D+V211T+L214I+E221R; N35D+Y72F+V74E+K83I+Y100F+K111E+K123E+E132D+E140D+K151E+D163E+Y165F+E190D+E194D+V211T+L214I+E221R; R36I+Y72F+V74E+K83E+Y100F+K111E+K123E+E132D+E140D+A149D+K151E+D163E+Y165F+E190D+E194D+V211T+L214I+M218I+E221R; R36I+Y72F+V74E+K83E+Y100F+K111E+K123E+E132D+E140D+K151E+D163E+Y165F+E190D+E194D+V211T+L214I+M218L+E221R; H12L+N35D+Y72F+V74E+K83E+Y100F+K111E+K123E+E132D+E140D+K151E+D163E+Y165F+E190D+E194D+V211T+L214I+E221R; Y72F+V74E+K83E+Y100F+K111E+K123E+G125D+E132D+E140D+K151E+D163E+Y165F+E190D+E194D+V211T+L214I+E221R; R36I+Y72F+V74E+K83E+Y100F+K111E+K123E+E132D+E140D+K151E+D163E+Y165F+E190D+E194D+V211T+L214I+E221R).

In another aspect, the variant comprises or consists of a substitution at a position corresponding to position 222 of the polypeptide of SEQ ID NO: 1. In another aspect, the amino acid at a position corresponding to position 222 is substituted with Ala, Arg, Asn, Asp, Cys, Gln, Glu, Gly, His, Ile, Leu, Lys, Met, Phe, Pro, Ser, Thr, Trp, Tyr, or Val, preferably with Leu. In another aspect, the variant comprises or consists of the substitution K222L corresponding to the polypeptide of SEQ ID NO: 1. In another aspect, the variant comprises or consists of a substitution at a position corresponding to position 222 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, and 221 of the polypeptide of SEQ ID NO: 1; and/or an insertion or one or more amino acids at a position corresponding to position 36 of the polypeptide of SEQ ID NO: 1.

In another aspect, the variant comprises or consists of a deletion at a position corresponding to position 222 of the polypeptide of SEQ ID NO: 1. In another aspect, the variant comprises or consists of deletion at a position corresponding to position 222 of the polypeptide of SEQ ID NO: 1, together with a substitution at one or more positions described herein, such as a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220 and 221 of the polypeptide of SEQ ID NO: 1; and/or an insertion or one or more amino acids at a position corresponding to position 36 of the polypeptide of SEQ ID NO: 1 (e.g., D39K+K222*; D39K+K41C+K222*; D39K+D161N+E188P+K222*; D39K+E186K+E221K+K222*; D39K+K41C+E186K+E221K+K222*; D39K+K41C+D161N+E188P+K222*; D39K+D161N+E186K+E188P+E221K+K222*; D39K+K41C+D161N+E186K+E188P+E221K+K222*; D18P+D39K+D161N+E186K+E188P+E221K+K222*; D39K+K43A+D161N+E186K+E188P+M218K+E221K+K222*; D18P+D39K+K41C+D161N+E186K+E188P+E221K+K222*; D18P+D39K+K43A+D161N+E186K+E188P+M218K+E221K+K222*; R36D+D39K+K43A+D161N+E186K+E188P+M218K+E221K+K222*; D39K+K41C+K43A+D161N+E186K+E188P+M218K+E221K+K222*; R36D+D39K+K41C+K43A+D161N+E186K+E188P+M218K+E221K+K222*; D18P+D39K+K41C+K43A+D161N+E186K+E188P+M218K+E221K+K222*; D18P+R36D+D39K+K41C+K43A+D161N+E186K+E188P+M218K+E221K+K222*).

In another aspect, the variant is selected from any one of the variants of Table 2 (e.g., any one of variants 1-87 or 89-183), Table 3 (e.g., any one of variants 1-802 or 804-941), Table 4 (e.g., any one of variants 1-119 or 121-293, Table 5 (e.g., any one of variants 1-150 or 152-261) or Table 6 (e.g., any one of variants 1-74 or 76-211).

The amino acid changes elsewhere in the polypeptide may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244:1081-1085). In the latter technique, single alanine mutations are introduced at every residue in the molecule, and the resultant molecules are tested for carbonic anhydrase activity to identify amino acid residues that are critical to the activity of the molecule. See also, Hilton et al., 1996, J. Biol. Chem. 271:4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255:306-312; Smith et al., 1992, J. Mol. Biol. 224:899-904; Wlodaver et al., 1992, FEBS Lett. 309:59-64. The identity of essential amino acids can also be inferred from an alignment with a related polypeptide, and/or be inferred from sequence homology and conserved catalytic machinery with a related polypeptide or within a polypeptide or protein family with polypeptides/proteins descending from a common ancestor, typically having similar three-dimensional structures, functions, and significant sequence similarity. Additionally or alternatively, protein structure prediction tools can be used for protein structure modelling to identify essential amino acids and/or active sites of polypeptides. See, for example, Jumper et al., 2021, “Highly accurate protein structure prediction with AlphaFold”, Nature 596:583-589.

Alpha-carbonic anhydrases are identified by their consensus sequence motif: S-E-[HN]-x-[LIVM]-x(4)-[FYH]-x(2)-E-[LIVMGA]-H-[LIVMFA] (2). The respective consensus residues corresponds to positions 91 to 115 in SEQ ID NO: 1. In one embodiment, the carbonic anhydrase variant comprises the consensus sequence motif S-E-[HN]-x-[LIVM]-x(4)-[FYH]-x(2)-E-[LIVMGA]-H-[LIVMFA] (2).

Additional guidance on the structure-activity relationship of the carbonic anhydrases herein can be determined using multiple sequence alignment (MSA) techniques well-known in the art. Based on the teachings herein, the skilled artisan could make similar alignments with any number of glycerol transporters described herein or known in the art. Such alignments aid the skilled artisan to determine potentially relevant domains (e.g., binding domains or catalytic domains), as well as which amino acid residues are conserved and not conserved among the different transporter sequences. It is appreciated in the art that changing an amino acid that is conserved at a particular position between disclosed polypeptides will more likely result in a change in biological activity (Bowie et al., 1990, Science 247:1306-1310: “Residues that are directly involved in protein functions such as binding or catalysis will certainly be among the most conserved”). In contrast, substituting an amino acid that is not highly conserved among the polypeptides will not likely or significantly alter the biological activity.

Even further guidance on the structure-activity relationship for the skilled artisan can be found in published x-ray crystallography studies known in the art. A crystal structure of the Persephonella marina α-carbonic anhydrase of SEQ ID NO: 1 identified active site residues, the calcium binding site, and disulfide residues important for thermostability (Kim et al., 2019, Mol. Cells. 42 (6): 460-469).

The following amino acid residues H86, H88, and H105 (numbering according to SEQ ID NO: 1) are predicted to form a histidine triad which is important for catalysis. In one embodiment of the present invention the carbonic anhydrase contains histidine residues positions corresponding to positions 86, 88, and 105 of SEQ ID NO: 1.

The following amino acid residues C23 and C176 (numbering according to SEQ ID NO: 1) are predicted to form a disulfide bridge to provide conformational and thermal stability by increasing structural rigidity. In one embodiment of the present invention the carbonic anhydrase contains cysteine residues at positions corresponding to positions 23 and 176 of SEQ ID NO: 1.

The following amino acid residues Y4, H61, E92 and T172 (using SEQ ID NO: 1 numbering) are predicted to participate in a proton shuttle mechanism, which also is relevant for the catalytic activity of the enzyme. In a further embodiment, the carbonic anhydrase contains a tyrosine at a position corresponding to position 61 of SEQ ID NO: 1, a histidine at a position corresponding to position 61 of SEQ ID NO: 1, a glutamic acid at a position corresponding to position 92 of SEQ ID NO: 1 and/or a threonine at a position corresponding to position 193 of SEQ ID NO: 1. Preferably, at least one of the proton shuttle positions are present, more preferably at least two proton shuttle positions are present, more preferably at least three proton shuttle positions are present and most preferably all the proton shuttle positions are present in the carbonic anhydrase.

The following amino acid residues V107, V117, L119, L171, V180 and W182 (using SEQ ID NO: 1 numbering) are predicted to be responsible for CO₂ binding. In a further embodiment, the carbonic anhydrase contains a valine at a position corresponding to position 107 of SEQ ID NO: 1, a valine at a position corresponding to position 117 of SEQ ID NO: 1, a leucine at a position corresponding to position 119 of SEQ ID NO: 1, a leucine at a position corresponding to position 171 of SEQ ID NO: 1, a valine at a position corresponding to position 180 of SEQ ID NO: 1 and/or a tryptophan at a position corresponding to position 182 of SEQ ID NO: 1. Preferably, at least one of the residues responsible for CO₂ binding present, more preferably at least, at least three, at least four, at least five, or all six residues.

Additional guidance on the structure-activity relationship for the skilled artisan can be found in published x-ray crystallography studies known in the art from the crystal structure of the carbonic anhydrase of SEQ ID NO: 2 (Fredslund et al., 2018, Enzyme Microb. Technol. 114:48-54).

The variant may be a fragment of any of the variants described herein, e.g., the number of amino acid residues in the fragment may be at least 75%, e.g., at least 80%, 85%, 90%, or 95% of the number of amino acid residues of the referenced variant or parent such as SEQ ID NO: 1, SEQ ID NO: 2, SEQ ID NO: 3, Or SEQ ID NO: 4 having carbonic anhydrase activity. In one embodiment, the fragment has e.g., at least 150 amino acids, 175 amino acids, 185 amino acids, 195 amino acids 205 amino acids, 215 amino acids, or at least 220 amino acids; or 150-175 amino acids, 175-185 amino acids, 185-195 amino acids, 195-205 amino acids, or 205-215 amino acids.

In one embodiment, the carbonic anhydrase variant has improved thermostability compared to the parent enzyme when tested under the same conditions (e.g., using an assay described in WO2018/017792, e.g., after incubation for the given time in 1 M NaHCO₃ buffer at pH 8 at the given elevated temperature). In one embodiment, the variant maintains at least 5% (e.g., at least 10%, at least 15%, at least 20%, at least 25%, at least 30%, at least 40%, at least 50%) higher residual activity at e.g., 1, 2, 3, 4, 5, 6, 7, 8, 9, 10, 15, or 20 days compared to the parent enzyme when tested under the same conditions (e.g., in 1 M NaHCO₃ buffer at pH 8 at elevated temperature, such as 50° C. or 80° C.).

In one embodiment, the carbonic anhydrase variants are capable of maintaining a residual activity of at least 30%, preferably above 40%, more preferably above 50%, more preferably above 60%, even more preferably above 70%, most preferably above 80%, most preferably above 85%, most preferably above 90%, most preferably above 95%, and even most preferably the residual activity is unchanged after incubation in 1 M NaHCO₃ buffer pH 8 at temperatures above 45° C., preferably above 50° C., above 55° C., above 60° C., above 65° C., more preferably above 70° C., most preferably above 80° C., most preferably above 90° C., most preferably above 100° C., most preferably above 105° C. and even most preferably above 110° C. for at least 15 minutes, preferably for at least 2 hours, more preferably for at least 24 hours, more preferably for at least 7 days, more preferably for at least 10 days, even more preferably for at least 14 days, most preferably for at least 30 days, even most preferably for at least 50 days at the elevated temperature.

In one embodiment, the variant maintains at least 50% residual activity in 30% methyldiethanolamine (MDEA) at 85° C. after five days. In one embodiment, the variant maintains at least 85% activity when incubated for 15 minutes in 1M NaHCO₃ solution (approximately pH 8-10) in the temperature range 25-90° C. In one embodiment, the enzyme maintains full activity at 50° C. over the pH range 4-11 for one day. In one embodiment, the enzyme maintains more than 50% activity over the pH range 4-11 after 10 days at 50° C.

In one embodiment, the carbonic anhydrase variant has a higher protein melting point (Tm) compared to the parent enzyme when tested under the same conditions (e.g., the conditions described in Example 2, such as 20% methyldiethanolamine (MDEA), pH 10). In one embodiment, the carbonic anhydrase variant has a higher Tm of at least 2° C., e.g., at least 5° C., at least 7° C., at least 10° C., at least 12° C., at least 15° C., or at least 17° C., compared to the parent enzyme when tested under the same conditions (e.g., the conditions described in Example 2, such as 20% methyldiethanolamine (MDEA), pH 10).

The polypeptide may be a fusion polypeptide comprising a variant of the invention.

In an aspect, the variant is isolated.

In another aspect, the variant is purified.

### Parent Carbonic Anhydrases

The parent may be obtained from microorganisms of any genus. For purposes of the present invention, the term “obtained from” as used herein in connection with a given source shall mean that the parent encoded by a polynucleotide is produced by the source or by a strain in which the polynucleotide from the source has been inserted. In one aspect, the parent is secreted extracellularly.

In one aspect, the parent is a Persephonella carbonic anhydrase, e.g., a carbonic anhydrase from Persephonella marina, Persephonella hydrogeniphila or Persephonella guaymasensis, such as the carbonic anhydrase of SEQ ID NO: 1, 3 or 4.

The parent carbonic anhydrase may be a polypeptide having at least 60% sequence identity to the polypeptide of SEQ ID NO: 1. In an aspect, the parent has a sequence identity to the polypeptide of SEQ ID NO: 1 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have carbonic anhydrase activity. In one aspect, the amino acid sequence of the parent differs by up to 10 amino acids, e.g., 1, 2, 3, 4, 5, 6, 7, 8, 9, or 10, from the polypeptide of SEQ ID NO: 1. In another aspect, the parent comprises or consists of the amino acid sequence of SEQ ID NO: 1. In another aspect, the parent is a fragment of the polypeptide of SEQ ID NO: 1 having carbonic anhydrase activity. In one embodiment, the number of amino acid residues in the fragment is at least 75%, e.g., at least 80%, 85%, 90%, or 95% of the number of amino acid residues of the referenced parent (e.g., SEQ ID NO: 1).

The parent carbonic anhydrase may be a polypeptide having at least 60% sequence identity to the polypeptide of SEQ ID NO: 3. In an aspect, the parent has a sequence identity to the polypeptide of SEQ ID NO: 3 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have carbonic anhydrase activity. In one aspect, the amino acid sequence of the parent differs by up to 10 amino acids, e.g., 1, 2, 3, 4, 5, 6, 7, 8, 9, or 10, from the polypeptide of SEQ ID NO: 3. In another aspect, the parent comprises or consists of the amino acid sequence of SEQ ID NO: 3. In another aspect, the parent is a fragment of the polypeptide of SEQ ID NO: 3 having carbonic anhydrase activity. In one embodiment, the number of amino acid residues in the fragment is at least 75%, e.g., at least 80%, 85%, 90%, or 95% of the number of amino acid residues of the referenced parent (e.g., SEQ ID NO: 3).

The parent carbonic anhydrase may be a polypeptide having at least 60% sequence identity to the polypeptide of SEQ ID NO: 4. In an aspect, the parent has a sequence identity to the polypeptide of SEQ ID NO: 4 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have carbonic anhydrase activity. In one aspect, the amino acid sequence of the parent differs by up to 10 amino acids, e.g., 1, 2, 3, 4, 5, 6, 7, 8, 9, or 10, from the polypeptide of SEQ ID NO: 4. In another aspect, the parent comprises or consists of the amino acid sequence of SEQ ID NO: 4. In another aspect, the parent is a fragment of the polypeptide of SEQ ID NO: 4 having carbonic anhydrase activity. In one embodiment, the number of amino acid residues in the fragment is at least 75%, e.g., at least 80%, 85%, 90%, or 95% of the number of amino acid residues of the referenced parent (e.g., SEQ ID NO: 4).

In another aspect, the parent is a metagenomic sequence isolated from the Logatchev hydrothermal vent (Perner et al., 2013, Environ. Microbiol. 15:1551-1560), such as the carbonic anhydrase of SEQ ID NO: 2.

The parent carbonic anhydrase may be a polypeptide having at least 60% sequence identity to the polypeptide of SEQ ID NO: 2. In an aspect, the parent has a sequence identity to the polypeptide of SEQ ID NO: 2 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have carbonic anhydrase activity. In one aspect, the amino acid sequence of the parent differs by up to 10 amino acids, e.g., 1, 2, 3, 4, 5, 6, 7, 8, 9, or 10, from the polypeptide of SEQ ID NO: 2. In another aspect, the parent comprises or consists of the amino acid sequence of SEQ ID NO: 2. In another aspect, the parent is a fragment of the polypeptide of SEQ ID NO: 2 having carbonic anhydrase activity. In one embodiment, the number of amino acid residues in the fragment is at least 75%, e.g., at least 80%, 85%, 90%, or 95% of the number of amino acid residues of the referenced parent (e.g., SEQ ID NO: 2).

The approximate percent identities among the mature parent carbonic anhydrase enzymes sequences of SEQ ID NOs: 1-4 is shown in Table 1 below.

TABLE 1

| | SEQ ID | SEQ ID | SEQ ID | SEQ ID |
|---|---|---|---|---|
| | NO: 1 | NO: 2 | NO: 3 | NO: 4 |
| SEQ ID NO: 1 | 100 | 79.3 | 63.9 | 78.7 |
| SEQ ID NO: 2 | 79.3 | 100 | 57.0 | 82.8 |
| SEQ ID NO: 3 | 63.9 | 57.0 | 100 | 54.4 |
| SEQ ID NO: 4 | 78.7 | 82.8 | 54.4 | 100 |

The parent may be a fusion polypeptide or cleavable fusion polypeptide. A fusion polypeptide is produced by fusing a polynucleotide encoding another polypeptide to a polynucleotide of the present invention. Techniques for producing fusion polypeptides are known in the art and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12:2575-2583; Dawson et al., 1994, Science 266:776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3:568-576; Svetina et al., 2000, J. Biotechnol. 76:245-251; Rasmussen-Wilson et al., 1997, Appl. Environ. Microbiol. 63:3488-3493; Ward et al., 1995, Biotechnology 13:498-503; and Contreras et al., 1991, Biotechnology 9:378-381; Eaton et al., 1986, Biochemistry 25:505-512; Collins-Racie et al., 1995, Biotechnology 13:982-987; Carter et al., 1989, Proteins: Structure, Function, and Genetics 6: 240-248; and Stevens, 2003, Drug Discovery World 4:35-48.

### Preparation of Variants

The present invention also relates to methods for obtaining a variant having carbonic anhydrase activity, comprising: (a) introducing into a parent carbonic anhydrase a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1, wherein the variant has carbonic anhydrase activity; and (b) recovering the variant.

The variants can be prepared using any mutagenesis procedure known in the art, such as site-directed mutagenesis, synthetic gene construction, semi-synthetic gene construction, random mutagenesis, shuffling, etc.

Site-directed mutagenesis is a technique in which one or more mutations are introduced at one or more defined sites in a polynucleotide encoding the parent.

Site-directed mutagenesis can be accomplished in vitro by PCR involving the use of oligonucleotide primers containing the desired mutation. Site-directed mutagenesis can also be performed in vitro by cassette mutagenesis involving the cleavage by a restriction enzyme at a site in the plasmid comprising a polynucleotide encoding the parent and subsequent ligation of an oligonucleotide containing the mutation in the polynucleotide. Usually the restriction enzyme that digests the plasmid and the oligonucleotide is the same, permitting sticky ends of the plasmid and the insert to ligate to one another. See, e.g., Scherer and Davis, 1979, Proc. Natl. Acad. Sci. USA 76:4949-4955; and Barton et al., 1990, Nucleic Acids Res. 18:7349-4966.

Site-directed mutagenesis can also be accomplished in vivo by methods known in the art. See, e.g., US 2004/0171154; Storici et al., 2001, Nature Biotechnol. 19:773-776; Kren et al., 1998, Nat. Med. 4:285-290; and Calissano and Macino, 1996, Fungal Genet. Newslett. 43:15-16.

Any site-directed mutagenesis procedure can be used in the present invention. There are many commercial kits available that can be used to prepare variants.

Synthetic gene construction entails in vitro synthesis of a designed polynucleotide molecule to encode a polypeptide of interest. Gene synthesis can be performed utilizing a number of techniques, such as the multiplex microchip-based technology described by Tian et al., 2004, Nature 432:1050-1054, and similar technologies wherein oligonucleotides are synthesized and assembled upon photo-programmable microfluidic chips.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241:53-57; Bowie and Sauer, 1989, Proc. Natl. Acad. Sci. USA 86:2152-2156; WO 95/17413; or WO 95/22625. Other methods that can be used include error-prone PCR, phage display (e.g., Lowman et al., 1991, Biochemistry 30:10832-10837; U.S. Pat. No. 5,223,409; WO 92/06204) and region-directed mutagenesis (Derbyshire et al., 1986, Gene 46:145; Ner et al., 1988, DNA 7:127).

Mutagenesis/shuffling methods can be combined with high-throughput, automated screening methods to detect activity of cloned, mutagenized polypeptides expressed by host cells (Ness et al., 1999, Nature Biotechnology 17:893-896). Mutagenized DNA molecules that encode active polypeptides can be recovered from the host cells and rapidly sequenced using standard methods in the art. These methods allow the rapid determination of the importance of individual amino acid residues in a polypeptide.

Semi-synthetic gene construction is accomplished by combining aspects of synthetic gene construction, and/or site-directed mutagenesis, and/or random mutagenesis, and/or shuffling. Semi-synthetic construction is typified by a process utilizing polynucleotide fragments that are synthesized, in combination with PCR techniques. Defined regions of genes may thus be synthesized de novo, while other regions may be amplified using site-specific mutagenic primers, while yet other regions may be subjected to error-prone PCR or non-error prone PCR amplification. Polynucleotide subsequences may then be shuffled.

### Polynucleotides

The present invention also relates to polynucleotides encoding a variant of the present invention. The polynucleotide may be a genomic DNA, a cDNA, a synthetic DNA, a synthetic RNA, a mRNA, or a combination thereof.

In an aspect, the polynucleotide is isolated.

In another aspect, the polynucleotide is purified.

### Nucleic Acid Constructs

The present invention also relates to nucleic acid constructs comprising a polynucleotide encoding a variant of the present invention operably linked to one or more control sequences that direct the expression of the coding sequence in a suitable host cell under conditions compatible with the control sequences.

The polynucleotide may be manipulated in a variety of ways to provide for expression of a variant. Manipulation of the polynucleotide prior to its insertion into a vector may be desirable or necessary depending on the expression vector. The techniques for modifying polynucleotides utilizing recombinant DNA methods are well known in the art.

### Promoters

The control sequence may be a promoter, a polynucleotide recognized by a host cell for expression of a polynucleotide encoding a variant of the present invention. The promoter contains transcriptional control sequences that mediate the expression of the variant. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the polynucleotide of the present invention in a bacterial host cell are described in Sambrook et al., 1989, Molecular Cloning: A Laboratory Manual, Cold Spring Harbor Lab., NY, Davis et al., 2012, Basic Methods in Molecular Biology, Elsevier, and Song et al., 2016, PLOS One 11 (7): e0158447.

Examples of suitable promoters for directing transcription of the polynucleotide of the present invention in a filamentous fungal host cell are promoters obtained from Aspergillus, Fusarium, Rhizomucor and Trichoderma cells, such as the promoters described in Mukherjee et al., 2013, “Trichoderma: Biology and Applications”, and by Schmoll and Dattenböck, 2016, “Gene Expression Systems in Fungi: Advancements and Applications”, Fungal Biology.

For expression in a yeast host, examples of useful promoters are described by Smolke et al., 2018, “Synthetic Biology: Parts, Devices and Applications” (Chapter 6: Constitutive and Regulated Promoters in Yeast: How to Design and Make Use of Promoters in S. cerevisiae), and by Schmoll and Dattenböck, 2016, “Gene Expression Systems in Fungi: Advancements and Applications”, Fungal Biology.

### Terminators

The control sequence may also be a transcription terminator, which is recognized by a host cell to terminate transcription. The terminator is operably linked to the 3′-terminus of the polynucleotide encoding the variant. Any terminator that is functional in the host cell may be used in the present invention.

Preferred terminators for bacterial host cells may be obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells may be obtained from Aspergillus or Trichoderma species, such as obtained from the genes for Aspergillus niger glucoamylase, Trichoderma reesei beta-glucosidase, Trichoderma reesei cellobiohydrolase I, and Trichoderma reesei endoglucanase I, such as the terminators described in Mukherjee et al., 2013, “Trichoderma: Biology and Applications”, and by Schmoll and Dattenböck, 2016, “Gene Expression Systems in Fungi: Advancements and Applications”, Fungal Biology.

Preferred terminators for yeast host cells may be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, Yeast 8:423-488.

mRNA Stabilizers

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, J. Bacteriol. 177:3465-3471).

Examples of mRNA stabilizer regions for fungal cells are described in Geisberg et al., 2014, Cell 156 (4): 812-824, and in Morozov et al., 2006, Eukaryotic Cell 5 (11): 1838-1846.

### Leader Sequences

The control sequence may also be a leader, a nontranslated region of an mRNA that is important for translation by the host cell. The leader is operably linked to the 5′-terminus of the polynucleotide encoding the variant. Any leader that is functional in the host cell may be used.

Suitable leaders for bacterial host cells are described by Hambraeus et al., 2000, Microbiology 146 (12): 3051-3059, and by Kaberdin and Bläsi, 2006, FEMS Microbiol. Rev. 30 (6): 967-979.

Preferred leaders for filamentous fungal host cells may be obtained from the genes for Aspergillus oryzae TAKA amylase and Aspergillus nidulans triose phosphate isomerase.

Suitable leaders for yeast host cells may be obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

### Polyadenylation Sequences

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase, Aspergillus oryzae TAKA amylase, and Fusarium oxysporum trypsin-like protease.

Useful polyadenylation sequences for yeast host cells are described by Guo and Sherman, 1995, Mol. Cellular Biol. 15:5983-5990.

### Signal Peptides

The control sequence may also be a signal peptide coding region that encodes a signal peptide linked to the N-terminus of a variant and directs the variant into the cell's secretory pathway. The 5′-end of the coding sequence of the polynucleotide may inherently contain a signal peptide coding sequence naturally linked in translation reading frame with the segment of the coding sequence that encodes the variant. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the variant. However, any signal peptide coding sequence that directs the expressed variant into the secretory pathway of a host cell may be used.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase, such as the signal peptide described by Xu et al., 2018, Biotechnology Letters 40:949-955.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

### Propeptides

The control sequence may also be a propeptide coding sequence that encodes a propeptide positioned at the N-terminus of a variant. The resultant polypeptide is known as a proenzyme or propolypeptide (or a zymogen in some cases). A propolypeptide is generally inactive and can be converted to an active variant by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a variant and the signal peptide sequence is positioned next to the N-terminus of the propeptide sequence.

### Regulatory Sequences

It may also be desirable to add regulatory sequences that regulate expression of the variant relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals.

### Transcription Factors

The control sequence may also be a transcription factor, a polynucleotide encoding a polynucleotide-specific DNA-binding polypeptide that controls the rate of the transcription of genetic information from DNA to mRNA by binding to a specific polynucleotide sequence. The transcription factor may function alone and/or together with one or more other polypeptides or transcription factors in a complex by promoting or blocking the recruitment of RNA polymerase. Transcription factors are characterized by comprising at least one DNA-binding domain which often attaches to a specific DNA sequence adjacent to the genetic elements which are regulated by the transcription factor. The transcription factor may regulate the expression of a protein of interest either directly, i.e., by activating the transcription of the gene encoding the protein of interest by binding to its promoter, or indirectly, i.e., by activating the transcription of a further transcription factor which regulates the transcription of the gene encoding the protein of interest, such as by binding to the promoter of the further transcription factor. Suitable transcription factors for fungal host cells are described in WO 2017/144177. Suitable transcription factors for prokaryotic host cells are described in Seshasayee et al., 2011, Subcellular Biochemistry 52:7-23, as well in Balleza et al., 2009, FEMS Microbiol. Rev. 33 (1): 133-151.

### Expression Vectors

The present invention also relates to recombinant expression vectors comprising a polynucleotide encoding a variant of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the variant at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

The vector preferably contains at least one element that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous recombination, such as homology-directed repair (HDR), or non-homologous recombination, such as non-homologous end-joining (NHEJ).

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

More than one copy of a polynucleotide of the present invention may be inserted into a host cell to increase production of a polypeptide. For example, 2 or 3 or 4 or 5 or more copies are inserted into a host cell. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

### Host Cells

The present invention also relates to recombinant host cells, comprising a polynucleotide of the present invention operably linked to one or more control sequences that direct the production of a variant of the present invention.

A construct or vector comprising a polynucleotide is introduced into a host cell so that the construct or vector is maintained as a chromosomal integrant or as a self-replicating extrachromosomal vector as described earlier. The choice of a host cell will to a large extent depend upon the gene encoding the variant and its source. The recombinant host cell may comprise a single copy, or at least two copies, e.g., three, four, five, or more copies of the polynucleotide of the present invention.

The host cell may be any cell useful in the recombinant production of a variant of the invention, e.g., a prokaryotic cell or a fungal cell.

The host cell may be any microbial cell useful in the recombinant production of a polypeptide of the present invention, e.g., a prokaryotic cell or a fungal cell.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells. In an embodiment, the Bacillus cell is a Bacillus amyloliquefaciens, Bacillus licheniformis and Bacillus subtilis cell.

For purposes of this invention, Bacillus classes/genera/species shall be defined as described in Patel and Gupta, 2020, Int. J. Syst. Evol. Microbiol. 70:406-438.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

Methods for introducing DNA into prokaryotic host cells are well-known in the art, and any suitable method can be used including but not limited to protoplast transformation, competent cell transformation, electroporation, conjugation, transduction, with DNA introduced as linearized or as circular polynucleotide. Persons skilled in the art will be readily able to identify a suitable method for introducing DNA into a given prokaryotic cell depending, e.g., on the genus. Methods for introducing DNA into prokaryotic host cells are for example described in Heinze et al., 2018, BMC Microbiology 18:56, Burke et al., 2001, Proc. Natl. Acad. Sci. USA 98:6289-6294, Choi et al., 2006, J. Microbiol. Methods 64:391-397, and Donald et al., 2013, J. Bacteriol. 195 (11): 2612-2620.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

Fungal cells may be transformed by a process involving protoplast-mediated transformation, Agrobacterium-mediated transformation, electroporation, biolistic method and shock-wave-mediated transformation as reviewed by Li et al., 2017, Microbial Cell Factories 16:168 and procedures described in EP 238023, Yelton et al., 1984, Proc. Natl. Acad. Sci. USA 81:1470-1474, Christensen et al., 1988, Bio/Technology 6:1419-1422, and Lubertozzi and Keasling, 2009, Biotechn. Advances 27:53-75. However, any method known in the art for introducing DNA into a fungal host cell can be used, and the DNA can be introduced as linearized or as circular polynucleotide.

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell. In a preferred embodiment, the yeast host cell is a Pichia or Komagataella cell, e.g., a Pichia pastoris cell (Komagataella phaffii).

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolismis obligately aerobic. In contrast, vegetative growth by yeasts such as Saccharomyces cerevisiae is by budding of a unicellular thallus and carbon catabolismmay be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell. In a preferred embodiment, the filamentous fungal host cell is an Aspergillus, Trichoderma or Fusarium cell. In a further preferred embodiment, the filamentous fungal host cell is an Aspergillus niger, Aspergillus oryzae, Trichoderma reesei, or Fusarium venenatum cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.

In an aspect, the host cell is isolated.

In another aspect, the host cell is purified.

### Methods of Production

The present invention also relates to methods of producing a variant of the present invention, comprising (a) cultivating a recombinant host cell of the present invention under conditions conducive for production of the variant; and optionally (b) recovering the variant.

The host cell is cultivated in a nutrient medium suitable for production of the variant using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the variant to be expressed and/or isolated. Suitable media are available from commercial suppliers or may be prepared according to published compositions (e.g., in catalogues of the American Type Culture Collection). If the variant is secreted into the nutrient medium, the variant can be recovered directly from the medium. If the variant is not secreted, it can be recovered from cell lysates.

The variant may be detected using methods known in the art that are specific for the variant, including, but not limited to, the use of specific antibodies, formation of an enzyme product, disappearance of an enzyme substrate, or an enzyme assay determining the relative or specific activity of the variant.

The variant may be recovered from the medium using methods known in the art, including, but not limited to, collection, centrifugation, filtration, extraction, spray-drying, evaporation, or precipitation. In one aspect, the whole fermentation broth is recovered. In another aspect, a cell-free fermentation broth comprising the polypeptide is recovered.

The variant may be purified by a variety of procedures known in the art to obtain substantially pure variants and/or fragments (see, e.g., Wingfield, 2015, Current Protocols in Protein Science; 80 (1): 6.1.1-6.1.35; Labrou, 2014, Protein Downstream Processing, 1129:3-10).

In an alternative aspect, the variant is not recovered.

Compositions Comprising Carbonic Anhydrase Variants and Methods for their Preparation

The invention provides a composition comprising the carbonic anhydrase variants of the present invention and preferably an excipient and a method for preparing such a composition comprising admixing the polypeptide of the invention with an excipient.

In a particular embodiment, a carbonic anhydrase variant of the invention is the major (polypeptide) component of the composition, e.g., a mono-component composition. In a mono-component composition the carbonic anhydrase variant of the invention preferably constitutes at least 80% of the cabonic anhydrase activity, more preferably at least 90%, even more preferably at least 95% and most preferably 100% of the carbonic anhydrase activity. The excipient in this context is to be understood as any auxiliary agent or compound used to formulate the composition and includes solvent (e.g., water, inorganic salts, fillers, pigments, waxes), carriers, stabilizers, cross-linking agents, adhesives, preservatives, buffers and the like.

The composition may further comprise one or more additional enzymes, such as one or more additional carbonic anhydrases, a decarboxylase, laccase, or oxidase.

The compositions may be prepared in accordance with methods known in the art and may be in the form of a liquid or a solid composition. For instance, the enzyme composition may be formulated using methods known to the art of formulating technical enzymes and/or pharmaceutical products, e.g., into coated or uncoated granules or micro-granules. The polypeptide of the invention may thus be provided in the form of a granule, preferably a non-dusting granule, a liquid, in particular a stabilized liquid, a slurry or a protected polypeptide.

For certain applications, immobilization of the polypeptide may be preferred. An immobilized enzyme comprises two essential functions, namely the non-catalytic functions that are designed to aid separation (e.g., isolation of catalysts from the application environment, reuse of the catalysts and control of the process) and the catalytic functions that are designed to convert the target compounds (or substrates) within the time and space desired (Cao, Carrier-bound Immobilized Enzymes: Principles, Applications and Design, Wiley-VCH Verlag Gmbh & Co. KGaA, Weinheim, Germany, 2005). When an enzyme is immobilized it is made insoluble to the target compounds (e.g., substrates) it aids converting and to the solvents used. An immobilized enzyme product can be separated from the application environment in order to facilitate its reuse, or to reduce the amount of enzyme needed, or to use the enzyme in a process where substrate is continuously delivered and product is continuously removed from proximity to the enzyme, which, e.g., reduces enzyme cost. Furthermore, enzymes are often stabilized by immobilization. A process involving immobilized enzymes is often continuous, which facilitates easy process control. The immobilized enzyme can be retained as a heterogeneous catalyst by mechanical means, or by inclusion in a definite space. The latter can be done by microencapsulation, e.g., in semi permeable membranes or by inclusion in UF systems using, e.g., hollow fiber modules, etc. Immobilization on porous carriers is also commonly used. This includes binding of the enzyme to the carrier, e.g., by adsorption, complex/ionic/covalent binding, or just simple absorption of soluble enzyme on the carrier and subsequent removal of solvent. Cross-linking of the enzyme can also be used as a means of immobilization. Immobilization of enzyme by inclusion into a carrier is also industrially applied. (Buchholz et al., Biocatalysts and Enzyme Technology, Wiley-VCH Verlag GmbH & Co. KGaA, Weinheim, Germany, 2005). Specific methods of immobilizing enzymes such as carbonic anhydrase include, but are not limited to, spraying of the enzyme together with a liquid medium comprising a polyfunctional amine and a liquid medium comprising a cross-linking agent onto a particulate porous carrier as described in WO2007/036235 (hereby incorporated by reference), linking of carbonic anhydrase with a cross-linking agent (e.g., glutaraldehyde) to an ovalbumin layer which in turn adhere to an adhesive layer on a polymeric support as described in WO2005/114417 (hereby incorporated by reference), or coupling of carbonic anhydrase to a silica carrier as described in U.S. Pat. No. 5,776,741 or to a silane, or a CNBr activated carrier surface such as glass, co-polymerization of carbonic anhydrase with methacrylate on polymer beads as described in Bhattacharya et al., 2003, Biotechnol. Appl. Biochem. 38:111-117 (hereby incorporated by reference), or using globular protein and adhesive as described in US 2010/068784. The carbonic anhydrase may also be immobilized using tags such as histidine-like tags (e.g., 6×His tag or HQ tag) or a cellulose binding module (CBM) (Liu et al, 2008, Biotechnol. Prog. 25:68-74).

An embodiment of the present invention is a composition comprising a matrix suitable for immobilization and a carbonic anhydrase variant described herein. In a further embodiment of the present invention the carbonic anhydrase variant is immobilized on a matrix. The matrix may for example be selected from the group beads, fabrics, fibers, hollow fibers, membranes, particulates, porous surfaces, rods, structured packing, and tubes. Specific examples of suitable matrices include alumina, bentonite, biopolymers, calcium carbonate, calcium phosphate gel, carbon, cellulose, ceramic supports, clay, collagen, glass, hydroxyapatite, ion-exchange resins, kaolin, nylon, phenolic polymers, polyaminostyrene, polyacrylamide, polypropylene, polymerhydrogels, sephadex, sepharose, silica gel, precipitated silica, and TEFLON-brand PTFE. In an embodiment of the present invention carbonic anhydrase is immobilized on a nylon matrix according to the techniques described in Methods in Enzymology volume XLIV (section in the chapter: Immobilized Enzymes, pages 118-134, edited by Klaus Mosbach, Academic Press, New York, 1976), hereby incorporated by reference. The polypeptide to be included in the composition may be stabilized in accordance with methods known in the art e.g., by stabilizing the polypeptide in the composition by adding an antioxidant or reducing agent to limit oxidation of the polypeptide or it may be stabilized by adding polymers such as PVP, PVA, PEG, sugars, oligomers, polysaccharides or other suitable polymers known to be beneficial to the stability of polypeptides in solid or liquid compositions or it may be stabilized by adding stabilizing ions, such as zinc (e.g. zinc chloride or zinc sulphate) which is present in the enzyme active site. A preservative, such as Proxel, or penicillin, can be added to extend shelf life or performance in application.

In embodiments of the present invention, the carbonic anhydrase variant is immobilized by adsorption onto a matrix, surface or substrate. Non-limiting examples of a matrix, surface or substrate include those from the group: beads, fabrics, fibers, hollow fibers, membranes, particulates, porous surfaces, rods, structured packing, and tubes. Specific examples of suitable matrices, surfaces or substrates include alumina, bentonite, biopolymers, calcium carbonate, calcium phosphate gel, carbon, cellulose, ceramic supports, clay, collagen, glass, hydroxyapatite, ion-exchange resins, kaolin, nylon, phenolic polymers, polyaminostyrene, polyacrylamide, polyacrylonitrile (acrylic), polyethylene, polypropylene, polyester, polyurethane, polymerhydrogels, sephadex, sepharose, silica gel, precipitated silica, and TEFLON-brand PTFE. In embodiments, the matrices, surfaces or substrates may be dried after adsorption of the enzyme.

In a further embodiment, the composition of the invention is a composition applicable in the capture of carbon dioxide.

### Uses

Management and control of carbon dioxide (CO₂) concentration is important for a broad range of industrial, agricultural, medical and other technical processes. Conversion of CO₂ to alternative chemical forms, such as bicarbonate and carbonate, is one way to remove, separate, or extract CO₂ from a CO₂-containing medium, thereby controlling the CO₂ concentration in that medium. Conversion of CO₂ to an alternative chemical form can also function to retain the CO₂ in a CO₂-containing medium for a period of time, which can, for example, be useful for transporting CO₂ and for utilizing CO₂ in chemical and biochemical conversions that depend on the presence of CO₂, bicarbonate, or carbonate. Conversion of CO₂ to an alternative chemical form, such as bicarbonate, and reconversion from that alternative chemical form back to CO₂ is another way to control and manage the CO₂ concentration in processes, and provides a means to separate and transport CO₂ from one location to another location, and provides a means to separate CO₂ from a medium in one time period, and release the CO₂ from the medium in a later time period.

Carbon dioxide emissions are a major contributor to the phenomenon of global warming. CO₂ is a by-product of combustion and it creates operational, economic, and environmental problems. CO₂ emissions may be controlled by capturing CO₂ gas before emitted into the atmosphere. There are several chemical approaches to control the CO₂ emissions (A. Kohl and R. Nielsen, Gas Purification, 5^{th} ed., Gulf Professional Publishing, Houston, TX, 1997). However, many of these approaches have drawbacks such as high energy consumption, slow processes, and use of ecologically questionable or toxic compounds.

Technical solutions for extracting CO₂ from gases, such as combustion gases, fuel gases, atmospheric gases or respiration gases, using carbonic anhydrases have been described in, for example, WO2006/089423, U.S. Pat. No. 6,524,842, WO2004/007058, WO2004/028667, US2004/0029257, U.S. Pat. No. 7,132,090, WO2005/114417, U.S. Pat. No. 6,143,556, WO2004/104160, US2005/0214936, WO2008/072979, WO2008/095057, WO2012/003336, WO2012/025577, WO2012/092984, WO 2012/154735, U.S. Pat. No. 7,998,714, WO 2013/151757, U.S. Pat. No. 8,871,008, WO2015/126925, US2015/0099289, and in the literature (e.g., Russo et al., 2013, Post-combustion carbon capture mediated by carbonic anhydrase, Sep. Purif. Technol. 107:331-339; S. Salmon and A. House, “Enzyme-catalyzed solvents for CO2 separation,” in Novel Materials for Carbon Dioxide Mitigation Technology, F. Shi and B. Morreale, Eds., Amsterdam, Elsevier B.V., 2015, pp. 23-86; and, S. Salmon and A. House, “Low-energy solvents for carbon dioxide capture enabled by a combination of enzymes and vacuum regeneration,” Final Scientific/Technical Report for DE-FE0007741, U.S. Department of Energy, National Energy Technology Laboratory, 2015, DOI: 10.2172/1222645), which are herein incorporated by reference.

Generally, CO₂ scrubbing techniques operate by bringing a soluble or immobilized carbonic anhydrase into contact with CO₂ which either may be in a gas phase or a liquid phase. In the presence of water, carbonic anhydrase catalyses the conversion of CO₂ into bicarbonate ions which may be further protonated or deprotonated to carbonic acid and/or carbonate ions depending on the pH of the medium. The ions may either be utilized to facilitate growth of algae or microorganisms that utilize bicarbonate/carbonate as a carbon source, to induce a pH change in a surrounding medium or supply buffering capacity, to provide bicarbonate/carbonate as an active agent for subsequent chemical processes, or precipitated as a carbonate salt, or converted back into pure CO₂, which can then be used (for example in enhanced oil recovery, for production of urea, for food and beverage processing, or to supply CO₂ to greenhouses or cultivation ponds), released (for example from a contained life support environment such as a submarine, spacecraft, or artificial lung), compressed (for example for transportation through pipelines), or stored (such as in geological or deep oceanic formations or saline aquifers).

Furthermore, the use of carbonic anhydrase to catalyse the conversion of CO₂ to bicarbonate in the presence of cations, such as sodium and potassium, can help accelerate subsequent processes, such as conversion of bicarbonates to carbonates, resulting in more rapid conversion of CO₂ into useful alternative chemical forms. CO₂ conversion to bicarbonate can improve the productivity of biological systems that utilize the carbon from CO₂ to produce chemical compounds (e.g., Atsumi et al., 2009, Direct photosynthetic recycling of carbon dioxide to isobutyraldehyde, Nature Biotechnology 27 (12): 1177-1180). CO₂ conversion to carbonate, and the combination of carbonate with divalent cations such as calcium and magnesium, for example, is useful for sequestering, or storing, CO₂ in large quantities (e.g., Favre et al., 2009, Biocatalytic capture of CO₂ with carbonic anhydrase and its transformation to solid carbonate, J. Molec. Cat. B: Enzymatic, 60 (3-4): 163-170). Production of carbonates is useful for the production of concrete, or for above-ground or below-ground CO₂ mineralization inside or as part of rocks or geologic formations, e.g., sub-surface limestone or basalt formations, also called “mineral sequestration,” which help mitigate the negative impacts of CO₂ release to the atmosphere (e.g., US 2014/0234193). Preferably, sufficient cations and alkalinity to enable the carbonation reaction would be available at the mineralization site, however, these materials could also be delivered to the site, e.g., in the form of seawater, industrial brines, or aqueous waste streams comprising cations and alkalinity.

The presence of carbonic anhydrase in the systems and processes for CO₂ utilization can improve reaction efficiencies. For example, by rapidly converting CO₂, which has low water solubility, into bicarbonate, which has high water solubility, the presence of CA can help increase the carbon concentration in water systems used for growing algae, especially saline water systems comprising sodium or potassium cations, thereby improving algae growth and productivity. Delivery of CA into CO₂ sequestration sites, such as underground storage locations rich in divalent cations, can accelerate the mineralization process by accelerating the first step of the CO₂ to bicarbonate to carbonate reaction sequence, and ultimately resulting in precipitated carbonate-based solids. Similarly, in the presence of CO₂, CA can accelerate the removal or recovery of cations, especially divalent cations, from aqueous liquids, by accelerating the rate at which CO₂ is converted to its ionic forms and made available for ionic complexation with the cations to form solid bicarbonate or carbonate-based products that precipitate in the liquid or can be recovered by filtration or other solid-liquid separation techniques. Conversion of CO₂ into its ionic forms, such as bicarbonate, helps dissolve CO₂ into water-containing liquids and therefore increases the amount of CO₂ that can be loaded into and carried by these liquids. By accelerating the rate of CO₂ conversion to bicarbonate, CA can improve processes, such as the CarbFix process (Oelkers et al., 2008, Mineral carbonation of CO₂, Elements 4:333-337), which requires large amounts of water to dissolve CO₂, by reducing the amount of water needed to transport a certain amount of CO₂ and by promoting the mineralization of CO₂.

The carbonic anhydrase variants described above are useful in a series of applications which are described in more detail below. When referring to carbonic anhydrase variants herein it is intended to include all the carbonic anhydrase variants described in the present invention in particular if they fall within the claimed identities.

In particular, carbonic anhydrases may be used for carbon dioxide extraction from CO₂ emission streams, e.g., from carbon-based or hydrocarbon-based combustion in electric generation power plants, or from flue gas stacks from such plants, industrial furnaces, stoves, ovens, or fireplaces or from airplane or car exhausts. Carbonic anhydrase variants may also be used to remove CO₂ in the preparation of industrial gases such as acetylene (C₂H₂), carbon monoxide (CO), chlorine (Cl₂), hydrogen (H₂), methane (CH₄), nitrous oxide (N₂O), propane (C₃H₈), sulfur dioxide (SO₂), argon (Ar), nitrogen (N₂), and oxygen (O₂). Carbonic anhydrase variants can also be used to remove CO₂ from a raw natural gas during the processing to natural gas. Removal of CO₂ from the raw natural gas will serve to enrich the methane (CH₄) content in the natural gas, thereby increasing the thermal units/m³. Raw natural gas is generally obtained from oil wells, gas wells, and condensate wells. Natural gas contains between 1% to 10% CO₂ when obtained from geological natural gas reservoirs by conventional methods, but depending on the natural source or recovery method used may contain up to 50% CO₂ or even higher. Carbonic anhydrase can also be used to purify the natural gas such that it is substantially free of CO₂, e.g., such that the CO₂ content is below 1%, preferably below 0.5%, 0.2%, 0.1%, 0.05% and most preferably below 0.02%. In resemblance to the methane enrichment of natural gases, carbonic anhydrases can also be used to enrich the methane content in biogases. Biogases will always contain a considerable degree of CO₂, since the bacteria used in the fermentation process produce methane (60-70%) and CO₂ (30-40%). Biogas production may be performed using mesophilic or thermophilic microorganisms. Thermophilic strains allow the fermentation to occur at elevated temperatures, e.g., from 40° C. to 80° C., or from 50° C. to 70° C., or from 55° C. to 60° C. In such processes a heat-stable carbonic anhydrase is particularly useful to remove CO₂ from the methane. The present invention provides for the use of a carbonic anhydrase variant to reduce the carbon dioxide content in a biogas, preferably the CO₂ content is reduced such that it constitutes less than 25%, more preferably less than 20%, 15%, 10%, 5%, 2%, 1%, 0.5% and most preferably less than 0.1%. In a preferred embodiment the carbonic anhydrase is heat-stable. Furthermore, carbonic anhydrase may be applied in the production of syngas by removing the CO₂ generated by the gasification of a carbon containing fuel (e.g., methane or natural gas) thereby enriching the CO, H2 content of the syngas. Where syngas production occurs at elevated temperatures the use of a heat-stable carbonic anhydrase is an advantage. The present invention provides for the use of a carbonic anhydrase to reduce the carbon dioxide content in a syngas production. Preferably, the CO₂ content is reduced such that it constitutes less than 25%, more preferably less than 20%, 15%, 10%, 5%, 2%, 1%, 0.5% and most preferably less than 0.1%. In a preferred embodiment the carbonic anhydrase is heat-stable.

In an aspect of the present invention the CO₂ extraction from a CO₂-containing medium is performed in enzyme-based bioreactors. Before the carbon dioxide-containing medium is processed in a bioreactor, it may be purified to free it from contaminants which may disturb the enzymatic reaction or interfere with bioreactor functionality in other ways, e.g., by clotting outlets or membranes. Gasses multiphase mixtures emitted from combustion processes, e.g., flue gases or exhausts, are preferably cleared of ash, particles, NOₓ and/or SO₂, before the gas/multiphase mixture is passed into the bioreactor. Alternatively, SO₂ separation and CO₂ extraction can occur in the same reactor to improve system efficiency, because both separations are typically operated at alkaline process conditions. The raw natural gas from different regions may have different compositions and separation requirements. Preferably, oil, condensate, water and natural gas liquids, if present in the raw natural gas, are removed prior to the extraction of CO₂ in an enzyme-based bioreactor. The CO₂ emitted from combustion processes or present in the raw natural gas may be extracted in the same process as the sulfur removal, or it may be extracted in a separate process. If the gas at this point exceeds the temperature tolerance of the carbonic anhydrase of the present invention, some degree of cooling may be needed. Preferably, the maximum temperature to which carbonic anhydrase is exposed during CO₂ extraction process whether it is the process temperature in the bioreactor or the feed gas temperature may be between 0° C. and 120° C. Preferably the maximum process temperature is between 40° C. and 120° C., more preferably between 45° C. and 110° C., more preferably between 50° C. and 100° C., more preferably between 55° C. and 90° C. even more preferably between 60° C. and 80° C., and most preferably between 65° C. and 75° C.

Reactors, systems and processes for gas separation, including CO₂ extraction, are well known in the art and are used commercially for various purposes (A. Kohl and R. Nielsen, Gas Purification, 5th ed., Gulf Professional Publishing, Houston, TX, 1997). By selecting CO₂ absorption liquids (also called CO₂ absorption solvents) and operational conditions that are compatible with enzyme limitations, CAs of the present invention can be used in any solvent-based CO₂ extraction reactor to generate a bioreactor (a reactor comprising biological material such as an enzyme) for extracting CO₂ from gases, such as combustion gases, atmospheric gases, fuel gases, or respiration gases. CA can be present in these systems in an aqueous-soluble form, can be present as a suspended protein-based solid, and can be present in a form that is chemically or biochemically modified or combined with other materials. These different forms are collectively called biocatalysts.

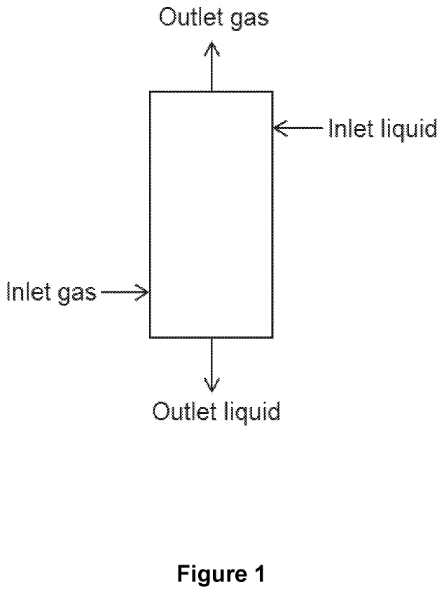

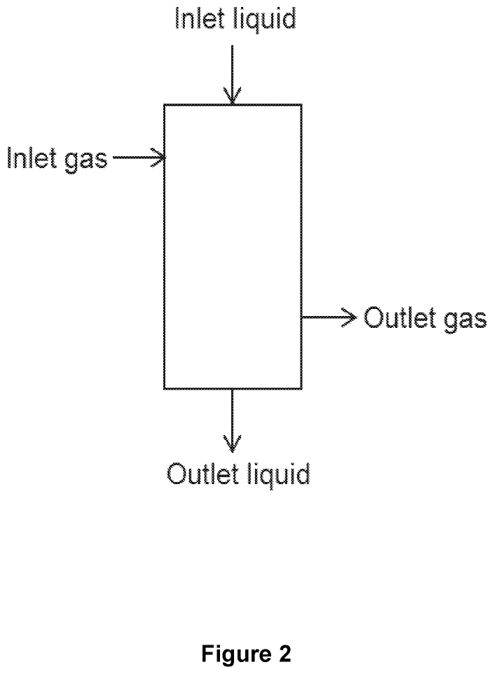

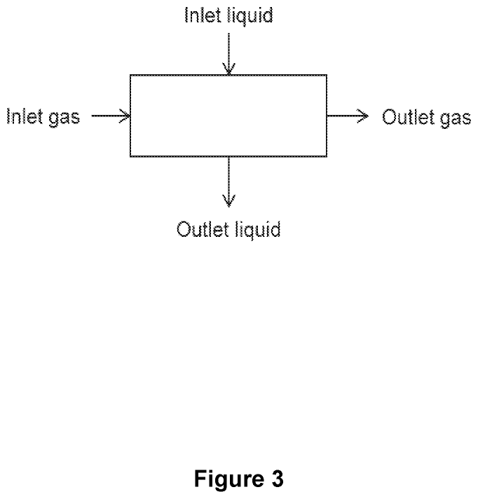

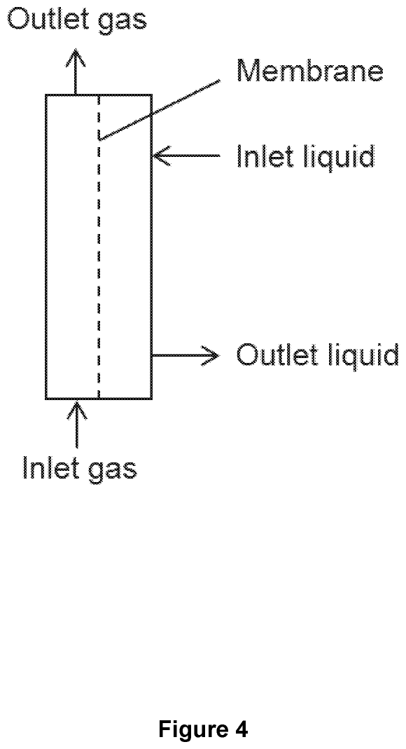

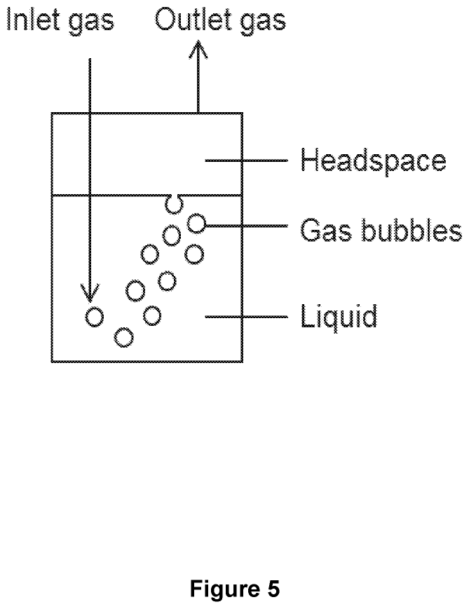

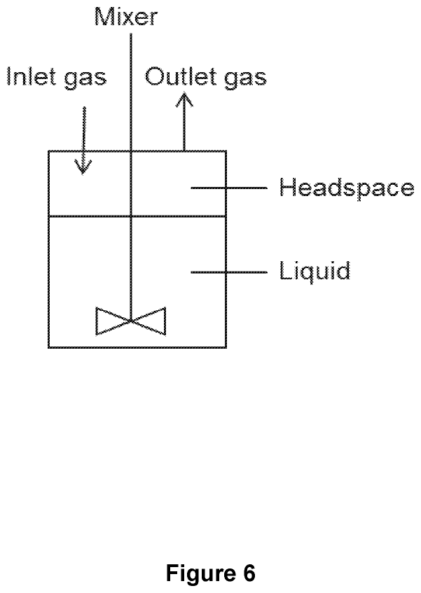

Schematic diagrams for several common reactors (or gas-liquid contactors) used for CO₂ separation systems and processes are shown in FIGS. 1 to 6. These reactors can be used for CO₂ absorption from a gas into a liquid and for CO₂ desorption from a liquid to a gas. The reactors can be used for a single once-through unit operation or the gas or liquid streams or both can be recirculated from the outlet to the inlet of a reactor to provide for multiple passes of gas and liquid streams through a reactor. Multiple reactors can be arranged in CO₂ gas scrubbing systems in sequential or parallel arrangements, or both, to enable handling of large volumes of gas and liquid or provide high efficiency of CO₂ removal. Different types of reactors can be used to form gas scrubbing systems with many different configurations. For example, a counter-current packed reactor (illustrated by FIG. 1) can be used for CO₂ gas absorption into a liquid and can be arranged in a recirculating system together with the same type or another type of reactor, e.g., a membrane-based reactor (illustrated by FIG. 4), used for removing (desorbing) CO₂ from the liquid, and the process is repeated when the CO₂-lean liquid returns to the counter-current reactor. Many combinations and variations are possible. For continuous flow, the outlet liquid from one reactor travels to the inlet liquid of another reactor in the schematics shown in FIGS. 1 to 6. The reactors can be large or small.

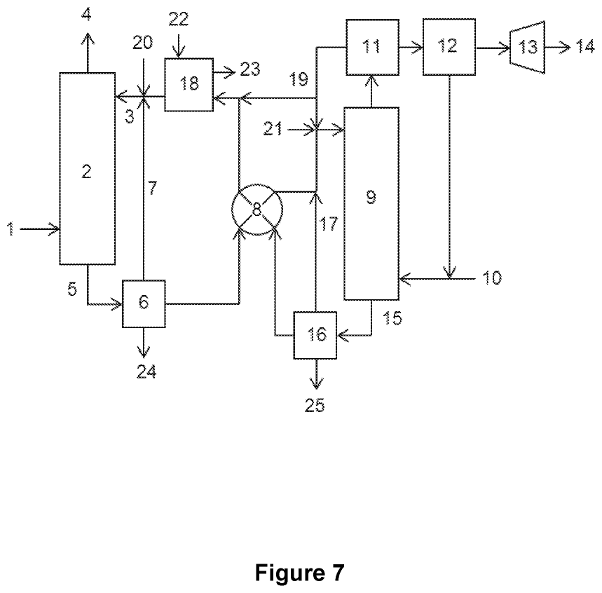

FIG. 7 shows an integrated CO₂ scrubbing system utilizing recirculation of the CO₂-absorption liquid between the absorber (2) and desorber (9) as well as optional units for biocatalyst separation (6 and 16) and recycling, optional utilization of sweep gas (10) in the desorption stage and optional utilization of secondary air sweep (18) prior to CO₂-lean liquid entry to the absorber. Because carbonic anhydrase improves the rate of CO₂ extraction, combining carbonic anhydrase with CO₂ extraction reactors enables reactor and process improvements such as smaller size and less expensive gas-liquid contactors (e.g., shorter absorption column), the use of process intensification approaches (e.g., horizontal spray reactors and rotating packed bed reactors), and use of low energy consuming and low volatility CO₂-absorption liquids, as well as overall lower operating temperatures compared to the conventional approaches.

One type of reactor uses liquid membranes. This may for example be reactors including hollow fiber membranes containing a liquid film as described in Majumdar et al., 1988, AIChE 34:1135-1145; U.S. Pat. Nos. 4,750,918; 6,156,096; WO04/104160. Such hollow fiber membrane-based designs are also sometimes termed hollow fiber liquid membranes (HFLM) and the CO₂ separation devices based on these have been termed hollow fiber contained liquid membrane (HFCLM) permeators. A common feature of HFCLM permeators is that the hollow fibers enclosing the feed and sweep gas streams are near (i.e., “tightly packed” or “immediately adjacent”) to one another and they are enclosed in a single rigid treatment chamber to form one complete permeator. In such a design, a liquid surrounds the shell side of the tightly packed feed and sweep hollow fibers. Because the distance between the outside wall of one hollow fiber is very close to adjacent hollow fibers the thickness of the liquid layer between them is thin, like a membrane, and the composition of the liquid only allows certain components to pass, hence the term “liquid membrane” has been used to describe the liquid surrounding the hollow fibers. Contained liquid membrane permeators where the liquid film is sandwiched between two structural support membranes have also been described in the art (Cowan et al., 2003, Ann. NY Acad. Sci. 984:453-469); this design essentially functions in the same way as the HFCLM. Contained liquid membrane permeators have also been used in combination with carbonic anhydrase as described in U.S. Pat. No. 6,143,556, WO 2004/104160, Cowan et al., 2003, Ann. NY Acad. Sci. 984:453-469; and Trachtenberg et al., 2003, SAE international Conference on Environmental Systems Docket number 2003-01-2499. In these cases, the CO₂ desorption step takes place in the same enclosed treatment chamber as the absorption step. Another example describes an amine-based CO₂ capture reactor based on absorber/desorber hollow fiber membrane modules (Kosaraju et al., 2005, Ind. Eng. Chem. Res. 44:1250-1258).

Another type of reactor uses direct gas-liquid contact. This may for example be conventional solvent-based CO₂ capture reactors that are based on absorber/desorber column reactors (US 2008/0056972, Reddy et al., Second National Conference on Carbon Sequestration, NETL/DOE, Alexandria, VA, May 5-8, 2003). Example flow schemes for commercial direct gas-liquid contactor reactors that use alkanolamines (such as monoethanolamine, diethanolamine, and methyldiethanolamine) for CO₂ extraction are shown in A. Kohl and R. Nielsen, Gas Purification, 5^{th} ed., Gulf Professional Publishing, Houston, TX, 1997:57-62. Example flow schemes for commercial direct gas-liquid contactor reactors that use alkaline salt solutions (such as potassium carbonate) for CO₂ extraction are shown in A. Kohl and R. Nielsen, Gas Purification, 5th ed., Gulf Professional Publishing, Houston, TX, 1997:334-340.

Direct gas-liquid contact reactors using carbonic anhydrase have been described in U.S. Pat. No. 6,524,843; WO2004/007058, WO2004/056455, U.S. Pat. No. 7,176,017, and US2004/0059231. In these types of reactors, the gas phase or multiphase mixture, is contacted with a liquid phase under conditions where the CO₂ in the gas phase is absorbed by the liquid phase where it is converted into bicarbonate by carbonic anhydrase. The bicarbonate enriched liquid is removed from the reactor by a continuous flow, to ensure that the equilibrium between CO₂ and bicarbonate is shifted towards continuous conversion of CO₂. The gas phase dissolution into the liquid phase is dependent on the surface contact area between the gas and liquid. A large contact area can for example be achieved by passing liquid and CO₂-containing gas through a high surface area packed column, tray or plate column or tower, by spraying small droplets of liquid through the CO₂-containing gas (i.e., a spray contactor), or by bubbling the CO₂-containing gas through the liquid (i.e., bubble tank or pond), or by combinations of these techniques. Packed columns can comprise packings such as raschig rings, berl saddles, lessing rings, intalox metal, intalox saddles, pall rings or engineered packings such as Q-PAC (Lantec Products, Inc., Agoura Hills, CA 91301). The packing materials may be comprised of a polymer such as nylon, polyester, polyethylene, polyetheretherketone, polypropylene, polystyrene or fluoropolymer (e.g., polytetrafluoroethylene), a ceramic such as silica, or a metal such as aluminium, carbon steel, or stainless steel, or a cellulose-based material such as wood or cotton fiber.

In reactor types where the liquid is continuously exchanged or when it is desirable to restrain carbonic anhydrase to one or more locations in the reactor, carbonic anhydrase may be retained in the reactor by various means. In packed columns the carbonic anhydrase can be immobilized on the packing material (for methods of immobilizing CA, see for example in WO2005/114417, WO2013/151757) or can be immobilized as particles (e.g., US2015/0099289 and Yan et al., 2007, Fabrication of single carbonic anhydrase nanogel against denaturation and aggregation at high temperature, Biomacromolecules 8:560-565) that recirculate from the outlet to the inlet of the packed column along with at least a portion of the liquid flow and avoiding travel through other parts of the system, or can circulate along with the CO₂-absorption liquid throughout the process, both absorption and desorption, due to enzyme stabilization imparted by the combination of enzyme with the immobilization matrix. The use of particles in gas-liquid contactors containing packing materials, baffles, and other internals is possible when the particles have the size and physical properties to flow along with the liquid. Small particles comprising CA are known to enhance CO₂ absorption, and is explained by the ability of carbonic anhydrase to rapidly catalyse the CO₂ hydration reaction together with the ability of small particles to be located and move around in the thin liquid film interface between the gas and liquid phases, allowing the substrate (CO₂) to rapidly come in contact with the CA catalyst (E. Alper and W. D. Deckwer, Some aspects of gas absorption mechanism in slurry reactors, in “Mass Transfer with Chemical Reaction in Multiphase Systems,” E. Alper (ed.), Springer Science & Business Media, Dordrecht, 1983, pp. 199-224).

Different sized screens, filters or solid-liquid separation techniques, can be used to restrain CA enzyme, chemically or physically modified CA enzyme, or immobilized CA enzyme to particular operational units, regions, or locations in the CO₂ scrubbing system. Such techniques can also be used to restrain one type or mixture of CA biocatalysts in one reactor and another type or mixture of CA biocatalysts in another reactor zone. For example, this technique can be used to localize different CAs in the absorber and in the desorber. In “bubbling” reactors the carbonic anhydrase can be entrapped in a porous substrate, for example, an insoluble gel particle such as silica, silicone, urethane, alginate, alginate/chitosan, alginate/carboxymethylcellulose, or the carbonic anhydrase can be immobilized (by covalent bonds, ionic charges, entrapment or encapsulation) on a fixed solid packing, or can be immobilized on or in particles in suspension in the liquid, or the carbonic anhydrase can be chemically linked in an albumin or PEG network. Carbonic anhydrase can also be restrained to a particular location in the reactor by entrapment in a polymeric immobilization material which may comprise a micellar or inverted micellar material, such as described in WO2010/037109, and may include chemical modification of the enzyme as part of the entrapment or immobilization technique (e.g., WO2012/122404). CAs of the present invention can be immobilized by these and other techniques known in the art.

Spray contactors may include vertical or horizontal spray chambers, countercurrent spray columns, venturi scrubbers, ejectors or jet scrubbers, cyclone scrubbers, and spray dryers (A. Kohl and R. Nielsen, Gas Purification, 5th ed., Gulf Professional Publishing, Houston, TX, 1997:418-427 and 604-616). Use of spray contactors is desirable for avoiding pressure drop and improving tolerance to solid particulates in the gas, such as may be important for atmospheric pressure post-combustion exhaust gas applications. However, to be most effective, the rate of CO₂ absorption in spray contactors must be fast, and carbonic anhydrase can provide the needed catalysis to achieve these fast rates.

CO₂ extraction in a direct gas-liquid contact reactor may involve a first absorption stage followed by optionally a subsequent desorption, precipitation, utilization, collection, regeneration or release stage. A general description of the absorption stage is as follows. When the absorption reactor is in operation, a water-containing liquid enters the reactor at one end, preferably the top, and flows to the other end, preferably the bottom, and the CO₂-containing gas stream (feed gas) enters the reactor at one end, preferably at the opposite end (the bottom) (“countercurrent”) from the liquid and the gas passes through the liquid and exits, minus the CO₂ extracted into the liquid, through a gas outlet at the opposite end (preferably, the top of the reactor). The liquid that exits the absorption reactor is enriched in bicarbonate/carbonate (CO₂-rich liquid) and the exit gas is reduced in the CO₂ content compared to the feed gas. The CO₂-rich liquid may be processed in subsequent reactions, for example to generate pure CO₂ by passing through a desorption reactor or produce carbonate precipitates such as CaCO₃. The CO₂-rich liquid from the absorption reactor can also be utilized, e.g., to enhance algae growth, collected, e.g., by pumping the CO₂-rich liquid into a contained geological formation, released, e.g., by pumping the CO₂-rich liquid into the environment, such as release of bicarbonate liquid into seawater from a submarine life support system, evaporated or desalinated. The CO₂-rich liquid containing bicarbonate anion can be used in industrial processes, such as in the manufacturing processes for ammonium carbonate and ammonium bicarbonate, which are useful as fertilizer, or in processes for the removal and neutralization of acid gases such as sulfur dioxide.

The reactors described herein may involve an absorption stage, a desorption stage or a sequence of absorption and desorption stages in which carbonic anhydrase may catalyze either the hydration of CO₂ to bicarbonate or the dehydration of bicarbonate to CO₂ or both. The reactors can be combined with each other where each reactor constitutes a module. For example, a liquid membrane reactor can function as absorption module and the direct gas-liquid contact reactor can function as a desorption module or vice versa.

The terms “CO₂-lean” and “CO₂-rich” absorption liquid are terms used in the present invention to describe the relative amount of carbon (e.g., in the form of dissolved CO₂, chemically reacted CO₂, bicarbonate, carbonic acid and/or carbonate salt) present in the absorption liquid as it circulates through the process. As used herein, the term “CO₂-lean liquid” generally refers to absorption liquid entering an absorption unit. The term “CO₂-rich liquid” generally refers to a absorption liquid entering a desorption unit. It is understood that the term “CO₂-lean liquid” can also be applied to absorption liquid exiting a desorption module, and the term “CO₂-rich liquid” can also be applied to absorption liquid exiting an absorption unit. CO₂-rich liquid contains more carbon compared to CO₂-lean liquid within a given system at a given point in time. As used herein, the term “CO₂-rich gas” generally refers to a gas mixture with a relatively high CO₂ content, or it can be a pure stream of CO₂ gas. A CO₂-rich gas can be a feed gas. The term “CO₂-lean gas” generally refers to a gas mixture that is depleted in CO₂ content compared to the CO₂-rich gas from which at least a portion of CO₂ was removed. A CO₂-lean gas can be a gas that does not comprise CO₂, e.g., a pure stream of nitrogen gas. A CO₂-lean gas can be used as a sweep gas to help remove CO₂ from a CO₂-rich liquid.

Without limiting the scope of the present invention, FIG. 7 is provided to illustrate a general schematic of a CO₂ extraction system comprising both absorption and desorption units through which the CO₂ absorption liquid circulates as it removes CO₂ from a CO₂-containing gaseous phase (feed gas, 1) in the absorber (2), releases purified CO₂ gas (14) from the desorber (9), and recirculates back to the absorber. The term “feed gas” is often used in relation to CO₂ extraction reactors where it implies that CO₂ is removed from the CO₂ containing gaseous phase by contact with a CO₂-lean absorption liquid in the reactor. The feed gas may be at atmospheric pressure, or at pressures above or below atmospheric pressure. Selective solubility of CO₂ in the absorption liquid causes extraction of CO₂ from the feed gas into the absorption liquid in the absorber. In the desorber, CO₂ is released from the CO₂-rich absorption liquid by introducing a pressure difference (for example, a lower partial pressure of CO₂ in the desorber gas phase compared to that in the feed gas, such as can be achieved by applying vacuum in the desorber, or can be achieved by passing a sweep gas through the desorber, such as air or a condensable sweep gas) that lowers the solubility of CO₂ in the carrier liquid and/or applying heat, e.g., via a reboiler, steam or a sweep gas to drive CO₂ into the gas phase in the desorber. Heat for desorption can also be applied by inducing cavitation, e.g., through application of ultrasonic or other acoustic or vibrational energy, and by applying microwave or infrared energy to the CO₂-rich liquid. More than one desorption stage can be used to optimize the efficiency of CO₂ release. For example, heat can be applied in one stage of the desorber to remove and capture the bulk of the CO₂, followed by one or more secondary desorption stages, e.g., using air sweep, to remove additional CO₂ and ‘polish’ the liquid to a more CO₂-lean loading. Heat energy alone can be used to drive desorption such as is commonly used in monoethanol amine-based CO₂ extraction processes. For example, the temperature in the desorber of a typical monoethanol amine-based CO₂ extraction is greater than 100° C. (e.g., 120° C.). Alternatively heat energy can be combined with pressure reduction to drive desorption. In this case the temperature in the desorber can be lowered. For example, together with a reduced pressure (e.g., vacuum) compared to the pressure in the absorber (e.g., atmospheric pressure), the desorber can be operated at 70° C. A difference in pH can be used to facilitate absorption and desorption, wherein CO₂ absorption into an aqueous medium is favored at more alkaline pH whereas CO₂ desorption from an aqueous medium is favored at a less alkaline (more acidic) pH. The range of relevant pH difference (“swing”) between absorption and desorption depends on the particular process. For example, for the sake of illustration, CO₂ absorption into a bicarbonate-based carrier liquid can occur at pH 9 or above resulting in a decrease in the pH of that carrier liquid to below pH 9. Desorption of CO₂ from that carrier liquid can then occur at pH below pH 9.

A pressure difference between the absorber and the desorber can be established/occur when the pressure of the feed gas passing through the absorber is higher than the pressure of the gas phase in the desorber. In some cases, such as for natural gas upgrading, the gas pressure in the absorber is higher than in the desorber and the gas pressures in both the absorber and the desorber may be above atmospheric pressure. In other cases, the gas pressure in the absorber is above atmospheric pressure and the gas pressure in the desorber is at or below atmospheric pressure (i.e., equal to or less than 100 kPa). Alternatively, a pressure difference between the absorber and the desorber can be established/occur when the pressure of the feed gas (such as a coal-fired post-combustion flue gas) passing through the absorber is approximately at atmospheric pressure and the pressure of the gas phase in the desorber is below atmospheric pressure. In one embodiment of the present invention, the total gas pressure difference between the absorber and the desorber is at least about 35 kPa. Alternatively, a sweep gas comprising no or low concentration of CO₂, such as air, can be used to provide the driving force needed to release CO₂ from the CO₂-rich liquid as it passes through the desorber. CO₂ absorption and desorption can be operated in batch mode in a single reactor by first exposing the lean liquid in the reactor to CO₂-rich gas for a period of time, then applying desorption driving force options, such as heat, vacuum, acoustic or cavitation effects, or sweep gas, or combinations of these, to release CO₂ from the CO₂-rich liquid for a period of time to regenerate the CO₂-lean liquid. The sweep gas can be heated to provide both thermal and partial pressure driving forces for CO₂ release. The cycle can be repeated. Air sweep desorption can be desirable for applications where CO₂ separation is needed and the CO₂ being separated can be released to the atmosphere, for example when the released CO₂ is regarded as CO₂-neutral emissions, such as the CO₂ separated from methane during biogas upgrading.

One embodiment of the invention encompasses the use of condensable sweep gas compounds in the desorption stage of a CO₂ gas separation reactor. A typical solvent-based CO₂ gas separation reactor has two main stages: 1) an absorption stage, and 2) a desorption stage. In the absorption stage, CO₂ from the gas phase is absorbed by the liquid phase. In the desorption stage, CO₂ in the liquid phase is released to the gas phase. In order for CO₂ to be released from the liquid to the gas phase, a concentration gradient must exist, wherein the concentration (partial pressure) of CO₂ in the gas phase is less than the concentration (partial pressure) of CO₂ in the liquid phase. A “sweep” stream of non-CO₂ gas applied in the desorption stage can assist the mass transfer of CO₂ from the liquid to the sweep gas phase because the incoming sweep gas has no or low concentration of CO₂, causing CO₂ from the liquid phase to move in the direction of the low partial pressure of CO₂. One type of sweep gas is water vapour (water in the gas phase), such as is produced when aqueous liquids are boiled. However, considerable energy is required to boil water. Air or nitrogen gas can also be used as sweep gases, however there is no easy way to separate these gases from CO₂ after the desorption step, which may be necessary to obtain an isolated or purified CO₂ product. It is therefore desirable to utilize compounds that can perform as a sweep gas without the energy cost of boiling water and also have properties that allow these compounds to be easily separated from CO₂ after the desorption step to provide a purified stream of CO₂ gas and recovered sweep gas compounds that can be used for subsequent desorption operations.

The use of certain non-water-miscible volatile carriers together with CO₂ absorption solvents, such as aqueous MEA, in CO₂ stripping processes has been described (R. A. Frimpong, J. E. Remias, J. K. Neathery, M. Liu and K. Liu, Enhancing solvent regeneration with a high volatility liquid as a stripping carrier, Tenth Annual Conference on Carbon Capture & Sequestration, May 2-5, 2011, Pittsburgh, Pennsylvania; Proceedings on CD-ROM, Exchange Monitor Publications & Forums, 4455 Connecticut Ave NW, Suite A700, Washington, DC 20008). These systems can include CA to enhance desorption efficiency by overcoming rate limitations that may exist in the conversion of bicarbonate to CO₂ during these processes, and CAs can be selected and engineered to be compatible with and withstand these processes whether or not they perform a specific catalytic role in these processes.

Condensable sweep gas compounds have the properties of a boiling temperature (equivalent to condensation temperature) that is substantially higher than the boiling temperature of CO₂, while at the same time, preferably, being lower than the boiling temperature of water. One such compound is dimethyl ether, which has a boiling point (b.p.) of −24° C. at 1 atmosphere pressure. This low boiling point facilitates the removal of dimethyl ether from reaction mixtures. Dimethyl ether is a gas at temperatures below the boiling point of water, and dimethyl ether will condense to a liquid at temperatures significantly higher than the sublimation temperature of CO₂ (−78.5° C., 1 atm). Therefore, a sweep gas containing a mixture of CO₂ and dimethyl ether can be passed through a chilled condenser at temperatures between −78° C. to −24° C. to cause the dimethyl ether to condense to a liquid and become trapped by the condenser while allowing the CO₂ to pass through the condenser as a purified gas stream. The dimethyl ether liquid can be recycled for reuse as a sweep gas. Dimethyl ether is a non-toxic, inexpensive compound, resistant to auto-oxidation compared to other alkyl ethers, and is considered as an alternative fuel or a renewable fuel (BioDME) from gasification of lignocellulosic biomass. Therefore, dimethyl ether is readily available for sweep gas applications. Dimethyl ether is combustible, therefore engineering controls are needed to prevent combustion during use. Such controls can be optimized to take advantage of the feature that the CO₂ compound being removed is itself non-combustible. Other examples of condensable sweep gas compounds are ethanol (b.p. 78.4° C.), methanol (b.p. 64.7° C.), acetone (b.p. 56° C.), propanol (b.p. 97-98° C.), isopropanol (b.p. 82.6° C.), tertiary butanol (b.p. 82.2° C.), and diethyl ether (b.p. 34.6° C.). In each case, appropriate engineering controls are needed to prevent unwanted combustion or decomposition, e.g., a risk associated with diethyl ether is decomposition to explosive peroxides. In a preferred embodiment, the condensable sweep gas compound is used in an amount that does not significantly diminish the CO₂-loading capacity achievable with the CO₂ absorption liquid.

In one preferred embodiment, dimethyl ether is the condensable sweep gas because the CO₂ desorption can be carried out at low temperatures, such as at ambient temperature, or in the range 0-90° C. or in the range 40-60° C., to enable a near-isothermal process between the absorber temperature (typically 40° C. for a post-flue gas desulfurization combustion flue gas, or a biogas) and the desorber temperature, which can be held at 40° C. or raised to a moderately higher temperature, for example to ensure solubility of the CO₂-rich liquid until desorption has occurred.

The condensable sweep gas can be contacted directly with the CO₂-containing liquid, or can be separated from the CO₂-containing liquid, such as by a CO₂-permeable membrane, analogous to an industrial process called “pervaporation,” where the liquid-phase feed is separated from the vapour-phase permeate by a membrane that is selective for the desired components. Vacuum is optionally applied on the permeate side to provide a low partial pressure of the desired component and drive the mass transfer of that component across the membrane.

In one embodiment, the condensable sweep gas compound directly contacts the liquid phase comprising the CO₂ to be removed. In a preferred embodiment, the presence of the condensable sweep has no or minimal impact on the amount of CO₂ that can be absorbed by the absorption solution (also called “loading capacity”).

In one embodiment, the condensable sweep gas compound is a volatile compound that is soluble in or miscible with water and has a lower boiling point than water, but does not form an azeotrope with water, e.g., methanol (b.p. 64.7° C.). The liquid phase comprising the CO₂ to be removed can comprise the water-soluble condensable sweep gas compound. In the reaction zone where desorption of CO₂ is carried out, the temperature is raised above the boiling point of the water-soluble condensable sweep gas compound, and the sweep gas compound evaporates from the water-based liquid, carrying CO₂ along in the vapour phase. Alternatively, the water-soluble condensable sweep gas compound can be raised to a temperature above its boiling point to convert it to a gaseous form, and the gaseous form can be contacted with the liquid phase comprising the CO₂ to be removed, preferably maintaining the temperature above the boiling point of the water-soluble condensable sweep gas.

In one embodiment, the condensable sweep gas compound is a volatile compound that may form an azeotropic mixture together with water and boils at a lower temperature than the boiling temperature of water, preferably in the presence of CO₂ absorption compounds. Upon exposure to heat, the volatile compound performs as a sweep gas as it vaporizes from the mixture. CO₂ dissolved in the liquid is carried along with the sweep gas at lower temperatures than if only water was present. This means less energy is required to remove CO₂ from the liquid. For example, water-miscible compositions, such as ethanol/water, when heated will volatilize towards a low-boiling azeotropic composition. In the case of ethanol/water, the azeotropic composition is 95.629 wt % ethanol, which can, for example, be prepared by mixing 95.629 g dry ethanol with pure water to make a total of 100 g solution (NIST Standard Reference Material 1828). At the azeotropic composition, the composition (of, e.g., ethanol and water) in the vapor phase is the same as the composition in liquid phase. Hence, boiling an azeotropic liquid at its azeotropic composition does not result in a change in the liquid composition. Ethanol boils at 78.4° C. and water boils at 100° C., whereas the azeotrope boils at 78.2° C., which is lower than either of its constituents. Upon heating (or distilling), compositions of ethanol and water, wherein the proportion of ethanol is less than the azeotropic composition, will release relatively more ethanol into the gas phase than water, and the composition of the boiling liquid phase will become less concentrated in ethanol and more concentrated in water. Meanwhile, the proportion of ethanol in the gas phase will increase, and the collected condensate will have a higher proportion of ethanol compared to the initial boiling liquid composition. Therefore, ethanol can perform the function of a sweep gas by vaporizing at a lower temperature compared to water.

In one embodiment, the condensable sweep gas has low solubility in water or is easily separated from water to avoid the sweep gas component being retained by the CO₂-lean liquid as it exits the desorption stage. For example, tertiary butanol is not miscible with water and has a boiling point of 82° C., which is lower than the boiling point of water. Therefore, tertiary butanol can function as a sweep gas and can be subsequently easily separated from water, e.g., in a condensation tank that provides for liquid-liquid separation. Additives, such as surfactants, can be present to enhance the miscibility of the sweep gas compound with water during the CO₂ extraction stage to enhance the effectiveness of the sweep gas in removing CO₂. Also, additives, such as surfactants, can be present to enhance the interaction of CO₂ with the sweep gas, or reduce the interaction of CO₂ with the CO₂ absorption solution, in either case leading to enhanced release of CO₂ from the CO₂ absorption solution.

The ability to conduct low temperature desorption with a condensable sweep gas is especially beneficial when combined with a catalyst, such as a carbonic anhydrase variant, to minimize rate limitations in the conversion of bicarbonate to CO₂ when CO₂ desorption is carried out at low temperature.

In one embodiment, the presence of carbonic anhydrase in the liquid or in contact with the liquid means that a constant supply of dissolved CO₂ will be available in the solution as the result of the CA catalyzed conversion of bicarbonate to CO₂ in aqueous solutions comprising bicarbonate. The resulting vapour phase contains CO₂, H2O and sweep gas compound (e.g., ethanol). Water and ethanol are recovered from the vapour phase in a cold trap, resulting in a pure CO₂ gas at the exit.

The absorber and desorber shown schematically in FIG. 7 can be at essentially the same (“isothermal”) temperature or at different temperatures. A carbonic anhydrase variant may be present in only the absorber or the desorber or both. Regeneration of CO₂ using vacuum (low pressure) at low temperatures, e.g., 70° C. in the desorber where a high temperature carbonic anhydrase such as carbonic anhydrase variant is present is a further embodiment of the present invention. Carbonic anhydrase in such a process catalyses both absorption and desorption of CO₂ to and from the absorption solvent. When the absorber and desorber are at different temperatures, a temperature regulator (e.g., heat exchanger) can be used to conserve energy in the process.

In a further illustration, a modification of the vacuum carbonate process for H₂S absorption (A. Kohl and R. Nielsen, Gas Purification, 5^{th} ed., Gulf Professional Publishing, Houston, TX, 1997:383-388) has been described for CO₂ extraction (US 2007/0256559) and disclosed in combination with carbonic anhydrase (Lu et al., DOE Project No. DE-FC26-08NT0005498, NETL CO₂ Capture Technology for Existing Plants R&D Meeting, Mar. 24-26, 2009, Pittsburgh, PA). In this illustration, atmospheric pressure power plant flue gas contacts aqueous potassium carbonate and carbonic anhydrase in the absorber column at temperatures in the range 40 to 60° C., where carbonic anhydrase improves the rate of CO₂ hydration to bicarbonate in the carrier liquid. The CO₂-rich absorption liquid is pumped to a desorber column (or “stripper”) where CO₂ is released from the absorption liquid by a combination of low pressure (e.g., 14-55 KPa) and the application of heat (e.g., 50-70° C.) obtained by directly injecting low pressure, low quality exhaust steam from a low pressure steam turbine of the power plant. Carbonic anhydrase variants of the present invention are especially suitable for use in the described modified vacuum carbonate process because the variants can tolerate temperatures both in the absorber and the desorber, meaning that, carbonic anhydrase variants can recirculate along with the absorption liquid through both absorption and desorption stages of the process.

A further type of reactor uses membranes in combination with CO₂ hydration catalysis by CA followed by precipitation. In one case, CO₂ is removed from a gaseous stream by passing the gaseous stream through a gas diffusion membrane into solution where conversion to bicarbonate and, subsequently, to carbonate is accelerated by passing the CO₂ solution over a matrix that contains CA and adding a mineral ion to cause precipitation of the carbonic acid salt (U.S. Pat. No. 7,132,090). It has been shown that CA can not only catalyse the CO₂ hydration/dehydration reaction but can also promote the precipitation of calcium carbonate (Mirjafari et al., 2007, Ind. Eng. Che. Res., 46:921-926).

A further type of reactor removes CO₂ from ambient air. A reactor designed to remove CO₂ from ambient air have been reported (Stolaroff et al. 2008 Environ. Sci. Technol., 42:2728-2735), however this reactor does not utilize carbonic anhydrase. Without being bound by the design of the reported ambient air reactor, a CA combined with suitable absorption liquids as disclosed in the present invention, could be used in such a reactor or in other reactor designs as described herein. A heat stable carbonic anhydrase is especially useful because exposure of the reactor to environmental conditions, such as sunlight, may increase the liquid temperature requiring the CA to have good thermostability, thereby avoiding the need to cool the reactor. This illustrates a situation where the process of extracting CO₂ from the CO₂-containing medium requires CA to function at or tolerate higher temperatures than the initial temperature of the CO₂-containing medium, such as ambient air, which may be cold at night (below 10° C.) and hot during the day (above 45° C.).

The different membrane reactors and direct gas-liquid contact reactors described herein as well as other alternatives may be applied in a carbon dioxide extraction process, where the absorption process and desorption process occur in at least two steps. Such reactors generally comprise the following elements: a) at least one absorption unit, which may comprise a gas inlet zone and/or a gas outlet zone; b) at least one desorption unit comprising a gas outlet zone; c) a CO₂ absorption liquid; and d) means for connecting the absorption unit(s) and the desorption unit(s) such that the absorption liquid can pass from the absorption unit(s) to the desorption unit(s). Optionally the means for connecting the absorption and desorption units is a circuit, allowing the absorption liquid to be returned to the absorption unit once it has passed through the desorption unit. One or both of the units may comprise at least one CO₂-permeable membrane which separates a gas phase from a liquid phase, such as described in WO 2010/014773 and WO 2010/014774. This type of membrane unit is also termed a gas-liquid membrane (GLM) unit. The GLM unit may, e.g., be in the form of a hollow fiber membrane, a flat sheet membrane or a spiral-wound membrane. The GLM unit may either function as an absorber unit and/or a desorber unit. Alternatively, one of the units may be a GLM unit and the other unit may be composed such that the gas and liquid phases are in direct contact or in other words the gas-liquid interface is not separated by a membrane. This type of unit is also termed a direct gas-liquid contact (DGLC) unit or just a direct contact (DC) unit. The DGLC unit may, e.g., be in the form of a column filled with packing material that allows for gas-liquid contact, and/or a liquid-containing vessel equipped with an inlet for exposing gas to the liquid (such as a bubble column), and/or a liquid-spray (such as a spray tower) and/or an aerator unit and/or a falling film. The DGLC unit may either function as an absorber unit or a desorber unit. Bubble cap system, sieve plate system, disk-and-doughnut column and packed column are examples of the internals found in DGLC units.

The reactor types described above may be operated at any desired temperature. In one embodiment, the reactor is operated with a temperature of the liquid in contact with and/or comprising carbonic anhydrase between 0° C. and 120° C. or 5° C. and 110° C., more preferably between 10° C. and 100° C., more preferably between 20° C. and 95° C., more preferably between 30° C. and 90° C., more preferably between 40° C. and 85° C., more preferably between 40° C. and 80° C., more preferably between 40° C. and 75° C., and more preferably between 40° C. and 70° C., and most preferably between 40° C. and 60° C.

The absorption and desorption rates of CO₂ are dependent on the pH in the absorption liquid. In the reactor types described in relation to the present invention the pH of the CO₂-lean absorption liquid is between pH 4 to12, preferably above pH 7 (as measured at room temperature, e.g., 20-25° C.), more preferably above pH 8, more preferably between 8 and 12, more preferably between 8 and 10.5, more preferably between 8.5 and 10, even more preferably between 9 and 9.5. The hydration of CO₂ to bicarbonate during absorption results in release of a proton causing the pH of the absorption liquid to decrease as the carbon content of the CO₂-rich absorption liquid increases. The extent of pH decrease depends on the buffering capacity of the absorption liquid and the amount of CO₂ absorbed. In a preferred embodiment of the present invention the absorption liquid is a bicarbonate-based buffer or a carbonate-based buffer, such as lithium bicarbonate, sodium bicarbonate, potassium bicarbonate, cesium bicarbonate, ammonium bicarbonate or another suitable salt of the bicarbonate, or lithium carbonate, sodium carbonate, potassium carbonate, cesium carbonate, ammonium carbonate or another suitable salt of the carbonate, or combinations of bicarbonate and carbonate compounds, where, depending on the pH, greater or lesser amount of carbonate and/or carbonic acid will exist together with bicarbonate.

In one embodiment of the present invention, the CO₂-rich absorption liquid passes through a desorption stage where the pH of the CO₂-rich absorption liquid will increase as the CO₂ is released. In order to recirculate absorption liquid through such an absorption-desorption system, it is preferred that the pH of the absorption liquid returns to the pH of the CO₂-lean absorption liquid before again passing through the absorption stage.

In a preferred embodiment of the present invention the reactor is equipped with means for regulating pH in the absorption liquid. This can be performed in several ways. One way is to add an alkaline substance to the absorption liquid, e.g., at one of the auxiliary component's addition points (20) indicated in FIG. 7, using automatic pH adjustment equipment such as an automatic titrator. The alkaline substance preferably has a similar composition (e.g., concentration of solvent, ionic strength, amount of carbonic anhydrase, etc.) as the absorption liquid circulating in the system and can be added at any time before absorption for adjustment of pH. Similarly, a neutral to acidic substance can be added to the absorption liquid any time before desorption, e.g., at one of the auxiliary component's addition points (21) indicated in FIG. 7. Extra absorption liquid can be removed from the system if needed, e.g., at one of the removal points (24 and 25) indicated in FIG. 7.

In the CO₂ capture processes described herein the carbonic anhydrase variants of the present invention may be combined with one or more other CAs. Different process steps in the overall CO₂ scrubbing process may require optimization of operating conditions, e.g., temperature, pH, carrier liquid compositions, pressure and so forth. The CAs of the present invention may be combined with other CAs operating at different optimal conditions and which are suitable for use in the CO₂ scrubbing process. For example, one CA can circulate throughout the system along with the absorption liquid and a different CA can be immobilized at one or more locations in the system.

The carbonic anhydrase variants of the present invention and biocatalyst based bioreactors described herein comprising a CA of the present invention also find more unconventional applications, such as in pilot cockpits, submarine vessels, aquatic gear, safety and firefighting gear, astronaut space suits and artificial lung devices to keep breathing air free of toxic CO₂ levels. Other applications are to remove CO₂ from confined spaces, such as to reduce hazardous CO₂ levels from inside breweries and enclosed buildings carrying out fermentation, and from CO₂ sensitive environments like museums and libraries, to prevent excessive CO₂ from causing acid damage to books and artwork. Another application is to remove CO₂ from hot ambient air, e.g., in a desert. In this case the carbonic anhydrase could for example be comprised in a reactor suitable for extracting CO₂ from ambient air as described in Stolaroff et al. 2008 Environ. Sci. Technol., 42, 2728-2735, such a reactor can, for example, take the form of an “artificial tree” or a windmill as described in WO2008/041920.

The carbonic anhydrase variants described herein can be used alone as a CO₂ extraction biocatalyst together with a water-based absorption liquid or it may optionally be combined with conventional CO₂ extraction technologies such as chemical absorption via amine-based solvents or aqueous ammonia or physical solvents such as Selexol™ (Union Carbide) or polyethylene glycol ethers. In a further embodiment of the present invention, a carbonic anhydrase variant is combined with one or more CO₂ absorbing compounds, such as amine-based compounds, for example, aqueous alkanolamines including monoethanolamine (MEA), diethanolamine (DEA), N-methyldiethanolamine (MDEA), 2-amino-2-hydroxymethyl-1,3-propanediol (Tris or AHPD), diglycolamine (DGA), 2-amino-2-methyl-1-propanol (AMP), Methylmonoethanolamine (MMEA), Dimethylmonoethanolamine (DMMEA), diethylmonoethanolamine (DEMEA), diisopropanol amine (DIPA), triisopropanolamine (TIPA), aqueous soluble salts (e.g., sodium or potassium salts) of N-methylaminopropionic acid or N,N-dimethylaminoacetic acid or N-methylalanine, N-methylglycine, N,N-dimethylglycine, beta-alanine (3-aminopropanoic acid) or other natural or modified amino acids (e.g., N-substituted amino acid derivatives), 2-(2-aminoethylamino) ethanol (AEE), triethanolamine (TEA) or other primary, secondary, tertiary or hindered amine-based solvents including those described on pages 7 to 9 of U.S. Pat. No. 4,112,052 (hereby incorporated by reference), or aqueous soluble salts of glycine (e.g., sodium or potassium glycinate) and taurine or other liquid CO₂ absorption compositions such as aqueous solutions comprising NaOH, KOH, LiOH, alkali-metal carbonate salts (e.g., lithium, sodium, potassium, or ammonium), alkali-metal bicarbonate salts, alkali-metal phosphate salts, or borate salts, such as sodium tetraborate decahydrate (borax), at different ionic strengths, molar concentrations (ranging from dilute solutions to highly concentrated solutions, up to the solubility limit of the salts, which may vary based on the temperature) or aqueous electrolyte solutions and promoters such as piperazine, or polyethylene glycol ethers, or a blend of them or analogs or blends thereof. The aqueous soluble salts and solvents may be used in combinations with each other and may be combined with pH buffering and mineral sequestering compounds, such as phosphate salts, polyphosphate salts and borate salts, to provide mixed salt solutions, such as potassium or sodium bicarbonate with potassium or sodium phosphate. The aqueous soluble salts and solvents may be combined with simple electrolytes (e.g., alkali halides, such as NaCl, KCl, and metal halides, such as ZnCl) and sulfate salts, such as sodium sulfate and potassium sulfate. Preferably the CO₂ absorption composition comprises sufficient concentration of aqueous-soluble salts, e.g., NaCl, to stabilize and optimize the catalytic activity of the carbonic anhydrase variant. The combination of a carbonic anhydrase variant with CO₂ absorption components may be applied in the bioreactors described herein and may be applied to already existing CO₂ scrubbing facilities based on conventional techniques. In conventional bioreactors, the concentration of alkanolamines is typically 15-30 weight percent. In an embodiment of the present invention the concentration of alkanolamines can be in the conventional range or preferably at a lower concentration, such as preferably below 15% (V/V), more preferably below 12%, 10%, 8%, 6%, 5%, 4%, 3%, 2%, 1%, 0.5%, 0.2% and most preferably below 0.1% (V/V).

Certain simple amino acids and amines are known to activate α-CAs (Akdemir et al., 2013, The extremo-α-carbonic anhydrase (CA) from Sulfurihydrogenibium azorense, the fastest CA known, is highly activated by amino acids and amines, Bioorg. Med. Chem. Lett. 23:1087-1090), and are herein incorporated by reference. They include D-Phe, L-DOPA, L- and D-Trp, dopamine, serotonin, L- and D-His, L-Phe, L-Tyr, 2-pyridyl-methylamine, L-adrenaline, D-DOPA, D-Tyr, and several heterocyclic amines.

In conventional processes, corrosion and oxidation inhibitors, such as contained in Fluor Daniel's proprietary EconAmine FG solvent, are added to provide for increasing the amine concentration while reducing the risk of corrosion. Inorganic corrosion inhibitors include vanadium (e.g., sodium metavanadate), antimony, copper, cobalt, tin, and sulfur compounds. Organic corrosion inhibitors include thiourea and salicylic acid.

Other auxiliary absorption liquid components can include wetting agents, chelating agents (e.g., ethylenediamine tetraacetic acid, polyphosphate salts), antifoams, viscosity reducers, and other compounds capable of increasing the flux of CO₂ into or out of the carrier liquid.

In conventional processes, techniques to reduce and/or avoid foam formation are commonly employed. These include removal of foam-causing impurities prior to CO₂ extraction and use of antifoaming agents and foam inhibitors such as silicone compounds or high-boiling alcohols such as oleyl alcohol or octylphenoxyethanol (A. Kohl and R. Nielsen, Gas Purification, 5^{th} ed., Gulf Professional Publishing, Houston, TX, 1997:224-230).

Another aspect of the present invention relates to biogas production where the CO₂ extraction is performed directly in the biogas fermentation broth, as an alternative to passing the biogas through a bioreactor as described above. By adding a carbonic anhydrase variant to the anaerobic broth, as an additive in a biogas fermentation medium, more CO₂ from the gas phase can be converted into bicarbonate, which is the substrate for methane production by the methanogenic Archaea. Particularly, the genus Methanosarcina is frequently present in thermophilic biogas digesters (Mladenovska and Ahring, 2000, FEMS Microbiol. Ecol. 3:225-229). It has been shown for Methanosarcina thermophila TM-1 that bicarbonate may be a limiting factor for the methane production, for example cultures of M. thermophila™-1 grown in low bicarbonate solution (0.6 mM) showed a considerable lag phase (i.e., methane production began later) when compared with cultures containing ten times higher bicarbonate dosages (6 mM). Additionally, the total yield of methane was 25 times less at the lower bicarbonate dosage (Murray and Zinder, 1985, Appl. Environ. Microbiol. 50:49-55). Consequently, a heat-stable carbonic anhydrase is particularly useful when the biogas production is performed at elevated temperatures using one or more thermophilic microorganisms, for example methanogens like Methanosarcina sp. that can use CO₂/biocarbonate as carbon source for growth and methanogenesis.

A further embodiment of the present invention is use of a carbonic anhydrase variant described herein to enhance growth of algae and other aquatic plants that utilize bicarbonate as a carbon source by catalyzing the conversion of CO₂ to bicarbonate in or for delivery to the aquatic plant environment. This approach can, for example, be used to simultaneously remove CO₂ from a combustion exhaust gas, such as a flue gas, and provide CO₂ for conversion to bicarbonate by contacting the exhaust gas with liquid from a cultivation pond. Certain approaches to cultivating algae and aquatic plants involve use of enclosed tubes or shallow troughs or ponds in which heat from sunlight raises the water temperature. Hence a heat stable carbonic anhydrase is particularly useful at the elevated cultivation temperatures.

The present invention is further described by the following examples that should not be construed as limiting the scope of the invention.

### EXAMPLES

### Example 1: Preparation of Carbonic Anhydrase Variants

Mutation and introduction of expression cassettes into Bacillus subtilis was performed by standard methods known in the art. All DNA manipulations were performed by PCR (e.g., as described by Sambrook et al., 1989, Molecular Cloning: A Laboratory Manual, Cold Spring Harbor Lab., NY, Davis et al., 2012, Basic Methods in Molecular Biology, Elsevier, and Song et al., 2016, PLOS One 11 (7): e0158447) using standard methods known to the skilled person. Recombinant B. subtilis constructs encoding carbonic anhydrase polypeptides were inoculated into and cultivated in a complex medium (TBgly) under antibiotic selection for 24 h at 37° C., 700 prm (Incubator Hood TH 15, Edmund Buhler). The 24 h cultures were inoculated in a ratio of 1:30 in rich media (PS-1:100 g/L Sucrose (Danisco cat. no. 109-0429), 40 g/L crust soy (soybean flour), 10 g/L Na₂HPO₄·12H₂O (Merck cat. no. 106579), 0.1 ml/L Dowfax63N10 (Dow)) and incubated for 3 days at 700 rpm at 37° C. Following incubation, the fermentation broths are centrifuged at 2500 rpm for 15 min (Thermo Scientific Heraeus Megafuge 40 centrifuge) and the supernatants are transferred to a PCR tray and heated at 80° C. for 30 min (Applied Biosystems GeneAMP PCR Systems 9700). The heat-treated samples are centrifuged at 2000 rpm for 15 min and the supernatants are mixed in a ratio of 1:1 with buffer (20% MDEA (N-methyl diethanolamine) 250 mM Glycine pH10).

### Example 2: Thermal Stability of Persephonella marina Carbonic Anhydrase Variants with a Single Alteration

The thermal stability of non-purified AoPDE variants of the Persephonella marina carbonic anhydrase of SEQ ID NO: 1 prepared as described in Example 1 was estimated by differential scanning fluorimetry (nanoDSF (nDSF), Prometheus NT.48 or NT.Plex, NanoTemper Technologies GmbH, Germany), which measures the change in intrinsic tryptophan and tyrosine fluorescence due to unfolding upon temperature increase (20° C.-110° C., ramp-rate 1° C. min). The supernatants from Example 1 were mixed in a ratio of 1:1 with buffer (20% MDEA (N-methyl diethanolamine) 250 mM Glycine pH10) and added to the nDSF capillaries. Melting temperatures of 182 variants were calculated from the inflection points in the melting curve using the first and second derivative and compared to the parent P. marina carbonic anhydrase (wt) shown in Table 2 below. Variants 1-87 showed improved stability compared to the parent carbonic anhydrase.

TABLE 2

| Entry | Carbonic Anhydrase Alteration | Tm |
|---|---|---|
| 1 | E186K | 95.9 |
| 2 | D39W | 93.5 |
| 3 | D39L | 93.4 |
| 4 | E186R | 93.2 |
| 5 | E221L | 92.6 |
| 6 | D39K | 92.0 |
| 7 | E221R | 91.5 |
| 8 | D39E | 91.3 |
| 9 | D39R | 89.5 |
| 10 | D39P | 89.4 |
| 11 | E221V | 88.4 |
| 12 | D39M | 88.3 |
| 13 | D39Y | 88.3 |
| 14 | E186I | 87.9 |
| 15 | M220L | 85.9 |
| 16 | D39A | 85.9 |
| 17 | D163K | 85.9 |
| 18 | S168K | 85.7 |
| 19 | D39N | 85.6 |
| 20 | D39F | 85.4 |
| 21 | K133M | 84.7 |
| 22 | T173I | 84.4 |
| 23 | D39S | 84.4 |
| 24 | M191I | 84.2 |
| 25 | M191N | 84.2 |
| 26 | M191P | 84.1 |
| 27 | K41A | 84.0 |
| 28 | H99P | 83.9 |
| 29 | M191G | 83.9 |
| 30 | K64L | 83.8 |
| 31 | H61Y | 83.7 |
| 32 | M191T | 83.7 |
| 33 | M191H | 83.6 |
| 34 | K55Y | 83.5 |
| 35 | H150V | 83.5 |
| 36 | D156L | 83.5 |
| 37 | M191L | 83.5 |
| 38 | H150V | 83.5 |
| 39 | M218V | 83.4 |
| 40 | M191V | 83.4 |
| 41 | M191R | 83.3 |
| 42 | H112D | 83.2 |
| 43 | M191Q | 83.2 |
| 44 | S168N | 83.1 |
| 45 | E187V | 83.1 |
| 46 | D18E | 83.0 |
| 47 | E221F | 82.9 |
| 48 | K79V | 82.9 |
| 49 | A149H | 82.8 |
| 50 | M185L | 82.8 |
| 51 | E127M | 82.8 |
| 52 | H61F | 82.6 |
| 53 | K41N | 82.6 |
| 54 | E81Y | 82.5 |
| 55 | H99Y | 82.5 |
| 56 | M191K | 82.5 |
| 57 | T207N | 82.4 |
| 58 | K201M | 82.4 |
| 59 | A53G | 82.4 |
| 60 | H112K | 82.4 |
| 61 | D156M | 82.3 |
| 62 | M191A | 82.3 |
| 63 | K79I | 82.2 |
| 64 | E127V | 82.1 |
| 65 | E127V | 82.1 |
| 66 | A104L | 82.0 |
| 67 | E48V | 82.0 |
| 68 | M189L | 82.0 |
| 69 | H112E | 82.0 |
| 70 | K55F | 82.0 |
| 71 | N59V | 81.9 |
| 72 | V137K | 81.9 |
| 73 | K46Y | 81.8 |
| 74 | K201I | 81.8 |
| 75 | G1M | 81.8 |
| 76 | G1D | 81.7 |
| 77 | S66F | 81.7 |
| 78 | H5S | 81.7 |
| 79 | E48S | 81.7 |
| 80 | N59W | 81.7 |
| 81 | H150S | 81.6 |
| 82 | D163R | 81.6 |
| 83 | K41M | 81.6 |
| 84 | E186L | 81.5 |
| 85 | K198F | 81.5 |
| 86 | D18P | 81.5 |
| 87 | F122Y | 81.5 |
| 88 | wt | 81.4 |
| 89 | T62M | 81.3 |
| 90 | K147I | 81.3 |
| 91 | Q98K | 81.3 |
| 92 | E221Y | 81.2 |
| 93 | M203I | 81.2 |
| 94 | A142K | 81.2 |
| 95 | T173V | 81.2 |
| 96 | K147M | 81.2 |
| 97 | R126K | 81.2 |
| 98 | K64Q | 81.1 |
| 99 | D18S | 81.1 |
| 100 | M191F | 81.1 |
| 101 | E187I | 81.1 |
| 102 | Q84Y | 81.0 |
| 103 | M189V | 81.0 |
| 104 | F102M | 81.0 |
| 105 | I130E | 81.0 |
| 106 | D18G | 81.0 |
| 107 | N59I | 81.0 |
| 108 | E48Y | 81.0 |
| 109 | N59H | 80.9 |
| 110 | K43H | 80.9 |
| 111 | E48N | 80.9 |
| 112 | S66W | 80.8 |
| 113 | M203L | 80.8 |
| 114 | E127L | 80.8 |
| 115 | D18K | 80.7 |
| 116 | D161N | 80.7 |
| 117 | T62N | 80.7 |
| 118 | D39G | 80.7 |
| 119 | H99L | 80.7 |
| 120 | E48D | 80.7 |
| 121 | L95I | 80.7 |
| 122 | S177W | 80.6 |
| 123 | E188V | 80.6 |
| 124 | S177M | 80.6 |
| 125 | S177F | 80.6 |
| 126 | A89T | 80.6 |
| 127 | M191W | 80.6 |
| 128 | T62W | 80.6 |
| 129 | K17S | 80.4 |
| 130 | S177Y | 80.4 |
| 131 | R181I | 80.3 |
| 132 | R181L | 80.3 |
| 133 | Y67V | 80.3 |
| 134 | A149P | 80.3 |
| 135 | K94M | 80.3 |
| 136 | K55H | 80.2 |
| 137 | K111E | 80.2 |
| 138 | K41D | 80.2 |
| 139 | R50K | 80.1 |
| 140 | S66I | 80.1 |
| 141 | N141K | 80.1 |
| 142 | N114P | 80.1 |
| 143 | K41S | 80.1 |
| 144 | E178S | 79.9 |
| 145 | G119A | 79.9 |
| 146 | F102L | 79.9 |
| 147 | L82P | 79.8 |
| 148 | L148P | 79.7 |
| 149 | K64I | 79.7 |
| 150 | K41L | 79.6 |
| 151 | K43Y | 79.6 |
| 152 | T62F | 79.5 |
| 153 | D18R | 79.5 |
| 154 | T62Y | 79.3 |
| 155 | N59L | 79.1 |
| 156 | A104V | 78.2 |
| 157 | N59S | 78.0 |
| 158 | Q84K | 77.4 |
| 159 | G119S | 77.4 |
| 160 | E48F | 77.2 |
| 161 | Q84W | 77.1 |
| 162 | G119T | 76.8 |
| 163 | Q84G | 76.0 |
| 164 | G119I | 75.4 |
| 165 | Q84E | 73.5 |
| 166 | Q84A | 73.0 |
| 167 | N59T | 72.9 |
| 168 | D163T | 72.8 |
| 169 | S66Y | 72.2 |
| 170 | T173W | 71.7 |
| 171 | G119C | 70.8 |
| 172 | S168G | 70.4 |
| 173 | S168A | 70.1 |
| 174 | E178P | 70.0 |
| 175 | S168T | 69.8 |
| 176 | S168V | 69.6 |
| 177 | E178Q | 69.5 |
| 178 | S168C | 69.4 |
| 179 | S168L | 69.2 |
| 180 | S168D | 68.6 |
| 181 | S168D | 68.6 |
| 182 | S168P | 68.0 |
| 183 | S168Q | 67.8 |

### Example 3: Thermal Stability of Persephonella marina Carbonic Anhydrase Variants with Multiple Alterations

Persephonella marina carbonic anhydrase variants of SEQ ID NO: 1 containing multiple alterations were prepared as described in Example 1 and the thermal stability measured according to the procedure of Example 2. Melting temperatures of 940 variants were calculated from the inflection points in the melting curve using the first and second derivative and compared to the parent Persephonella marina carbonic anhydrase (wt) shown in Table 3 below. Variants 1-322 were too stable for detection by nDSF (indicated with **). Variants 1-802 showed improved stability compared to the parent carbonic anhydrase.

TABLE 3

| Entry | Carbonic Anhydrase Alteration | Tm |
|---|---|---|
| 1 | D18P + D39L + K64L + E186K + E188P + M220L | ** |
| 2 | D18P + D39L + K64L + E186K + E188P + K198L | ** |
| 3 | D18P + D39L + K64L + D156L + E186K + E188P | ** |
| 4 | D18P + D39L + K64L + E186K + E188P + K198L + M220L | ** |
| 5 | D18P + D39L + K64L + D156L + E186K + E188P + M220L | ** |
| 6 | D18P + D39L + K55Y + K64L + E186K + E188P + M220L | ** |
| 7 | D18P + D39L + K64L + E186K + E188P + M189L + M220L | ** |
| 8 | D18P + D39L + K55Y + K64L + E186K + E188P + M189L | ** |
| 9 | D18P + D39L + K64L + D156L + E186K + E188P + K198L + M220L | ** |
| 10 | D18P + D39L + K55Y + K64L + E186K + E188P + K198L + M220L | ** |
| 11 | D18P + D39L + K64L + E186K + E188P + M189L + K198L + M220L | ** |
| 12 | D18P + D39L + K55Y + K64L + D156L + E186K + E188P + M220L | ** |
| 13 | D18P + D39L + K55Y + K64L + E186K + E188P + M189L + M220L | ** |
| 14 | D18P + D39L + K55Y + K64L + D156L + E186K + E188P + K198L | ** |
| 15 | D18P + D39L + K64L + D156L + E186K + E188P + M189L + K198L | ** |
| 16 | D18P + D39L + K55Y + K64L + D156L + E186K + E188P + K198L + M220L | ** |
| 17 | D18P + D39L + K64L + D156L + E186K + E188P + M189L + K198L + M220L | ** |
| 18 | D18P + D39L + K55Y + K64L + D156L + E186K + E188P + M189L + M220L | ** |
| 19 | D18P + D39L + K55Y + K64L + D156L + E186K + E188P + M189L + K198L | ** |
| 20 | E7K + D18P + D39L + E186K + E188P + M189L + M220L | ** |
| 21 | E7K + D18P + D39L + E186K + E188P + M189L + K198L + M220L | ** |
| 22 | E7K + D18P + D39L + D156L + E186K + E188P + M189L + M220L | ** |
| 23 | E7K + D18P + D39L + K55Y + E186K + E188P + M189L + M220L | ** |
| 24 | E7K + D18P + D39L + K64L + E186K + E188P + M189L + M220L | ** |
| 25 | E7K + D18P + D39L + K64L + D156L + E186K + E188P + M189L | ** |
| 26 | E7K + D18P + D39L + K55Y + K64L + E186K + E188P + M189L | ** |
| 27 | E7K + D18P + D39L + D156L + E186K + E188P + M189L + K198L + M220L | ** |
| 28 | E7K + D18P + D39L + K55Y + E186K + E188P + M189L + K198L + M220L | ** |
| 29 | E7K + D18P + D39L + K64L + E186K + E188P + M189L + K198L + M220L | ** |
| 30 | E7K + D18P + D39L + K55Y + D156L + E186K + E188P + M189L + M220L | ** |
| 31 | E7K + D18P + D39L + K64L + D156L + E186K + E188P + M189L + M220L | ** |
| 32 | E7K + D18P + D39L + K55Y + K64L + E186K + E188P + M189L + M220L | ** |
| 33 | E7K + D18P + D39L + K55Y + H150R + D156L + E186K + E188P + M189L + K198L | ** |
| 34 | E7K + D18P + D39L + K64L + D156L + E186K + E188P + M189L + K198L | ** |
| 35 | E7K + D18P + D39L + K55Y + K64L + E186K + E188P + M189L + K198L | ** |
| 36 | E7K + D18P + D39L + K55Y + D156L + E186K + E188P + M189L + K198L + M220L | ** |
| 37 | E7K + D18P + D39L + K64L + D156L + E186K + E188P + M189L + K198L + M220L | ** |
| 38 | E7K + D18P + D39L + K55Y + K64L + E186K + E188P + M189L + K198L + M220L | ** |
| 39 | E7K + D18P + D39L + K55Y + K64L + D156L + E186K + E188P + M189L + M220L | ** |
| 40 | D18P + D39L + D156L + E186K + E188P + M189L + M220L | ** |
| 41 | D18P + D39L + K64L + E186K + E188P + M189L + K198L | ** |
| 42 | D18P + D39L + K55Y + D156L + E186K + E188P + M189L | ** |
| 43 | D18P + D39L + K64L + D156L + E186K + E188P + M189L | ** |
| 44 | D18P + D39L + D156L + E186K + E188P + M189L + K198L + M220L | ** |
| 45 | D18P + D39L + K55Y + E186K + E188P + M189L + K198L + M220L | ** |
| 46 | D18P + D39L + K55Y + D156L + E186K + E188P + M189L + M220L | ** |
| 47 | D18P + D39L + K64L + D156L + E186K + E188P + M189L + M220L | ** |
| 48 | D18P + D39L + K55Y + D156L + E186K + E188P + M189L + K198L | ** |
| 49 | D18P + D39L + K55Y + K64L + E186K + E188P + M189L + K198L | ** |
| 50 | D18P + D39L + K55Y + K64L + D156L + E186K + E188P + M189L | ** |
| 51 | D18P + D39L + K55Y + D156L + E186K + E188P + M189L + K198L + M220L | ** |
| 52 | D18P + D39L + K55Y + K64L + E186K + E188P + M189L + K198L + M220L | ** |
| 53 | D18P + D39L + K55Y + K64L + D156L + E186K + E188P + M189L + K198L + M220L | ** |
| 54 | D18P + N28R + D39L + K64L + S168P + E186K + E188P | ** |
| 55 | D18P + N28E + D39L + K64L + S168D + E186K + E188P | ** |
| 56 | D18P + N28R + D39L + K64L + E186K + E188P | ** |
| 57 | D18P + N28Q + D39L + K64L + S168Q + E186K + E188P | ** |
| 58 | D18P + N28R + D39L + K55Y + K64L + D156L + E186K + E188P + M189L + K198L + M220L | ** |
| 59 | D18P + N28H + D39L + K64L + S168T + E186K + E188P | ** |
| 60 | D18P + N28R + D39L + K64L + S168Q + E186K + E188P | ** |
| 61 | D18P + N28M + D39L + K64L + S168M + E186K + E188P | ** |
| 62 | D18P + N28S + D39L + K64L + E186K + E188P | ** |
| 63 | D18P + N28R + D39L + K64L + S168M + E186K + E188P | ** |
| 64 | D18P + N28Q + D39L + K64L + S168P + E186K + E188P | ** |
| 65 | D18P + N28H + D39L + K64L + S168E + E186K + E188P | ** |
| 66 | D18P + N28Q + D39L + K64L + S168E + E186K + E188P | ** |
| 67 | D18P + N28L + D39L + K64L + S168V + E186K + E188P | ** |
| 68 | D18P + N28Y + D39L + K64L + S168E + E186K + E188P | ** |
| 69 | D18P + N28F + D39L + K64L + S168E + E178Q + E186K + E188P | ** |
| 70 | D18P + N28M + D39L + K64L + S168I + E186K + E188P | ** |
| 71 | D18P + N28Q + D39L + K64L + E186K + E188P | ** |
| 72 | D18P + N28H + D39L + K64L + S168D + E186K + E188P | ** |
| 73 | D18P + N28K + D39L + K64L + E186K + E188P | ** |
| 74 | D18P + D39L + K64L + E178Q + E186K + E188P | ** |
| 75 | D18P + N28L + D39L + K64L + S168I + E186K + E188P | ** |
| 76 | D18P + N28E + D39L + K64L + S168M + E186K + E188P | ** |
| 77 | D18P + D39L + N59D + K64L + E186K + E188P | ** |
| 78 | D18P + D39L + N59D + K64L + Q84E + E186K + E188P | ** |
| 79 | D18P + D39L + K64L + Q84G + E186K + E188P | ** |
| 80 | D18P + D39L + K64L + Q84A + E186K + E188P | ** |
| 81 | D18P + N28E + D39L + K64L + V75I + S168D + E186K + E188P | ** |
| 82 | D18P + N28H + D39L + K64L + V75I + E186K + E188P | ** |
| 83 | D18P + N28R + D39L + K64L + V75I + S168D + E186K + E188P | ** |
| 84 | D18P + N28R + D39L + K64L + V75I + E186K + E188P | ** |
| 85 | D18P + N28Q + D39L + K64L + V75I + S168Q + E186K + E188P | ** |
| 86 | D18P + N28H + D39L + K64L + V75I + S168T + E186K + E188P | ** |
| 87 | D18P + N28M + D39L + K64L + V75I + E186K + E188P | ** |
| 88 | D18P + N28S + D39L + K64L + V75I + E186K + E188P | ** |
| 89 | D18P + N28Q + D39L + K64L + V75I + S168P + E186K + E188P | ** |
| 90 | D18P + N28H + D39L + K64L + V75I + S168E + E186K + E188P | ** |
| 91 | D18P + N28Q + D39L + K64L + V75I + S168E + E186K + E188P | ** |
| 92 | D18P + N28L + D39L + K64L + V75I + F87L + V146A + S168V + E186K + E188P | ** |
| 93 | D18P + N28Y + D39L + K64L + V75I + S168E + E186K + E188P | ** |
| 94 | D18P + N28F + D39L + K64L + V75I + S168E + E178Q + E186K + E188P | ** |
| 95 | D18P + N28M + D39L + K64L + V75I + S168I + E186K + E188P | ** |
| 96 | D18P + N28Q + D39L + K64L + V75I + E186K + E188P | ** |
| 97 | D18P + N28H + D39L + K64L + V75I + S168D + E186K + E188P | ** |
| 98 | D18P + N28K + D39L + K64L + V75I + E186K + E188P | ** |
| 99 | D18P + D39L + K64L + V75I + E178Q + E186K + E188P | ** |
| 100 | D18P + N28Q + D39L + K64L + V75I + S168K + E186K + E188P | ** |
| 101 | D18P + N28L + D39L + K64L + V75I + S168I + E186K + E188P | ** |
| 102 | D18P + D39L + K64L + V75I + S168A + E186K + E188P | ** |
| 103 | D18P + N28Q + D39L + K64L + V75I + E186K + E188P + M189L + K198L + M220L | ** |
| 104 | D18P + N28Q + D39L + K64L + V75I + S168T + E186K + E188P | ** |
| 105 | D18P + D39L + N59R + K64L + V75I + E186K + E188P | ** |
| 106 | D18P + D39L + N59T + K64L + V75I + Q84E + E186K + E188P | ** |
| 107 | D18P + D39L + N59V + K64L + V75I + E186K + E188P | ** |
| 108 | D18P + D39L + N59D + K64L + V75I + E186K + E188P | ** |
| 109 | D18P + D39L + N59D + K64L + V75I + Q84E + E186K + E188P | ** |
| 110 | D18P + D39L + K64L + V75I + Q84G + E186K + E188P | ** |
| 111 | D18P + D39L + K64L + V75I + Q84A + E186K + E188P | ** |
| 112 | D18P + D39L + K64L + V75I + E186K + E188P | ** |
| 113 | D18P + D39L + K64L + V75L + E186K + E188P | ** |
| 114 | D18P + N28E + D39L + D156L + S168D + E186K + E188P + M189L | ** |
| 115 | D18P + N28H + D39L + V75I + E186K + E188P + M189L | ** |
| 116 | D18P + N28H + D39L + V75I + S168T + E186K + E188P + M189L | ** |
| 117 | D18P + N28M + D39L + V75I + S168M + E186K + E188P | ** |
| 118 | D18P + N28S + D39L + V75I + E186K + E188P + M189L | ** |
| 119 | D18P + N28Q + D39L + S168P + E186K + E188P + M189L | ** |
| 120 | D18P + N28Q + D39L + K55Y + K64L + S168P + E186K + E188P + M189L | ** |
| 121 | D18P + N28R + D39L + V75I + S168E + E186K + E188P + M189L | ** |
| 122 | D18P + N28H + D39L + V75I + S168E + E186K + E188P + M189L | ** |
| 123 | D18P + N28Q + D39L + V75I + S168E + E186K + E188P + M189L | ** |
| 124 | D18P + N28L + D39L + V75I + S168V + E186K + E188P + M189L | ** |
| 125 | D18P + N28Y + D39L + V75I + S168E + E186K + E188P + M189L | ** |
| 126 | D18P + N28F + D39L + V75I + S168E + E178Q + E186K + E188P + M189L | ** |
| 127 | D18P + N28Q + D39L + V75I + E186K + E188P + M189L | ** |
| 128 | D18P + N28H + D39L + V75I + S168D + E186K + E188P + M189L | ** |
| 129 | D18P + N28K + D39L + V75I + E186K + E188P + M189L | ** |
| 130 | D18P + D39L + V75I + E178Q + E186K + E188P + M189L | ** |
| 131 | D18P + N28Q + D39L + V75I + S168K + E186K + E188P + M189L + K198L + M220L | ** |
| 132 | D18P + N28Q + D39L + V75I + S168K + E186K + E188P | ** |
| 133 | D18P + N28Q + D39L + K55Y + K64L + N153D + D156L + S168T + E186K + E188P + M189L | ** |
| 134 | D18P + D39L + N59T + V75I + Q84K + E186K + E188P + M189L | ** |
| 135 | D18P + D39L + N59I + V75I + E186K + E188P + M189L | ** |
| 136 | D18P + D39L + V75I + Q84G + E186K + E188P + M189L | ** |
| 137 | D18P + D39L + V75I + Q84A + L171F + E186K + E188P + M189L | ** |
| 138 | D18P + N28R + D39L + N59L + K64L + D156L + S168P + E186K + E188P + M189L | ** |
| 139 | D18P + N28E + D39L + N59L + V75I + S168D + E186K + E188P + M189L | ** |
| 140 | D18P + N28R + D39L + K64L + S168D + E186K + E188P + M189L | ** |
| 141 | D18P + N28R + D39L + N59L + V75I + E186K + E188P + M189L | ** |
| 142 | D18P + N28Q + D39L + N59L + K64L + S168Q + E186K + E188P + M189L | ** |
| 143 | D18P + N28Q + D39L + N59L + D156L + S168Q + E186K + E188P + M189L | ** |
| 144 | D18P + N28R + D39L + N59R + V75I + S168P + E186K + E188P + M189L | ** |
| 145 | D18P + N28E + D39L + N59R + V75I + S168D + E186K + E188P + M189L | ** |
| 146 | D18P + N28R + D39L + N59R + V75I + S168D + E186K + E188P + M189L | ** |
| 147 | D18P + N28R + D39L + N59R + V75I + E186K + E188P + M189L | ** |
| 148 | D18P + N28R + D39L + K55Y + K64L + D156L + S168P + E186K + E188P + M189L + K198L + M220L | ** |
| 149 | D18P + D39L + K55Y + K64L + D156L + S168D + E186K + E188P + M189L + K198L + M220L | ** |
| 150 | D18P + N28R + D39L + K55Y + K64L + S168D + E186K + E188P + M189L + K198L + M220L | ** |
| 151 | D18P + N28R + D39L + K64L + D156L + S168D + E186K + E188P + M189L + K198L + M220L | ** |
| 152 | D18P + N28Q + D39L + K55Y + K64L + D156L + S168Q + E186K + E188P + M189L + K198L + M220L | ** |
| 153 | D18P + N28H + D39L + K55Y + K64L + D156L + S168T + E186K + E188P + M189L + K198L + M220L | ** |
| 154 | D18P + D39L + K55Y + K64L + D156L + S168M + E186K + E188P + M189L + K198L + M220L | ** |
| 155 | D18P + N28S + D39L + K55Y + K64L + D156L + E186K + E188P + M189L + K198L + M220L | ** |
| 156 | D18P + N28R + D39L + K64L + D156L + S168M + E186K + E188P + M189L + K198L + M220L | ** |
| 157 | D18P + N28Q + D39L + K55Y + K64L + D156L + S168P + E186K + E188P + M189L + K198L + M220L | ** |
| 158 | D18P + N28R + D39L + S168E + E186K + E188P + M189L + K198L + M220L | ** |
| 159 | D18P + N28H + D39L + K55Y + K64L + D156L + S168E + E186K + E188P + M189L + K198L + M220L | ** |
| 160 | D18P + N28Q + D39L + K55Y + K64L + D156L + S168E + E186K + E188P + M189L + K198L + M220L | ** |
| 161 | D18P + N28L + D39L + K55Y + K64L + D156L + S168V + E186K + E188P + M189L + K198L + M220L | ** |
| 162 | D18P + N28Y + D39L + K55Y + K64L + D156L + S168E + E186K + E188P + M189L + K198L + M220L | ** |
| 163 | D18P + N28F + D39L + K55Y + K64L + D156L + S168E + E178Q + E186K + E188P + M189L + K198L + M220L | ** |
| 164 | D18P + N28Q + D39L + K55Y + K64L + D156L + E186K + E188P + M189L + K198L + M220L | ** |
| 165 | D18P + N28H + D39L + K55Y + K64L + D156L + S168D + E186K + E188P + M189L + K198L + M220L | ** |
| 166 | D18P + N28K + D39L + K55Y + K64L + D156L + E186K + E188P + M189L + K198L + M220L | ** |
| 167 | D18P + D39L + K55Y + K64L + D156L + E178Q + E186K + E188P + M189L + K198L + M220L | ** |
| 168 | D18P + N28Q + D39L + K55Y + K64L + D156L + S168K + E186K + E188P + M189L + K198L + M220L | ** |
| 169 | D18P + N28L + D39L + K55Y + K64L + D156L + S168I + E186K + E188P + M189L + K198L + M220L | ** |
| 170 | D18P + N28Q + D39L + K55Y + K64L + D156L + S168L + E186K + E188P + M189L + K198L + M220L | ** |
| 171 | D18P + N28Q + D39L + K55Y + K64L + D156L + S168T + E186K + E188P + M189L + K198L + M220L | ** |
| 172 | D18P + D39L + K55Y + N59L + K64L + D156L + E186K + E188P + M189L + K198L + M220L | ** |
| 173 | D18P + D39L + K55Y + N59R + K64L + D156L + E186K + E188P + M189L + K198L + M220L | ** |
| 174 | D18P + D39L + K55Y + N59T + K64L + Q84E + D156L + E186K + E188P + M189L + K198L + M220L | ** |
| 175 | D18P + D39L + K55Y + N59T + K64L + D156L + E186K + E188P + M189L + K198L + M220L | ** |
| 176 | D18P + D39L + K55Y + N59V + K64L + D156L + E186K + E188P + M189L + K198L + M220L | ** |
| 177 | D18P + D39L + K55Y + N59T + K64L + Q84K + D156L + E186K + E188P + M189L + K198L + M220L | ** |
| 178 | D18P + D39L + K55Y + N59D + K64L + D156L + E186K + E188P + M189L + K198L + M220L | ** |
| 179 | D18P + D39L + K55Y + N59D + K64L + Q84E + D156L + E186K + E188P + M189L + K198L + M220L | ** |
| 180 | D18P + D39L + K55Y + N59I + K64L + D156L + E186K + E188P + M189L + K198L + M220L | ** |
| 181 | D18P + D39L + K55Y + K64L + Q84A + D156L + E186K + E188P + M189L + K198L + M220L | ** |
| 182 | D18P + D39L + K55Y + K64L + V75L + G77D + D156L + E186K + E188P + M189L + K198L + M220L | ** |
| 183 | D18P + N28E + D39L + K55Y + K64L + D156L + S168D + E186K + E188P + M189L + K198L + M220L | ** |
| 184 | D18P + N28R + D39L + K55Y + K64L + D156L + S168D + E186K + E188P + M189L + K198L + M220L | ** |
| 185 | D18P + N28R + D39L + K55Y + K64L + D156L + E186K + E188P + M189L + K198L + M220L | ** |
| 186 | D18P + N28R + D39L + K55Y + K64L + D156L + S168L + E186K + E188P + M189L + K198L + M220L | ** |
| 187 | D18P + N28R + D39L + K55Y + K64L + D156L + S168Q + E186K + E188P + M189L + K198L + M220L | ** |
| 188 | D18P + N28M + D39L + K55Y + K64L + D156L + S168M + E186K + E188P + M189L + K198L + M220L | ** |
| 189 | D18P + N28R + D39L + K55Y + K64L + D156L + S168M + E186K + E188P + M189L + K198L + M220L | ** |
| 190 | D18P + N28R + D39L + K55Y + K64L + D156L + S168E + E186K + E188P + M189L + K198L + M220L | ** |
| 191 | D18P + N28M + D39L + K55Y + K64L + D156L + S168I + E186K + E188P + M189L + K198L + M220L | ** |
| 192 | D18P + N28E + D39L + K55Y + K64L + D156L + S168M + E186K + E188P + M189L + K198L + M220L | ** |
| 193 | D18P + D39L + N59V + K64L + E186K + E188P + M189L + M220L | ** |
| 194 | D18P + D39L + N59S + K64L + E186K + E188P + M189L + M220L | ** |
| 195 | D18P + D39L + N59T + K64L + E186K + E188P + M189L + M220L | ** |
| 196 | D18P + D39L + K64L + V75I + E186K + E188P + M189L + M220L | ** |
| 197 | D18P + D39L + K64L + Q84E + E186K + E188P + M189L + M220L | ** |
| 198 | D18P + D39L + K55Y + N59V + K64L + E186K + E188P + M189L + M220L | ** |
| 199 | D18P + D39L + K55Y + N59S + K64L + E186K + E188P + M189L + M220L | ** |
| 200 | D18P + D39L + K55Y + N59T + K64L + E186K + E188P + M189L + M220L | ** |
| 201 | D18P + D39L + K55Y + K64L + V75I + E186K + E188P + M189L + M220L | ** |
| 202 | D18P + D39L + K55Y + K64L + Q84E + E186K + E188P + M189L + M220L | ** |
| 203 | D18P + D39L + N59V + K64L + V75I + E186K + E188P + M189L + M220L | ** |
| 204 | D18P + D39L + N59V + K64L + Q84E + E186K + E188P + M189L + M220L | ** |
| 205 | D18P + D39L + N59S + K64L + V75I + E186K + E188P + M189L + M220L | ** |
| 206 | D18P + D39L + N59S + K64L + Q84E + E186K + E188P + M189L + M220L | ** |
| 207 | D18P + D39L + N59T + K64L + V75I + E186K + E188P + M189L + M220L | ** |
| 208 | D18P + D39L + N59T + K64L + Q84E + E186K + E188P + M189L + M220L | ** |
| 209 | D18P + D39L + K64L + V75I + Q84E + E186K + E188P + M189L + M220L | ** |
| 210 | D18P + D39L + K55Y + N59V + K64L + V75I + E186K + E188P + M189L + M220L | ** |
| 211 | D18P + D39L + K55Y + N59V + K64L + Q84E + E186K + E188P + M189L + M220L | ** |
| 212 | D18P + D39L + K55Y + N59S + K64L + V75I + E186K + E188P + M189L + M220L | ** |
| 213 | D18P + D39L + K55Y + N59S + K64L + Q84E + E186K + E188P + M189L + M220L | ** |
| 214 | D18P + D39L + K55Y + N59T + K64L + V75I + E186K + E188P + M189L + M220L | ** |
| 215 | D18P + D39L + K55Y + N59T + K64L + Q84E + E186K + E188P + M189L + M220L | ** |
| 216 | D18P + D39L + K55Y + K64L + V75I + Q84E + E186K + E188P + M189L + M220L | ** |
| 217 | D18P + D39L + N59V + K64L + V75I + Q84E + E186K + E188P + M189L + M220L | ** |
| 218 | D18P + D39L + N59S + K64L + V75I + Q84E + E186K + E188P + M189L + M220L | ** |
| 219 | D18P + D39L + N59T + K64L + V75I + Q84E + E186K + E188P + M189L + M220L | ** |
| 220 | D18P + D39L + K55Y + N59V + K64L + V75I + Q84E + E186K + E188P + M189L + M220L | ** |
| 221 | D18P + D39L + K55Y + N59S + K64L + V75I + Q84E + E186K + E188P + M189L + M220L | ** |
| 222 | D18P + D39L + K55Y + N59T + K64L + V75I + Q84E + E186K + E188P + M189L + M220L | ** |
| 223 | E7K + D18P + D39L + K55Y + N59V + K64L + E186K + E188P + M189L + M220L | ** |
| 224 | E7K + D18P + D39L + K55Y + N59S + K64L + E186K + E188P + M189L + M220L | ** |
| 225 | E7K + D18P + D39L + K55Y + N59T + K64L + E186K + E188P + M189L + M220L | ** |
| 226 | E7K + D18P + D39L + K55Y + K64L + V75I + E186K + E188P + M189L + M220L | ** |
| 227 | E7K + D18P + D39L + K55Y + K64L + Q84E + E186K + E188P + M189L + M220L | ** |
| 228 | E7K + D18P + D39L + K55Y + N59V + K64L + V75I + E186K + E188P + M189L + M220L | ** |
| 229 | E7K + D18P + D39L + K55Y + N59S + K64L + V75I + E186K + E188P + M189L + M220L | ** |
| 230 | E7K + D18P + D39L + K55Y + N59S + K64L + Q84E + E186K + E188P + M189L + M220L | ** |
| 231 | E7K + D18P + D39L + K55Y + N59T + K64L + V75I + E186K + E188P + M189L + M220L | ** |
| 232 | E7K + D18P + D39L + K55Y + N59T + K64L + Q84E + E186K + E188P + M189L + M220L | ** |
| 233 | E7K + D18P + D39L + K55Y + K64L + V75I + Q84E + E186K + E188P + M189L + M220L | ** |
| 234 | E7K + D18P + D39L + K55Y + N59V + K64L + V75I + Q84E + E186K + E188P + M189L + M220L | ** |
| 235 | E7K + D18P + D39L + K55Y + N59S + K64L + V75I + Q84E + E186K + E188P + M189L + M220L | ** |
| 236 | E7K + D18P + D39L + K55Y + N59T + K64L + V75I + Q84E + E186K + E188P + M189L + M220L | ** |
| 237 | D18P + D39L + K55Y + K64L + V75I + E186K + E188P | ** |
| 238 | D18P + D39L + K64L + V75I + Q84E + E186K + E188P | ** |
| 239 | D18P + D39L + K55Y + N59V + K64L + V75I + E186K + E188P | ** |
| 240 | D18P + D39L + K55Y + N59S + K64L + V75I + E186K + E188P | ** |
| 241 | D18P + D39L + K55Y + N59T + K64L + V75I + E186K + E188P | ** |
| 242 | D18P + D39L + K55Y + K64L + V75I + Q84E + E186K + E188P | ** |
| 243 | D18P + D39L + N59V + K64L + V75I + Q84E + E186K + E188P | ** |
| 244 | D18P + D39L + N59S + K64L + V75I + Q84E + E186K + E188P | ** |
| 245 | D18P + D39L + K55Y + N59V + K64L + V75I + Q84E + E186K + E188P | ** |
| 246 | D18P + D39L + K55Y + N59S + K64L + V75I + Q84E + E186K + E188P | ** |
| 247 | D18P + D39L + K55Y + N59T + K64L + V75I + Q84E + E186K + E188P | ** |
| 248 | D18P + D39L + K55Y + V75I + E186K + E188P + M189L | ** |
| 249 | D18P + D39L + K64L + V75I + E186K + E188P + M218V + M220L | ** |
| 250 | D18P + D39L + K64L + V75I + Q84Y + E186K + E188P | ** |
| 251 | D18P + D39L + K64L + V75I + Q84F + E186K + E188P | ** |
| 252 | D18P + D39L + K64L + S66W + V75I + E186K + E188P | ** |
| 253 | D18P + D39L + H61Y + K64L + V75I + E186K + E188P | ** |
| 254 | D18P + D39L + K64L + S66F + V75I + E186K + E188P | ** |
| 255 | D18P + D39L + K64L + V75I + E186K + E188P + E221V | ** |
| 256 | D18P + D39L + K64L + S66V + V75I + E186K + E188P | ** |
| 257 | D18P + D39L + K64L + V75I + S177W + E186K + E188P | ** |
| 258 | D18P + D39L + K64L + V75I + T173W + E186K + E188P | ** |
| 259 | D18P + D39L + H61F + K64L + V75I + E186K + E188P | ** |
| 260 | D18P + M22F + N28D + D39L + K64L + V75I + E178T + E186K + E188P | ** |
| 261 | D18P + M22F + N28S + D39L + K64L + V75I + E178P + E186K + E188P | ** |
| 262 | E7K + D18P + N28Y + D39L + K55Y + N59S + K64L + V75I + Q84E + S168A + E186K + E188P + M189L + | ** |
| | M220L | |
| 263 | E7K + D18P + D39L + K55Y + N59S + K64L + V75I + Q84E + R166Y + E186K + E188P + M189L + M218V + | ** |
| | M220L | |
| 264 | E7K + D18P + D39L + K55Y + N59S + K64L + V75I + Q84Y + E186K + E188P + M189L + M220L | ** |
| 265 | E7K + D18P + D39L + K55Y + N59S + K64L + S66Y + V75I + Q84E + E186K + E188P + M189L + M220L | ** |
| 266 | E7K + D18P + D39L + K55Y + N59S + K64L + S66Q + V75I + Q84E + E186K + E188P + M189L + M220L | ** |
| 267 | E7K + D18P + D39L + K55Y + N59S + K64L + V75I + Q84W + E186K + E188P + M189L + M220L | ** |
| 268 | E7K + D18P + D39L + K55Y + N59S + K64L + S66L + V75I + Q84E + E186K + E188P + M189L + M220L | ** |
| 269 | E7K + D18P + D39L + K55Y + N59S + K64L + V75I + Q84E + G119V + E186K + E188P + M189L + M220L | ** |
| 270 | E7K + D18P + D39L + K55Y + N59S + K64L + Y67W + V75I + Q84E + E186K + E188P + M189L + M220L | ** |
| 271 | E7K + D18P + D39L + K55Y + N59S + K64L + V75I + Q84E + D156Q + E186K + E188P + M189L + M220L | ** |
| 272 | E7K + D18P + D39L + K55Y + N59S + T62Y + K64L + V75I + Q84E + E186K + E188P + M189L + M220L | ** |
| 273 | E7K + D18P + D39L + K55Y + N59S + K64L + S66I + V75I + Q84E + E186K + E188P + M189L + M220L | ** |
| 274 | E7K + D18P + D39L + K55Y + N59S + K64L + V75I + Q84E + A104M + E186K + E188P + M189L + M220L | ** |
| 275 | E7K + D18P + D39L + K55Y + N59S + K64L + E68T + V75I + Q84E + E186K + E188P + M189L + M220L | ** |
| 276 | E7K + D18P + D39L + K55Y + N59S + K64L + V75I + L82F + Q84E + E186K + E188P + M189L + M220L | ** |
| 277 | E7K + D18P + D39L + K55Y + N59S + K64L + E68H + V75I + Q84E + E186K + E188P + M189L + M220L | ** |
| 278 | E7K + D18P + D39L + K55Y + N59S + K64L + V75I + Q84E + D156N + E186K + E188P + M189L + M220L | ** |
| 279 | E7K + D18P + D39L + K55Y + N59S + K64L + V75I + Q84E + L115Q + E186K + E188P + M189L + M220L | ** |
| 280 | E7K + D18P + D39L + K55Y + N59S + K64L + V75I + Q84E + L115W + E186K + E188P + M189L + M220L | ** |
| 281 | E7K + D18P + D39L + K43Y + K55Y + N59S + K64L + V75I + Q84E + E186K + E188P + M189L + M220L | ** |
| 282 | E7K + D18P + D39L + K55Y + N59S + K64L + V75I + Q84E + L115Y + E186K + E188P + M189L + M220L | ** |
| 283 | E7K + D18P + D39L + K55Y + N59S + K64L + S71A + V75I + Q84E + E186K + E188P + M189L + M220L | ** |
| 284 | E7K + D18P + D39L + K55Y + N59S + K64L + V75I + Q84E + F87W + E186K + E188P + M189L + M220L | ** |
| 285 | E7K + D18P + D39L + K55Y + N59S + K64L + Y67V + V75I + Q84E + E186K + E188P + M189L + M220L | ** |
| 286 | E7K + D18P + D39L + K55Y + N59S + K64L + V75I + Q84E + L115I + E186K + E188P + M189L + M220L | ** |
| 287 | E7K + D18P + D39L + K55Y + N59S + K64L + V75I + Q84E + H86W + E186K + E188P + M189L + M220L | ** |
| 288 | E7K + D18P + D39L + K55Y + N59S + K64L + V75I + Q84E + H86I + E186K + E188P + M189L + M220L | ** |
| 289 | E7K + D18P + D39L + K55Y + N59S + K64L + Y67F + V75I + Q84E + E186K + E188P + M189L + M220L | ** |
| 290 | E7K + D18P + D39L + R50Y + K55Y + N59S + K64L + V75I + Q84E + E186K + E188P + M189L + M220L | ** |
| 291 | E7K + D18P + D39L + K55Y + N59S + T62F + K64L + V75I + Q84E + E186K + E188P + M189L + M220L | ** |
| 292 | E7K + D18P + D39L + K55Y + N59S + K64L + V75I + Q84E + A149H + E186K + E188P + M189L + M220L | ** |
| 293 | E7K + D18P + D39L + K55Y + N59S + K64L + E68R + V75I + Q84E + E186K + E188P + M189L + M220L | ** |
| 294 | E7K + D18P + D39L + K55Y + N59S + K64L + V75I + Q84E + E186K + E188P + M189L + N208L + M220L | ** |
| 295 | E7K + D18P + D39L + K55Y + N59S + K64L + V75I + Q84E + M185F + E186K + E188P + M189L + M220L | ** |
| 296 | E7K + D18P + D39L + K55Y + N59S + K64L + S66V + V75I + Q84E + E186K + E188P + M189L + M220L | ** |
| 297 | E7K + D18P + D39L + K55Y + N59S + K64L + S66M + V75I + Q84E + E186K + E188P + M189L + M220L | ** |
| 298 | E7K + D18P + D39L + K55Y + N59S + K64L + Y67I + V75I + Q84E + E186K + E188P + M189L + M220L | ** |
| 299 | E7K + D18P + D39L + K55Y + N59S + K64L + V75I + Q84E + H86Y + E186K + E188P + M189L + M220L | ** |
| 300 | E7K + D18P + D39L + K55Y + N59S + K64L + V75I + Q84E + E145Y + E186K + E188P + M189L + M220L | ** |
| 301 | E7K + D18P + D39L + K43F + K55Y + N59S + K64L + V75I + Q84E + E186K + E188P + M189L + M220L | ** |
| 302 | E7K + D18P + D39L + K55Y + N59S + K64L + V75I + Q84E + A104C + E186K + E188P + M189L + M220L | ** |
| 303 | E7K + D18P + D39L + K55Y + N59S + K64L + V75I + Q84E + E178S + E186K + E188P + M189L + M220L | ** |
| 304 | E7K + D18P + D39L + K55Y + N59S + K64L + V75I + Q84E + S168A + E178P + E186K + E188P + M189L + | ** |
| | M220L | |
| 305 | E7K + D18P + D39L + K55Y + N59S + K64L + V75I + Q84E + S168C + E186K + E188P + M189L + A216C + | ** |
| | M220L | |
| 306 | E7K + D18P + M22F + N28D + D39L + K55Y + N59S + K64L + V75I + Q84E + E178T + E186K + E188P + M189L + | ** |
| | M220L | |
| 307 | E7K + D18P + M22F + N28D + D39L + K55Y + N59S + K64L + V75I + Q84E + E178P + E186K + E188P + M189L + | ** |
| | M220L | |
| 308 | E7K + D18P + M22F + N28D + D39L + K55Y + N59S + K64L + V75I + Q84E + E178S + E186K + E188P + M189L + | ** |
| | M220L | |
| 309 | E7K + D18P + R36N + D39L + K55Y + N59S + K64L + V75I + D76K + Q84E + R181A + E186K + E188P + M189L + | ** |
| | M220L | |
| 310 | E7K + D18P + R36N + D39L + K55Y + N59S + K64L + V75I + D76N + Q84E + R181A + E186K + E188P + M189L + | ** |
| | M220L | |
| 311 | E7K + D18P + R36N + D39L + K55Y + N59S + K64L + V75I + Q84E + S168A + R181T + E186K + E188P + M189L + | ** |
| | M218V + M220L | |
| 312 | E7K + D18P + *36aE + *36bN + D39L + K55Y + N59S + K64L + V75I + Q84E + E186K + E188P + M189L + | ** |
| | A216G + M218Y + M220L + E221K | |
| 313 | E7K + D18P + *36aE + *36bN + D39L + K55Y + N59S + K64L + V75I + Q84E + E186K + E188P + M189L + | ** |
| | A216G + R217N + M218Y + M220L + E221V | |
| 314 | E7K + D18P + *36aE + *36bN + D39L + K55Y + N59S + K64L + V75I + Q84E + E186K + E188P + M189L + | ** |
| | A216G + R217N + M218Y + M220L + E221T | |
| 315 | E7K + D18P + *36aE + *36bN + D39L + K55Y + N59S + K64L + V75I + Q84E + E186K + E188P + M189L + | ** |
| | A216G + R217N + M218Y + M220L | |
| 316 | E7K + D18P + *36aE + *36bN + D39L + K55Y + N59S + K64L + V75I + Q84E + E186K + E188P + M189L + | ** |
| | A216G + R217N + M218A + M220L + E221V | |
| 317 | E7K + D18P + *36aE + *36bN + D39L + K55Y + N59S + K64L + V75I + Q84E + E186K + E188P + M189L + | ** |
| | A216G + R217N + M218T + M220L + E221K | |
| 318 | E7K + D18P + *36aA + *36bY + D39L + K55Y + N59S + K64L + V75I + Q84E + E186K + E188P + M189L + | ** |
| | M220L | |
| 319 | E7K + D18P + *36aA + *36bV + D39L + K55Y + N59S + K64L + V75I + Q84E + E186K + E188P + M189L + | ** |
| | M220L | |
| 320 | E7K + D18P + *36aE + *36bN + D39L + K41C + K55Y + N59S + K64L + V75I + Q84E + E186K + E188P + M189L + | ** |
| | M220L | |
| 321 | E7K + D18P + *36aE + *36bN + D39L + K55Y + N59S + K64L + V75I + Q84E + E186K + E188P + M189L + | ** |
| | A216G + M218A + M220L + E221V | |
| 322 | E7K + D18P + *36aE + *36bN + D39L + K55Y + N59S + K64L + V75I + Q84E + E186K + E188P + M189L + | ** |
| | A216G + R217N + M218Y + M220L + E221K | |
| 323 | D18P + D39L + V75I + E186K + E188P + M189L | 105.7 |
| 324 | E7K + D18P + D39L + E186K + E188P + M189L | 105.6 |
| 325 | E7K + D18P + *36aE + *36bN + D39L + K55Y + N59S + K64L + V75I + Q84E + E186K + E188P + M189L + | 105.6 |
| | A216G + R217N + M218T + M220L + E221T | |
| 326 | H5E + D18P + D39L + E186K + E188P + M189L | 105.4 |
| 327 | D18P + D39L + K55T + E186K + E188P + M189L | 105.2 |
| 328 | D18P + D39L + E127A + E186K + E188P + M189L | 105.1 |
| 329 | D18P + D39L + Q98E + E186K + E188P + M189L | 105.1 |
| 330 | D18P + Y20F + D39L + E186K + E188P + M189L | 105.0 |
| 331 | D18P + D39L + E186K + E188P + M189L + E194A | 105.0 |
| 332 | D18P + D39L + I118V + E186K + E188P + M189L | 105.0 |
| 333 | D18P + D39L + L57V + E186K + E188P + M189L | 105.0 |
| 334 | D18P + D39L + H99L + E186K + E188P + M189L | 105.0 |
| 335 | D18P + D39L + N59L + V75I + E186K + E188P + M189L | 105.0 |
| 336 | D18P + D39L + K55Y + N59H + K64L + D156L + E186K + E188P + M189L + K198L + M220L | 105.0 |
| 337 | D18P + D39L + N59T + V75I + Q84E + E186K + E188P + M189L | 105.0 |
| 338 | D18P + D39L + N59T + V75I + E186K + E188P + M189L | 105.0 |
| 339 | D18P + D39L + K55Y + N59W + K64L + Q84G + D156L + E186K + E188P + M189L + K198L + M220L | 105.0 |
| 340 | D18P + D39L + T62W + K64L + V75I + E186K + E188P | 104.9 |
| 341 | D18P + D39L + N59S + K64L + V75I + E186K + E188P | 104.9 |
| 342 | D18P + D39L + N141H + E186K + E188P + M189L | 104.9 |
| 343 | D18P + D39L + H150Q + E186K + E188P + M189L | 104.9 |
| 344 | D18P + D39L + N59I + K64L + V75I + E186K + E188P | 104.9 |
| 345 | D18P + D39L + N59S + V75I + E186K + E188P + M189L | 104.9 |
| 346 | D18P + D39L + A89S + E186K + E188P + M189L | 104.8 |
| 347 | D18P + D39L + N59T + K64L + V75I + E186K + E188P | 104.8 |
| 348 | D18P + D39L + K55Y + N59S + K64L + D156L + E186K + E188P + M189L + K198L + M220L | 104.8 |
| 349 | D18P + D39L + E186K + E188P + M189L + G205H | 104.7 |
| 350 | D18P + D39L + T54K + E186K + E188P + M189L | 104.7 |
| 351 | D18P + D39L + E48H + E186K + E188P + M189L | 104.7 |
| 352 | D18P + D39L + I152L + E186K + E188P + M189L | 104.7 |
| 353 | D18P + D39L + G52V + E186K + E188P + M189L | 104.7 |
| 354 | D18P + D39L + K64L + E186K + E188P | 104.6 |
| 355 | D18P + D39L + Q98V + E186K + E188P + M189L | 104.6 |
| 356 | D18P + D39L + A154P + E186K + E188P + M189L | 104.6 |
| 357 | D18P + D39L + F102I + E186K + E188P + M189L | 104.5 |
| 358 | D18P + D39L + E186K + E188P + M189L + G205E | 104.5 |
| 359 | D18P + D39L + R50S + E186K + E188P + M189L | 104.5 |
| 360 | D18P + D39L + E186K + E188P + M189L + K201S | 104.5 |
| 361 | D18E + D39L + N141K + E186K | 104.5 |
| 362 | D18P + D39L + E186K + E188S + M189L | 104.5 |
| 363 | D39L + E197V | 104.5 |
| 364 | D18P + D39L + N59L + K64L + V75I + E186K + E188P | 104.5 |
| 365 | D18P + D39L + N59D + V75I + Q84E + E186K + E188P + M189L | 104.5 |
| 366 | D18P + D39L + N59H + K64L + V75I + E186K + E188P | 104.4 |
| 367 | D18P + D39L + E140K + E186K + E188P + M189L | 104.4 |
| 368 | D18P + D39L + S66N + E186K + E188P + M189L | 104.4 |
| 369 | D18P + D39L + R162K + E186K + E188P + M189L | 104.4 |
| 370 | D18P + D39L + A51P + E186K + E188P + M189L | 104.4 |
| 371 | D15E + D18P + D39L + E186K + E188P + M189L | 104.4 |
| 372 | D18P + D39L + N59H + K64L + E186K + E188P | 104.4 |
| 373 | D18P + D39L + E186K + E188P + M189L + K198L + M220L | 104.4 |
| 374 | D18P + D39L + N59R + V75I + E186K + E188P + M189L | 104.4 |
| 375 | D18P + D39L + K64L + V75I + G119S + E186K + E188P | 104.4 |
| 376 | D18P + D39L + K64L + V75I + G119A + E186K + E188P | 104.3 |
| 377 | D18K + D39L + E186K + M189L | 104.3 |
| 378 | D18P + D39L + E140S + E186K + E188P + M189L | 104.3 |
| 379 | E7K + D18P + D39L + K55Y + N59V + K64L + Q84E + E186K + E188P + M189L + M220L | 104.3 |
| 380 | D18A + D39L + E186K + E188P + M189L | 104.2 |
| 381 | E7K + D18P + M22Y + N28F + D39L + K55Y + N59S + K64L + V75I + Q84E + E186K + E188P + M189L + M220L | 104.2 |
| 382 | D18P + D39L + N59I + K64L + E186K + E188P | 104.1 |
| 383 | D18P + D39L + E186K + E188A + M189L | 104.1 |
| 384 | D18P + D39L + E48Y + E186K + E188P + M189L | 104.1 |
| 385 | D18P + D39L + E155A + E186K + E188P + M189L | 104.1 |
| 386 | D18P + D39L + D161K + E186K + E188P + M189L | 104.1 |
| 387 | D18P + D39L + N59L + K64L + E186K + E188P | 104.1 |
| 388 | D18P + D39L + K55Y + K64L + E186K + E188P + K198L | 104.0 |
| 389 | D18P + D39L + N59T + K64L + Q84E + E186K + E188P | 104.0 |
| 390 | D18P + R36N + D39L + K64L + V75I + S168A + R181T + E186K + E188P + M218I + M220L | 104.0 |
| 391 | D18P + D39L + A149K + E186K + E188P + M189L | 104.0 |
| 392 | D18P + D39L + R126A + E186K + E188P + M189L | 104.0 |
| 393 | E7K + D18P + D39L + K43P + K55Y + N59S + K64L + V75I + Q84E + E186K + E188P + M189L + M220L | 104.0 |
| 394 | D18P + D39L + N59S + K64L + E186K + E188P | 104.0 |
| 395 | D18P + D39L + K55Y + K64L + Q84G + D156L + E186K + E188P + M189L + K198L + M220L | 104.0 |
| 396 | D18P + D39L + K64L + V75I + R181I + E186K + E188P | 103.9 |
| 397 | D18P + D39L + N59T + K64L + V75I + Q84K + E186K + E188P | 103.9 |
| 398 | D18P + D39L + Y72S + E186K + E188P + M189L | 103.9 |
| 399 | D18P + D39L + A109Q + E186K + E188P + M189L | 103.9 |
| 400 | D18P + D39L + A51S + E186K + E188P + M189L | 103.9 |
| 401 | D18P + D39L + E186K + E188P + M189L | 103.9 |
| 402 | D18P + D39L + K55Y + K64L + V75I + D156L + E186K + E188P + M189L + K198L + M220L | 103.9 |
| 403 | D18P + D39L + N59T + K64L + E186K + E188P | 103.8 |
| 404 | D18P + D39L + E186K + E188P + M189L + K201E | 103.8 |
| 405 | D39L + E186K + M191V | 103.8 |
| 406 | D18P + D39L + H112T + E186K + E188P + M189L | 103.8 |
| 407 | D18P + D39L + N141E + E186K + E188P + M189L | 103.8 |
| 408 | D18P + D39L + E186K + E188P + M189L + I202L | 103.8 |
| 409 | D18P + D39L + T54A + E186K + E188P + M189L | 103.8 |
| 410 | D18P + D39L + K55Y + E186K + E188P + M189L + M220L | 103.8 |
| 411 | D18P + D39L + N59V + K64L + E186K + E188P | 103.8 |
| 412 | D18P + D39L + E48V + K64L + V75I + E186K + E188P | 103.7 |
| 413 | D18P + D39L + D163A + E186K + E188P + M189L | 103.7 |
| 414 | D18P + D39L + K64L + V75I + G119I + E186K + E188P | 103.7 |
| 415 | E7K + D18P + D39L + K64L + E186K + E188P + M189L | 103.6 |
| 416 | D18P + D39L + K55Y + K64L + E186K + E188P | 103.6 |
| 417 | D18P + D39L + K55Y + E186K + E188P + M189L + K198L | 103.6 |
| 418 | D18P + D39L + N59R + K64L + E186K + E188P | 103.6 |
| 419 | D18P + D39L + N59W + K64L + Q84G + E186K + E188P | 103.6 |
| 420 | S3G + D18P + D39L + E186K + E188P + M189L | 103.5 |
| 421 | D18P + D39L + N59V + V75I + E186K + E188P + M189L | 103.5 |
| 422 | E186R + M189L + M218V + E221L | 103.4 |
| 423 | D18P + D39L + N59H + V75I + E186K + E188P + M189L | 103.4 |
| 424 | D18P + D39L + E48Y + K64L + V75I + E186K + E188P | 103.3 |
| 425 | D18P + D39L + R50D + E186K + E188P + M189L | 103.3 |
| 426 | D18P + D39L + K64L + E186K + E188P + M189L | 103.3 |
| 427 | D18P + D39L + K55Y + K64L + V75L + D156L + E186K + E188P + M189L + K198L + M220L | 103.3 |
| 428 | D18P + D39L + V75I + Q84A + E186K + E188P + M189L | 103.2 |
| 429 | D18P + D39L + N59D + V75I + E186K + E188P + M189L | 103.2 |
| 430 | D18P + D39L + L95V + E186K + E188P + M189L | 103.2 |
| 431 | D18P + D39L + N59W + V75I + Q84G + E186K + E188P + M189L | 103.2 |
| 432 | D18P + D39L + E48F + K64L + V75I + E186K + E188P | 103.1 |
| 433 | D18P + D39L + F121M + E186K + E188P + M189L | 103.1 |
| 434 | D18P + D39L + A142V + E186K + E188P + M189L | 103.1 |
| 435 | D18P + D39L + K55Y + K64L + D156L + E186K + E188P | 103.0 |
| 436 | D18P + N35K + D39L + E186K + E188P + M189L | 103.0 |
| 437 | D18P + D39L + E186K + E188P + M189Q | 103.0 |
| 438 | D18P + D39L + F121L + E186K + E188P + M189L | 103.0 |
| 439 | D18P + D39L + Y72T + E186K + E188P + M189L | 103.0 |
| 440 | E7K + D18P + D39L + E48Y + K55Y + N59S + K64L + V75I + Q84E + E186K + E188P + M189L + M220L | 103.0 |
| 441 | E7K + D18P + D39L + K64L + E186K + E188P + M189L + K198L | 103.0 |
| 442 | D18P + D39L + K64L + V75I + A104L + E186K + E188P | 103.0 |
| 443 | D18P + D39L + I130A + E186K + E188P + M189L | 102.9 |
| 444 | E7K + D18P + D39L + D156L + E186K + E188P + M189L + K198L | 102.9 |
| 445 | D18P + D39L + N59W + K64L + V75I + Q84G + E186K + E188P | 102.9 |
| 446 | E7K + D18P + D39L + K55Y + N59S + K64L + S66F + V75I + Q84E + E186K + E188P + M189L + M220L | 102.9 |
| 447 | D39L + S177Y + E186K + M191V | 102.8 |
| 448 | D39L + K147I + D163K + E186K | 102.8 |
| 449 | D18P + D39L + K64L + V75I + A149H + E186K + E188P | 102.8 |
| 450 | D18P + D39L + K43H + K64L + V75I + E186K + E188P | 102.7 |
| 451 | D18P + D39L + K64L + D156L + E186K + E188P + K198L | 102.6 |
| 452 | E7K + D18P + D39L + D156L + E186K + E188P + M189L | 102.6 |
| 453 | D18P + D39L + D156L + E186K + E188P + M189L + K198L | 102.6 |
| 454 | D18P + D39L + K64L + V75I + G119C + E186K + E188P | 102.5 |
| 455 | H5G + D18P + D39L + E186K + E188P + M189L | 102.5 |
| 456 | D18P + D39L + A53K + E186K + E188P + M189L | 102.4 |
| 457 | E7K + D18P + D39L + K55Y + E186K + E188P + M189L | 102.4 |
| 458 | E7K + D18P + D39L + E186K + E188P + M189L + K198L | 102.4 |
| 459 | D18P + D39L + V75I + S168A + E186K + E188P + M189L | 102.4 |
| 460 | D18P + D39L + K64L + V75I + Q84W + E186K + E188P | 102.3 |
| 461 | D18P + D39L + K64L + V75I + A104V + E186K + E188P | 102.3 |
| 462 | D18P + D39L + K43Y + K64L + V75I + E186K + E188P | 102.3 |
| 463 | E7K + D18P + D39L + K55Y + E186K + E188P + M189L + K198L | 102.2 |
| 464 | E7K + D18P + D39L + K55Y + K64L + D156L + E186K + E188P + M189L + K198L | 102.2 |
| 465 | E7K + D18P + D39L + K55Y + K64L + D156L + E186K + E188P + M189L | 102.2 |
| 466 | D18P + D39L + K55Y + K64L + D156L + S168A + E186K + E188P + M189L + K198L + M220L | 102.1 |
| 467 | E7K + D18P + D39L + K55Y + N59S + K64L + V75I + Q84E + E186K + E188P + M189L + M220L + E221L | 102.1 |
| 468 | D18P + D39L + K64L + S168A + E186K + E188P | 102.1 |
| 469 | D18P + D39L + K64L + V75I + E186K + E188P + K198Y | 102.0 |
| 470 | D18P + N35V + D39L + E186K + E188P + M189L | 102.0 |
| 471 | D18P + D39L + I78T + E186K + E188P + M189L | 102.0 |
| 472 | E7K + D18P + D39L + K55Y + K64L + D156L + E186K + E188P + M189L + K198L + M220L | 102.0 |
| 473 | D18P + D39L + T62Y + K64L + V75I + E186K + E188P | 101.9 |
| 474 | D18P + D39L + K46Y + K64L + V75I + E186K + E188P | 101.9 |
| 475 | D39L + H99L + E186K + M218V | 101.9 |
| 476 | D18P + D39L + E155R + E186K + E188P + M189L | 101.9 |
| 477 | E7K + D18P + D39L + K55Y + D156L + E186K + E188P + M189L | 101.9 |
| 478 | D18P + D39L + I78L + E186K + E188P + M189L | 101.6 |
| 479 | D18K + D39L + K147I + E186R | 101.5 |
| 480 | D18P + D39L + K64L + V75I + G119T + E186K + E188P | 101.5 |
| 481 | D18P + D39L + N59T + K64L + Q84K + E186K + E188P | 101.4 |
| 482 | D18P + D39L + K64L + V75I + E186K + E187I + E188P | 101.2 |
| 483 | E7K + D18P + D39L + K55Y + N59S + K64L + V75I + Q84F + E186K + E188P + M189L + M220L | 101.1 |
| 484 | E7K + D18P + D39L + K55Y + N59S + K64L + V75I + Q84E + E145L + E186K + E188P + M189L + M220L | 101.0 |
| 485 | D18P + D39L + V75L + E186K + E188P + M189L | 100.9 |
| 486 | E7K + D18P + D39L + K55Y + N59S + H61Y + K64L + V75I + Q84E + E186K + E188P + M189L + M220L | 100.9 |
| 487 | D39L + K147I + E186R + M218V | 100.7 |
| 488 | D39L + D163K + E186R + M191V + M218V + E221R | 100.6 |
| 489 | D18P + M22F + N28D + D39L + K64L + V75I + E178P + E186K + E188P | 100.4 |
| 490 | A53G + D163K + E186R + E221R | 100.3 |
| 491 | D39L + K64L + H99L + E186R | 100.3 |
| 492 | D39L + E186R | 100.3 |
| 493 | D18K + D39L + E186R + M189L | 100.2 |
| 494 | D39L + A53G + E186R + M189I | 100.2 |
| 495 | D39L + N141K + E186R + M191V | 100.1 |
| 496 | D39L + K64L + S177Y + E186R | 100.1 |
| 497 | D18P + D39L + K64L + S66I + V75I + E186K + E188P | 100.0 |
| 498 | D18E + D163K + E186R + E221R | 100.0 |
| 499 | D18E + D39L + E186R + M189L | 100.0 |
| 500 | D18K + D39L + E186R + M218V | 100.0 |
| 501 | D18P + D39L + K64L + V75I + E186K + E188P + E221L | 99.9 |
| 502 | D18P + D39L + K64L + V75I + E186I + E188P | 99.9 |
| 503 | D39L + K147I + E186R + M189I | 99.7 |
| 504 | D18P + N28R + D39L + V75I + S168Q + E186K + E188P + M189L | 99.7 |
| 505 | D18P + M22F + N28D + D39L + K64L + V75I + E178S + E186K + E188P | 99.6 |
| 506 | A53G + E186R + M189L + E221R | 99.4 |
| 507 | D39L + D163K + E186R + M218V | 99.4 |
| 508 | D39R + K64L + S177Y + E186K | 99.3 |
| 509 | D39L + D163K + S177Y + E186R | 99.2 |
| 510 | D18P + D39L + K64L + V75I + E186K + E188P + K198F | 99.1 |
| 511 | D39L + D163K + E186R + M191V | 99.1 |
| 512 | D39R + E186R + M191V + M218V | 99.1 |
| 513 | D18P + D39L + A53G + E186R + E188P | 99.1 |
| 514 | D18E + D39L + S177Y + E186R | 99.1 |
| 515 | D39L + A53G + E186R + M189L | 99.0 |
| 516 | D39R + A53G + H99L + E186R | 98.8 |
| 517 | D39R + H99L + E186R + M191V | 98.7 |
| 518 | D18K + D39L + S177Y + E186R | 98.6 |
| 519 | D39R + K64L + E221L | 98.5 |
| 520 | D39L + D163K + E186R + E221L | 98.5 |
| 521 | D39R + E186R + M191V | 98.4 |
| 522 | D39L + H99L + E186R + M189I | 98.3 |
| 523 | D39L + D163K + E186K + M191V + M218V + E221R | 98.2 |
| 524 | D18E + D39R + D163K + E186R | 98.0 |
| 525 | D18P + D39L + N141K + E186R + E188P | 98.0 |
| 526 | D39L + E186R + M191V + E221L | 98.0 |
| 527 | D39L + H99L + E186K + E221R | 98.0 |
| 528 | D39L + E186R + M191V + E221R | 98.0 |
| 529 | D39R + H99L + D163K + E186R | 97.9 |
| 530 | D39R + E186R | 97.8 |
| 531 | D18K + D39R + E186R + M218V | 97.8 |
| 532 | D39R + A53G + K64L + E186R | 97.8 |
| 533 | E186R + M189I + M218V + E221R | 97.8 |
| 534 | D18K + D39L + N141K + E221L | 97.5 |
| 535 | D18P + I37V + D39L + E186K + E188P + M189L | 97.5 |
| 536 | E7K + D18P + D39L + K55Y + N59S + K64L + V75I + Q84M + E186K + E188P + M189L + M220L | 97.5 |
| 537 | D39R + K64L + S177Y + E186R | 97.4 |
| 538 | A53G + K147I + E186K | 97.4 |
| 539 | D18P + N28H + D39L + K55Y + K64L + D156L + E186K + E188P + M189L + K198L + M220L | 97.4 |
| 540 | D39R + K64L + K147I + E186R | 97.3 |
| 541 | A142K + E186K + M191V | 97.1 |
| 542 | D39R + E186R + M189I + M191V | 97.1 |
| 543 | E186K + I202T + E221K | 97.1 |
| 544 | D39L + D163K + E186K + E221L | 97.0 |
| 545 | S177Y + E186R + M191V + E221R | 97.0 |
| 546 | D39R + N141K + E186R + M189I | 96.9 |
| 547 | D18P + N28H + D39L + N59R + V75I + E186K + E188P + M189L | 96.9 |
| 548 | D18P + D39L + K64L + V75I + E186K + E187V + E188P | 96.8 |
| 549 | H99L + K147I + E186R + E221R | 96.7 |
| 550 | K64L + S177Y + E186R + E221R | 96.7 |
| 551 | D39L + H99L + E186R + E221R | 96.5 |
| 552 | E186K + M191V + M218V | 96.5 |
| 553 | E186K + E221K | 96.5 |
| 554 | D39L + K147I + S177Y + E221L | 96.4 |
| 555 | F102L + E186K + M191V | 96.4 |
| 556 | D18P + K64L + E186R + E188P + E221R | 96.2 |
| 557 | A53G + N141K + E186R + E221L | 96.2 |
| 558 | D18P + N28H + D39L + N59L + V75I + E186K + E188P + M189L | 96.2 |
| 559 | D18P + D39L + K64L + V75I + H105F + E186K + E188P | 96.2 |
| 560 | E186K + M191V | 96.0 |
| 561 | D39L + K147I + E186L + E221R | 96.0 |
| 562 | D39L + A53G + M189L + E221L | 96.0 |
| 563 | E7R + E186K + E221K | 96.0 |
| 564 | Q98K + A142K + E186K | 95.9 |
| 565 | V75I + E186K + E221K | 95.9 |
| 566 | Q98K + E186K + M191V | 95.8 |
| 567 | V75L + E186K + E221K | 95.7 |
| 568 | K64L + S177Y + E186K + M189L | 95.7 |
| 569 | I130E + A142K + E186K | 95.7 |
| 570 | E127K + E186K + E221K | 95.6 |
| 571 | D39L + H99L + D163K + E221L | 95.6 |
| 572 | D18P + D39R + E186R + E188P + E221L | 95.6 |
| 573 | A89V + E186K + E221K | 95.5 |
| 574 | E186K + M191V + E221L | 95.5 |
| 575 | D18P + M22F + N28T + D39L + K64L + V75I + E178P + E186K + E188P | 95.5 |
| 576 | E7Q + E186K + E221K | 95.4 |
| 577 | E186R + M191V + E221L | 95.4 |
| 578 | E127Q + E186K + E221K | 95.4 |
| 579 | E127T + E186K + E221K | 95.4 |
| 580 | D39L + K64L + D163K + M191V | 95.3 |
| 581 | I118L + E186K + E221K | 95.3 |
| 582 | E7K + E186K + E221K | 95.2 |
| 583 | N141Q + E186K + E221K | 95.1 |
| 584 | D39L + S177Y + E186K + E221R | 95.1 |
| 585 | K64L + E186R | 95.0 |
| 586 | E127A + E186K + E221K | 95.0 |
| 587 | E186K + I202L + E221K | 94.9 |
| 588 | D18K + D39L + D163K + M218V | 94.9 |
| 589 | D18K + E186R + M189L + E221L | 94.9 |
| 590 | E7D + E186K + E221K | 94.9 |
| 591 | E7H + E186K + E221K | 94.9 |
| 592 | E186K + I202V + E221K | 94.9 |
| 593 | A154I + E186K + E221K | 94.9 |
| 594 | N141T + E186K + E221K | 94.8 |
| 595 | D18E + D39R + S177Y + E186R | 94.8 |
| 596 | K64L + K147I + E186R + M218V | 94.8 |
| 597 | N141E + E186K + E221K | 94.7 |
| 598 | D18E + K64L + E186R + M189I | 94.7 |
| 599 | D18K + E186R + M191V + E221L | 94.7 |
| 600 | D39L + K147I + E186L + E221L | 94.7 |
| 601 | D18E + A53G + D163K + E186R | 94.4 |
| 602 | E127R + E186K + E221K | 94.4 |
| 603 | D18P + N28H + D39L + K64L + E186K + E188P | 94.3 |
| 604 | G60D + I118L + E186K + E221K | 94.3 |
| 605 | K64L + S177Y + M218V + E221L | 94.3 |
| 606 | A89T + E186K + E221K | 94.2 |
| 607 | K147I + D163K + E186R + E221L | 94.2 |
| 608 | D18E + D39L + D163K + E221R | 94.1 |
| 609 | D39L + K64L + N141K + D163K | 94.1 |
| 610 | D18E + E186R + M189I + E221L | 94.0 |
| 611 | A154V + E186K + E221K | 94.0 |
| 612 | E7K + D18P + R36N + D39L + K55Y + N59S + K64L + V75I + D76N + Q84E + R181T + E186K + E188P + M189L + | 93.9 |
| | M220L | |
| 613 | D18P + V38I + D39L + E186K + E188P + M189L | 93.9 |
| 614 | E7K + D18P + D39L + K55Y + N59S + K64L + V75I + Q84I + E186K + E188P + M189L + M220L | 93.9 |
| 615 | E7K + D18P + *36aE + *36bN + D39L + K55Y + N59S + K64L + V75I + Q84E + R181I + E186K + E188P + | 93.9 |
| | M189L + M220L | |
| 616 | E186K + I202A + E221K | 93.9 |
| 617 | D18K + K64L + D163K + E186R | 93.8 |
| 618 | D39L + K64L + M218V + E221L | 93.7 |
| 619 | I118V + E186K + E221K | 93.7 |
| 620 | A154P + E186K + E221K | 93.7 |
| 621 | K64L + S177Y + E186R + E221L | 93.6 |
| 622 | D18E + N141K + E186R + E221L | 93.6 |
| 623 | H99L + N141K + E186R + E221L | 93.5 |
| 624 | D18K + A53G + K147I + E186R | 93.5 |
| 625 | D18K + D39L + M189I + E221R | 93.5 |
| 626 | E186R + M218V | 93.5 |
| 627 | H8N + A154P + E186K + E221K | 93.4 |
| 628 | D39L + E140A | 93.4 |
| 629 | D18E + A53G + K64L + E186R | 93.4 |
| 630 | D39L + K64L + N141K + E221R | 93.3 |
| 631 | D39R + K147I + E186R + E221R | 93.3 |
| 632 | A53G + N141K + E186R + M191V | 93.3 |
| 633 | D161N + E186K + E188P + E221K | 93.3 |
| 634 | A89S + E186K + E221K | 93.2 |
| 635 | D18K + K64L + E186R + M191V | 93.2 |
| 636 | D39L + A53G + E186R + E221R | 93.2 |
| 637 | K147I + S177Y + E186R + E221L | 93.0 |
| 638 | A53G + E186R + M189I + M218V | 93.0 |
| 639 | M191V + E221L | 93.0 |
| 640 | D18P + E186R | 92.9 |
| 641 | D18E + K147I + E186R + M191V | 92.9 |
| 642 | K147I + E186R + M189L + M191V | 92.9 |
| 643 | D18P + D39L + K64L + V75I + E127P + E186K + E188P | 92.9 |
| 644 | H99L + D163K + E186R + M189L | 92.8 |
| 645 | D18K + D39R + E186R + E221R | 92.8 |
| 646 | D39R + M218V | 92.8 |
| 647 | A154L + E186K + E221K | 92.7 |
| 648 | D39L + K64L + K147I + M189L | 92.7 |
| 649 | D39L + A53G + K64L + H99L | 92.6 |
| 650 | D18K + D163K + E186R + M189I | 92.6 |
| 651 | D18P + D39L + K64L + V75I + E186K + E188P + K222L | 92.5 |
| 652 | D18P + K27Y + D39L + K64L + V75I + N114L + E186K + E188P + N215L | 92.3 |
| 653 | D39L + H99L + N141K + E221R | 92.3 |
| 654 | D39L + M218V | 92.3 |
| 655 | D18K + D39L + M189L + M191V | 92.3 |
| 656 | D18E + H99L + E186R + M218V | 92.3 |
| 657 | D18K + H99L + E186R + E221L | 92.2 |
| 658 | D39L + E48R | 92.1 |
| 659 | E186R + M191V | 92.1 |
| 660 | S177Y + E186R | 92.1 |
| 661 | D18P + D39L | 92.0 |
| 662 | K64L + H99L + N141K + E186R | 92.0 |
| 663 | D39L + S66G | 91.9 |
| 664 | D39L + K64L + M189I + M191V | 91.9 |
| 665 | A53G + H99L + S177Y + E186R | 91.9 |
| 666 | D163K + S177Y + E186R + M218V | 91.9 |
| 667 | D39L + N141K + E186L + M218V | 91.8 |
| 668 | D18P + I37T + D39L + E186K + E188P + M189L | 91.8 |
| 669 | D39L + D163K + E186L + M189I | 91.8 |
| 670 | K64L + S177Y + E186R + M218V | 91.8 |
| 671 | D39L + E190G | 91.8 |
| 672 | D18K + H99L + E186R + M189L | 91.8 |
| 673 | D18P + D39L + E186K + E188P + M189L + M218T | 91.7 |
| 674 | K64L + N141K + E186R + M189L | 91.7 |
| 675 | D18K + K64L + S177Y + E186R | 91.7 |
| 676 | D18E + S177Y + E186R + M189L | 91.6 |
| 677 | D18P + D39L + K64L + V75I + E186K + E188P + N208L | 91.6 |
| 678 | D18K + N141K + K147I + E186R | 91.5 |
| 679 | D39R + K64L + S177Y + E221R | 91.5 |
| 680 | D18E + K147I + E186R + M189I | 91.5 |
| 681 | D18K + D39L + N141K + K147I | 91.4 |
| 682 | N141K + D163K + S177Y + E186R | 91.4 |
| 683 | D39L + E221I | 91.3 |
| 684 | D39R + K147I + M191V | 91.3 |
| 685 | D18P + D39L + E188P + M189L + E221R | 91.3 |
| 686 | D39L + R50P | 91.2 |
| 687 | D18P + D39L + K64L + S66Y + V75I + E186K + E188P | 91.2 |
| 688 | D18E + D39L + E186L + M191V | 91.0 |
| 689 | S177Y + E186R + M191V + M218V | 90.9 |
| 690 | D39L + H99L + K147I + M189L | 90.9 |
| 691 | K64L + H99L + E186R + M218V | 90.7 |
| 692 | D18P + A53G + E186R + E188P + E221L | 90.7 |
| 693 | D18E + N141K + E186R + M218V | 90.7 |
| 694 | D18K + D39R + K64L | 90.5 |
| 695 | D18E + D39L + K64L + E186L | 90.5 |
| 696 | D18P + E186R + E188P + M189L + M191V | 90.5 |
| 697 | D18P + D163K + E186R + E188P + M191V | 90.4 |
| 698 | D39R + M191V | 90.4 |
| 699 | D18P + D39L + N59S + K64L + V75I + E186K + E188P | 90.4 |
| 700 | D18P + D39L + K64L + V75I + R166Y + E186K + E188P + M218V + M220L | 90.2 |
| 701 | D18K + D39L + A53G + E186L | 90.1 |
| 702 | D39R + K64L + S177Y + M218V | 90.1 |
| 703 | D39L + A53G + N141K + S177Y | 90.1 |
| 704 | N141K + S177Y + E186R + M189L | 89.9 |
| 705 | E7K + D18P + *36aA + *36bW + D39L + K55Y + N59S + K64L + V75I + Q84E + E186K + E188P + M189L + | 89.9 |
| | M220L | |
| 706 | E7K + D18P + K27T + D39L + K55Y + N59S + K64L + V75I + Q84E + N114R + E186K + E188P + M189L + | 89.8 |
| | M220L | |
| 707 | D18P + D39L + F80Y + E186K + E188P + M189L | 89.7 |
| 708 | D18K + D39L + H99L + E186L | 89.5 |
| 709 | D18P + N28F + D39L + K64L + V75I + S168A + E186K + E188P | 89.5 |
| 710 | E7K + D18P + N28F + D39L + K55Y + N59S + K64L + V75I + Q84E + S168A + E186K + E188P + M189L + | 89.4 |
| | M220L | |
| 711 | E7K + D18P + *36aE + *36bN + D39L + K55Y + N59S + K64L + V75I + D76N + Q84E + L95F + E186K + E188P + | 89.1 |
| | M189L + M220L | |
| 712 | D18P + N141K + E186R + E188P + M218V | 89.0 |
| 713 | D18P + H99L + K147I + E186R + E188P | 88.6 |
| 714 | E7K + D18P + *36aE + *36bN + D39L + K55Y + N59S + K64L + V75I + Q84E + R166A + R181L + E186K + | 88.6 |
| | E188P + M189L + M220L | |
| 715 | D18P + K147I + E186R + E188P + M189L | 88.5 |
| 716 | D18K + D39L + S177Y + M189I | 88.2 |
| 717 | D18P + D39R + S177Y + E186R + E188P | 88.1 |
| 718 | D18E + D39R + K147I | 88.0 |
| 719 | D18P + *36aE + *36bN + D39L + K64L + V75I + D76N + L95F + E186K + E188P | 87.9 |
| 720 | D18E + D39L + M189L + M191V | 87.9 |
| 721 | D39L + S177Y + E186R + E221R | 87.6 |
| 722 | D18P + D39L + K147I + E188P + M191V | 87.6 |
| 723 | D18P + *36aA + *36bW + D39L + K64L + V75I + E186K + E188P | 86.9 |
| 724 | A89C + R200C | 86.4 |
| 725 | E7K + D18P + D39L + K55Y + N59S + K64L + V75I + Q84E + R166Y + E186K + E188P + M189L + M218L + | 86.3 |
| | M220L | |
| 726 | D18P + D39L + K64L + E186R + E188P | 86.2 |
| 727 | E7K + D18P + *36aE + *36bN + D39L + K55Y + N59S + K64L + V75I + D76N + I78V + Q84E + L95V + E186K + | 86.1 |
| | E188P + M189L + M220L | |
| 728 | D18P + D39L + S177Y + E188P + M191V | 85.8 |
| 729 | D18P + R36T + D39L + E186K + E188P + M189L | 85.7 |
| 730 | D163K + M218V | 85.5 |
| 731 | D18P + D39K + D161N + E186K + E188P + E221K + K222* | 85.5 |
| 732 | D18P + D163K | 85.4 |
| 733 | E7K + D18P + *36aE + *36bN + D39L + K55Y + N59S + K64L + V75I + I78V + Q84E + L95F + E186K + E188P + | 85.2 |
| | M189L + M220L | |
| 734 | D18P + D39L + H99L + E188P + M189I | 85.2 |
| 735 | D18P + *36aA + *36bF + D39L + K64L + V75I + E186K + E188P | 85.2 |
| 736 | D18P + H99L + S177Y + E186R + E188P | 84.8 |
| 737 | E7K + D18P + *36aE + *36bN + D39L + K55Y + N59S + K64L + V75I + D76N + I78V + Q84E + L95I + E186K + | 84.7 |
| | E188P + M189L + M220L | |
| 738 | R36D + D39K + K43A + D161N + E186K + E188P + M218K + E221K + K222* | 84.4 |
| 739 | R126C + E190C | 84.3 |
| 740 | K64L + D163K + E186R + M189L | 84.3 |
| 741 | D39K + D161N + E186K + E188P + E221K + K222* | 84.2 |
| 742 | D39K + K43A + D161N + E186K + E188P + M218K + E221K + K222* | 84.2 |
| 743 | E7K + D18P + *36aE + *36bN + D39L + K55Y + N59S + K64L + V75I + Q84E + E186K + E188P + M189L + | 84.2 |
| | A216G + M218I + M220L + E221T | |
| 744 | D18P + D39L + K64L + V75I + R166Y + E186K + E188P + M218I + M220L | 84.1 |
| 745 | D39K + K41C + K43A + D161N + E186K + E188P + M218K + E221K + K222* | 84.1 |
| 746 | D18P + D163K + E186R + E188P + M189I | 84.1 |
| 747 | Y72F + V74E + K83E + Y100F + K111E + K123E + E132D + E140D + A149D + K151E + D163E + Y165F + | 84.1 |
| | E190D + E194D + V211T + L214I + E221R | |
| 748 | Y49C + S71C | 84.0 |
| 749 | R36D + D39K + K41C + K43A + D161N + E186K + E188P + M218K + E221K + K222* | 83.9 |
| 750 | D18P + R36G + D39L + E186K + E188P + M189L | 83.9 |
| 751 | P139C + E144C | 83.6 |
| 752 | A51T + Y72F + V74E + K83E + Y100F + K111E + K123E + E132D + E140D + K151E + D163E + Y165F + E190D + | 83.6 |
| | E194D + V211T + L214I + E221R | |
| 753 | D18P + D39L + E186K + E188P + M189L + M218Q | 83.6 |
| 754 | E7K + D18P + *36aE + *36bN + D39L + K55Y + N59S + K64L + V75I + I78V + Q84E + L95V + E186K + E188P + | 83.6 |
| | M189L + M220L | |
| 755 | D18P + D39L + K64L + V75I + E186K + E188P + E221Y | 83.5 |
| 756 | D18P + *36aE + *36bN + D39L + K64L + V75I + R181I + E186K + E188P | 83.5 |
| 757 | I130C + D156C | 83.5 |
| 758 | D18P + D39K + K41C + K43A + D161N + E186K + E188P + M218K + E221K + K222* | 83.4 |
| 759 | M191V + M218V | 83.4 |
| 760 | E7K + D18P + *36aE + *36bN + D39L + K55Y + N59S + K64L + V75I + I78V + Q84E + L95I + E186K + E188P + | 83.3 |
| | M189L + M220L | |
| 761 | D18P + *36aA + *36bY + D39L + K64L + V75I + E186K + E188P | 83.3 |
| 762 | S3C + H8C | 83.3 |
| 763 | D18P + *36aE + *36bN + D39L + K64L + V75I + D76N + I78V + L95V + E186K + E188P | 83.2 |
| 764 | A51C + K151C | 83.1 |
| 765 | D18P + D39L + K64L + V75I + R166Y + E186K + E188P + M218I | 83.1 |
| 766 | D18P + D39L + K64L + V75I + R166Y + E186K + E188P + M218L | 83.0 |
| 767 | K41C + D161N + E188P | 82.9 |
| 768 | E7K + D18P + *36aE + *36bN + D39L + K55Y + N59S + K64L + V75I + Q84E + R181L + E186K + E188P + | 82.9 |
| | M189L + M220L | |
| 769 | D18P + *36aE + *36bN + D39L + K64L + V75I + I78V + L95I + E186K + E188P | 82.8 |
| 770 | D18P + *36aE + *36bN + D39L + K64L + V75I + E186K + E188P + A216G + M218I + E221T | 82.8 |
| 771 | D161N + E188P | 82.8 |
| 772 | D18P + D39K + K43A + D161N + E186K + E188P + M218K + E221K + K222* | 82.8 |
| 773 | F85C + F106C | 82.7 |
| 774 | E7K + D18P + *36aE + *36bN + D39L + K55Y + N59S + K64L + V75I + Q84E + R166M + R181L + E186K + | 82.7 |
| | E188P + M189L + M220L | |
| 775 | A51C + E68C | 82.6 |
| 776 | D18P + N28R + D39L + K64L + S168D + E186K + E188P | 82.6 |
| 777 | D18P + *36aE + *36bN + D39L + K64L + V75I + D76N + I78V + L95I + E186K + E188P | 82.5 |
| 778 | E124C + K193C | 82.5 |
| 779 | D18P + *36aE + *36bN + D39L + K64L + V75I + I78V + L95V + E186K + E188P | 82.5 |
| 780 | D18P + *36aE + *36bN + D39L + K64L + V75I + I78V + L95F + E186K + E188P | 82.5 |
| 781 | D18P + R36D + D39K + K41C + K43A + D161N + E186K + E188P + M218K + E221K + K222* | 82.4 |
| 782 | V38C + A40C | 82.4 |
| 783 | D18P + N28M + D39L + V75I + S168I + E186K + E188P + M189L | 82.3 |
| 784 | D39K + E186K + E221K + K222* | 82.3 |
| 785 | D18P + *36aE + *36bN + D39L + K64L + V75I + R181L + E186K + E188P | 82.1 |
| 786 | E7K + D18P + *36aE + *36bN + D39L + K55Y + N59S + K64L + V75I + Q84E + E186K + E188P + M189L + | 82.0 |
| | A216G + M218Y + M220L + E221T | |
| 787 | D18P + A53P | 82.0 |
| 788 | D39K + K41C + D161N + E186K + E188P + E221K + K222* | 81.9 |
| 789 | T62M + V117F | 81.8 |
| 790 | D18P + N28R + D39L + K64L + S168E + E186K + E188P | 81.7 |
| 791 | D18P + D39K + K41C + D161N + E186K + E188P + E221K + K222* | 81.7 |
| 792 | D39K + K41C + D161N + E188P + K222* | 81.7 |
| 793 | K41C + D161N + E186K + E188P + E221K | 81.7 |
| 794 | F122C + M191C | 81.7 |
| 795 | S91C + Y100C | 81.6 |
| 796 | D18P + D39L + K64L + V75I + D163T + E186K + E188P | 81.6 |
| 797 | D18P + K151P + E188P | 81.5 |
| 798 | K94C + H99C | 81.5 |
| 799 | A51C + Y72C + K151C | 81.5 |
| 800 | D18P + M218V | 81.5 |
| 801 | T62C + M203C | 81.4 |
| 802 | E188P + M218I | 81.4 |
| 803 | wt | 81.4 |
| 804 | K94L + H99P | 81.4 |
| 805 | D18P + N28R + D39L + K64L + S168L + E186K + E188P | 81.3 |
| 806 | K41C + E186K + E221K | 81.3 |
| 807 | D18P + M191V | 81.3 |
| 808 | D18P + *36aE + *36bN + D39L + K64L + V75I + R181T + E186K + E188P | 81.2 |
| 809 | D18P + N28Q + D39L + K64L + S168K + E186K + E188P | 81.2 |
| 810 | G52C + I152C | 81.2 |
| 811 | D39K + D161N + E188P + K222* | 81.2 |
| 812 | D18P + N28L + D39L + V75I + S168I + E186K + E188P + M189L | 81.2 |
| 813 | D18P + N28Q + D39L + V75I + S168K + E186K + E188P + M189L | 81.2 |
| 814 | D18P + N28R + D39L + N59L + V75I + S168P + E186K + E188P + M189L | 81.1 |
| 815 | D18P + N28R + D39L + V75I + S168L + E186K + E188P + M189L | 81.1 |
| 816 | D18P + E186R + E188P + M189I + E221L | 81.0 |
| 817 | D39L + N141K + E186R + M189I | 81.0 |
| 818 | Q98K + H112K + R126K | 81.0 |
| 819 | D39L + K41G | 81.0 |
| 820 | D18P + D39L + K64L + V75I + G119V + E186K + E188P | 81.0 |
| 821 | E7K + D18P + D39L + K55Y + N59S + K64L + V75I + Q84E + E145F + E186K + E188P + M189L + M220L | 80.9 |
| 822 | D18P + D39L + K64L + V75I + E186K + E188P + E221F | 80.9 |
| 823 | E7K + D18P + *36aE + *36bN + D39L + K55Y + N59S + K64L + V75I + Q84E + E186K + E188P + M189L + | 80.9 |
| | A216G + M218S + M220L + E221T | |
| 824 | Q98K + F102L + H112K | 80.9 |
| 825 | D18P + N28R + D39L + N59L + V75I + S168D + E186K + E188P + M189L | 80.8 |
| 826 | D18P + *36aE + *36bN + D39L + K64L + V75I + E186K + E188P + A216G + M218Y + E221T | 80.8 |
| 827 | D18P + *36aE + *36bN + D39L + K64L + V75I + R166Y + R181T + E186K + E188P | 80.8 |
| 828 | D18P + D39L + K43P + K64L + V75I + E186K + E188P | 80.7 |
| 829 | L95I + H112K + E188P | 80.7 |
| 830 | D18P + N28E + D39L + V75I + S168M + E186K + E188P + M189L | 80.7 |
| 831 | D18P + *36aE + *36bN + D39L + K64L + V75I + E186K + E188P + A216G + M218A | 80.7 |
| 832 | D18P + N28R + D39L + V75I + S168D + E186K + E188P + M189L | 80.6 |
| 833 | D18P + D39Y + K64L + V75I + E186K + E188P | 80.6 |
| 834 | D18P + K27T + D39L + K64L + V75I + N114R + E186K + E188P | 80.6 |
| 835 | D18P + N28M + D39L + K64L + V75I + S168M + E186K + E188P | 80.6 |
| 836 | D18P + N28Q + D39L + N59L + V75I + S168Q + E186K + E188P + M189L | 80.5 |
| 837 | D161C + M185C | 80.5 |
| 838 | S91C + H99C | 80.5 |
| 839 | Q98K + H112D + E188P | 80.4 |
| 840 | D18P + N28R + D39L + K64L + V75I + S168L + E186K + E188P | 80.4 |
| 841 | D18P + *36aE + *36bN + D39L + K64L + V75I + E186K + E188P + A216G + M218Y + E221K | 80.4 |
| 842 | D18P + N28R + D39L + K64L + V75I + S168M + E186K + E188P | 80.3 |
| 843 | H112D + E188P + M189I | 80.3 |
| 844 | D18P + H112E + R126K | 80.3 |
| 845 | H61C + M203C | 80.3 |
| 846 | D18P + *36aE + *36bN + D39L + K64L + V75I + E186K + E188P + A216G + M218A + E221T | 80.3 |
| 847 | D18P + N28E + D39L + V75I + S168D + E186K + E188P + M189L | 80.3 |
| 848 | E7K + D18P + *36aE + *36bN + D39L + K55Y + N59S + K64L + V75I + Q84E + E186K + E188P + M189L + | 80.2 |
| | A216G + M218A + M220L + E221T | |
| 849 | D18P + N28Q + D39L + K64L + S168T + E186K + E188P | 80.2 |
| 850 | D18P + N28Q + D39L + V75I + S168L + E186K + E188P + M189L | 80.2 |
| 851 | F102L + P129L | 80.2 |
| 852 | D39R + K41G | 80.2 |
| 853 | H112E + R126K + M191V | 80.2 |
| 854 | D18P + R36N + D39L + K64L + V75I + D76K + R181A + E186K + E188P | 80.2 |
| 855 | D18P + *36aE + *36bN + D39L + K64L + V75I + E186K + E188P + A216G + M218A + E221V | 80.2 |
| 856 | D18P + *36aE + *36bN + D39L + K64L + V75I + E186K + E188P + A216G + M218A + E221K | 80.2 |
| 857 | E7K + D18P + D39L + K55Y + N59S + K64L + V75I + Q84E + D156M + E186K + E188P + M189L + M220L | 80.1 |
| 858 | D18P + N28R + D39L + K64L + V75I + S168E + E186K + E188P | 80.1 |
| 859 | E7K + D18P + *36aE + *36bN + D39L + K55Y + N59S + K64L + V75I + Q84E + E186K + E188P + M189L + | 80.0 |
| | A216G + M218N + M220L + E221T | |
| 860 | D18P + N28R + D39L + V75I + S168P + E186K + E188P + M189L | 80.0 |
| 861 | E7K + D18P + *36aE + *36bN + D39L + K55Y + N59S + K64L + V75I + Q84E + R166M + R181T + E186K + | 80.0 |
| | E188P + M189L + M220L | |
| 862 | D18P + M22F + N28F + D39L + K64L + V75I + E186K + E188P | 80.0 |
| 863 | D18P + N28E + D39L + K64L + V75I + S168M + E186K + E188P | 80.0 |
| 864 | D18P + E188P | 79.9 |
| 865 | D18P + K147P + A149P + E188P | 79.9 |
| 866 | K147E + K151C | 79.9 |
| 867 | F102L + H112K + E188P | 79.9 |
| 868 | E7K + D18P + *36aE + *36bN + D39L + K55Y + N59S + K64L + V75I + Q84E + R166E + E186K + E188P + | 79.9 |
| | M189L + M220L | |
| 869 | E7K + D18P + *36aE + *36bN + D39L + K55Y + N59S + K64L + V75I + Q84E + E186K + E188P + M189L + | 79.9 |
| | A216G + M218A + M220L | |
| 870 | R36D + K43A | 79.8 |
| 871 | D39K + K41C + E186K + E221K + K222* | 79.8 |
| 872 | D18P + A89E + K151P + E188P + D206P | 79.8 |
| 873 | E7K + D18P + *36aE + *36bN + D39L + K55Y + N59S + K64L + V75I + Q84E + E186K + E188P + M189L + | 79.8 |
| | A216G + M218N + M220L | |
| 874 | D18P + N28Q + D39L + K64L + S168L + E186K + E188P | 79.8 |
| 875 | D18P + N28R + D39L + K64L + V75I + S168P + E186K + E188P | 79.7 |
| 876 | D18P + N28R + D39L + K64L + V75I + S168Q + E186K + E188P | 79.7 |
| 877 | L95I + F102L + H112D | 79.7 |
| 878 | K41G + D163K | 79.7 |
| 879 | E7K + D18P + *36aE + *36bN + D39L + K55Y + N59S + K64L + V75I + Q84E + R166K + R181T + E186K + | 79.7 |
| | E188P + M189L + M220L | |
| 880 | D18P + *36aE + *36bN + D39L + K64L + V75I + E186K + E188P + A216G + M218S | 79.6 |
| 881 | D18P + *36aE + *36bN + D39L + K64L + V75I + E186K + E188P + A216G + M218N | 79.6 |
| 882 | V137K + H150V + M189V | 79.6 |
| 883 | D18P + N28R + D39L + V75I + S168M + E186K + E188P + M189L | 79.6 |
| 884 | D18P + N28L + D39L + K64L + V75I + S168V + E186K + E188P | 79.5 |
| 885 | E7K + D18P + *36aE + *36bN + D39L + K55Y + N59S + K64L + V75I + Q84E + E186K + E188P + M189L + | 79.5 |
| | A216G + M218S + M220L | |
| 886 | E7K + D18P + *36aE + *36bN + D39L + K55Y + N59S + K64L + V75I + Q84E + R166Y + R181T + E186K + | 79.5 |
| | E188P + M189L + M220L | |
| 887 | E7K + D18P + *36aE + *36bN + D39L + K55Y + N59S + K64L + V75I + Q84E + E186K + E188P + M189L + | 79.4 |
| | A216G + M218A + M220L + E221K | |
| 888 | D18P + R36N + D39L + K64L + V75I + D76K + R181T + E186K + E188P | 79.3 |
| 889 | D18P + *36aE + *36bN + D39L + K64L + V75I + E186K + E188P + A216G + R217N + M218Y | 79.3 |
| 890 | E7K + D18P + M22F + N28T + D39L + K55Y + N59S + K64L + V75I + Q84E + E178P + E186K + E188P + M189L + | 79.3 |
| | M220L | |
| 891 | E7K + D18P + *36aE + *36bN + D39L + K55Y + N59S + K64L + V75I + Q84E + R181T + E186K + E188P + | 79.2 |
| | M189L + M220L | |
| 892 | D18P + N28Q + D39L + K64L + V75I + S168L + E186K + E188P | 79.2 |
| 893 | D18P + *36aE + *36bN + D39L + K64L + V75I + E186K + E188P + A216N + E221T | 79.2 |
| 894 | D18P + *36aE + *36bN + D39L + K64L + V75I + E186K + E188P + A216N + M218A + E221T | 79.0 |
| 895 | D18P + R36N + D39L + K64L + V75I + D76N + R181T + E186K + E188P | 78.9 |
| 896 | D18P + N28Q + D39L + V75I + S168T + E186K + E188P + M189L | 78.9 |
| 897 | D18P + R36S + *36aA + *36bD + I37A + D39K + K41C + K43A + K64L + V75I + E186K + E188P | 78.9 |
| 898 | D18P + *36aE + *36bN + D39L + K64L + V75I + E186K + E188P + A216N + M218Y + E221K | 78.9 |
| 899 | D18P + *36aE + *36bN + D39L + K64L + V75I + E186K + E188P + A216N + M218A | 78.8 |
| 900 | E7K + D18P + *36aE + *36bN + D39L + K55Y + N59S + K64L + V75I + Q84E + E186K + E188P + M189L + | 78.8 |
| | A216N + M218S + M220L + E221T | |
| 901 | D18P + M22Y + D39L + K64L + V75I + E178Q + E186K + E188P | 78.8 |
| 902 | D18P + N28R + D39L + V75I + E186K + E188P + M189L | 78.8 |
| 903 | E7K + D18P + *36aE + *36bN + D39L + K55Y + N59S + K64L + V75I + Q84E + E186K + E188P + M189L + | 78.7 |
| | A216G + R217N + M218V + M220L + E221K | |
| 904 | D18P + M22W + D39L + K64L + V75I + E178Q + E186K + E188P | 78.7 |
| 905 | E7K + D18P + *36aE + *36bN + D39L + K55Y + N59S + K64L + V75I + Q84E + E186K + E188P + M189L + | 78.7 |
| | A216G + R217N + M218L + M220L | |
| 906 | E7K + D18P + *36aE + *36bN + D39L + K55Y + N59S + K64L + V75I + Q84E + E186K + E188P + M189L + | 78.6 |
| | A216G + M218N + M220L + E221K | |
| 907 | E7K + D18P + *36aE + *36bK + D39L + K55Y + N59S + K64L + V75I + Q84E + R181T + E186K + E188P + | 78.6 |
| | M189L + M220L | |
| 908 | D18P + *36aE + *36bN + D39L + K41C + K64L + V75I + E186K + E188P | 78.5 |
| 909 | D18P + *36aE + *36bN + D39L + K64L + V75I + E186K + E188P + A216G + M218N + E221K | 78.5 |
| 910 | D18P + N28M + D39L + V75I + S168M + E186K + E188P + M189L | 78.4 |
| 911 | D18P + *36aE + *36bN + D39L + K64L + V75I + E186K + E188P + A216N + M218G + E221V | 78.4 |
| 912 | D18P + *36aE + *36bN + D39L + K64L + V75I + E186K + E188P + A216G + M218G | 78.4 |
| 913 | E7K + D18P + *36aK + *36bN + D39L + K55Y + N59S + K64L + V75I + Q84E + R181T + E186K + E188P + | 78.3 |
| | M189L + M220L | |
| 914 | E7K + D18P + *36aE + *36bN + D39L + K55Y + N59S + K64L + V75I + Q84E + E186K + E188P + M189L + | 78.3 |
| | A216N + M220L + E221T | |
| 915 | D18P + *36aE + *36bN + D39L + K64L + V75I + E186K + E188P + A216G + R217N + M218L | 78.2 |
| 916 | E7K + D18P + *36aE + *36bN + D39L + K55Y + N59S + K64L + V75I + Q84E + E186K + E188P + M189L + | 78.2 |
| | A216G + M218G + M220L + E221R | |
| 917 | D18P + D39L + N59V + K64L + V75I + E186K + E188P | 78.1 |
| 918 | E7K + D18P + *36aE + *36bN + D39L + K55Y + N59S + K64L + V75I + Q84E + E186K + E188P + M189L + | 78.1 |
| | A216N + M218A + M220L + E221T | |
| 919 | D18P + N28Q + D39L + V75I + S168Q + E186K + E188P + M189L | 77.9 |
| 920 | E7K + D18P + *36aE + *36bN + D39L + K55Y + N59S + K64L + V75I + Q84E + E186K + E188P + M189L + | 77.8 |
| | A216G + M218G + M220L + E221T | |
| 921 | D18P + N28Q + D39L + V75I + S168P + E186K + E188P + M189L | 77.8 |
| 922 | D18P + M22Y + N28F + D39L + K64L + V75I + E186K + E188P | 77.7 |
| 923 | E7K + D18P + *36aE + *36bN + D39L + K55Y + N59S + K64L + V75I + Q84E + E186K + E188P + M189L + | 77.7 |
| | A216N + M218Y + M220L + E221K | |
| 924 | D18P + N28Y + D39L + K64L + V75I + S168A + E186K + E188P | 77.6 |
| 925 | D18P + M22F + D39L + K64L + V75I + E178Q + E186K + E188P | 77.6 |
| 926 | E7K + D18P + *36aE + *36bN + D39L + K55Y + N59S + K64L + V75I + Q84E + E186K + E188P + M189L + | 77.5 |
| | A216N + R217N + M218Y + M220L | |
| 927 | D18P + D39L + K64L + V75I + H88Y + E186K + E188P | 77.4 |
| 928 | E7K + D18P + *36aE + *36bN + D39L + K55Y + N59S + K64L + V75I + Q84E + E186K + E188P + M189L + | 77.4 |
| | A216G + M218G + M220L + E221V | |
| 929 | E7K + D18P + R36S + *36aA + *36bD + I37A + D39K + K41C + K43A + K55Y + N59S + K64L + V75I + Q84E + | 77.2 |
| | E186K + E188P + M189L + M220L | |
| 930 | E7K + D18P + *36aE + *36bN + D39L + K55Y + N59S + K64L + V75I + Q84E + E186K + E188P + M189L + | 77.1 |
| | A216N + M218G + M220L + E221V | |
| 931 | D18P + N28S + D39L + K64L + V75I + S168C + E186K + E188P + A216C | 77.1 |
| 932 | E7K + D18P + *36aE + *36bN + D39L + K55Y + N59S + K64L + V75I + Q84E + E186K + E188P + M189L + | 77.0 |
| | A216N + R217N + M218Y + M220L + E221K | |
| 933 | E7K + D18P + *36aE + *36bN + D39L + K55Y + N59S + K64L + V75I + Q84E + E186K + E188P + M189L + | 76.9 |
| | A216N + M218A + M220L | |
| 934 | N141T + R166C + E186K + E221K | 76.9 |
| 935 | E7K + D18P + *36aE + *36bN + D39L + K55Y + N59S + K64L + V75I + Q84E + E186K + E188P + M189L + | 76.8 |
| | A216G + M218G + M220L + E221K | |
| 936 | D18P + D39L + K43F + K64L + V75I + E186K + E188P | 76.8 |
| 937 | D18P + D39L + K64L + V75I + E178S + E186K + E188P | 76.8 |
| 938 | E7K + D18P + *36aE + *36bN + D39L + K55Y + N59S + K64L + V75I + Q84E + E186K + E188P + M189L + | 76.6 |
| | A216G + R217N + M218F + M220L + E221V | |
| 939 | D18P + R36N + D39L + K64L + V75I + D76N + R181A + E186K + E188P | 76.5 |
| 940 | D18P + R36N + D39L + K64L + V75I + S168A + R181T + E186K + E188P + M218V + M220L | 76.1 |
| 941 | E7K + D18P + D39L + K55Y + N59S + K64L + V75I + Q84E + R166Y + E186K + E188P + M189L + M218I + | 75.8 |
| | M220L | |

### Example 4: Thermal Stability of Logatchev Hydrothermal Carbonic Anhydrase Variants

The Logatchev hydrothermal carbonic anhydrase variants of SEQ ID NO: 2 were prepared as described in Example 1 and the thermal stability measured according to the procedure of Example 2. Melting temperatures of 292 variants were calculated from the inflection points in the melting curve using the first and second derivative and compared to the parent Logatchev hydrothermal carbonic anhydrase (wt) shown in Table 4 below. Variants 1-119 showed improved stability compared to the parent carbonic anhydrase.

TABLE 4

| | Carbonic | Corresponding | |
|---|---|---|---|
| | Anhydrase | position in | |
| Entry | Alteration | SEQ ID NO: 1 | Tm |
| 1 | G123T | 119 | 80.1 |
| 2 | E189K | 186 | 79.4 |
| 3 | D114S | 110 | 79.2 |
| 4 | E224R | 221 | 79.0 |
| 5 | E224K | 221 | 78.6 |
| 6 | G123S | 119 | 78.2 |
| 7 | R184I | 181 | 78.2 |
| 8 | G123A | 119 | 78.1 |
| 9 | Q88Y | 84 | 77.9 |
| 10 | W139L | 135 | 77.9 |
| 11 | N63L | 59 | 77.9 |
| 12 | S55P | 51 | 77.7 |
| 13 | Q88W | 84 | 77.4 |
| 14 | R184L | 181 | 77.4 |
| 15 | Q88F | 84 | 77.2 |
| 16 | N63T | 59 | 77.0 |
| 17 | E224F | 221 | 76.9 |
| 18 | E189R | 186 | 76.9 |
| 19 | E189I | 186 | 76.6 |
| 20 | S54K | 50 | 76.6 |
| 21 | Q88E | 84 | 76.6 |
| 22 | S59Y | 55 | 76.5 |
| 23 | E224I | 221 | 76.4 |
| 24 | M223L | 220 | 76.3 |
| 25 | E189L | 186 | 76.3 |
| 26 | E191V | 188 | 76.1 |
| 27 | M221L | 218 | 76.1 |
| 28 | E224V | 221 | 76.0 |
| 29 | S171I | 168 | 75.8 |
| 30 | N63D | 59 | 75.8 |
| 31 | M221K | 218 | 75.7 |
| 32 | S55T | 51 | 75.5 |
| 33 | M192V | 189 | 75.4 |
| 34 | S171T | 168 | 75.4 |
| 35 | T66Y | 62 | 75.4 |
| 36 | Q88A | 84 | 75.3 |
| 37 | S70L | 66 | 75.3 |
| 38 | M221V | 218 | 75.3 |
| 39 | T66F | 62 | 75.3 |
| 40 | G208E | 205 | 75.3 |
| 41 | T176I | 173 | 75.2 |
| 42 | M188F | 185 | 75.2 |
| 43 | M221I | 218 | 75.2 |
| 44 | M225L | 222 | 75.1 |
| 45 | K87I | 83 | 75.1 |
| 46 | M225*, D226* | 222 | 75.1 |
| 47 | S171M | 168 | 75.1 |
| 48 | S70Y | 66 | 75.0 |
| 49 | S171V | 168 | 75.0 |
| 50 | A219P | 216 | 75.0 |
| 51 | K154P | 151 | 75.0 |
| 52 | T176W | 173 | 75.0 |
| 53 | A219H | 219 | 75.0 |
| 54 | K166T | 163 | 75.0 |
| 55 | S70W | 66 | 74.9 |
| 56 | A219F | 216 | 74.9 |
| 57 | R184T | 181 | 74.9 |
| 58 | M206L | 203 | 74.9 |
| 59 | S171E | 168 | 74.9 |
| 60 | K131P | 127 | 74.8 |
| 61 | E224Y | 221 | 74.8 |
| 62 | S171L | 168 | 74.7 |
| 63 | N63H | 59 | 74.7 |
| 64 | Q88G | 84 | 74.7 |
| 65 | S171K | 168 | 74.7 |
| 66 | Y71V | 67 | 74.7 |
| 67 | Y71M | 67 | 74.6 |
| 68 | S180M | 177 | 74.6 |
| 69 | K68L | 64 | 74.6 |
| 70 | S171N | 168 | 74.6 |
| 71 | N22K | 18 | 74.6 |
| 72 | S171Q | 168 | 74.5 |
| 73 | M206I | 203 | 74.5 |
| 74 | S171D | 168 | 74.4 |
| 75 | E181Q | 178 | 74.4 |
| 76 | K21S | 17 | 74.4 |
| 77 | N63V | 59 | 74.4 |
| 78 | S70I | 66 | 74.4 |
| 79 | T66W | 62 | 74.4 |
| 80 | L194V | 191 | 74.3 |
| 81 | N52Y | 48 | 74.3 |
| 82 | K100N | 96 | 74.3 |
| 83 | A152D | 149 | 74.3 |
| 84 | Q88K | 84 | 74.2 |
| 85 | S58A | 54 | 74.2 |
| 86 | Y168F | 165 | 74.2 |
| 87 | T66M | 62 | 74.2 |
| 88 | V79I | 75 | 74.2 |
| 89 | N52V | 48 | 74.2 |
| 90 | M192L | 189 | 74.2 |
| 91 | A152H | 149 | 74.2 |
| 92 | H153S | 150 | 74.1 |
| 93 | H16L | 12 | 74.1 |
| 94 | A108V | 104 | 74.1 |
| 95 | I125M | 121 | 74.1 |
| 96 | A145K | 142 | 74.1 |
| 97 | F106M | 102 | 74.1 |
| 98 | F84Y | 80 | 74.1 |
| 99 | G1D | 1 | 74.1 |
| 100 | S70F | 66 | 74.1 |
| 101 | E197D | 194 | 74.1 |
| 102 | T210N | 207 | 74.0 |
| 103 | T12C | 8 | 74.0 |
| 104 | D209P | 206 | 74.0 |
| 105 | E181P | 178 | 74.0 |
| 106 | S59H | 55 | 74.0 |
| 107 | T176V | 173 | 73.9 |
| 108 | H65Y | 61 | 73.9 |
| 109 | N63I | 59 | 73.9 |
| 110 | G123C | 119 | 73.9 |
| 111 | I29T | 25 | 73.9 |
| 112 | S7G | 3 | 73.9 |
| 113 | R169Y | 166 | 73.9 |
| 114 | I82K | 78 | 73.8 |
| 115 | M192I | 189 | 73.8 |
| 116 | Y158A | 155 | 73.8 |
| 117 | H9R | 5 | 73.8 |
| 118 | E43K | 39 | 73.8 |
| 119 | R83I | 79 | 73.8 |
| 120 | WT | N/A | 73.7 |
| 121 | E11K | 7 | 73.7 |
| 122 | K204M | 201 | 73.7 |
| 123 | N63W | 59 | 73.7 |
| 124 | S70G | 66 | 73.7 |
| 125 | I125L | 121 | 73.7 |
| 126 | K50Y | 46 | 73.7 |
| 127 | S171A | 168 | 73.6 |
| 128 | H9S | 5 | 73.6 |
| 129 | A152P | 149 | 73.6 |
| 130 | R169V | 166 | 73.6 |
| 131 | G129A | 125 | 73.6 |
| 132 | K166L | 163 | 73.6 |
| 133 | Y71I | 67 | 73.6 |
| 134 | A44M | 40 | 73.6 |
| 135 | S171P | 168 | 73.6 |
| 136 | E43S | 39 | 73.6 |
| 137 | K137M | 133 | 73.5 |
| 138 | N52R | 48 | 73.5 |
| 139 | E144A | 140 | 73.5 |
| 140 | R83V | 79 | 73.5 |
| 141 | A113Q | 109 | 73.5 |
| 142 | E181S | 178 | 73.5 |
| 143 | K164N | 161 | 73.5 |
| 144 | R169F | 166 | 73.5 |
| 145 | I99F | 95 | 73.5 |
| 146 | E85Y | 81 | 73.4 |
| 147 | K165L | 162 | 73.4 |
| 148 | S58K | 54 | 73.4 |
| 149 | T98L | 94 | 73.4 |
| 150 | G172D | 169 | 73.4 |
| 151 | Y158R | 155 | 73.4 |
| 152 | H153V | 150 | 73.4 |
| 153 | M188L | 185 | 73.3 |
| 154 | V214T | 211 | 73.3 |
| 155 | S171G | 168 | 73.3 |
| 156 | K201Y | 198 | 73.3 |
| 157 | G10P | 6 | 73.3 |
| 158 | K204I | 201 | 73.2 |
| 159 | T98M | 94 | 73.2 |
| 160 | G56V | 52 | 73.2 |
| 161 | E144D | 140 | 73.2 |
| 162 | K150E | 147 | 73.2 |
| 163 | K201F | 198 | 73.2 |
| 164 | S180F | 177 | 73.2 |
| 165 | K165M | 162 | 73.2 |
| 166 | H65F | 61 | 73.2 |
| 167 | I78E | 74 | 73.2 |
| 168 | K68Q | 64 | 73.2 |
| 169 | N52F | 48 | 73.2 |
| 170 | A108L | 104 | 73.2 |
| 171 | I25A | 21 | 73.1 |
| 172 | K137I | 133 | 73.1 |
| 173 | I99Y | 95 | 73.1 |
| 174 | N63S | 59 | 73.0 |
| 175 | L217I | 214 | 73.0 |
| 176 | I41V | 37 | 73.0 |
| 177 | A152L | 149 | 73.0 |
| 178 | K166E | 163 | 73.0 |
| 179 | G1M | 1 | 72.9 |
| 180 | R40L | 36 | 72.9 |
| 181 | K150I | 147 | 72.9 |
| 182 | R40D | 36 | 72.9 |
| 183 | K115E | 111 | 72.9 |
| 184 | E47H | 43 | 72.9 |
| 185 | T61V | 57 | 72.8 |
| 186 | A44C | 40 | 72.8 |
| 187 | E47P | 47 | 72.8 |
| 188 | S55C | 51 | 72.8 |
| 189 | A93S | 89 | 72.7 |
| 190 | E43L | 39 | 72.7 |
| 191 | E43M | 39 | 72.7 |
| 192 | E200V | 197 | 72.7 |
| 193 | Y24F | 20 | 72.7 |
| 194 | R40I | 36 | 72.7 |
| 195 | K150M | 147 | 72.7 |
| 196 | A93T | 89 | 72.6 |
| 197 | S180Y | 177 | 72.6 |
| 198 | M26P | 22 | 72.6 |
| 199 | K137V | 133 | 72.6 |
| 200 | T66N | 62 | 72.6 |
| 201 | E45G | 41 | 72.6 |
| 202 | S39D | 35 | 72.6 |
| 203 | E136D | 132 | 72.6 |
| 204 | E43G | 39 | 72.5 |
| 205 | N211L | 208 | 72.5 |
| 206 | K100D | 96 | 72.5 |
| 207 | E45C | 41 | 72.5 |
| 208 | N52S | 48 | 72.5 |
| 209 | I155L | 152 | 72.5 |
| 210 | E43R | 39 | 72.5 |
| 211 | E23P | 19 | 72.5 |
| 212 | S171C | 168 | 72.5 |
| 213 | V42C | 38 | 72.5 |
| 214 | N22E | 18 | 72.5 |
| 215 | D116E | 112 | 72.4 |
| 216 | E43C | 39 | 72.4 |
| 217 | A157P | 154 | 72.4 |
| 218 | S59F | 55 | 72.4 |
| 219 | D159L | 156 | 72.4 |
| 220 | E43P | 39 | 72.3 |
| 221 | S7C | 3 | 72.3 |
| 222 | *144aK | 141 | 72.3 |
| 223 | E47A | 43 | 72.3 |
| 224 | N22P | 18 | 72.3 |
| 225 | S180W | 177 | 72.3 |
| 226 | K154C | 151 | 72.3 |
| 227 | Y104F | 100 | 72.2 |
| 228 | E43A | 39 | 72.2 |
| 229 | S95H | 91 | 72.2 |
| 230 | E43N | 39 | 72.2 |
| 231 | K165F | 162 | 72.2 |
| 232 | M26N | 22 | 72.2 |
| 233 | E191P | 188 | 72.1 |
| 234 | N141K | 137 | 72.1 |
| 235 | K154E | 151 | 72.0 |
| 236 | A93Y | 89 | 72.0 |
| 237 | A44F | 40 | 72.0 |
| 238 | D116N | 112 | 72.0 |
| 239 | E43F | 39 | 72.0 |
| 240 | D80L | 76 | 71.9 |
| 241 | S103L | 99 | 71.9 |
| 242 | K147C | 144 | 71.9 |
| 243 | S103Y | 99 | 71.9 |
| 244 | K131L | 127 | 71.9 |
| 245 | K201L | 198 | 71.9 |
| 246 | E197A | 194 | 71.8 |
| 247 | I82E | 78 | 71.8 |
| 248 | E107Q | 103 | 71.8 |
| 249 | I122V | 118 | 71.7 |
| 250 | K131C | 127 | 71.6 |
| 251 | E144EN | 141 | 71.4 |
| 252 | K131V | 127 | 71.4 |
| 253 | V121F | 117 | 71.4 |
| 254 | E128C | 124 | 71.4 |
| 255 | P133L | 129 | 71.3 |
| 256 | K166R | 163 | 71.3 |
| 257 | K196C | 193 | 71.2 |
| 258 | E72C | 68 | 71.2 |
| 259 | R203C | 200 | 71.2 |
| 260 | G56C | 52 | 71.1 |
| 261 | G57P | 53 | 71.1 |
| 262 | E193D | 190 | 71.0 |
| 263 | N52D | 48 | 71.0 |
| 264 | L151P | 148 | 70.9 |
| 265 | A93E | 89 | 70.7 |
| 266 | K87E | 83 | 70.5 |
| 267 | S54P | 50 | 70.1 |
| 268 | S195C | 192 | 70.1 |
| 269 | F126Y | 122 | 70.0 |
| 270 | I134E | 130 | 69.9 |
| 271 | W185K | 182 | 69.7 |
| 272 | E190V | 187 | 69.7 |
| 273 | E190I | 187 | 69.5 |
| 274 | E224L | 221 | 69.4 |
| 275 | E193G | 190 | 69.4 |
| 276 | Y76E | 72 | 69.3 |
| 277 | N118G | 114 | 69.1 |
| 278 | I155C | 152 | 69.0 |
| 279 | K68I | 64 | 69.0 |
| 280 | G129S | 125 | 68.9 |
| 281 | G123I | 119 | 68.8 |
| 282 | N62D | 58 | 68.6 |
| 283 | P143C | 139 | 68.4 |
| 284 | S75C | 71 | 68.2 |
| 285 | A93C | 89 | 68.1 |
| 286 | G129D | 125 | 68.0 |
| 287 | L86P | 82 | 67.8 |
| 288 | K127E | 123 | 67.6 |
| 289 | Y76F | 72 | 67.6 |
| 290 | Y53C | 49 | 67.4 |
| 291 | D159M | 156 | 67.3 |
| 292 | Y76C | 72 | 65.0 |
| 293 | F89C | 85 | 64.6 |

### Example 5: Thermal Stability of Persephonella hydrogeniphilia Carbonic Anhydrase Variants

The Persephonella hydrogeniphilia carbonic anhydrase variants of SEQ ID NO: 4 were prepared as described in Example 1 and the thermal stability measured according to the procedure of Example 2. Melting temperatures (Tm1, 260 variants; and Tm2; 210 variants) were calculated from the inflection points in the melting curve using the first and second derivative and compared to the parent P. hydrogeniphilia carbonic anhydrase (wt). Tm1 and Tm2 values for the variants are shown in Tables 5 and 6 below, respectively. Variants 1-146 of Table 5 and 1-74 of Table 6 showed improved stability compared to the parent carbonic anhydrase.

TABLE 5

| | Carbonic | Corresponding | |
|---|---|---|---|
| | Anhydrase | position in SEQ | |
| Entry | Alteration | ID NO: 1 | Tm1 |
| 1 | R183L | 181 | 79.4 |
| 2 | S170T | 168 | 79.0 |
| 3 | G121V | 119 | 78.7 |
| 4 | S68F | 66 | 78.5 |
| 5 | S170P | 168 | 78.1 |
| 6 | E190P | 188 | 78.0 |
| 7 | R183I | 181 | 77.9 |
| 8 | S52K | 50 | 77.6 |
| 9 | S68L | 66 | 77.0 |
| 10 | G121T | 119 | 76.9 |
| 11 | S170I | 168 | 76.9 |
| 12 | S68I | 66 | 76.8 |
| 13 | R183T | 181 | 76.7 |
| 14 | T188L | 186 | 76.6 |
| 15 | K66I | 64 | 76.2 |
| 16 | S170V | 168 | 76.1 |
| 17 | K165T | 163 | 76.1 |
| 18 | T175V | 173 | 76.1 |
| 19 | E180S | 178 | 76.0 |
| 20 | G121I | 119 | 75.9 |
| 21 | S170D | 168 | 75.9 |
| 22 | S57H | 55 | 75.9 |
| 23 | G121A | 119 | 75.8 |
| 24 | S170M | 168 | 75.7 |
| 25 | I23A | 21 | 75.7 |
| 26 | M191V | 189 | 75.5 |
| 27 | E190V | 188 | 75.5 |
| 28 | T188K | 186 | 75.4 |
| 29 | T175I | 173 | 75.4 |
| 30 | S68W | 66 | 75.3 |
| 31 | H152V | 150 | 75.3 |
| 32 | T56A | 54 | 75.3 |
| 33 | K149E | 147 | 75.3 |
| 34 | I27T | 25 | 75.3 |
| 35 | N61L | 59 | 75.2 |
| 36 | T64W | 62 | 75.2 |
| 37 | K149M | 147 | 75.2 |
| 38 | N61T | 59 | 75.1 |
| 39 | T64F | 62 | 75.1 |
| 40 | K153P | 151 | 75.0 |
| 41 | K48Y | 46 | 75.0 |
| 42 | K149I | 147 | 74.9 |
| 43 | G121S | 119 | 74.9 |
| 44 | S170E | 168 | 74.8 |
| 45 | H63Y | 61 | 74.8 |
| 46 | K165R | 163 | 74.8 |
| 47 | S57F | 55 | 74.8 |
| 48 | S170Q | 168 | 74.8 |
| 49 | H50Y | 48 | 74.8 |
| 50 | E180Q | 178 | 74.7 |
| 51 | T64Y | 62 | 74.7 |
| 52 | A151P | 149 | 74.7 |
| 53 | M24P | 22 | 74.6 |
| 54 | S57Y | 55 | 74.6 |
| 55 | S179Y | 177 | 74.6 |
| 56 | N61I | 59 | 74.6 |
| 57 | S37D | 35 | 74.6 |
| 58 | A151H | 149 | 74.5 |
| 59 | R163N | 161 | 74.5 |
| 60 | S170C | 168 | 74.5 |
| 61 | A151D | 149 | 74.5 |
| 62 | S170N | 168 | 74.5 |
| 63 | R168Y | 166 | 74.4 |
| 64 | Y167F | 165 | 74.3 |
| 65 | T56K | 54 | 74.3 |
| 66 | Q86Y | 84 | 74.3 |
| 67 | S170K | 168 | 74.3 |
| 68 | A151L | 149 | 74.2 |
| 69 | M187F | 185 | 74.2 |
| 70 | R38I | 36 | 74.2 |
| 71 | W137L | 135 | 74.1 |
| 72 | A55G | 53 | 74.1 |
| 73 | S170L | 168 | 74.1 |
| 74 | N61V | 59 | 74.1 |
| 75 | K165E | 163 | 74.1 |
| 76 | K200F | 198 | 74.0 |
| 77 | N61W | 59 | 74.0 |
| 78 | S179F | 177 | 74.0 |
| 79 | K200Y | 198 | 74.0 |
| 80 | E180P | 178 | 74.0 |
| 81 | D43G | 41 | 74.0 |
| 82 | A42F | 40 | 73.9 |
| 83 | K19S | 17 | 73.9 |
| 84 | S179M | 177 | 73.9 |
| 85 | K165L | 163 | 73.8 |
| 86 | D208P | 206 | 73.8 |
| 87 | E45P | 43 | 73.8 |
| 88 | H63F | 61 | 73.8 |
| 89 | H50V | 48 | 73.8 |
| 90 | R38L | 36 | 73.8 |
| 91 | T64M | 62 | 73.7 |
| 92 | M205L | 203 | 73.7 |
| 93 | E45Y | 43 | 73.7 |
| 94 | A42M | 40 | 73.7 |
| 95 | T175W | 173 | 73.6 |
| 96 | N210L | 208 | 73.5 |
| 97 | E41G | 39 | 73.5 |
| 98 | E41N | 39 | 73.4 |
| 99 | S179W | 177 | 73.4 |
| 100 | H50R | 48 | 73.4 |
| 101 | N61S | 59 | 73.4 |
| 102 | M205I | 203 | 73.4 |
| 103 | E45A | 43 | 73.3 |
| 104 | K153E | 151 | 73.3 |
| 105 | K200L | 198 | 73.3 |
| 106 | S3D | 1 | 73.3 |
| 107 | S68G | 66 | 73.3 |
| 108 | R168F | 166 | 73.3 |
| 109 | Q86W | 84 | 73.2 |
| 110 | H152S | 150 | 73.2 |
| 111 | S68Y | 66 | 73.2 |
| 112 | L59V | 57 | 73.2 |
| 113 | P53T | 51 | 73.1 |
| 114 | K203I | 201 | 73.1 |
| 115 | D20E | 18 | 73.1 |
| 116 | E41S | 39 | 73.1 |
| 117 | E41M | 39 | 73.1 |
| 118 | T10C | 8 | 73.1 |
| 119 | E41L | 39 | 73.1 |
| 120 | N61D | 59 | 73.1 |
| 121 | V213T | 211 | 73.0 |
| 122 | E45H | 43 | 73.0 |
| 123 | Q86F | 84 | 73.0 |
| 124 | H14L | 12 | 73.0 |
| 125 | P53S | 51 | 72.9 |
| 126 | M191L | 189 | 72.9 |
| 127 | D20K | 18 | 72.9 |
| 128 | T209N | 207 | 72.9 |
| 129 | E41K | 39 | 72.9 |
| 130 | H50S | 48 | 72.8 |
| 131 | M220K | 218 | 72.8 |
| 132 | F104M | 102 | 72.8 |
| 133 | K66Q | 64 | 72.7 |
| 134 | D20P | 18 | 72.7 |
| 135 | D43C | 41 | 72.7 |
| 136 | E21P | 19 | 72.7 |
| 137 | E41R | 39 | 72.7 |
| 138 | I154L | 152 | 72.7 |
| 139 | K224* | 222 | 72.6 |
| 140 | E41F | 39 | 72.6 |
| 141 | N61R | 59 | 72.5 |
| 142 | A218H | 216 | 72.5 |
| 143 | A42C | 40 | 72.5 |
| 144 | K203M | 201 | 72.4 |
| 145 | Q86E | 84 | 72.4 |
| 146 | Y157A | 155 | 72.4 |
| 147 | E41A | 39 | 72.3 |
| 148 | K98D | 96 | 72.3 |
| 149 | T64N | 62 | 72.3 |
| 150 | S5G | 3 | 72.3 |
| 151 | WT | N/A | 72.3 |
| 152 | N61H | 59 | 72.3 |
| 153 | S3M | 1 | 72.2 |
| 154 | H50D | 48 | 72.2 |
| 155 | L150P | 148 | 72.1 |
| 156 | T188R | 186 | 72.1 |
| 157 | R168V | 166 | 72.1 |
| 158 | I223L | 221 | 72.1 |
| 159 | G171D | 169 | 72.1 |
| 160 | Q86K | 84 | 72.1 |
| 161 | F104L | 102 | 72.1 |
| 162 | A218F | 216 | 72.0 |
| 163 | M24N | 22 | 71.9 |
| 164 | M220V | 218 | 71.9 |
| 165 | S5C | 3 | 71.9 |
| 166 | E196D | 194 | 71.9 |
| 167 | E41C | 39 | 71.8 |
| 168 | H7S | 5 | 71.8 |
| 169 | H7R | 5 | 71.8 |
| 170 | G121C | 119 | 71.6 |
| 171 | M193V | 191 | 71.6 |
| 172 | A9K | 7 | 71.6 |
| 173 | K134D | 132 | 71.6 |
| 174 | K224L | 222 | 71.6 |
| 175 | I223F | 221 | 71.6 |
| 176 | I223Y | 221 | 71.6 |
| 177 | I223V | 221 | 71.6 |
| 178 | I223K | 221 | 71.6 |
| 179 | K98N | 96 | 71.4 |
| 180 | M220I | 218 | 71.4 |
| 181 | M220L | 218 | 71.3 |
| 182 | V97I | 95 | 71.2 |
| 183 | Y157R | 155 | 71.2 |
| 184 | G8P | 6 | 71.2 |
| 185 | V97F | 95 | 71.1 |
| 186 | K153C | 151 | 71.0 |
| 187 | A106V | 104 | 71.0 |
| 188 | E192D | 190 | 71.0 |
| 189 | P53C | 51 | 70.9 |
| 190 | T144K | 142 | 70.7 |
| 191 | E129P | 127 | 70.6 |
| 192 | I120V | 118 | 70.5 |
| 193 | H143K | 141 | 70.4 |
| 194 | W184K | 182 | 70.4 |
| 195 | Q86A | 84 | 70.4 |
| 196 | S142A | 140 | 70.3 |
| 197 | E126C | 124 | 70.3 |
| 198 | D114N | 112 | 70.3 |
| 199 | K85I | 83 | 70.2 |
| 200 | F82Y | 80 | 70.1 |
| 201 | G54C | 52 | 70.1 |
| 202 | V77I | 75 | 70.1 |
| 203 | K195C | 193 | 70.1 |
| 204 | V97Y | 95 | 70.1 |
| 205 | P131L | 129 | 70.1 |
| 206 | Y74F | 72 | 70.0 |
| 207 | Y102F | 100 | 69.9 |
| 208 | R81V | 79 | 69.9 |
| 209 | E129C | 127 | 69.8 |
| 210 | Y69V | 67 | 69.8 |
| 211 | N116G | 114 | 69.8 |
| 212 | S68V | 66 | 69.8 |
| 213 | L123M | 121 | 69.8 |
| 214 | S101L | 99 | 69.8 |
| 215 | S101Y | 99 | 69.7 |
| 216 | E192G | 190 | 69.7 |
| 217 | V76E | 74 | 69.7 |
| 218 | R81I | 79 | 69.6 |
| 219 | D114E | 112 | 69.6 |
| 220 | I154C | 152 | 69.5 |
| 221 | K134I | 132 | 69.5 |
| 222 | Q86G | 84 | 69.5 |
| 223 | E129V | 127 | 69.4 |
| 224 | V132E | 130 | 69.3 |
| 225 | I80K | 78 | 69.3 |
| 226 | E83Y | 81 | 69.2 |
| 227 | A91S | 89 | 69.2 |
| 228 | A111Q | 109 | 69.2 |
| 229 | S73C | 71 | 69.2 |
| 230 | H107F | 105 | 69.0 |
| 231 | E129L | 127 | 69.0 |
| 232 | K135V | 133 | 68.9 |
| 233 | D114K | 112 | 68.9 |
| 234 | K113E | 111 | 68.8 |
| 235 | Y69I | 67 | 68.7 |
| 236 | T96M | 94 | 68.6 |
| 237 | K135M | 133 | 68.6 |
| 238 | V119F | 117 | 68.6 |
| 239 | K135I | 133 | 68.5 |
| 240 | E146C | 144 | 68.5 |
| 241 | N139K | 137 | 68.5 |
| 242 | I80E | 78 | 68.3 |
| 243 | Y69M | 67 | 68.2 |
| 244 | G127D | 125 | 68.1 |
| 245 | A91T | 89 | 68.0 |
| 246 | D112S | 110 | 67.9 |
| 247 | G127A | 125 | 67.7 |
| 248 | T96L | 94 | 67.7 |
| 249 | G127S | 125 | 67.6 |
| 250 | F124Y | 122 | 67.5 |
| 251 | A91C | 89 | 67.4 |
| 252 | S142D | 140 | 67.4 |
| 253 | K125E | 123 | 67.4 |
| 254 | A91Y | 89 | 67.3 |
| 255 | E70C | 68 | 66.9 |
| 256 | K85E | 83 | 65.7 |
| 257 | A91E | 89 | 65.6 |
| 258 | Y74E | 72 | 65.2 |
| 259 | Y74C | 72 | 64.1 |
| 260 | D78L | 76 | 64.1 |
| 261 | P141C | 139 | 52.0 |

TABLE 6

| | Carbonic | Corresponding | |
|---|---|---|---|
| | Anhydrase | position in SEQ | |
| Entry | Alteration | ID NO: 1 | Tm2 |
| 1 | E41R | 39 | 98.9 |
| 2 | E41K | 39 | 97.4 |
| 3 | R168F | 166 | 96.7 |
| 4 | E41L | 39 | 96.7 |
| 5 | E41N | 39 | 96.3 |
| 6 | E41S | 39 | 95.9 |
| 7 | E41M | 39 | 95.7 |
| 8 | E41A | 39 | 95.3 |
| 9 | E41F | 39 | 95.1 |
| 10 | I223L | 221 | 95.1 |
| 11 | D43G | 41 | 95.0 |
| 12 | I223K | 221 | 94.1 |
| 13 | T10C | 8 | 93.5 |
| 14 | P53S | 51 | 92.8 |
| 15 | S52K | 50 | 92.8 |
| 16 | P53T | 51 | 92.6 |
| 17 | M220L | 218 | 92.6 |
| 18 | K134I | 132 | 92.5 |
| 19 | E129P | 127 | 92.4 |
| 20 | A55G | 53 | 92.4 |
| 21 | E129V | 127 | 92.4 |
| 22 | E190V | 188 | 92.3 |
| 23 | T56K | 54 | 92.3 |
| 24 | K149E | 147 | 92.3 |
| 25 | N61S | 59 | 92.3 |
| 26 | S68F | 66 | 92.2 |
| 27 | H63Y | 61 | 92.2 |
| 28 | M191I | 189 | 92.2 |
| 29 | S57H | 55 | 92.1 |
| 30 | G127S | 125 | 92.1 |
| 31 | R163N | 161 | 92.1 |
| 32 | G127A | 125 | 92.1 |
| 33 | H152V | 150 | 92.0 |
| 34 | S68W | 66 | 92.0 |
| 35 | G127D | 125 | 91.9 |
| 36 | H50S | 48 | 91.9 |
| 37 | T175V | 173 | 91.9 |
| 38 | K66I | 64 | 91.9 |
| 39 | N61T | 59 | 91.8 |
| 40 | K165T | 163 | 91.8 |
| 41 | M220I | 218 | 91.8 |
| 42 | K165R | 163 | 91.8 |
| 43 | K134D | 132 | 91.8 |
| 44 | I223Y | 221 | 91.8 |
| 45 | K203M | 201 | 91.8 |
| 46 | Y167F | 165 | 91.7 |
| 47 | E129L | 127 | 91.7 |
| 48 | V132E | 130 | 91.7 |
| 49 | K153E | 151 | 91.7 |
| 50 | I154L | 152 | 91.7 |
| 51 | A151D | 149 | 91.7 |
| 52 | K48Y | 46 | 91.7 |
| 53 | A151L | 149 | 91.7 |
| 54 | T189I | 187 | 91.6 |
| 55 | K203I | 201 | 91.6 |
| 56 | N61L | 59 | 91.6 |
| 57 | K153C | 151 | 91.5 |
| 58 | S68G | 66 | 91.5 |
| 59 | A151H | 149 | 91.5 |
| 60 | I223F | 221 | 91.4 |
| 61 | T209N | 207 | 91.4 |
| 62 | T175I | 173 | 91.4 |
| 63 | N61I | 59 | 91.4 |
| 64 | S194C | 192 | 91.4 |
| 65 | E196D | 194 | 91.4 |
| 66 | K19S | 17 | 91.4 |
| 67 | S3M | 1 | 91.4 |
| 68 | A151P | 149 | 91.4 |
| 69 | K149M | 147 | 91.3 |
| 70 | S179W | 177 | 91.3 |
| 71 | P131L | 129 | 91.3 |
| 72 | E190P | 188 | 91.3 |
| 73 | H143K | 141 | 91.3 |
| 74 | H50D | 48 | 91.3 |
| 75 | WT | N/A | 91.2 |
| 76 | K149I | 147 | 91.2 |
| 77 | E41G | 39 | 91.2 |
| 78 | H50V | 48 | 91.2 |
| 79 | I154C | 152 | 91.2 |
| 80 | I223V | 221 | 91.1 |
| 81 | T189V | 187 | 91.1 |
| 82 | V76E | 74 | 91.1 |
| 83 | H152S | 150 | 91.1 |
| 84 | A9K | 7 | 91.1 |
| 85 | H50R | 48 | 91.0 |
| 86 | K200L | 198 | 91.0 |
| 87 | H7R | 5 | 91.0 |
| 88 | M191L | 189 | 91.0 |
| 89 | L59V | 57 | 91.0 |
| 90 | D158L | 156 | 91.0 |
| 91 | H7S | 5 | 91.0 |
| 92 | K200F | 198 | 91.0 |
| 93 | S52P | 50 | 91.0 |
| 94 | E45P | 43 | 91.0 |
| 95 | N61V | 59 | 91.0 |
| 96 | I120V | 118 | 90.9 |
| 97 | S68L | 66 | 90.9 |
| 98 | S68I | 66 | 90.9 |
| 99 | D208P | 206 | 90.9 |
| 100 | T188K | 186 | 90.9 |
| 101 | S5G | 3 | 90.8 |
| 102 | E192D | 190 | 90.8 |
| 103 | K200Y | 198 | 90.8 |
| 104 | Y157A | 155 | 90.8 |
| 105 | K66Q | 64 | 90.7 |
| 106 | T188R | 186 | 90.7 |
| 107 | T175W | 173 | 90.7 |
| 108 | S68Y | 66 | 90.7 |
| 109 | L150P | 148 | 90.6 |
| 110 | V213T | 211 | 90.6 |
| 111 | A55P | 53 | 90.6 |
| 112 | S179M | 177 | 90.6 |
| 113 | V77I | 75 | 90.6 |
| 114 | G54V | 52 | 90.6 |
| 115 | N61H | 59 | 90.6 |
| 116 | S3D | 1 | 90.6 |
| 117 | S142D | 140 | 90.6 |
| 118 | H50Y | 48 | 90.6 |
| 119 | Y74F | 72 | 90.6 |
| 120 | M191V | 189 | 90.6 |
| 121 | N61R | 59 | 90.5 |
| 122 | K125E | 123 | 90.5 |
| 123 | E192G | 190 | 90.5 |
| 124 | T56A | 54 | 90.4 |
| 125 | S57F | 55 | 90.4 |
| 126 | K153P | 151 | 90.4 |
| 127 | K195C | 193 | 90.3 |
| 128 | D20K | 18 | 90.3 |
| 129 | T188L | 186 | 90.3 |
| 130 | D20E | 18 | 90.2 |
| 131 | N61W | 59 | 90.2 |
| 132 | T144K | 142 | 90.2 |
| 133 | E83Y | 81 | 90.2 |
| 134 | F124Y | 122 | 90.2 |
| 135 | A91E | 89 | 90.1 |
| 136 | M205I | 203 | 90.1 |
| 137 | K164M | 162 | 90.1 |
| 138 | L123M | 121 | 90.1 |
| 139 | K85E | 83 | 90.1 |
| 140 | R81V | 79 | 90.0 |
| 141 | M187F | 185 | 90.0 |
| 142 | K165E | 163 | 89.9 |
| 143 | S142A | 140 | 89.9 |
| 144 | K85I | 83 | 89.9 |
| 145 | D158M | 156 | 89.9 |
| 146 | Y102F | 100 | 89.9 |
| 147 | H63F | 61 | 89.9 |
| 148 | S179Y | 177 | 89.9 |
| 149 | T96M | 94 | 89.8 |
| 150 | N60D | 58 | 89.8 |
| 151 | D114E | 112 | 89.8 |
| 152 | K135V | 133 | 89.8 |
| 153 | T188I | 186 | 89.8 |
| 154 | R81I | 79 | 89.7 |
| 155 | S170N | 168 | 89.7 |
| 156 | M220V | 218 | 89.6 |
| 157 | Y74E | 72 | 89.6 |
| 158 | M205L | 203 | 89.6 |
| 159 | S68V | 66 | 89.6 |
| 160 | S101L | 99 | 89.5 |
| 161 | K164L | 162 | 89.5 |
| 162 | T96L | 94 | 89.4 |
| 163 | S179F | 177 | 89.4 |
| 164 | K164F | 162 | 89.4 |
| 165 | N61D | 59 | 89.2 |
| 166 | D114N | 112 | 89.1 |
| 167 | T64N | 62 | 89.0 |
| 168 | K113E | 111 | 89.0 |
| 169 | D20P | 18 | 89.0 |
| 170 | K165L | 163 | 88.9 |
| 171 | K135I | 133 | 88.9 |
| 172 | Q86E | 84 | 88.7 |
| 173 | N116G | 114 | 88.7 |
| 174 | V119F | 117 | 88.7 |
| 175 | D114K | 112 | 88.7 |
| 176 | T64M | 62 | 88.6 |
| 177 | F104L | 102 | 88.5 |
| 178 | A111Q | 109 | 88.5 |
| 179 | Y157R | 155 | 88.3 |
| 180 | E45H | 43 | 88.3 |
| 181 | V97F | 95 | 88.3 |
| 182 | S73C | 71 | 88.2 |
| 183 | V97I | 95 | 88.2 |
| 184 | V97Y | 95 | 88.1 |
| 185 | Y69V | 67 | 88.0 |
| 186 | E45A | 43 | 87.8 |
| 187 | N116P | 114 | 87.8 |
| 188 | I80E | 78 | 87.5 |
| 189 | A156P | 154 | 87.5 |
| 190 | G121A | 119 | 87.4 |
| 191 | K135M | 133 | 87.4 |
| 192 | H14L | 12 | 87.2 |
| 193 | A91S | 89 | 87.1 |
| 194 | Y69M | 67 | 87.1 |
| 195 | Y69I | 67 | 86.8 |
| 196 | A91T | 89 | 86.8 |
| 197 | A106V | 104 | 86.8 |
| 198 | K98N | 96 | 86.2 |
| 199 | D112S | 110 | 86.2 |
| 200 | E45Y | 43 | 86.2 |
| 201 | T64W | 62 | 86.2 |
| 202 | A91Y | 89 | 86.0 |
| 203 | I80K | 78 | 85.6 |
| 204 | S170A | 162 | 85.3 |
| 205 | K224L | 222 | 84.9 |
| 206 | L84P | 82 | 84.5 |
| 207 | I23A | 21 | 84.3 |
| 208 | S37D | 35 | 83.9 |
| 209 | E180S | 178 | 81.9 |
| 210 | N210L | 208 | 80.7 |
| 211 | S170G | 168 | 79.5 |

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

The invention is further defined by the following numbered paragraphs:
Paragraph [1]. A carbonic anhydrase variant, comprising a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1, wherein the variant has at least 65%, e.g., at least 70%, at least 75%, at least 80%, at least 85%, at least 90%, at least 95%, at least 96%, at least 97%, at least 98%, or at least 99% sequence identity, but less than 100% sequence identity, to the polypeptide of SEQ ID NO: 1, SEQ ID NO: 2, SEQ ID NO: 3, or SEQ ID NO: 4, or the mature polypeptide sequence thereof, wherein the variant optionally comprises an extension of one or more amino acids at the N-terminal and/or C-terminal ends or a truncation of one or more amino acids at the N-terminal and/or C-terminal ends and wherein the variant has carbonic anhydrase activity.
Paragraph [2]. The variant of paragraph [1], which comprises a substitution at a position corresponding to position 1 of the polypeptide of SEQ ID NO: 1, preferably with Met or Asp.
Paragraph [3]. The variant of paragraph [1] or [2], which comprises a substitution at a position corresponding to position 3 of the polypeptide of SEQ ID NO: 1, preferably with Gly or Cys.
Paragraph [4]. The variant of any one of the previous paragraphs, which comprises a substitution at a position corresponding to position 5 of the polypeptide of SEQ ID NO: 1, preferably with Arg or Ser.
Paragraph [5]. The variant of any one of the previous paragraphs, which comprises a substitution at a position corresponding to position 6 of the polypeptide of SEQ ID NO: 1, preferably with Pro.
Paragraph [6]. The variant of any one of the previous paragraphs, which comprises a substitution at a position corresponding to position 7 of the polypeptide of SEQ ID NO: 1, preferably with Lys, Asp, His, Gln or Arg.
Paragraph [7]. The variant of any one of the previous paragraphs, which comprises a substitution at a position corresponding to position 8 of the polypeptide of SEQ ID NO: 1, preferably with Cys or Asn.
Paragraph [8]. The variant of any one of the previous paragraphs, which comprises of a substitution at a position corresponding to position 12 of the polypeptide of SEQ ID NO: 1, preferably with Leu.
Paragraph [9]. The variant of any one of the previous paragraphs, which comprises a substitution at a position corresponding to position 17 of the polypeptide of SEQ ID NO: 1, preferably with Ser.
Paragraph [10]. The variant of any one of the previous paragraphs, which comprises a substitution at a position corresponding to position 18 of the polypeptide of SEQ ID NO: 1, preferably with Lys, Glu, Arg, Gly, Ser or Pro.
Paragraph [11]. The variant of any one of the previous paragraphs, which comprises a substitution at a position corresponding to position 19 of the polypeptide of SEQ ID NO: 1, preferably with Pro.
Paragraph [12]. The variant of any one of the previous paragraphs, which comprises a substitution at a position corresponding to position 20 of the polypeptide of SEQ ID NO: 1, preferably with Phe.
Paragraph [13]. The variant of any one of the previous paragraphs, which comprises a substitution at a position corresponding to position 21 of the polypeptide of SEQ ID NO: 1, preferably with Ala.
Paragraph [14]. The variant of any one of the previous paragraphs, which comprises a substitution at a position corresponding to position 22 of the polypeptide of SEQ ID NO: 1, preferably with Pro, Phe, Trp, Tyr or Asn.
Paragraph [15]. The variant of any one of the previous paragraphs, which comprises a substitution at a position corresponding to position 25 of the polypeptide of SEQ ID NO: 1, preferably with Thr.
Paragraph [16]. The variant of any one of the previous paragraphs, which comprises a substitution at a position corresponding to position 27 of the polypeptide of SEQ ID NO: 1, preferably with Thr or Tyr.
Paragraph [17]. The variant of any one of the previous paragraphs, which comprises a substitution at a position corresponding to position 28 of the polypeptide of SEQ ID NO: 1, preferably with Arg, Glu, Leu, Gln, Met, Ser, Tyr, Phe, Lys, Thr, Phe, Asp or His.
Paragraph [18]. The variant of any one of the previous paragraphs, which comprises a substitution at a position corresponding to position 35 of the polypeptide of SEQ ID NO: 1, preferably with Asp.
Paragraph [19]. The variant of any one of the previous paragraphs, which comprises a substitution at a position corresponding to position 36 of the polypeptide of SEQ ID NO: 1, preferably with Asp, Leu, Ser, Asn or Ile.
Paragraph [20]. The variant of any one of the previous paragraphs, which comprises a substitution at a position corresponding to position 37 of the polypeptide of SEQ ID NO: 1, preferably with Val.
Paragraph [21]. The variant of any one of the previous paragraphs, which comprises a substitution at a position corresponding to position 38 of the polypeptide of SEQ ID NO: 1, preferably with Cys.
Paragraph [22]. The variant of any one of the previous paragraphs, which comprises a deletion or substitution at a position corresponding to position 39 of the polypeptide of SEQ ID NO: 1, preferably a substitution with Ala, Cys, Glu, Phe, Gly, Lys, Leu, Met, Asn, Pro, Arg, Ser, Trp or Tyr.
Paragraph [23]. The variant of any one of the previous paragraphs, which comprises a substitution at a position corresponding to position 40 of the polypeptide of SEQ ID NO: 1, preferably with Cys.
Paragraph [24]. The variant of any one of the previous paragraphs, which comprises a substitution at a position corresponding to position 41 of the polypeptide of SEQ ID NO: 1, preferably with Cys, Ala, Met, Asn, Ser, Asp, Leu or Gly.
Paragraph [25]. The variant of any one of the previous paragraphs, which comprises a substitution at a position corresponding to position 43 of the polypeptide of SEQ ID NO: 1, preferably with Ala, His, Phe, Pro or Tyr.
Paragraph [26]. The variant of any one of the previous paragraphs, which comprises a substitution at a position corresponding to position 46 of the polypeptide of SEQ ID NO: 1, preferably with Tyr.
Paragraph [27]. The variant of any one of the previous paragraphs, which comprises a substitution at a position corresponding to position 48 of the polypeptide of SEQ ID NO: 1, preferably with Asp, Ser,

### Asn, Tyr, Val, Phe or Arg.

Paragraph [28]. The variant of any one of the previous paragraphs, which comprises a substitution at a position corresponding to position 49 of the polypeptide of SEQ ID NO: 1, preferably with Cys.
Paragraph [29]. The variant of any one of the previous paragraphs, which comprises a substitution at a position corresponding to position 50 of the polypeptide of SEQ ID NO: 1, preferably with Lys, Tyr or Pro.
Paragraph [30]. The variant of any one of the previous paragraphs, which comprises a substitution at a position corresponding to position 51 of the polypeptide of SEQ ID NO: 1, preferably with Pro, Ser, Thr or Cys.
Paragraph [31]. The variant of any one of the previous paragraphs, which comprises a substitution at a position corresponding to position 52 of the polypeptide of SEQ ID NO: 1, preferably with Val or Cys.
Paragraph [32]. The variant of any one of the previous paragraphs, which comprises a substitution at a position corresponding to position 53 of the polypeptide of SEQ ID NO: 1, preferably with Gly or Pro.
Paragraph [33]. The variant of any one of the previous paragraphs, which comprises a substitution at a position corresponding to position 54 of the polypeptide of SEQ ID NO: 1, preferably with Lys.
Paragraph [34]. The variant of any one of the previous paragraphs, which comprises a substitution at a position corresponding to position 55 of the polypeptide of SEQ ID NO: 1, preferably with His, Tyr or Phe.
Paragraph [35]. The variant of any one of the previous paragraphs, which comprises a substitution at a position corresponding to position 57 of the polypeptide of SEQ ID NO: 1, preferably with Val.
Paragraph [36]. The variant of any one of the previous paragraphs, which comprises a substitution at a position corresponding to position 58 of the polypeptide of SEQ ID NO: 1, preferably with Asp.
Paragraph [37]. The variant of any one of the previous paragraphs, which comprises a substitution at a position corresponding to position 59 of the polypeptide of SEQ ID NO: 1, preferably with Asp, Trp, Ile, Arg, His, Ser, Thr, Val or Leu.
Paragraph [38]. The variant of any one of the previous paragraphs, which comprises a substitution at a position corresponding to position 60 of the polypeptide of SEQ ID NO: 1, preferably with Asp.
Paragraph [39]. The variant of any one of the previous paragraphs, which comprises a substitution at a position corresponding to position 61 of the polypeptide of SEQ ID NO: 1, preferably with Tyr, Trp or Phe.
Paragraph [40]. The variant of any one of the previous paragraphs, which comprises a substitution at a position corresponding to position 62 of the polypeptide of SEQ ID NO: 1, preferably with Asn, Tyr,

### Trp, Phe or Met.

Paragraph [41]. The variant of any one of the previous paragraphs, which comprises a substitution at a position corresponding to position 64 of the polypeptide of SEQ ID NO: 1, preferably with Ile, Gln, or Leu.
Paragraph [42]. The variant of any one of the previous paragraphs, which comprises a substitution at a position corresponding to position 66 of the polypeptide of SEQ ID NO: 1, preferably with Leu, Ile, Phe, Met, His, Val, Gln, Tyr, Trp or Gly.
Paragraph [43]. The variant of any one of the previous paragraphs, which comprises a substitution at a position corresponding to position 67 of the polypeptide of SEQ ID NO: 1, preferably with Met, Ile, Phe, Trp or Val.
Paragraph [44]. The variant of any one of the previous paragraphs, which comprises a substitution at a position corresponding to position 68 of the polypeptide of SEQ ID NO: 1, preferably with Cys, His, Asp, Arg or Thr.
Paragraph [45]. The variant of any one of the previous paragraphs, which comprises a substitution at a position corresponding to position 71 of the polypeptide of SEQ ID NO: 1, preferably with Ala or Cys.
Paragraph [46]. The variant of any one of the previous paragraphs, which comprises a substitution at a position corresponding to position 72 of the polypeptide of SEQ ID NO: 1, preferably with Phe, Cys,

### Ser, Thr or Glu.

Paragraph [47]. The variant of any one of the previous paragraphs, which comprises a substitution at a position corresponding to position 74 of the polypeptide of SEQ ID NO: 1, preferably with Glu.
Paragraph [48]. The variant of any one of the previous paragraphs, which comprises a substitution at a position corresponding to position 75 of the polypeptide of SEQ ID NO: 1, preferably with Ile or Leu.
Paragraph [49]. The variant of any one of the previous paragraphs, which comprises a substitution at a position corresponding to position 76 of the polypeptide of SEQ ID NO: 1, preferably with Asn, Lys or

### Leu.

Paragraph [50]. The variant of any one of the previous paragraphs, which comprises a substitution at a position corresponding to position 77 of the polypeptide of SEQ ID NO: 1, preferably with Asp.
Paragraph [51]. The variant of any one of the previous paragraphs, which comprises a substitution at a position corresponding to position 78 of the polypeptide of SEQ ID NO: 1, preferably with Val, Glu or Lys.
Paragraph [52]. The variant of any one of the previous paragraphs, which comprises a substitution at a position corresponding to position 79 of the polypeptide of SEQ ID NO: 1, preferably with Ile or Val.
Paragraph [53]. The variant of any one of the previous paragraphs, which comprises a substitution at a position corresponding to position 80 of the polypeptide of SEQ ID NO: 1, preferably with Ile or Tyr
Paragraph [54]. The variant of any one of the previous paragraphs, which comprises a substitution at a position corresponding to position 81 of the polypeptide of SEQ ID NO: 1, preferably with Tyr.
Paragraph [55]. The variant of any one of the previous paragraphs, which comprises a substitution at a position corresponding to position 82 of the polypeptide of SEQ ID NO: 1, preferably with Pro or Phe.
Paragraph [56]. The variant of any one of the previous paragraphs, which comprises a substitution at a position corresponding to position 83 of the polypeptide of SEQ ID NO: 1, preferably with Glu or Ile.
Paragraph [57]. The variant of any one of the previous paragraphs, which comprises a substitution at a position corresponding to position 84 of the polypeptide of SEQ ID NO: 1, preferably with Phe, Ala, Lys, Gly, Glu, Ile, Met, Leu, Trp or Tyr.
Paragraph [58]. The variant of any one of the previous paragraphs, which comprises a substitution at a position corresponding to position 85 of the polypeptide of SEQ ID NO: 1, preferably with Cys.
Paragraph [59]. The variant of any one of the previous paragraphs, which comprises a substitution at a position corresponding to position 86 of the polypeptide of SEQ ID NO: 1, preferably with Ile, Tyr or Trp.
Paragraph [60]. The variant of any one of the previous paragraphs, which comprises a substitution at a position corresponding to position 87 of the polypeptide of SEQ ID NO: 1, preferably with Trp or Leu.
Paragraph [61]. The variant of any one of the previous paragraphs, which comprises a substitution at a position corresponding to position 88 of the polypeptide of SEQ ID NO: 1, preferably with Tyr.
Paragraph [62]. The variant of any one of the previous paragraphs, which comprises a substitution at a position corresponding to position 89 of the polypeptide of SEQ ID NO: 1, preferably with Glu, Tyr, Ser, Thr, Cys, Val or Phe.
Paragraph [63]. The variant of any one of the previous paragraphs, which comprises a substitution at a position corresponding to position 91 of the polypeptide of SEQ ID NO: 1, preferably with His.
Paragraph [64]. The variant of any one of the previous paragraphs, which comprises a substitution at a position corresponding to position 94 of the polypeptide of SEQ ID NO: 1, preferably with Leu, Met, or Thr.
Paragraph [65]. The variant of any one of the previous paragraphs, which comprises a substitution at a position corresponding to position 95 of the polypeptide of SEQ ID NO: 1, preferably with Val, Ile, Phe, or Tyr.
Paragraph [66]. The variant of any one of the previous paragraphs, which comprises a substitution at a position corresponding to position 96 of the polypeptide of SEQ ID NO: 1, preferably with Asn or Asp.
Paragraph [67]. The variant of any one of the previous paragraphs, which comprises a substitution at a position corresponding to position 98 of the polypeptide of SEQ ID NO: 1, preferably with Lys.
Paragraph [68]. The variant of any one of the previous paragraphs, which comprises a substitution at a position corresponding to position 99 of the polypeptide of SEQ ID NO: 1, preferably with Tyr, Pro or Leu.
Paragraph [69]. The variant of any one of the previous paragraphs, which comprises a substitution at a position corresponding to position 100 of the polypeptide of SEQ ID NO: 1, preferably with Phe.
Paragraph [70]. The variant of any one of the previous paragraphs, which comprises a substitution at a position corresponding to position 102 of the polypeptide of SEQ ID NO: 1, preferably with Leu or Met.
Paragraph [71]. The variant of any one of the previous paragraphs, which comprises a substitution at a position corresponding to position 104 of the polypeptide of SEQ ID NO: 1, preferably with Leu, Cys, Met or Val.
Paragraph [72]. The variant of any one of the previous paragraphs, which comprises a substitution at a position corresponding to position 105 of the polypeptide of SEQ ID NO: 1, preferably with Phe.
Paragraph [73]. The variant of any one of the previous paragraphs, which comprises a substitution at a position corresponding to position 106 of the polypeptide of SEQ ID NO: 1, preferably with Cys.
Paragraph [74]. The variant of any one of the previous paragraphs, which comprises a substitution at a position corresponding to position 109 of the polypeptide of SEQ ID NO: 1, preferably with Gln.
Paragraph [75]. The variant of any one of the previous paragraphs, which comprises a substitution at a position corresponding to position 110 of the polypeptide of SEQ ID NO: 1, preferably with Ser.
Paragraph [76]. The variant of any one of the previous paragraphs, which comprises a substitution at a position corresponding to position 111 of the polypeptide of SEQ ID NO: 1, preferably with Glu.
Paragraph [77]. The variant of any one of the previous paragraphs, which comprises a substitution at a position corresponding to position 112 of the polypeptide of SEQ ID NO: 1, preferably with Glu, Asp, Lys, or Asn.
Paragraph [78]. The variant of any one of the previous paragraphs, which comprises a substitution at a position corresponding to position 114 of the polypeptide of SEQ ID NO: 1, preferably with Leu, Arg, Gly or Pro.
Paragraph [79]. The variant of any one of the previous paragraphs, which comprises a substitution at a position corresponding to position 115 of the polypeptide of SEQ ID NO: 1, preferably with Gln, Trp, Tyr or Ile.
Paragraph [80]. The variant of any one of the previous paragraphs, which comprises a substitution at a position corresponding to position 117 of the polypeptide of SEQ ID NO: 1, preferably with Phe.
Paragraph [81]. The variant of any one of the previous paragraphs, which comprises a substitution at a position corresponding to position 118 of the polypeptide of SEQ ID NO: 1, preferably with Val or Leu.
Paragraph [82]. The variant of any one of the previous paragraphs, which comprises a substitution at a position corresponding to position 119 of the polypeptide of SEQ ID NO: 1, preferably with Ala, Ile, Cys, Thr, Ser or Val.
Paragraph [83]. The variant of any one of the previous paragraphs, which comprises a substitution at a position corresponding to position 121 of the polypeptide of SEQ ID NO: 1, preferably with Met or

### Leu.

Paragraph [84]. The variant of any one of the previous paragraphs, which comprises a substitution at a position corresponding to position 122 of the polypeptide of SEQ ID NO: 1, preferably with Tyr.
Paragraph [85]. The variant of any one of the previous paragraphs, which comprises a substitution at a position corresponding to position 123 of the polypeptide of SEQ ID NO: 1, preferably with Glu.
Paragraph [86]. The variant of any one of the previous paragraphs, which comprises a substitution at a position corresponding to position 124 of the polypeptide of SEQ ID NO: 1, preferably with Cys.
Paragraph [87]. The variant of any one of the previous paragraphs, which comprises a substitution at a position corresponding to position 125 of the polypeptide of SEQ ID NO: 1, preferably with Ser, Ala, or Asp.
Paragraph [88]. The variant of any one of the previous paragraphs, which comprises a substitution at a position corresponding to position 126 of the polypeptide of SEQ ID NO: 1, preferably with Lys.
Paragraph [89]. The variant of any one of the previous paragraphs, which comprises a substitution at a position corresponding to position 127 of the polypeptide of SEQ ID NO: 1, preferably with Pro, Leu, Cys, Met, Lys, Gln, Thr, Ala, Arg or Val.
Paragraph [90]. The variant of any one of the previous paragraphs, which comprises a substitution at a position corresponding to position 129 of the polypeptide of SEQ ID NO: 1, preferably with Leu.
Paragraph [91]. The variant of any one of the previous paragraphs, which comprises a substitution at a position corresponding to position 130 of the polypeptide of SEQ ID NO: 1, preferably with Glu.
Paragraph [92]. The variant of any one of the previous paragraphs, which comprises a substitution at a position corresponding to position 132 of the polypeptide of SEQ ID NO: 1, preferably with Ile or Asp.
Paragraph [93]. The variant of any one of the previous paragraphs, which comprises a substitution at a position corresponding to position 133 of the polypeptide of SEQ ID NO: 1, preferably with Met, Ile, or Val.
Paragraph [94]. The variant of any one of the previous paragraphs, which comprises a substitution at a position corresponding to position 135 of the polypeptide of SEQ ID NO: 1, preferably with Leu.
Paragraph [95]. The variant of any one of the previous paragraphs, which comprises a substitution at a position corresponding to position 137 of the polypeptide of SEQ ID NO: 1, preferably with Lys.
Paragraph [96]. The variant of any one of the previous paragraphs, which comprises a substitution at a position corresponding to position 139 of the polypeptide of SEQ ID NO: 1, preferably with Cys.
Paragraph [97]. The variant of any one of the previous paragraphs, which comprises a substitution at a position corresponding to position 140 of the polypeptide of SEQ ID NO: 1, preferably with Ala or Asp.
Paragraph [98]. The variant of any one of the previous paragraphs, which comprises a substitution at a position corresponding to position 141 of the polypeptide of SEQ ID NO: 1, preferably with Lys, Thr, Glu or Gln.
Paragraph [99]. The variant of any one of the previous paragraphs, which comprises a substitution at a position corresponding to position 142 of the polypeptide of SEQ ID NO: 1, preferably with Lys.
Paragraph [100]. The variant of any one of the previous paragraphs, which comprises a substitution at a position corresponding to position 144 of the polypeptide of SEQ ID NO: 1, preferably with Cys.
Paragraph [101]. The variant of any one of the previous paragraphs, which comprises a substitution at a position corresponding to position 145 of the polypeptide of SEQ ID NO: 1, preferably with Tyr, Phe or Leu.
Paragraph [102]. The variant of any one of the previous paragraphs, which comprises a substitution at a position corresponding to position 146 of the polypeptide of SEQ ID NO: 1, preferably with Ala.
Paragraph [103]. The variant of any one of the previous paragraphs, which comprises a substitution at a position corresponding to position 147 of the polypeptide of SEQ ID NO: 1, preferably with Ile, Glu, or Met.
Paragraph [104]. The variant of any one of the previous paragraphs, which comprises a substitution at a position corresponding to position 148 of the polypeptide of SEQ ID NO: 1, preferably with Pro.
Paragraph [105]. The variant of any one of the previous paragraphs, which comprises a substitution at a position corresponding to position 149 of the polypeptide of SEQ ID NO: 1, preferably with Leu, Pro, His, or Asp.
Paragraph [106]. The variant of any one of the previous paragraphs, which comprises a substitution at a position corresponding to position 150 of the polypeptide of SEQ ID NO: 1, preferably with Val, Arg or Ser.
Paragraph [107]. The variant of any one of the previous paragraphs, which comprises a substitution at a position corresponding to position 151 of the polypeptide of SEQ ID NO: 1, preferably with Cys, Glu, or Pro.
Paragraph [108]. The variant of any one of the previous paragraphs, which comprises a substitution at a position corresponding to position 151 of the polypeptide of SEQ ID NO: 1, preferably with Cys or Leu.
Paragraph [109]. The variant of any one of the previous paragraphs, which comprises a substitution at a position corresponding to position 153 of the polypeptide of SEQ ID NO: 1, preferably with Asp.
Paragraph [110]. The variant of any one of the previous paragraphs, which comprises a substitution at a position corresponding to position 154 of the polypeptide of SEQ ID NO: 1, preferably with Pro, Val, Leu or Ile.
Paragraph [111]. The variant of any one of the previous paragraphs, which comprises a substitution at a position corresponding to position 155 of the polypeptide of SEQ ID NO: 1, preferably with Ala, Ile or Arg.
Paragraph [112]. The variant of any one of the previous paragraphs, which comprises a substitution at a position corresponding to position 156 of the polypeptide of SEQ ID NO: 1, preferably with Met, Asn, Gln or Leu.
Paragraph [113]. The variant of any one of the previous paragraphs, which comprises a substitution at a position corresponding to position 161 of the polypeptide of SEQ ID NO: 1, preferably with Asn.
Paragraph [114]. The variant of any one of the previous paragraphs, which comprises a substitution at a position corresponding to position 162 of the polypeptide of SEQ ID NO: 1, preferably with Phe, Met, or Leu.
Paragraph [115]. The variant of any one of the previous paragraphs, which comprises a substitution at a position corresponding to position 163 of the polypeptide of SEQ ID NO: 1, preferably with Lys, Thr, Leu, Arg, or Glu.
Paragraph [116]. The variant of any one of the previous paragraphs, which comprises a substitution at a position corresponding to position 165 of the polypeptide of SEQ ID NO: 1, preferably with Phe.
Paragraph [117]. The variant of any one of the previous paragraphs, which comprises a substitution at a position corresponding to position 166 of the polypeptide of SEQ ID NO: 1, preferably with Ala, Met, Lys, Glu, Phe, Val, Cys or Tyr.
Paragraph [118]. The variant of any one of the previous paragraphs, which comprises a substitution at a position corresponding to position 168 of the polypeptide of SEQ ID NO: 1, preferably with Asn, Cys, Ala, Asp, Glu, Leu, Pro, Gln, Thr, Val, Met, or Ile.
Paragraph [119]. The variant of any one of the previous paragraphs, which comprises a substitution at a position corresponding to position 169 of the polypeptide of SEQ ID NO: 1, preferably with Asp.
Paragraph [120]. The variant of any one of the previous paragraphs, which comprises a substitution at a position corresponding to position 171 of the polypeptide of SEQ ID NO: 1, preferably with Phe.
Paragraph [121]. The variant of any one of the previous paragraphs, which comprises a substitution at a position corresponding to position 173 of the polypeptide of SEQ ID NO: 1, preferably with Val, Trp or Ile.
Paragraph [122]. The variant of any one of the previous paragraphs, which comprises a substitution at a position corresponding to position 177 of the polypeptide of SEQ ID NO: 1, preferably with Tyr, Met, Trp or Phe.
Paragraph [123]. The variant of any one of the previous paragraphs, which comprises a substitution at a position corresponding to position 178 of the polypeptide of SEQ ID NO: 1, preferably with Ser, Pro, Thr or Gln.
Paragraph [124]. The variant of any one of the previous paragraphs, which comprises a substitution at a position corresponding to position 181 of the polypeptide of SEQ ID NO: 1, preferably with Ala, Thr, Ile, or Leu.
Paragraph [125]. The variant of any one of the previous paragraphs, which comprises a substitution at a position corresponding to position 182 of the polypeptide of SEQ ID NO: 1, preferably with Lys.
Paragraph [126]. The variant of any one of the previous paragraphs, which comprises a substitution at a position corresponding to position 185 of the polypeptide of SEQ ID NO: 1, preferably with Phe or Leu.
Paragraph [127]. The variant of any one of the previous paragraphs, which comprises a substitution at a position corresponding to position 186 of the polypeptide of SEQ ID NO: 1, preferably with Lys, Leu, Arg.
Paragraph [128]. The variant of any one of the previous paragraphs, which comprises a substitution at a position corresponding to position 187 of the polypeptide of SEQ ID NO: 1, preferably with Ile or Val.
Paragraph [129]. The variant of any one of the previous paragraphs, which comprises a substitution at a position corresponding to position 188 of the polypeptide of SEQ ID NO: 1, preferably with Val or Pro.
Paragraph [130]. The variant of any one of the previous paragraphs, which comprises a substitution at a position corresponding to position 189 of the polypeptide of SEQ ID NO: 1, preferably with Ile, Val, Leu.
Paragraph [131]. The variant of any one of the previous paragraphs, which comprises a substitution at a position corresponding to position 190 of the polypeptide of SEQ ID NO: 1, preferably with Gly or Asp.
Paragraph [132]. The variant of any one of the previous paragraphs, which comprises a substitution at a position corresponding to position 191 of the polypeptide of SEQ ID NO: 1, preferably with Val, Thr, Trp, His, Leu, Ala, Gly, Arg, Lys, Pro, Phe, Gln, Asn or Ile.
Paragraph [133]. The variant of any one of the previous paragraphs, which comprises a substitution at a position corresponding to position 192 of the polypeptide of SEQ ID NO: 1, preferably with Cys.
Paragraph [134]. The variant of any one of the previous paragraphs, which comprises a substitution at a position corresponding to position 193 of the polypeptide of SEQ ID NO: 1, preferably with Cys.
Paragraph [135]. The variant of any one of the previous paragraphs, which comprises a substitution at a position corresponding to position 194 of the polypeptide of SEQ ID NO: 1, preferably with Asp or Ala.
Paragraph [136]. The variant of any one of the previous paragraphs, which comprises a substitution at a position corresponding to position 197 of the polypeptide of SEQ ID NO: 1, preferably with Val.
Paragraph [137]. The variant of any one of the previous paragraphs, which comprises a substitution at a position corresponding to position 198 of the polypeptide of SEQ ID NO: 1, preferably with Tyr, Phe or Leu.
Paragraph [138]. The variant of any one of the previous paragraphs, which comprises a substitution at a position corresponding to position 200 of the polypeptide of SEQ ID NO: 1, preferably with Cys.
Paragraph [139]. The variant of any one of the previous paragraphs, which comprises a substitution at a position corresponding to position 201 of the polypeptide of SEQ ID NO: 1, preferably with Met or Ile.
Paragraph [140]. The variant of any one of the previous paragraphs, which comprises a substitution at a position corresponding to position 202 of the polypeptide of SEQ ID NO: 1, preferably with Leu, Val, Ala or Thr.
Paragraph [141]. The variant of any one of the previous paragraphs, which comprises a substitution at a position corresponding to position 203 of the polypeptide of SEQ ID NO: 1, preferably with Leu or Ile.
Paragraph [142]. The variant of any one of the previous paragraphs, which comprises a substitution at a position corresponding to position 205 of the polypeptide of SEQ ID NO: 1, preferably with Glu.
Paragraph [143]. The variant of any one of the previous paragraphs, which comprises a substitution at a position corresponding to position 206 of the polypeptide of SEQ ID NO: 1, preferably with Pro.
Paragraph [144]. The variant of any one of the previous paragraphs, which comprises a substitution at a position corresponding to position 207 of the polypeptide of SEQ ID NO: 1, preferably with Asn.
Paragraph [145]. The variant of any one of the previous paragraphs, which comprises a substitution at a position corresponding to position 208 of the polypeptide of SEQ ID NO: 1, preferably with Leu.
Paragraph [146]. The variant of any one of the previous paragraphs, which comprises a substitution at a position corresponding to position 211 of the polypeptide of SEQ ID NO: 1, preferably with Thr.
Paragraph [147]. The variant of any one of the previous paragraphs, which comprises a substitution at a position corresponding to position 214 of the polypeptide of SEQ ID NO: 1, preferably with Ile.
Paragraph [148]. The variant of any one of the previous paragraphs, which comprises a substitution at a position corresponding to position 215 of the polypeptide of SEQ ID NO: 1, preferably with Leu.
Paragraph [149]. The variant of any one of the previous paragraphs, which comprises a substitution at a position corresponding to position 216 of the polypeptide of SEQ ID NO: 1, preferably with His, Cys, Asn, Gly, Phe, or Pro.
Paragraph [150]. The variant of any one of the previous paragraphs, which comprises a substitution at a position corresponding to position 217 of the polypeptide of SEQ ID NO: 1, preferably with Asn.
Paragraph [151]. The variant of any one of the previous paragraphs, which comprises a substitution at a position corresponding to position 218 of the polypeptide of SEQ ID NO: 1, preferably with Lys, Val, Ile, Phe, Thr, Asn, Ser, Ala, Gly, Tyr or Leu.
Paragraph [152]. The variant of any one of the previous paragraphs, which comprises a substitution at a position corresponding to position 220 of the polypeptide of SEQ ID NO: 1, preferably with Leu.
Paragraph [153]. The variant of any one of the previous paragraphs, which comprises a substitution at a position corresponding to position 221 of the polypeptide of SEQ ID NO: 1, preferably with Arg, Leu, Ile, Phe, Tyr, Thr, Val or Lys.
Paragraph [154]. The variant of any one of the previous paragraphs, which comprises a substitution at a position corresponding to position 222 of the polypeptide of SEQ ID NO: 1, preferably with Leu.
Paragraph [155]. The variant of any one of the previous paragraphs, which comprises a deletion at a position corresponding to position 222 of the polypeptide of SEQ ID NO: 1.
Paragraph [156]. The variant of any one of the previous paragraphs, wherein the variant comprises or consists of a substitution at a position corresponding to position 18 of the polypeptide of SEQ ID NO: 1 (e.g., D18K, D18E, D18R, D18G, D18S or D18P), position 39 of the polypeptide of SEQ ID NO: 1 (e.g., D39A, D39C, D39E, D39F, D39G, D39K, D39L, D39M, D39N, D39P, D39R, D39S, D39W or D39Y), position 64 of the polypeptide of SEQ ID NO: 1 (e.g., K64I, K64Q or K64L), position 75 of the polypeptide of SEQ ID NO: 1 (e.g., V75I or V75L) position 186 of the polypeptide of SEQ ID NO: 1 (e.g., E186K, E186L, or E186R) and/or position 188 of the polypeptide of SEQ ID NO: 1 (e.g., E188V or E188P).
Paragraph [157]. The variant of any one of the previous paragraphs, wherein the variant comprises or consists of a substitution at a position corresponding to position 18 of the polypeptide of SEQ ID NO: 1 (e.g., D18K, D18E, D18R, D18G, D18S or D18P) and position 39 of the polypeptide of SEQ ID NO: 1 (e.g., D39A, D39C, D39E, D39F, D39G, D39K, D39L, D39M, D39N, D39P, D39R, D39S, D39W or D39Y).
Paragraph [158]. The variant of any one of the previous paragraphs, wherein the variant comprises or consists of a substitution at a position corresponding to position 18 of the polypeptide of SEQ ID NO: 1 (e.g., D18K, D18E, D18R, D18G, D18S or D18P) and position 64 of the polypeptide of SEQ ID NO: 1 (e.g., K64I, K64Q or K64L).
Paragraph [159]. The variant of any one of the previous paragraphs, wherein the variant comprises or consists of a substitution at a position corresponding to position 18 of the polypeptide of SEQ ID NO: 1 (e.g., D18K, D18E, D18R, D18G, D18S or D18P) and position 75 of the polypeptide of SEQ ID NO: 1 (e.g., V75I or V75L).
Paragraph [160]. The variant of any one of the previous paragraphs, wherein the variant comprises or consists of a substitution at a position corresponding to position 18 of the polypeptide of SEQ ID NO: 1 (e.g., D18K, D18E, D18R, D18G, D18S or D18P) and position 186 of the polypeptide of SEQ ID NO: 1 (e.g., E186K, E186L, or E186R).
Paragraph [161]. The variant of any one of the previous paragraphs, wherein the variant comprises or consists of a substitution at a position corresponding to position 18 of the polypeptide of SEQ ID NO: 1 (e.g., D18K, D18E, D18R, D18G, D18S or D18P) and position 188 of the polypeptide of SEQ ID NO: 1 (e.g., E188V or E188P).
Paragraph [162]. The variant of any one of the previous paragraphs, wherein the variant comprises or consists of a substitution at a position corresponding to position 39 of the polypeptide of SEQ ID NO: 1 (e.g., D39A, D39C, D39E, D39F, D39G, D39K, D39L, D39M, D39N, D39P, D39R, D39S, D39W or D39Y) and position 64 of the polypeptide of SEQ ID NO: 1 (e.g., K64I, K64Q or K64L).
Paragraph [163]. The variant of any one of the previous paragraphs, wherein the variant comprises or consists of a substitution at a position corresponding to position 39 of the polypeptide of SEQ ID NO: 1 (e.g., D39A, D39C, D39E, D39F, D39G, D39K, D39L, D39M, D39N, D39P, D39R, D39S, D39W or D39Y) and position 75 of the polypeptide of SEQ ID NO: 1 (e.g., V75I or V75L).
Paragraph [164]. The variant of any one of the previous paragraphs, wherein the variant comprises or consists of a substitution at a position corresponding to position 39 of the polypeptide of SEQ ID NO: 1 (e.g., D39A, D39C, D39E, D39F, D39G, D39K, D39L, D39M, D39N, D39P, D39R, D39S, D39W or D39Y) and position 186 of the polypeptide of SEQ ID NO: 1 (e.g., E186K, E186L, or E186R).
Paragraph [165]. The variant of any one of the previous paragraphs, wherein the variant comprises or consists of a substitution at a position corresponding to position 39 of the polypeptide of SEQ ID NO: 1 (e.g., D39A, D39C, D39E, D39F, D39G, D39K, D39L, D39M, D39N, D39P, D39R, D39S, D39W or D39Y) and position 188 of the polypeptide of SEQ ID NO: 1 (e.g., E188V or E188P).
Paragraph [166]. The variant of any one of the previous paragraphs, wherein the variant comprises or consists of a substitution at a position corresponding to position 64 of the polypeptide of SEQ ID NO: 1 (e.g., K64I, K64Q or K64L) and position 75 of the polypeptide of SEQ ID NO: 1 (e.g., V75I or V75L).
Paragraph [167]. The variant of any one of the previous paragraphs, wherein the variant comprises or consists of a substitution at a position corresponding to position 64 of the polypeptide of SEQ ID NO: 1 (e.g., K64I, K64Q or K64L) and position 186 of the polypeptide of SEQ ID NO: 1 (e.g., E186K, E186L, or E186R).
Paragraph [168]. The variant of any one of the previous paragraphs, wherein the variant comprises or consists of a substitution at a position corresponding to position 64 of the polypeptide of SEQ ID NO: 1 (e.g., K64I, K64Q or K64L) and position 188 of the polypeptide of SEQ ID NO: 1 (e.g., E188V or E188P).
Paragraph [169]. The variant of any one of the previous paragraphs, wherein the variant comprises or consists of a substitution at a position corresponding to position 75 of the polypeptide of SEQ ID NO: 1 (e.g., V75I or V75L) and position 186 of the polypeptide of SEQ ID NO: 1 (e.g., E186K, E186L, or E186R).
Paragraph [170]. The variant of any one of the previous paragraphs, wherein the variant comprises or consists of a substitution at a position corresponding to position 75 of the polypeptide of SEQ ID NO: 1 (e.g., V75I or V75L) and position 188 of the polypeptide of SEQ ID NO: 1 (e.g., E188V or E188P).
Paragraph [171]. The variant of any one of the previous paragraphs, wherein the variant comprises or consists of a substitution at a position corresponding to position 186 of the polypeptide of SEQ ID NO: 1 (e.g., E186K, E186L, or E186R) and/or position 188 of the polypeptide of SEQ ID NO: 1 (e.g., E188V or E188P).
Paragraph [172]. The variant of any one of the previous paragraphs, which comprises an insertion or one or more amino acids at a position corresponding to position 36 of the polypeptide of SEQ ID NO: 1 selected from Glu, Asn, Ala, Val, Trp, Phe, Lys or Asp, preferably R36RE, R36RN, R36RA, R36RV, R36RW, R36RF, R36REK, R36RKN or R36RD.
Paragraph [173]. The variant of any one of the previous paragraphs, which comprises a deletion at a position corresponding to position 222 of the polypeptide of SEQ ID NO: 1.
Paragraph [174]. The variant of any one of the previous paragraphs, which is selected from any one of the variants described in Tables 2, 3, 4, 5 or 6.
Paragraph [175]. The variant of any one the previous paragraphs, wherein the variant's parent carbonic anhydrase has at least 65%, e.g., at least 70%, at least 75%, at least 80%, at least 85%, at least 90%, at least 95% identity, at least 96%, at least 97%, at least 98%, or at least 99% sequence identity to the amino acid sequence of SEQ ID NO: 1, SEQ ID NO: 2, SEQ ID NO: 3, or SEQ ID NO: 4, or the mature polypeptide sequence thereof.
Paragraph [176]. The variant of any one the previous paragraphs, which has an improved thermostability relative to the parent when tested under the same conditions.
Paragraph [177]. The variant of any one the previous paragraphs, which has an increased protein melting point (Tm) relative to the parent when tested under the same conditions.
Paragraph [178]. A fusion polypeptide comprising the variant of any one the previous paragraphs and a second polypeptide.
Paragraph [179]. The variant or fusion polypeptide of any one the previous paragraphs, which is isolated.
Paragraph [180]. The variant or fusion polypeptide of any one the previous paragraphs, which is purified.
Paragraph [181]. A composition comprising the variant or fusion polypeptide of any one the previous paragraphs.
Paragraph [182]. The composition of paragraph [181], where the composition comprises a matrix suitable for immobilization.
Paragraph [183]. The composition of paragraph [182], where that matrix is selected from the group consisting of beads, fabrics, fibers, hollow fibers, membranes, particulates, porous surfaces, rods, and tubes.
Paragraph [184]. A polynucleotide encoding the variant or fusion polypeptide of any one the previous paragraphs.
Paragraph [185]. The polynucleotide of paragraph [184], which is isolated.
Paragraph [186]. The polynucleotide of paragraph or [185], which is purified.
Paragraph [187]. A nucleic acid construct or expression vector comprising the polynucleotide of any one of paragraphs to [186].
Paragraph [188]. A recombinant host cell transformed with the polynucleotide, nucleic acid construct, or expression vector of any one of paragraphs to [187].
Paragraph [189]. The recombinant host cell of paragraph [188], which comprises at least two copies, e.g., three, four, or five, or more copies of the polynucleotide.
Paragraph [190]. The recombinant host cell of paragraph or [189], which is a yeast recombinant host cell, e.g., a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.
Paragraph [191]. The recombinant host cell of paragraph or [189], which is a filamentous fungal recombinant host cell, e.g., an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell, in particular, an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Talaromyces emersonii, Thielavia terrestris, Trametes villosa, Trametes versicolor, Trichoderma harzianum, Trichoderma koningii, Trichoderma longibrachiatum, Trichoderma reesei, or Trichoderma viride cell.
Paragraph [192]. The recombinant host cell of paragraph or [189], which is a prokaryotic recombinant host cell, e.g., a Gram-positive cell selected from the group consisting of Bacillus, Clostridium, Enterococcus, Geobacillus, Lactobacillus, Lactococcus, Oceanobacillus, Staphylococcus, Streptococcus, or Streptomyces cells, or a Gram-negative bacteria selected from the group consisting of Campylobacter, E. coli, Flavobacterium, Fusobacterium, Helicobacter, Ilyobacter, Neisseria, Pseudomonas, Salmonella, and Ureaplasma cells, such as Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, Bacillus thuringiensis, Streptococcus equisimilis, Streptococcus pyogenes, Streptococcus uberis, and Streptococcus equi subsp. Zooepidemicus, Streptomyces achromogenes, Streptomyces avermitilis, Streptomyces coelicolor, Streptomyces griseus, and Streptomyces lividans cells.
Paragraph [193]. The recombinant host cell of any one of paragraphs to [192], which is isolated.
Paragraph [194]. The recombinant host cell of any one of paragraphs to [192], which is purified.
Paragraph [195]. A method of producing a carbonic anhydrase variant or fusion polypeptide, comprising:
   a. cultivating the host cell of any one of paragraphs to under conditions suitable for expression of the variant; and
   b. recovering the variant.
Paragraph [196]. A method for obtaining a carbonic anhydrase variant, comprising introducing into a parent carbonic anhydrase a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1; an insertion or one or more amino acids at a position corresponding to position 36 of the polypeptide of SEQ ID NO: 1; and/or a deletion at a position corresponding to position 222 of the polypeptide of SEQ ID NO: 1, wherein the variant has carbonic anhydrase activity; and recovering the variant.
Paragraph [197]. A method of extracting carbon dioxide, the method comprising treating a carbon dioxide-containing medium with a variant of any one the previous paragraphs.
Paragraph [198]. The method of paragraph [197], where carbon dioxide-containing gas or multiphase mixture is emitted from combustion.
Paragraph [199]. The method of paragraph [198], wherein the gas is a flue gas.
Paragraph [200]. The method of paragraph [198], wherein the carbon dioxide-containing gas or multiphase mixture is a raw natural gas or a syngas.
Paragraph [201]. The method of paragraph [198], wherein the carbon dioxide-containing gas or multiphase mixture is a biogas.
Paragraph [202]. The method of any one of paragraphs to [201], wherein the carbon dioxide-containing medium is a bicarbonate-containing liquid and the carbon dioxide extraction is the conversion of bicarbonate to carbon dioxide.
Paragraph [203]. The method of paragraph [202], wherein the carbon dioxide-containing medium further comprises an amine-based compound.
Paragraph [204]. The method of paragraph [203], wherein the amine-based compound is Tris or MDEA.
Paragraph [205]. The method of any one of paragraphs to [204], wherein the carbon dioxide-containing medium further comprises HPO₄²⁻, such as K2HPO₄ or Na₂HPO₄.
Paragraph [206]. The method of any one of paragraphs to [205], wherein the extraction of carbon dioxide is performed at temperatures between 55° C. and 120° C.
Paragraph [207]. A bioreactor for extracting carbon dioxide, where said bioreactor comprises a carbonic anhydrase variant or fusion protein of any one of paragraphs [1] to [180].
Paragraph [208]. The bioreactor according to paragraph [207], where said bioreactor comprises the following elements:
   a) at least one absorption module;
   b) at least one precipitation stage;
   c) a carrier liquid; and
   d) means for connecting the absorption module(s) to the precipitation stage(s) such that the carrier liquid can be passed from the absorption module(s) to the precipitation stage(s).
Paragraph [209]. The bioreactor according to paragraph or [208], where said reactor comprises the following elements:
   a) at least one absorption module;
   b) at least one desorption module;
   c) a carrier liquid; and
   d) means for connecting the absorption module(s) and the desorption module(s) such that the carrier liquid can be passed from the absorption module(s) to the desorption module(s).
Paragraph [210]. The bioreactor according to any one of paragraphs to [209], wherein the carbonic anhydrase is entrapped in the absorption module or in the desorption module or in both modules, or in the precipitation stage.
Paragraph [211]. The bioreactor according to any one of paragraphs to [210], wherein the reactor further comprises a bicarbonate-based carrier liquid.
Paragraph [212]. The bioreactor according to any one of paragraphs to [211], wherein the reactor further comprises an amine-based carrier liquid.
Paragraph [213]. The bioreactor according to paragraph [212], where the amine-based carrier liquid includes Tris or MDEA.
Paragraph [214]. The bioreactor according to any one of paragraphs to [213], wherein the reactor further comprises HPO₄²⁻, such as K₂HPO₄ or Na₂HPO₄.

1-15. (canceled)

16. A carbonic anhydrase variant, comprising
a substitution at one or more positions corresponding to positions 1, 3, 5, 6, 7, 8, 12, 17, 18, 19, 20, 21, 22, 25, 27, 28, 35, 36, 37, 38, 39, 40, 41, 43, 46, 48, 49, 50, 51, 52, 53, 54, 55, 57, 58, 59, 60, 61, 62, 64, 66, 67, 68, 71, 72, 74, 75, 76, 77, 78, 79, 80, 81, 82, 83, 84, 85, 86, 87, 88, 89, 91, 94, 95, 96, 98, 99, 100, 102, 104, 105, 106, 109, 110, 111, 112, 114, 115, 117, 118, 119, 121, 122, 123, 124, 125, 126, 127, 129, 130, 132, 133, 135, 137, 139, 140, 141, 142, 144, 145, 146, 147, 148, 149, 150, 151, 152, 153, 154, 155, 156, 161, 162, 163, 165, 166, 168, 169, 171, 173, 177, 178, 181, 182, 185, 186, 187, 188, 189, 190, 191, 192, 193, 194, 197, 198, 200, 201, 202, 203, 205, 206, 207, 208, 211, 214, 215, 216, 217, 218, 220, 221 and 222 of the polypeptide of SEQ ID NO: 1;
an insertion or one or more amino acids at a position corresponding to position 36 of the polypeptide of SEQ ID NO: 1; and/or
a deletion at a position corresponding to position 222 of the polypeptide of SEQ ID NO: 1;
wherein the variant has at least 80% sequence identity, but less than 100% sequence identity, to the polypeptide of SEQ ID NO: 1, SEQ ID NO: 2, SEQ ID NO: 3, or SEQ ID NO: 4, or the mature polypeptide sequence thereof,
wherein the variant has carbonic anhydrase activity.

17. The variant of claim 16, which comprises a substitution selected from the group consisting of:
a substitution at a position corresponding to position 1 with Met or Asp;
a substitution at a position corresponding to position 3 with Gly or Cys;
a substitution at a position corresponding to position 5 with Arg or Ser;
a substitution at a position corresponding to position 6 with Pro;
a substitution at a position corresponding to position 7 with Lys, Asp, His, Gln or Arg;
a substitution at a position corresponding to position 8 with Cys or Asn;
a substitution at a position corresponding to position 12 with Leu;
a substitution at a position corresponding to position 17 with Ser;
a substitution at a position corresponding to position 18 with Lys, Glu, Arg, Gly, Ser or Pro;
a substitution at a position corresponding to position 19 with Pro;
a substitution at a position corresponding to position 20 with Phe;
a substitution at a position corresponding to position 21 with Ala;
a substitution at a position corresponding to position 22 with Pro, Phe, Trp, Tyr or Asn;
a substitution at a position corresponding to position 25 with Thr;
a substitution at a position corresponding to position 27 with Thr or Tyr;
a substitution at a position corresponding to position 28 with Arg, Glu, Leu, Gln, Met, Ser, Tyr, Phe, Lys, Thr, Phe, Asp or His;
a substitution at a position corresponding to position 35 with Asp;
a substitution at a position corresponding to position 36 with Asp, Leu, Ser, Asn or Ile;
a substitution at a position corresponding to position 37 with Ala or Val;
a substitution at a position corresponding to position 38 with Cys;
a substitution at a position corresponding to position 39 with Ala, Cys, Glu, Phe, Gly, Lys, Leu, Met, Asn, Pro, Arg, Ser, Trp or Tyr;
a substitution at a position corresponding to position 40 with Cys;
a substitution at a position corresponding to position 41 with Cys, Ala, Met, Asn, Ser, Asp, Leu or Gly;
a substitution at a position corresponding to position 43 with Ala, His, Phe, Pro or Tyr;
a substitution at a position corresponding to position 46 with Tyr;
a substitution at a position corresponding to position 48 with Asp, Ser, Asn, Tyr, Val, Phe or Arg;
a substitution at a position corresponding to position 49 with Cys;
a substitution at a position corresponding to position 50 with Lys, Tyr or Pro;
a substitution at a position corresponding to position 51 with Pro, Ser, Thr or Cys;
a substitution at a position corresponding to position 52 with Val or Cys;
a substitution at a position corresponding to position 53 with Gly or Pro;
a substitution at a position corresponding to position 54 with Lys or Ala;
a substitution at a position corresponding to position 55 with His, Tyr or Phe;
a substitution at a position corresponding to position 57 with Val;
a substitution at a position corresponding to position 58 with Asp;
a substitution at a position corresponding to position 59 with Asp, Trp, Ile, Arg, His, Ser, Thr, Val or Leu;
a substitution at a position corresponding to position 60 with Asp;
a substitution at a position corresponding to position 61 with Tyr, Trp or Phe;
a substitution at a position corresponding to position 62 with Asn, Tyr, Trp, Phe or Met;
a substitution at a position corresponding to position 64 with Ile, Gln, or Leu;
a substitution at a position corresponding to position 66 with Leu, Ile, Phe, Met, His, Val, Gln, Tyr, Trp or Gly;
a substitution at a position corresponding to position 67 with Met, Ile, Phe, Trp or Val;
a substitution at a position corresponding to position 68 with Cys, His, Asp, Arg or Thr;
a substitution at a position corresponding to position 71 with Ala or Cys;
a substitution at a position corresponding to position 72 with Phe, Cys, Ser, Thr or Glu;
a substitution at a position corresponding to position 74 with Glu;
a substitution at a position corresponding to position 75 with Ile or Leu;
a substitution at a position corresponding to position 76 with Asn, Lys or Leu;
a substitution at a position corresponding to position 77 with Asp;
a substitution at a position corresponding to position 78 with Val, Glu or Lys;
a substitution at a position corresponding to position 79 with Ile or Val;
a substitution at a position corresponding to position 80 with Tyr;
a substitution at a position corresponding to position 81 with Tyr;
a substitution at a position corresponding to position 82 with Pro or Phe;
a substitution at a position corresponding to position 83 with Glu or Ile;
a substitution at a position corresponding to position 84 with Phe, Ala, Lys, Gly, Glu, Ile, Met, Leu, Trp or Tyr;
a substitution at a position corresponding to position 85 with Cys;
a substitution at a position corresponding to position 86 with Ile, Tyr or Trp;
a substitution at a position corresponding to position 87 with Trp or Leu;
a substitution at a position corresponding to position 88 with Tyr;
a substitution at a position corresponding to position 89 with Glu, Tyr, Ser, Thr, Cys, Val or Phe;
a substitution at a position corresponding to position 91 with His;
a substitution at a position corresponding to position 94 with Leu, Met, or Thr;
a substitution at a position corresponding to position 95 with Val, Ile, Phe, or Tyr;
a substitution at a position corresponding to position 96 with Asn or Asp;
a substitution at a position corresponding to position 98 with Lys;
a substitution at a position corresponding to position 99 with Tyr, Pro or Leu;
a substitution at a position corresponding to position 100 with Phe;
a substitution at a position corresponding to position 102 with Leu or Met;
a substitution at a position corresponding to position 104 with Leu, Cys, Met or Val;
a substitution at a position corresponding to position 105 with Phe;
a substitution at a position corresponding to position 106 with Cys;
a substitution at a position corresponding to position 109 with Gln;
a substitution at a position corresponding to position 110 with Ser;
a substitution at a position corresponding to position 111 with Glu;
a substitution at a position corresponding to position 112 with Glu, Asp, Lys, or Asn;
a substitution at a position corresponding to position 114 with Leu, Arg, Gly or Pro;
a substitution at a position corresponding to position 115 with Gln, Trp, Tyr or Ile;
a substitution at a position corresponding to position 117 with Phe;
a substitution at a position corresponding to position 118 with Val or Leu;
a substitution at a position corresponding to position 119 with Ala, Ile, Cys, Thr, Ser or Val;
a substitution at a position corresponding to position 121 with Met or Leu;
a substitution at a position corresponding to position 122 with Tyr;
a substitution at a position corresponding to position 123 with Glu;
a substitution at a position corresponding to position 124 with Cys;
a substitution at a position corresponding to position 125 with Ser, Ala, or Asp;
a substitution at a position corresponding to position 126 with Lys;
a substitution at a position corresponding to position 127 with Pro, Leu, Cys, Met, Lys, Gln, Thr, Ala, Arg or Val;
a substitution at a position corresponding to position 129 with Leu;
a substitution at a position corresponding to position 130 with Glu;
a substitution at a position corresponding to position 132 with Ile or Asp;
a substitution at a position corresponding to position 133 with Met, Ile, or Val;
a substitution at a position corresponding to position 135 with Leu;
a substitution at a position corresponding to position 137 with Lys;
a substitution at a position corresponding to position 139 with Cys;
a substitution at a position corresponding to position 140 with Ala or Asp;
a substitution at a position corresponding to position 141 with Lys, Thr, Glu or Gln;
a substitution at a position corresponding to position 142 with Lys;
a substitution at a position corresponding to position 144 with Cys;
a substitution at a position corresponding to position 145 with Tyr, Phe or Leu;
a substitution at a position corresponding to position 146 with Ala;
a substitution at a position corresponding to position 147 with Ile, Glu, or Met;
a substitution at a position corresponding to position 148 with Pro;
a substitution at a position corresponding to position 149 with Leu, Pro, His, or Asp;
a substitution at a position corresponding to position 150 with Val, Arg or Ser;
a substitution at a position corresponding to position 151 with Cys, Glu, or Pro;
a substitution at a position corresponding to position 152 with Cys or Leu;
a substitution at a position corresponding to position 153 with Asp;
a substitution at a position corresponding to position 154 with Pro, Val, Leu or Ile;
a substitution at a position corresponding to position 155 with Ala, Ile or Arg;
a substitution at a position corresponding to position 156 with Met, Asn, Gln or Leu;
a substitution at a position corresponding to position 161 with Asn;
a substitution at a position corresponding to position 162 with Phe, Met, or Leu;
a substitution at a position corresponding to position 163 with Lys, Thr, Leu, Arg, or Glu;
a substitution at a position corresponding to position 165 with Phe;
a substitution at a position corresponding to position 166 with Ala, Met, Lys, Glu, Phe, Val, Cys or Tyr;
a substitution at a position corresponding to position 168 with Asn, Cys, Ala, Asp, Glu, Leu, Pro, Gln, Thr, Val, Met, or Ile;
a substitution at a position corresponding to position 169 with Asp;
a substitution at a position corresponding to position 171 with Phe;
a substitution at a position corresponding to position 173 with Val, Trp or Ile;
a substitution at a position corresponding to position 177 with Tyr, Met, Trp or Phe;
a substitution at a position corresponding to position 178 with Ser, Pro, Thr or Gln;
a substitution at a position corresponding to position 181 with Ala, Thr, Ile, or Leu;
a substitution at a position corresponding to position 182 with Lys;
a substitution at a position corresponding to position 185 with Phe or Leu;
a substitution at a position corresponding to position 186 with Lys, Leu, Arg;
a substitution at a position corresponding to position 187 with Ile or Val;
a substitution at a position corresponding to position 188 with Val or Pro;
a substitution at a position corresponding to position 189 with Ile, Val, Leu;
a substitution at a position corresponding to position 190 with Gly or Asp;
a substitution at a position corresponding to position 191 with Val, Thr, Trp, His, Leu, Ala, Gly, Arg, Lys, Pro, Phe, Gln, Asn or Ile;
a substitution at a position corresponding to position 192 with Cys;
a substitution at a position corresponding to position 193 with Cys;
a substitution at a position corresponding to position 194 with Asp or Ala;
a substitution at a position corresponding to position 197 with Val;
a substitution at a position corresponding to position 198 with Tyr, Phe or Leu;
a substitution at a position corresponding to position 200 with Cys;
a substitution at a position corresponding to position 201 with Met or Ile;
a substitution at a position corresponding to position 202 with Leu, Val, Ala or Thr;
a substitution at a position corresponding to position 203 with Leu or Ile;
a substitution at a position corresponding to position 205 with Glu;
a substitution at a position corresponding to position 206 with Pro;
a substitution at a position corresponding to position 207 with Asn;
a substitution at a position corresponding to position 208 with Leu;
a substitution at a position corresponding to position 211 with Thr;
a substitution at a position corresponding to position 214 with Ile;
a substitution at a position corresponding to position 215 with Leu;
a substitution at a position corresponding to position 216 with His, Cys, Asn, Gly, Phe, or Pro;
a substitution at a position corresponding to position 217 with Asn;
a substitution at a position corresponding to position 218 with Lys, Val, Ile, Phe, Thr, Asn, Ser, Ala, Gly, Tyr or Leu;
a substitution at a position corresponding to position 220 with Leu;
a substitution at a position corresponding to position 221 with Arg, Leu, Ile, Phe, Tyr, Thr, Val or Lys; and
a substitution at a position corresponding to position 222 with Leu.

18. The variant of claim 16, which comprises a substitution corresponding to: G1M, G1D; S3C, S3G; H5R, H5S; G6P; E7K, E7D, E7H, E7Q, E7R; H8C, H8N; H12L; K17S; D18K, D18E, D18R, D18G, D18S, D18P; E19P; Y20F; 121A; M22P, M22F, M22W, M22Y, M22N; 25T; K27T, K27Y; N28R, N28E, N28L, N28Q, N28M, N28S, N28Y, N28F, N28K, N28T, N28D, N28F, N28H; N35D; R36D, R36L, R36S, R36N, R36I; I37A, I37V; V38C; D39A, D39C, D39E, D39F, D39G, D39K, D39L, D39M, D39N, D39P, D39R, D39S, D39W, D39Y; A40C; K41C, K41A, K41M, K41N, K41S, K41D, K41L, K41G; K43A, K43H, K43F, K43P, K43Y; K46Y; E48D, E48S, E48N, E48F, E48V, E48Y, E48R; Y49C; R50K, R50Y, R50P; A51P, A51S, A51T, A51C; G52V, G52C; A53G, A53P; T54K, T54A; K55H, K55Y, K55F; L57V; N58D; N59D, N59S, N59R, N59H, N59I, N59T, N59V, N59W, N59L; G60D; H61Y, H61W, H61F; T62N, T62Y, T62F, T62W, T62M; K64I, K64Q, K64L; S66L, S66I, S66F, S66M, S66H, S66V, S66Q, S66Y, S66W, S66G; Y67M, Y67I, Y67F, Y67W, Y67V; E68C, E68H, E68R, E68D, E68T; S71A, S71C; Y72F, Y72C, Y72S, Y72T, Y72E; V74E; V75I, V75L; D76K, D76N, D76L; G77D; 178V, 178E, 178K; K791, K79V; F80Y; E81Y; L82P, L82F; K83E, K83I; Q84F, Q84A, Q84K, Q84G, Q84E, Q84M, Q84I, Q84L, Q84W, Q84Y; F85C; H86I, H86Y, H86W; F87W, F87L; H88Y; A89E, A89Y, A89S, A89T, A89C, A89V, A89F; S91H; K94L, K94M, K94T; L95V, L95I, L95F, L95Y; K96N, K96D; Q98K; H99Y, H99P, H99L; Y100F; F102L, F102M; A104L, A104M, A104C, A104V; H105F; F106C; A109Q; D110S; K111E; H112E, H112D, H112K, H112N; N114L, N114R, N114G, N114P; L115W, L115Y, L115I, L115Q; V117F; I118V, I118L; G119A, G199C, G119T, G119S, G119I, G119V; F121M, F121L; F122Y; K123E; E124C; G125S, G125A, G125D; R126K; E127P, E127L, E127C, E127M, E127K, E127Q, E127T, E127A, E127R, E127V; P129L; 1130E; E132I, E132D; K133M, K133I, K133V; W135L; V137K; P139C; E140A, E140D; N141K, N141E, N141T, N141Q; A142K; E144C; E145L, E145F, E145Y; V146A; K147I, K147E, K147M; L148P; A149L, A149P, A149H, A149D; H150V, H150R, H150S; K151C, K151E, K151P; 1152C, 1152L; N153D; A154P, A154V, A154L, A154I; E155A, E155I, E155R; D156M, D156Q, D156N, D156L; D161N; R162F, R162M, R162L; D163K, D163T, D163L, D163R, D163E; Y165F; R166A, R166M, R166K, R166E, R166F, R166V, R166C, R166Y; S168N, S168C, S168A, S168D, S168E, S168L, S168K, S168P, S168Q, S168T, S168V, S168M, S168I; G169D; L171F; T173V, T173W, T1731; S177Y, S177M, S177W, S177F; E178S, E178P, E178T, E178Q; R181A, R181T, R181I, R181L; W182K; M185F, M185L; E186K, E186L, E186R; E187I, E187V; E188V, E188P; M189I, M189V, M189L; E190G, E190D; M191V, M191A, M191H, M191G, M191L, M191K, M191P, M191N, M191R, M191T, M191F, M191Q, M191W, M1911; S192C; K193C; E194D, E194A; E197V; K198Y, K198F, K198L; R200C; K201M, K201I; I202L, I202V, I202A, I202T; M203L, M203I; G205E; D206P; T207N; N208L; V211T; L214I; N215L; A216H, A216F, A216G, A216N, A216C, A216P; R217N; M218K, M218V, M218I, M218N, M218S, M218F, M218A, M218G, M218T, M218Y, M218L; M220L; E221R, E221L, E221I, E221Y, E221F, E221T, E221V, E221K; or K222L.

19. The variant of claim 16, wherein the variant comprises or consists of a substitution at position 18, 39, 64, 75, 186 and/or 188, corresponding to the polypeptide of SEQ ID NO: 1.

20. The variant of claim 16, wherein the variant comprises or consists of a substitution at D18P, D39L, K64L, V75I, E186K and/or E188P corresponding to the polypeptide of SEQ ID NO: 1.

21. The variant of claim 16, which comprises an insertion or one or more amino acids at a position corresponding to position 36 of the polypeptide of SEQ ID NO: 1 selected from Glu, Asn, Ala, Val, Trp, Phe, Lys or Asp, preferably R36RE, R36RN, R36RA, R36RV, R36RW, R36RF, R36REK, R36RKN or R36RD.

22. The variant of claim 16, which comprises an insertion or one or more amino acids at a position corresponding to position 36 of the polypeptide of SEQ ID NO: 1 corresponding to R36RE, R36RN, R36RA, R36RV, R36RW, R36RF, R36REK, R36RKN or R36RD.

23. The variant of claim 16, which comprises a deletion at a position corresponding to position 222 of the polypeptide of SEQ ID NO: 1.

24. The variant of claim 16, wherein the variant has an improved thermostability relative to the parent when tested under the same conditions.

25. The variant of claim 17, wherein the variant has an increased protein melting point (Tm) relative to the parent when tested under the same conditions.

26. A composition comprising the variant of claim 16.

27. A method of extracting carbon dioxide, the method comprising treating a carbon dioxide-containing medium with the variant of claim 16.

28. A bioreactor for extracting carbon dioxide, where said bioreactor comprises the variant of claim 16.

29. A polynucleotide encoding the variant of claim 16.

30. A nucleic acid construct or expression vector comprising the polynucleotide of claim 29.

31. A recombinant host cell transformed with the polynucleotide of claim 29.

32. A method of producing a carbonic anhydrase variant, comprising:
a. cultivating the recombinant host cell of claim 31 under conditions suitable for expression of the variant; and
b. recovering the variant.
